(19) United States
(12) Patent Application Publication
(10) Pub. No.: US 2024/0408174 A1
(43) Pub. Date: Dec. 12, 2024

(54) SILK STIMULATED COLLAGEN AND CLAUDIN-1 EXPRESSION, AND SILK STIMULATED ANTI-INFLAMMATORY EFFECTS

(71) Applicant: EVOLVED BY NATURE, INC., Medford, MA (US)

(72) Inventors: Gregory H. Altman, PROVIDENCE, RI (US); MARIUS COSTACHE, LEXINGTON, MA (US); EVA GENEL, MEDFORD, MA (US); CARLOS J. BOSQUES, ARLINGTON, MA (US); JUTAMAS SUWANPRADID, MEDFORD, MA (US); AVINASH JAIGANESH, MEDFORD, MA (US); LAURA R. MUOLLO, DRACUT, MA (US)

(21) Appl. No.: 18/702,304

(22) PCT Filed: Oct. 18, 2022
(86) PCT No.: PCT/US2022/078314

§ 371 Date: Apr. 17, 2024

Related U.S. Application Data
(60) Provisional application No. 63/256,942, filed on Oct. 18, 2021.
(60) Provisional application No. 63/256,896, filed on Oct. 18, 2021.

(51) Int. Cl. A61K 38/17; A61K 8/19; A61K 8/64; A61K 8/98; A61K 47/02; A61P 17/02; A61Q 19/08

(52) U.S. Cl. CPC A61K 38/1767; A61K 8/19; A61K 8/64; A61K 8/987; A61K 47/02; A61P 17/02; A61Q 19/08

(57) ABSTRACT

The disclosure provides silk fibroin compositions for stimulating collagen expression, claudin-1, and-or an anti-inflammatory effect in a subject, and methods of use thereof.

### CROSS-REFERENCE TO RELATED APPLICATION

This application claims the benefit of U.S. Provisional Patent Application No. 63/256,942, filed Oct. 18, 2021, and U.S. Provisional Patent Application No. 63/256,896, filed Oct. 18, 2021, both of which are incorporated by reference herein in their entireties.

### FIELD

This disclosure is in the field of silk fibroin compositions and methods for stimulating collagen expression.

### BACKGROUND

Silk is a natural polymer produced by a variety of insects and spiders. Silk comprises a filament core protein, silk fibroin, and a glue-like coating consisting of a nonfilamentous protein, sericin.

There exists a need for stable silk fibroin peptide solution suitable for collagen stimulation by topical or parenteral administration.

### SUMMARY

The disclosure provides a method of treatment or prevention of a disorder, disease, or condition alleviated by stimulating or modulating collagen expression in a subject in need thereof, the method comprising administering to the subject a composition comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 14 kDa and about 30 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5. In some embodiments, the composition further comprises 0 to 500 ppm lithium bromide. In some embodiments, the composition further comprises 0 to 500 ppm sodium carbonate. In some embodiments, the silk fibroin fragments have a polydispersity between 1 and about 1.5. In some embodiments, the silk fibroin fragments have a polydispersity between about 1.5 and about 2.0. In some embodiments, the silk fibroin fragments have a polydispersity between about 1.5 and about 3.0. In some embodiments, the silk fibroin fragments have a polydispersity between about 2.0 and about 2.5. In some embodiments, the silk fibroin fragments have a polydispersity between about 2.5 and about 3.0. In some embodiments, the silk fibroin fragments are present in the composition at about 0.001 wt. % to about 10.0 wt. % relative to the total weight of the composition. In some embodiments, the composition further comprises about 0.001% (w/w) to about 10% (w/w) sericin relative to the total weight of the composition. In some embodiments, the composition further comprises about 0.001% (w/w) to about 10% (w/w) sericin relative to the silk fibroin fragments. In some embodiments, the silk fibroin fragments do not spontaneously or gradually gelate and do not visibly change in color or turbidity when in an aqueous solution for at least 10 days prior to formulation into the composition. In some embodiments, the silk fibroin fragments are present in the composition at about 0.01 wt. % to about 10.0 wt. % relative to the total weight of the composition. In some embodiments, the silk fibroin fragments are present in the composition at about 0.01 wt. % to about 1.0 wt. % relative to the total weight of the composition. In some embodiments, the silk fibroin fragments are present in the composition at about 1.0 wt. % to about 2.0 wt. % relative to the total weight of the composition. In some embodiments, the silk fibroin fragments are present in the composition at about 2.0 wt. % to about 3.0 wt. % relative to the total weight of the composition. In some embodiments, the silk fibroin fragments are present in the composition at about 3.0 wt. % to about 4.0 wt. % relative to the total weight of the composition. In some embodiments, the silk fibroin fragments are present in the composition at about 4.0 wt. % to about 5.0 wt. % relative to the total weight of the composition. In some embodiments, the silk fibroin fragments are present in the composition at about 5.0 wt. % to about 6.0 wt. % relative to the total weight of the composition. In some embodiments, the composition is formulated as an injectable composition or as a topical composition. In some embodiments, the composition further comprises a pharmaceutically acceptable carrier. In some embodiments, the composition further comprises a dermatologically acceptable carrier. In some embodiments, the composition further comprises an injectable acceptable carrier. In some embodiments, the pharmaceutically acceptable carrier comprises one or more of a suspension, an emulsion, a powder, a solution, a dispersion, or an elixir. In some embodiments, the pharmaceutically acceptable carrier comprises or is formulated as one or more of a gel, a jelly, a cream, a lotion, a foam, a slurry, an ointment, an oil, a paste, a suppository, a spray, a semisolid composition, a solid composition, a stick, or a mousse. In some embodiments, the pharmaceutically acceptable carrier comprises one or more of sesame oil, corn oil, cottonseed oil, or peanut oil. In some embodiments, the pharmaceutically acceptable carrier comprises one or more of mannitol or dextrose. In some embodiments, the pharmaceutically acceptable carrier comprises about 0.001% to about 10% (w/v) hyaluronic acid. In some embodiments, the pharmaceutically acceptable carrier comprises about 1% to about 10% (w/v), about 10% to about 25% (w/v), about 25% to about 50% (w/v), or about 50% to about 99.99% (w/v) hyaluronic acid.

In some embodiments, the pharmaceutically acceptable carrier comprises one or more of aliphatic oil, a fatty alcohol, a fatty acid, a glyceride, an acylglycerol, and a phospholipid. In some embodiments, the pharmaceutically acceptable carrier comprises one or more of a monoglyceride, a diglyceride, or a triglyceride. In some embodiments, the pharmaceutically acceptable carrier comprises an aqueous phase. In some embodiments, the pharmaceutically acceptable carrier comprises an oil-in-water emulsion or a water-in-oil emulsion. In some embodiments, the pharmaceutically acceptable carrier comprises one or more of a hydrocarbon oil, a fatty acid, a fatty oil, a fatty acid ester, or a cationic quaternary ammonium salt. In some embodiments, a portion of the pharmaceutically acceptable carrier is modified with a cross-linking agent, a cross-linking precursor, or an activating agent selected from a polyepoxy linker, a diepoxy linker, a polyepoxy-PEG, a diepoxy-PEG, a polyglycidyl-PEG, a diglycidyl-PEG, a poly acrylate PEG, a diacrylate PEG, 1,4-bis(2,3-epoxypropoxy)butane, 1,4-bisglycidyloxybutane, divinyl sulfone (DVS), 1,4-butanediol diglycidyl ether (BDDE), UV light, glutaraldehyde, 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), 1-(2,3-epoxypropyl)-2,3-epoxycyclohexane, a carbodiimide, and any combinations thereof. In some embodiments, the polyepoxy linker is selected from 1,4-butanediol diglycidyl ether (BDDE), ethylene glycol diglycidyl ether (EGDGE), 1,6-hexanediol diglycidyl ether, polyethylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylolpropane polyglycidyl ether, pentaerythritol polyglycidyl ether, and sorbitol polyglycidyl ether. In some embodiments, the composition is administered parenterally. In some embodiments, the composition is an injectable composition. In some embodiments, the composition is administered by injection. In some embodiments, the composition is administered by subcutaneous injection, intradermal injection, transdermal injection, or subdermal injection. In some embodiments, the composition is administered by intramuscular injection, intravenous injection, intraperitoneal injection, intraosseous injection, intracardiac injection, intraarticular injection, or intracavernous injection. In some embodiments, the composition is administered by depot injection. In some embodiments, the composition is administered by infiltration injection. In some embodiments, the composition is administered by an indwelling catheter. In some embodiments, the composition is administered by microneedling.

In some embodiments, administering the composition decreases expression of one or more metalloproteinases (MMP) in the subject. In some embodiments, stimulating or modulating collagen expression comprises increasing collagen expression. In some embodiments, collagen expression is increased over a base level by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In some embodiments, collagen expression is increased over a base level by about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, about 150%, about 151%, about 152%, about 153%, about 154%, about 155%, about 156%, about 157%, about 158%, about 159%, about 160%, about 161%, about 162%, about 163%, about 164%, about 165%, about 166%, about 167%, about 168%, about 169%, about 170%, about 171%, about 172%, about 173%, about 174%, about 175%, about 176%, about 177%, about 178%, about 179%, about 180%, about 181%, about 182%, about 183%, about 184%, about 185%, about 186%, about 187%, about 188%, about 189%, about 190%, about 191%, about 192%, about 193%, about 194%, about 195%, about 196%, about 197%, about 198%, about 199%, or about 200%.

In some embodiments, administering the composition results in one or more of preventing or reversing wrinkles in the subject, preventing or reversing age spots in the subject, preventing or reversing dry skin in the subject, or increasing uneven skin tone in the subject. In some embodiments, administering the composition results in one or more of preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject. In some embodiments, the disorder, disease, or condition comprises wrinkles, age spots, dry skin, uneven skin tone, skin sagging, skin aging, reduced skin tensile strength, photodamaged skin, or striae distensae (stretch marks). In some embodiments, the disorder, disease, or condition comprises thyroid hormone-induced myocardial hypertrophy. In some embodiments, the disorder, disease, or condition comprises a tendon rupture, damage, or tear. In some embodiments, the tendon is selected from Teres minor tendons, Infraspinatus tendons, Supraspinatus tendons, Subscapularis tendons, Deltoid tendons, Biceps tendons, Triceps tendons, Brachioradialis tendons, Supinator tendons, Flexor carpi radialis tendons, Flexor carpi ulnaris tendons, Extensor carpi radialis tendons, Extensor carpi radialis brevis tendons, Iliopsoas tendons, Obturator internus tendons, Adductor longus, brevis or magnus tendons, Gluteus maximus or gluteus medius tendons, Quadriceps tendons, patellar tendon, Hamstring tendons, Sartorius tendons, Gastrocnemius tendons, Achilles tendon, Soleus tendons, Tibialis anterior tendons, Peroneus longus tendons, Flexor digitorum longus tendons, Interosseus tendons, Flexor digitorum profundus tendons, Abductor digiti minimi tendons, Opponens pollicis tendons, Flexor pollicis longus tendons, Extensor or abductor pollicis tendons, Flexor hallucis longus tendons, Flexor digitorum brevis tendons, Lumbrical tendons, Abductor hallucis tendons, Flexor digitorum longus tendons, Abductor digiti minimi tendons, Ocular tendons, Levator palpebrae tendons, Masseter tendons, Temporalis tendons, Trapezius tendons, Sternocleidomastoid tendons, Semispinalis capitis or splenius capitis tendons, Mylohyoid or thyrohyoid tendons, Sternohyoid tendons, Rectus abdominis tendons, External oblique tendons, Transversus abdominis tendons, Latissimus dorsi tendons, and Erector spinae tendons. In some embodiments, the disorder, disease, or condition comprises Werner's syndrome. In some embodiments, the disorder, disease, or condition comprises diminished diabetic skin integrity. In some embodiments, the disorder, disease, or condition comprises arthritis. In some embodiments, the disorder, disease, or condition comprises rheumatoid arthritis. In some embodiments, the disorder, disease, or condition comprises tumor progression or tumor growth. In some embodiments, the disorder, disease, or condition comprises diminished cardiac function. In some embodiments, the disorder, disease, or condition comprises Ehlers-Danlos syndrome. In some embodiments, the disorder, disease, or condition comprises abdominal aortic aneurysms. In some embodiments, the disorder, disease, or condition comprises a wound. In some embodiments, the disorder, disease, or condition comprises a skin or connective tissue disease. In some embodiments, the disorder, disease, or condition comprises a cartilage disease. In some embodiments, the disorder, disease, or condition is selected from relapsing polychondritis, Tietze's Syndrome, cellulitis, Ehler's Danlos syndrome, keloids (including acne keloids), mucopolysaddaridosis I, necrobiotic disorders (including granuloma annulare, necrobiosis lipoidica), osteogenesis imperfect, cutis laxa, dermatomyositis, Dupytren's contracture, homocystinuria, lupus erythematosis (including cutaneous, discoid, panniculitis, systemic and nephritis), marfan syndrome, mixed connective tissue disease, mucinosis (including follicular), mucopolysaccaridoses (I, II, UU, IV, IV, and VII), myxedema, scleredemo adultorum and synovial cysts, connective tissue neoplasms, Noonan syndrome, osteopoikilosis, panniculitis, including erythema induratum, nodular nonsuppurative and peritoneal, penile induration, pseudoxanthoma elasticum, rheumatic diseases, including arthritis (rheumatoid, juvenile rheumatoid, Caplan's syndrome, Felty's syndrome, rheumatoid nodule, ankylosing spondylitis, and still's disease), hyperostosis, polymyalgia rheumatics, circumscribed scleroderma, and systemic scleroderma (CREST syndrome). In some embodiments, the disorder, disease, or condition is selected from angiolymphoid hyperplasia with eosinophilia; cicatix (including hypertophic); cutaneous fistula, cuis laxa; dermatitis, including acrodermatitis, atopic dermatitis, contact dermatitis (allergic contact, photoallergic, toxicodendron), irritant dermatitis (phototoxic, diaper rash), occupational dermatitis; exfoliative dermatitis, herpetiformis dermatitis, seborrheic dermatitis, drug eruptions (such as toxic epidermal necrolysis, erythema nodosum, serum sickness) eczema, including dyshidrotic, intertrigo, neurodermatitis, and radiodermatitis; dermatomyositis; erythema, including chronicum migrans, induratum, infectiosum, multiforme (Stevens-Johnson syndrome), and nodosum (Sweet's syndrome); exanthema, including subitum; facial dermatosis, including acneiform eruptions (keloid, rosacea, vulgaris and Favre-Racouchot syndrome); foot dermatosis, including tinea pedis; hand dermatoses; keratoacanthoma; keratosis, including callosities, cholesteatoma (including middle ear), ichthyosis (including congenital ichtyosiform erythroderms, epidermolytic hyperkeratosis, lamellar ichthyosis, ichthyosis vulgaris, X-linked ichthyosis, and Sjogren-Larsson syndrome), keratoderma blennorrhagicum, palmoplantar keratoderms, follicularis keratosis, seborrheic keratosis, parakeratosis and porokeratosis; leg dermatosis, mastocytosis (urticaria pigmentosa), necrobiotic disorders (granuloma annulare and necrobiosis lipoidica), photosensitivity disorders (photoallergic or photoxic dermatitis, hydroa vacciniforme, sundurn, and xeroderma pigmentosum); pigmentation disorders, including argyria, hyperpigmentation, melanosis, aconthosis nigricans, lentigo, Peutz-Jeghers syndrome, hypopigmentation, albinism, pibaldism, vitiligo, incontinentia pigmenti, urticaria pigmentosa, xeroderma pigmentosum, prurigo; pruritis (including ani and vulvae); pyoderma, including ecthyma and pyoderma gangrenosum; sclap dermatoses; sclerodema adultorum; sclerma neonatorum; skin appenage diseases, including hair diseases (alopecia, folliculitis, hirsutism, hypertichosis, Kinky hair syndrome), nail diseases (nail-patella syndrome, ingrown or malformed nails, onychomycosis, paronychia), sebaceous gland diseases (rhinophyma, neoplasms), sweat gland diseases (hidradenitis, hyperhidrosis, hypohidrosis, miliara, Fox-Fordyce disease, neoplasms); genetic skin diseases, including alfinism, cutis laxa, benign familial pemphigis, porphyria, acrodermatitis, ectodermal dysplasia, Ellis-Van Creveld syndrome, focal dermal hypoplasia, Ehlers-Danlos syndrome, epidermolysis bullosa, ichtysosis; infectious skin diseases, including dermatomycoses, blastomycosis, candidiasis, chromoblastomycosis, maduromycosis, paracoccidioidomycosis, sporotrichosis, tinea; bacterial skin diseases, such as cervicofacial actinomycosis, bacilliary angiomatosis, ecthyma, erysipelas, erythema chronicum migrans, erythrasma, granuloma inguinale, hidradenitis suppurativa, maduromycosis, paronychia, pinta, rhinoscleroma, staphylococcal skin infections (furuncolosis, carbuncle, impetigo, scalded skin syndrome), cutaneous syphilis, cutaneous tuberculosis, yaws; parasitic skin diseases, including larva migrans, Leishmaniasis, pediculosis, and scabies; viral skin diseases, including erythema infectiosum, exanthema subitum, herpes simplex, moolusum contagiosum, and warts.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIGS. 1A-1C: illustrate a schematic representation of collagen synthesis in youthful and aging skin and proposed role for silk fibroin in stimulating collagen synthesis. FIG. 1A: In healthy young skin, dermal fibroblasts in a dense collagen matrix continually reinforce the matrix by producing new collagen. In young skin, intact collagen within the dermal extracellular matrix (ECM) provides attachment sites and mechanical resistance for fibroblasts. Fibroblasts are able to stretch and produce new collagen (green), promoting ECM integrity and stability. FIG. 1B: With age, production of new collagen by fibroblasts decreases and the collagen matrix degrades. With aging, reductions in collagen synthesis and increases in MMP activity result in fragmented collagen fibrils. This leads to a loss of mechanical tension for fibroblasts and a loss of ECM integrity and stability. FIG. 1C: The addition of silk fibroin to the matrix stimulates collagen production by fibroblasts, restoring the structural integrity of the matrix. Added silk fibroin stimulates fibroblasts to produce collagen, possibly by direct interaction with fibroblasts as well as cross-linking of collagen fragments. This is predicted to promote the restoration of ECM integrity and a more youthful skin appearance. (Adapted from Varani et al. Am J Pathol. 2006, 168:1861).

FIG. 2: illustrates that collagen production is dependent on the silk composition. Intracellular collagen production at various silk concentrations is shown as a function of silk type. Percent stimulation is the increase in collagen formation compared to the negative control. Silk average MW compositions: silk A=low MW (average weight average molecular weight selected from between about 14 kDa and about 30 kDa); silk B=mid MW (average weight average molecular weight selected from between about 39 kDa and about 54 kDa).

FIG. 3: In vitro model: extracellular matrix generation. The timeline indicates experimental chronology and treatment conditions.

FIG. 4: In vitro model: collagen production. The positive control treatment was TGF-β (10 ng/mL+Vit C (20 μg/mL).

FIG. 5A-5B: Treating human dermal fibroblasts with silk in the presence of Vit C increases total collagen production. FIG. 5A. is Sirius red staining of human dermal fibroblasts with Vit C and co-treatment with TGF-b (served as positive control), media control, retinoic acid, Mid MW silk, or Low MW silk for 5 days. Scale bars represent 650 m. FIG. 5B is spectrophotometric analysis of Sirius red-stained cells in FIG. 5A; n=1.

FIG. 6: Mid and Low MW silks enhance total collagen production. Spectrophotometric analysis of Sirius red on human dermal fibroblasts 24 hr post-stimulation (n=2).

FIG. 7: Mid and Low MW silks upregulated COL1A1 gene expression in human dermal fibroblasts. Quantitative PCR on COL1A1 in silk- and retinoic acid-treated human dermal fibroblasts at 8 h after treatment; n=2 per group.>8-fold increase in TGF-b+Vit. C treated human dermal fibroblasts (served as positive control).

FIGS. 8A-8B: Low MW silk upregulates collagen 1 protein expression. FIG. 8A is a histogram representative flow cytometry analysis for collagen 1 of retinoic acid-treated, vehicle-treated, and Low MW silk-treated human dermal fibroblasts gated on live cells. The data shown are representative of n=3 per group. FIG. 8B shows percent increase in the collagen 1 mean fluorescent intensity (MFI) in the retinoic acid-treated and Low MW silk-treated cells compared to the vehicle controls. Data are summarized as mean±SEM, *p<0.05 with one-way ANOVA followed by the post hoc t-test with Bonferroni correction; n=3 per group: 34.5% increase in collagen 1 MFI in TGF-β+Vit C-treated human dermal fibroblasts (served as a positive control). Representative immunohistochemistry depicting collagen 1 (green staining) and Hoechst (blue) co-localization in retinoic acid-treated and Low MW silk-treated human dermal fibroblasts (n=1).

FIG. 9: Low MW silk does not alter COL4A1 protein expression. Quantitative analysis of COL4Al⁺ cell frequency (in living cells) in retinoic acid-treated, vehicle-treated, and Low MW silk-treated human dermal fibroblasts; n=2 per group.

FIG. 10: Activated Silk™ molecules exhibit similar stimulation of Collagen 1 in human dermal fibroblasts as retinoic acid.

FIG. 11: Activated Silk's™ 33B upregulates COL1A1 gene expression in human dermal fibroblasts. Quantitative PCR on COL1A1 in silk and retinoic acid-treated human dermal fibroblasts at 8 h after treatment; n=2 per group.

FIG. 12: A flow chart showing various embodiments for producing silk fibroin protein fragments (SPFs) of the present disclosure.

FIG. 13: A flow chart showing various parameters that can be modified during the process of producing a silk protein fragment solution of the present disclosure during the extraction and the dissolution steps.

FIGS. 14A-14B: illustrate the cross sections of EFT-400 tissues exposed to low MW Silk (RITC labeled) for 2×5 hrs counterstained with DAPI. 5× magnification image (FIG. 14A) shows full tissue thickness and 10× magnification image (FIG. 14B) focuses on epidermis.

FIGS. 15A-15B: illustrate the cross sections of EFT-400 tissues exposed to mid MW Silk (FITC labeled) for 2×5 hrs counterstained with DAPI. 5× magnification image (FIG. 15A) shows full tissue thickness and 10× magnification image (FIG. 15B) focuses on epidermis.

FIG. 16: is a microscopic cross-section of silk fibroin described herein (Activated Silk™)—treated EpiDermFT tissue; fluorescence imaging of fluorescently tagged silk fibroin.

FIGS. 17A-17N: illustrates that silk fibroin described herein restores claudin-1 expression in damaged human skin (N=1, 52-year-old Caucasian woman).

FIG. 18: illustrate that silk fibroin described herein restores claudin-1 expression in damaged human skin.

FIG. 19: illustrates how silk fibroin described herein restores claudin-1 expression to improve skin barrier.

FIGS. 20A-21H: illustrates how Mid Skid silk increases Claudin-1 protein expression in human neonatal epidermal keratinocytes in vitro. (20A-20H) Representative immunohistochemistry images of keratinocyte cells (˜80-90% confluence) treated (20A, 20G) without, or (20B-20F, 20H) with mid skid (33B) silk polypeptides (0.5 mg/mL-6 mg/mL=0.05-0.6% w/v) for 24 hrs. Claudin-1 expression (red) increases with addition of MID SKID silk. Panels G and H were treated with normal rabbit IgG isotype control antibody to indicate non-specific binding of target primary antibody.

FIGS. 21A-21D: illustrates how Low Skid silk increases Claudin-1 protein expression in human neonatal epidermal keratinocytes in vitro. Representative immunohistochemistry images of keratinocyte cells (˜80-90% confluence) treated (21A) without, or (21B-21D) with low skid (27p) silk polypeptides (0.5 mg/mL-7 mg/mL=0.05-0.7% w/v) for 24 hrs. Claudin-1 expression (red) increases with addition of LOW SKID silk.

FIGS. 22A-22B: illustrates the results for the experiment for the detection of claudin-1 upregulation in skin biopsies. Puncture skin biopsies were acquired from human donors with an age range of 30-60 years. 22A, Skin biopsies were pre-treated with acetone as described in the materials and methods and then silk or other reagents were added on them as indicated in the diagram. 22B, when skin biopsies were treated first with acetone and then with vehicle, claudin-1 (orange stain) was abolished and not regenerated. However, when after the acetone treatment mid skin (33B) silk was applied claudin-1 expression was restored.

FIG. 23: illustrates the results of the experiment for Low (27P) and Mid (33B) skid silk restore claudin-1 expression in human skin. Puncture skin biopsies were acquired from human donors with an age range of 30-52 years. Skin biopsies were treated with acetone as described in the materials and methods and then silk or other reagents were added on them as indicated in the diagram. Sections of the skin were stained for claudin-1 (orange stain) and cell nuclei (blue stain). Skin biopsies treated with Low (27P) and Mid (33B) silk polypeptides upregulated claudin-1 expression after treatment with acetone. (Representative experiments are shown in this figure). (2 mg/mL=0.2%, 3 mg/mL=0.3%, 4 mg/mL=0.4%).

FIGS. 24A-24D: Quantification of claudin-1 upregulation. A, B Claudin-1 (red intensity) to DAPI (number of cells) ratio within each experiment was averaged to represent Claudin-1 expression per cell. Data was normalized to no treatment samples and error bars represent standard deviation of normalized data. C, D Analysis depicting total area of claudin-1 in human skin samples. Data are expressed as percentage SEM, *p<0.05 (see materials and methods for more details). (2 mg/mL=0.2%, 3 mg/mL=0.3%, 4 mg/mL=0.4%, 5 mg/mL=0.5%, 6 mg/mL=0.6%, 7 mg/mL=0.7%, 60 mg/mL=6%).

FIG. 25: Low skid (27P) silk upregulates collagen expression in dermal skin fibroblast. Human dermal fibroblasts were treated with various concentrations of low (27P) skid silk polypeptides and expression of collagen was visualized. Collagen expression was upregulated at 2 mg/mL low (27P) skid silk polypeptides (0.2%).

FIG. 26: Low skid (27P) silk upregulates collagen expression in dermal skin fibroblast at 2 mg/mL (0.2% w/v). Human dermal fibroblasts were treated with various concentrations of low (27P) skid silk polypeptides and expression of collagen was visualized and quantified. Collagen expression was significantly upregulated at 2 mg/mL low (27P) skid silk polypeptides. (0.25 mg/mL=0.025%, 0.5 mg/mL=0.05%, 2 mg/mL=0.2%, 7 mg/mL=0.7%).

FIG. 27: Low skid (27P) silk accelerates cell migration in a wound closure assay. Human primary keratinocytes were grown in medium without serum and growth factors (see materials & methods for more details) (negative control). After they formed a layer, a scratch was created that disrupted the continuity of the layer. Cells were allowed to migrate to the generated “wound” (gap) and the rate of filling it was measured. Keratinocytes treated with medium without serum and growth factors refilled about 20% of the total generated gap (“wound closure”). When keratinocytes were treated with medium that contained serum and growth factors wound closure was almost complete (positive control). Keratinocytes treated with medium and 0.5 mg/mL (0.05%) Low Skid (27P) silk polypeptides, wound closure was also almost complete.

FIG. 28: CD44 interaction with silk polypeptides. Solid phase protein-protein interaction assay results. Low skid silk (27P) and mid skid silk (33B) was immobilized on a high binding 96 well plates. Human CD44-hFc protein bound on both the low (27P) and mid (33B) silk polypeptides compositions (compare lanes 3 with 4 and 7 with 6). Mid skid (33B) silk had higher nonspecific binding on the secondary antibody (compare lanes 6 with 2) but when CD44-hFc was added then the resulting signal was much higher (compare lane 7 with 6). Absorbances are averages of three technical repeats. Independ experiments demonstrated similar results (not shown).

FIG. 29: Is a graph illustrating a summary of expert evaluation of fine lines and wrinkles.

FIG. 30: Is a graph illustrating a summary of expert evaluation of skin firmness.

FIG. 31: Is a graph illustrating a summary of expert evaluation of redness.

FIG. 32: Is a graph illustrating a summary of TEWL data (measured by Tewameter®).

FIG. 33: Is a graph illustrating a summary of NumWr Data (measured by silicone profilometry).

FIG. 34: Is a graph illustrating a summary of Shadows Data (measured by silicone profilometry).

FIG. 35: Is a graph illustrating top box responses from the self-perception questionnaire for the 33B study.

### DETAILED DESCRIPTION

Methods of making silk fibroin or silk fibroin fragments are known and are described for example in U.S. Pat. Nos. 9,187,538, 9,511,012, 9,517,191, 9,522,107, 9,522,108, 9,545,369, and 10,166,177. Methods of using silk fibroin or silk fibroin fragments in coating applications, including coating applications of animal hair, are known and are described for example in U.S. Patent Application Publications Nos. 20160222579, and 20160281294. Compositions and methods of using silk fibroin or silk fibroin fragments in cosmetic applications are known and are described for example in U.S. Patent Application Publications Nos. 20180280274 and 20180008522, and International Patent Application Publication No. WO 2019005848. All of the publications cited herein are incorporated by reference herein in their entireties.

### Definitions

As used in the preceding sections and throughout the rest of this specification, unless defined otherwise, all technical and scientific terms used herein have the same meaning as is commonly understood by one skilled in the art to which this invention belongs. All patents and publications referred to herein are incorporated by reference in their entireties.

All percentages, parts and ratios are based upon the total weight of the collagen boosting compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or % w/w herein.

As used herein, the term “a”, “an”, or “the” generally is construed to cover both the singular and the plural forms.

As used herein, the term “about” generally refers to a particular numeric value that is within an acceptable error range as determined by one of ordinary skill in the art, which will depend in part on how the numeric value is measured or determined, i.e., the limitations of the measurement system. For example, “about” can mean a range of 20%, ±10%, or ±5% of a given numeric value.

As used herein, the term “dermatologically acceptable carrier” means a carrier suitable for use in contact with mammalian keratinous tissue without causing any adverse effects such as undue toxicity, incompatibility, instability, allergic response, for example. A dermatologically acceptable carrier may include, without limitations, water, liquid or solid emollients, humectants, solvents, and the like.

As used herein, the term “hydrophilic-lipophilic balance” (HLB) of a surfactant is a measure of the degree to which it is hydrophilic or hydrophobic, as determined by calculating values for the different regions of the molecule, as described by Griffin's method HLB=20* Mₕ/M, where Mₕ is the molecular mass of the hydrophilic portion of the surfactant, and M is the molecular mass of the entire surfactant molecule, giving a result on a scale of 0 to 20. A HLB value of 0 corresponds to a completely lipophilic molecule, and a value of 20 corresponds to a completely hydrophilic molecule. The HLB value can be used to predict the surfactant properties of a molecule: HLB<10: Lipid-soluble (water-insoluble), HLB>10: Water-soluble (lipid-insoluble), HLB=1-3: anti-foaming agent, 3-6: W/O (water-in-oil) emulsifier, 7-9: wetting and spreading agent, 8-16: O/W (oil-in-water) emulsifier, 13-16: detergent, 16-18: solubilizer or hydrotrope.

As used herein, “average weight average molecular weight” refers to an average of two or more values of weight average molecular weight of silk fibroin or fragments thereof of the same compositions, the two or more values determined by two or more separate experimental readings.

As used herein, the term polymer “polydispersity (PD)” is generally used as a measure of the broadness of a molecular weight distribution of a polymer, and is defined by the formula polydispersity PD=Mw/Mn.

As used herein, the term “substantially homogeneous” may refer to silk fibroin-based protein fragments that are distributed in a normal distribution about an identified molecular weight. As used herein, the term “substantially homogeneous” may refer to an even distribution of a component or an additive, for example, silk fibroin fragments, dermatologically acceptable carrier, etc., throughout a composition of the present disclosure.

As used herein, the terms “silk fibroin peptide,” “silk fibroin protein fragment,” and “silk fibroin fragment” are used interchangeably. Molecular weight or number of amino acids units are defined when molecular size becomes an important parameter.

### SPF Definitions and Properties

As used herein, “silk protein fragments” (SPF) include one or more of: “silk fibroin fragments” as defined herein; “recombinant silk fragments” as defined herein; “spider silk fragments” as defined herein; “silk fibroin-like protein fragments” as defined herein; and/or “chemically modified silk fragments” as defined herein. SPF may have any molecular weight values or ranges described herein, and any polydispersity values or ranges described herein. As used herein, in some embodiments the term “silk protein fragment” also refers to a silk protein that comprises or consists of at least two identical repetitive units which each independently selected from naturally-occurring silk polypeptides or of variations thereof, amino acid sequences of naturally-occurring silk polypeptides, or of combinations of both.

### SPF Molecular Weight and Polydispersity

In an embodiment, a composition of the present disclosure includes SPF having an average weight average molecular weight selected from between about 1 to about 5 kDa. In an embodiment, a composition of the present disclosure includes SPF having an average weight average molecular weight selected from between about 5 to about 10 kDa. In an embodiment, a composition of the present disclosure includes SPF having an average weight average molecular weight selected from between about 10 to about 15 kDa. In an embodiment, a composition of the present disclosure includes SPF having an average weight average molecular weight selected from between about 15 to about 20 kDa. In an embodiment, a composition of the present disclosure includes SPF having an average weight average molecular weight selected from between about 14 to about 30 kDa. In an embodiment, a composition of the present disclosure includes SPF having an average weight average molecular weight selected from between about 20 to about 25 kDa. In an embodiment, a composition of the present disclosure includes SPF having an average weight average molecular weight selected from between about 25 to about 30 kDa. In an embodiment, a composition of the present disclosure includes SPF having an average weight average molecular weight selected from between about 30 to about 35 kDa. In an embodiment, a composition of the present disclosure includes SPF having an average weight average molecular weight selected from between about 35 to about 40 kDa. In an embodiment, a composition of the present disclosure includes SPF having an average weight average molecular weight selected from between about 39 to about 54 kDa. In an embodiment, a composition of the present disclosure includes SPF having an average weight average molecular weight selected from between about 40 to about 45 kDa. In an embodiment, a composition of the present disclosure includes SPF having an average weight average molecular weight selected from between about 45 to about 50 kDa. In an embodiment, a composition of the present disclosure includes SPF having an average weight average molecular weight selected from between about 50 to about 55 kDa. In an embodiment, a composition of the present disclosure includes SPF having an average weight average molecular weight selected from between about 55 to about 60 kDa. In an embodiment, a composition of the present disclosure includes SPF having an average weight average molecular weight selected from between about 60 to about 65 kDa. In an embodiment, a composition of the present disclosure includes SPF having an average weight average molecular weight selected from between about 65 to about 70 kDa. In an embodiment, a composition of the present disclosure includes SPF having an average weight average molecular weight selected from between about 70 to about 75 kDa. In an embodiment, a composition of the present disclosure includes SPF having an average weight average molecular weight selected from between about 75 to about 80 kDa. In an embodiment, a composition of the present disclosure includes SPF having an average weight average molecular weight selected from between about 80 to about 85 kDa. In an embodiment, a composition of the present disclosure includes SPF having an average weight average molecular weight selected from between about 85 to about 90 kDa. In an embodiment, a composition of the present disclosure includes SPF having an average weight average molecular weight selected from between about 90 to about 95 kDa. In an embodiment, a composition of the present disclosure includes SPF having an average weight average molecular weight selected from between about 95 to about 100 kDa. In an embodiment, a composition of the present disclosure includes SPF having an average weight average molecular weight selected from between about 100 to about 105 kDa. In an embodiment, a composition of the present disclosure includes SPF having an average weight average molecular weight selected from between about 105 to about 110 kDa. In an embodiment, a composition of the present disclosure includes SPF having an average weight average molecular weight selected from between about 110 to about 115 kDa. In an embodiment, a composition of the present disclosure includes SPF having an average weight average molecular weight selected from between about 115 to about 120 kDa. In an embodiment, a composition of the present disclosure includes SPF having an average weight average molecular weight selected from between about 120 to about 125 kDa. In an embodiment, a composition of the present disclosure includes SPF having an average weight average molecular weight selected from between about 125 to about 130 kDa. In an embodiment, a composition of the present disclosure includes SPF having an average weight average molecular weight selected from between about 130 to about 135 kDa. In an embodiment, a composition of the present disclosure includes SPF having an average weight average molecular weight selected from between about 135 to about 140 kDa. In an embodiment, a composition of the present disclosure includes SPF having an average weight average molecular weight selected from between about 140 to about 145 kDa. In an embodiment, a composition of the present disclosure includes SPF having an average weight average molecular weight selected from between about 145 to about 150 kDa. In an embodiment, a composition of the present disclosure includes SPF having an average weight average molecular weight selected from between about 150 to about 155 kDa. In an embodiment, a composition of the present disclosure includes SPF having an average weight average molecular weight selected from between about 155 to about 160 kDa. In an embodiment, a composition of the present disclosure includes SPF having an average weight average molecular weight selected from between about 160 to about 165 kDa. In an embodiment, a composition of the present disclosure includes SPF having an average weight average molecular weight selected from between about 165 to about 170 kDa. In an embodiment, a composition of the present disclosure includes SPF having an average weight average molecular weight selected from between about 170 to about 175 kDa. In an embodiment, a composition of the present disclosure includes SPF having an average weight average molecular weight selected from between about 175 to about 180 kDa. In an embodiment, a composition of the present disclosure includes SPF having an average weight average molecular weight selected from between about 180 to about 185 kDa. In an embodiment, a composition of the present disclosure includes SPF having an average weight average molecular weight selected from between about 185 to about 190 kDa. In an embodiment, a composition of the present disclosure includes SPF having an average weight average molecular weight selected from between about 190 to about 195 kDa. In an embodiment, a composition of the present disclosure includes SPF having an average weight average molecular weight selected from between about 195 to about 200 kDa. In an embodiment, a composition of the present disclosure includes SPF having an average weight average molecular weight selected from between about 200 to about 205 kDa. In an embodiment, a composition of the present disclosure includes SPF having an average weight average molecular weight selected from between about 205 to about 210 kDa. In an embodiment, a composition of the present disclosure includes SPF having an average weight average molecular weight selected from between about 210 to about 215 kDa. In an embodiment, a composition of the present disclosure includes SPF having an average weight average molecular weight selected from between about 215 to about 220 kDa. In an embodiment, a composition of the present disclosure includes SPF having an average weight average molecular weight selected from between about 220 to about 225 kDa. In an embodiment, a composition of the present disclosure includes SPF having an average weight average molecular weight selected from between about 225 to about 230 kDa. In an embodiment, a composition of the present disclosure includes SPF having an average weight average molecular weight selected from between about 230 to about 235 kDa. In an embodiment, a composition of the present disclosure includes SPF having an average weight average molecular weight selected from between about 235 to about 240 kDa. In an embodiment, a composition of the present disclosure includes SPF having an average weight average molecular weight selected from between about 240 to about 245 kDa. In an embodiment, a composition of the present disclosure includes SPF having an average weight average molecular weight selected from between about 245 to about 250 kDa. In an embodiment, a composition of the present disclosure includes SPF having an average weight average molecular weight selected from between about 250 to about 255 kDa. In an embodiment, a composition of the present disclosure includes SPF having an average weight average molecular weight selected from between about 255 to about 260 kDa. In an embodiment, a composition of the present disclosure includes SPF having an average weight average molecular weight selected from between about 260 to about 265 kDa. In an embodiment, a composition of the present disclosure includes SPF having an average weight average molecular weight selected from between about 265 to about 270 kDa. In an embodiment, a composition of the present disclosure includes SPF having an average weight average molecular weight selected from between about 270 to about 275 kDa. In an embodiment, a composition of the present disclosure includes SPF having an average weight average molecular weight selected from between about 275 to about 280 kDa. In an embodiment, a composition of the present disclosure includes SPF having an average weight average molecular weight selected from between about 280 to about 285 kDa. In an embodiment, a composition of the present disclosure includes SPF having an average weight average molecular weight selected from between about 285 to about 290 kDa. In an embodiment, a composition of the present disclosure includes SPF having an average weight average molecular weight selected from between about 290 to about 295 kDa. In an embodiment, a composition of the present disclosure includes SPF having an average weight average molecular weight selected from between about 295 to about 300 kDa. In an embodiment, a composition of the present disclosure includes SPF having an average weight average molecular weight selected from between about 300 to about 305 kDa. In an embodiment, a composition of the present disclosure includes SPF having an average weight average molecular weight selected from between about 305 to about 310 kDa. In an embodiment, a composition of the present disclosure includes SPF having an average weight average molecular weight selected from between about 310 to about 315 kDa. In an embodiment, a composition of the present disclosure includes SPF having an average weight average molecular weight selected from between about 315 to about 320 kDa. In an embodiment, a composition of the present disclosure includes SPF having an average weight average molecular weight selected from between about 320 to about 325 kDa. In an embodiment, a composition of the present disclosure includes SPF having an average weight average molecular weight selected from between about 325 to about 330 kDa. In an embodiment, a composition of the present disclosure includes SPF having an average weight average molecular weight selected from between about 330 to about 335 kDa. In an embodiment, a composition of the present disclosure includes SPF having an average weight average molecular weight selected from between about 335 to about 340 kDa. In an embodiment, a composition of the present disclosure includes SPF having an average weight average molecular weight selected from between about 340 to about 345 kDa. In an embodiment, a composition of the present disclosure includes SPF having an average weight average molecular weight selected from between about 345 to about 350 kDa.

In some embodiments, compositions of the present disclosure include SPF compositions selected from compositions #1001 to #2450, having weight average molecular weights selected from about 1 kDa to about 145 kDa, and a polydispersity selected from between 1 and about 5 (including, without limitation, a polydispersity of 1), between 1 and about 1.5 (including, without limitation, a polydispersity of 1), between about 1.5 and about 2, between about 1.5 and about 3, between about 2 and about 2.5, between about 2.5 and about 3, between about 3 and about 3.5, between about 3.5 and about 4, between about 4 and about 4.5, and between about 4.5 and about 5:

| | | PDI | | | | | | | | | |
|---|---|---|---|---|---|---|---|---|---|---|---|
| MW | | (about) | | | | | | | | | |
| (about) | | 1-5 | 1-1.5 | 1.5-2 | 1.5-3 | 2-2.5 | 2.5-3 | 3-3.5 | 3.5-4 | 4-4.5 | 4.5-5 |
| 1 | kDa | 1001 | 1002 | 1003 | 1004 | 1005 | 1006 | 1007 | 1008 | 1009 | 1010 |
| 2 | kDa | 1011 | 1012 | 1013 | 1014 | 1015 | 1016 | 1017 | 1018 | 1019 | 1020 |
| 3 | kDa | 1021 | 1022 | 1023 | 1024 | 1025 | 1026 | 1027 | 1028 | 1029 | 1030 |
| 4 | kDa | 1031 | 1032 | 1033 | 1034 | 1035 | 1036 | 1037 | 1038 | 1039 | 1040 |
| 5 | kDa | 1041 | 1042 | 1043 | 1044 | 1045 | 1046 | 1047 | 1048 | 1049 | 1050 |
| 6 | kDa | 1051 | 1052 | 1053 | 1054 | 1055 | 1056 | 1057 | 1058 | 1059 | 1060 |
| 7 | kDa | 1061 | 1062 | 1063 | 1064 | 1065 | 1066 | 1067 | 1068 | 1069 | 1070 |
| 8 | kDa | 1071 | 1072 | 1073 | 1074 | 1075 | 1076 | 1077 | 1078 | 1079 | 1080 |
| 9 | kDa | 1081 | 1082 | 1083 | 1084 | 1085 | 1086 | 1087 | 1088 | 1089 | 1090 |
| 10 | kDa | 1091 | 1092 | 1093 | 1094 | 1095 | 1096 | 1097 | 1098 | 1099 | 1100 |
| 11 | kDa | 1101 | 1102 | 1103 | 1104 | 1105 | 1106 | 1107 | 1108 | 1109 | 1110 |
| 12 | kDa | 1111 | 1112 | 1113 | 1114 | 1115 | 1116 | 1117 | 1118 | 1119 | 1120 |
| 13 | kDa | 1121 | 1122 | 1123 | 1124 | 1125 | 1126 | 1127 | 1128 | 1129 | 1130 |
| 14 | kDa | 1131 | 1132 | 1133 | 1134 | 1135 | 1136 | 1137 | 1138 | 1139 | 1140 |
| 15 | kDa | 1141 | 1142 | 1143 | 1144 | 1145 | 1146 | 1147 | 1148 | 1149 | 1150 |
| 16 | kDa | 1151 | 1152 | 1153 | 1154 | 1155 | 1156 | 1157 | 1158 | 1159 | 1160 |
| 17 | kDa | 1161 | 1162 | 1163 | 1164 | 1165 | 1166 | 1167 | 1168 | 1169 | 1170 |
| 18 | kDa | 1171 | 1172 | 1173 | 1174 | 1175 | 1176 | 1177 | 1178 | 1179 | 1180 |
| 19 | kDa | 1181 | 1182 | 1183 | 1184 | 1185 | 1186 | 1187 | 1188 | 1189 | 1190 |
| 20 | kDa | 1191 | 1192 | 1193 | 1194 | 1195 | 1196 | 1197 | 1198 | 1199 | 1200 |
| 21 | kDa | 1201 | 1202 | 1203 | 1204 | 1205 | 1206 | 1207 | 1208 | 1209 | 1210 |
| 22 | kDa | 1211 | 1212 | 1213 | 1214 | 1215 | 1216 | 1217 | 1218 | 1219 | 1220 |
| 23 | kDa | 1221 | 1222 | 1223 | 1224 | 1225 | 1226 | 1227 | 1228 | 1229 | 1230 |
| 24 | kDa | 1231 | 1232 | 1233 | 1234 | 1235 | 1236 | 1237 | 1238 | 1239 | 1240 |
| 25 | kDa | 1241 | 1242 | 1243 | 1244 | 1245 | 1246 | 1247 | 1248 | 1249 | 1250 |
| 26 | kDa | 1251 | 1252 | 1253 | 1254 | 1255 | 1256 | 1257 | 1258 | 1259 | 1260 |
| 27 | kDa | 1261 | 1262 | 1263 | 1264 | 1265 | 1266 | 1267 | 1268 | 1269 | 1270 |
| 28 | kDa | 1271 | 1272 | 1273 | 1274 | 1275 | 1276 | 1277 | 1278 | 1279 | 1280 |
| 29 | kDa | 1281 | 1282 | 1283 | 1284 | 1285 | 1286 | 1287 | 1288 | 1289 | 1290 |
| 30 | kDa | 1291 | 1292 | 1293 | 1294 | 1295 | 1296 | 1297 | 1298 | 1299 | 1300 |
| 31 | kDa | 1301 | 1302 | 1303 | 1304 | 1305 | 1306 | 1307 | 1308 | 1309 | 1310 |
| 32 | kDa | 1311 | 1312 | 1313 | 1314 | 1315 | 1316 | 1317 | 1318 | 1319 | 1320 |
| 33 | kDa | 1321 | 1322 | 1323 | 1324 | 1325 | 1326 | 1327 | 1328 | 1329 | 1330 |
| 34 | kDa | 1331 | 1332 | 1333 | 1334 | 1335 | 1336 | 1337 | 1338 | 1339 | 1340 |
| 35 | kDa | 1341 | 1342 | 1343 | 1344 | 1345 | 1346 | 1347 | 1348 | 1349 | 1350 |
| 36 | kDa | 1351 | 1352 | 1353 | 1354 | 1355 | 1356 | 1357 | 1358 | 1359 | 1360 |
| 37 | kDa | 1361 | 1362 | 1363 | 1364 | 1365 | 1366 | 1367 | 1368 | 1369 | 1370 |
| 38 | kDa | 1371 | 1372 | 1373 | 1374 | 1375 | 1376 | 1377 | 1378 | 1379 | 1380 |
| 39 | kDa | 1381 | 1382 | 1383 | 1384 | 1385 | 1386 | 1387 | 1388 | 1389 | 1390 |
| 40 | kDa | 1391 | 1392 | 1393 | 1394 | 1395 | 1396 | 1397 | 1398 | 1399 | 1400 |
| 41 | kDa | 1401 | 1402 | 1403 | 1404 | 1405 | 1406 | 1407 | 1408 | 1409 | 1410 |
| 42 | kDa | 1411 | 1412 | 1413 | 1414 | 1415 | 1416 | 1417 | 1418 | 1419 | 1420 |
| 43 | kDa | 1421 | 1422 | 1423 | 1424 | 1425 | 1426 | 1427 | 1428 | 1429 | 1430 |
| 44 | kDa | 1431 | 1432 | 1433 | 1434 | 1435 | 1436 | 1437 | 1438 | 1439 | 1440 |
| 45 | kDa | 1441 | 1442 | 1443 | 1444 | 1445 | 1446 | 1447 | 1448 | 1449 | 1450 |
| 46 | kDa | 1451 | 1452 | 1453 | 1454 | 1455 | 1456 | 1457 | 1458 | 1459 | 1460 |
| 47 | kDa | 1461 | 1462 | 1463 | 1464 | 1465 | 1466 | 1467 | 1468 | 1469 | 1470 |
| 48 | kDa | 1471 | 1472 | 1473 | 1474 | 1475 | 1476 | 1477 | 1478 | 1479 | 1480 |
| 49 | kDa | 1481 | 1482 | 1483 | 1484 | 1485 | 1486 | 1487 | 1488 | 1489 | 1490 |
| 50 | kDa | 1491 | 1492 | 1493 | 1494 | 1495 | 1496 | 1497 | 1498 | 1499 | 1500 |
| 51 | kDa | 1501 | 1502 | 1503 | 1504 | 1505 | 1506 | 1507 | 1508 | 1509 | 1510 |
| 52 | kDa | 1511 | 1512 | 1513 | 1514 | 1515 | 1516 | 1517 | 1518 | 1519 | 1520 |
| 53 | kDa | 1521 | 1522 | 1523 | 1524 | 1525 | 1526 | 1527 | 1528 | 1529 | 1530 |
| 54 | kDa | 1531 | 1532 | 1533 | 1534 | 1535 | 1536 | 1537 | 1538 | 1539 | 1540 |
| 55 | kDa | 1541 | 1542 | 1543 | 1544 | 1545 | 1546 | 1547 | 1548 | 1549 | 1550 |
| 56 | kDa | 1551 | 1552 | 1553 | 1554 | 1555 | 1556 | 1557 | 1558 | 1559 | 1560 |
| 57 | kDa | 1561 | 1562 | 1563 | 1564 | 1565 | 1566 | 1567 | 1568 | 1569 | 1570 |
| 58 | kDa | 1571 | 1572 | 1573 | 1574 | 1575 | 1576 | 1577 | 1578 | 1579 | 1580 |
| 59 | kDa | 1581 | 1582 | 1583 | 1584 | 1585 | 1586 | 1587 | 1588 | 1589 | 1590 |
| 60 | kDa | 1591 | 1592 | 1593 | 1594 | 1595 | 1596 | 1597 | 1598 | 1599 | 1600 |
| 61 | kDa | 1601 | 1602 | 1603 | 1604 | 1605 | 1606 | 1607 | 1608 | 1609 | 1610 |
| 62 | kDa | 1611 | 1612 | 1613 | 1614 | 1615 | 1616 | 1617 | 1618 | 1619 | 1620 |
| 63 | kDa | 1621 | 1622 | 1623 | 1624 | 1625 | 1626 | 1627 | 1628 | 1629 | 1630 |
| 64 | kDa | 1631 | 1632 | 1633 | 1634 | 1635 | 1636 | 1637 | 1638 | 1639 | 1640 |
| 65 | kDa | 1641 | 1642 | 1643 | 1644 | 1645 | 1646 | 1647 | 1648 | 1649 | 1650 |
| 66 | kDa | 1651 | 1652 | 1653 | 1654 | 1655 | 1656 | 1657 | 1658 | 1659 | 1660 |
| 67 | kDa | 1661 | 1662 | 1663 | 1664 | 1665 | 1666 | 1667 | 1668 | 1669 | 1670 |
| 68 | kDa | 1671 | 1672 | 1673 | 1674 | 1675 | 1676 | 1677 | 1678 | 1679 | 1680 |
| 69 | kDa | 1681 | 1682 | 1683 | 1684 | 1685 | 1686 | 1687 | 1688 | 1689 | 1690 |
| 70 | kDa | 1691 | 1692 | 1693 | 1694 | 1695 | 1696 | 1697 | 1698 | 1699 | 1700 |
| 71 | kDa | 1701 | 1702 | 1703 | 1704 | 1705 | 1706 | 1707 | 1708 | 1709 | 1710 |
| 72 | kDa | 1711 | 1712 | 1713 | 1714 | 1715 | 1716 | 1717 | 1718 | 1719 | 1720 |
| 73 | kDa | 1721 | 1722 | 1723 | 1724 | 1725 | 1726 | 1727 | 1728 | 1729 | 1730 |
| 74 | kDa | 1731 | 1732 | 1733 | 1734 | 1735 | 1736 | 1737 | 1738 | 1739 | 1740 |
| 75 | kDa | 1741 | 1742 | 1743 | 1744 | 1745 | 1746 | 1747 | 1748 | 1749 | 1750 |
| 76 | kDa | 1751 | 1752 | 1753 | 1754 | 1755 | 1756 | 1757 | 1758 | 1759 | 1760 |
| 77 | kDa | 1761 | 1762 | 1763 | 1764 | 1765 | 1766 | 1767 | 1768 | 1769 | 1770 |
| 78 | kDa | 1771 | 1772 | 1773 | 1774 | 1775 | 1776 | 1777 | 1778 | 1779 | 1780 |
| 79 | kDa | 1781 | 1782 | 1783 | 1784 | 1785 | 1786 | 1787 | 1788 | 1789 | 1790 |
| 80 | kDa | 1791 | 1792 | 1793 | 1794 | 1795 | 1796 | 1797 | 1798 | 1799 | 1800 |
| 81 | kDa | 1801 | 1802 | 1803 | 1804 | 1805 | 1806 | 1807 | 1808 | 1809 | 1810 |
| 82 | kDa | 1811 | 1812 | 1813 | 1814 | 1815 | 1816 | 1817 | 1818 | 1819 | 1820 |
| 83 | kDa | 1821 | 1822 | 1823 | 1824 | 1825 | 1826 | 1827 | 1828 | 1829 | 1830 |
| 84 | kDa | 1831 | 1832 | 1833 | 1834 | 1835 | 1836 | 1837 | 1838 | 1839 | 1840 |
| 85 | kDa | 1841 | 1842 | 1843 | 1844 | 1845 | 1846 | 1847 | 1848 | 1849 | 1850 |
| 86 | kDa | 1851 | 1852 | 1853 | 1854 | 1855 | 1856 | 1857 | 1858 | 1859 | 1860 |
| 87 | kDa | 1861 | 1862 | 1863 | 1864 | 1865 | 1866 | 1867 | 1868 | 1869 | 1870 |
| 88 | kDa | 1871 | 1872 | 1873 | 1874 | 1875 | 1876 | 1877 | 1878 | 1879 | 1880 |
| 89 | kDa | 1881 | 1882 | 1883 | 1884 | 1885 | 1886 | 1887 | 1888 | 1889 | 1890 |
| 90 | kDa | 1891 | 1892 | 1893 | 1894 | 1895 | 1896 | 1897 | 1898 | 1899 | 1900 |
| 91 | kDa | 1901 | 1902 | 1903 | 1904 | 1905 | 1906 | 1907 | 1908 | 1909 | 1910 |
| 92 | kDa | 1911 | 1912 | 1913 | 1914 | 1915 | 1916 | 1917 | 1918 | 1919 | 1920 |
| 93 | kDa | 1921 | 1922 | 1923 | 1924 | 1925 | 1926 | 1927 | 1928 | 1929 | 1930 |
| 94 | kDa | 1931 | 1932 | 1933 | 1934 | 1935 | 1936 | 1937 | 1938 | 1939 | 1940 |
| 95 | kDa | 1941 | 1942 | 1943 | 1944 | 1945 | 1946 | 1947 | 1948 | 1949 | 1950 |
| 96 | kDa | 1951 | 1952 | 1953 | 1954 | 1955 | 1956 | 1957 | 1958 | 1959 | 1960 |
| 97 | kDa | 1961 | 1962 | 1963 | 1964 | 1965 | 1966 | 1967 | 1968 | 1969 | 1970 |
| 98 | kDa | 1971 | 1972 | 1973 | 1974 | 1975 | 1976 | 1977 | 1978 | 1979 | 1980 |
| 99 | kDa | 1981 | 1982 | 1983 | 1984 | 1985 | 1986 | 1987 | 1988 | 1989 | 1990 |
| 100 | kDa | 1991 | 1992 | 1993 | 1994 | 1995 | 1996 | 1997 | 1998 | 1999 | 2000 |
| 101 | kDa | 2001 | 2002 | 2003 | 2004 | 2005 | 2006 | 2007 | 2008 | 2009 | 2010 |
| 102 | kDa | 2011 | 2012 | 2013 | 2014 | 2015 | 2016 | 2017 | 2018 | 2019 | 2020 |
| 103 | kDa | 2021 | 2022 | 2023 | 2024 | 2025 | 2026 | 2027 | 2028 | 2029 | 2030 |
| 104 | kDa | 2031 | 2032 | 2033 | 2034 | 2035 | 2036 | 2037 | 2038 | 2039 | 2040 |
| 105 | kDa | 2041 | 2042 | 2043 | 2044 | 2045 | 2046 | 2047 | 2048 | 2049 | 2050 |
| 106 | kDa | 2051 | 2052 | 2053 | 2054 | 2055 | 2056 | 2057 | 2058 | 2059 | 2060 |
| 107 | kDa | 2061 | 2062 | 2063 | 2064 | 2065 | 2066 | 2067 | 2068 | 2069 | 2070 |
| 108 | kDa | 2071 | 2072 | 2073 | 2074 | 2075 | 2076 | 2077 | 2078 | 2079 | 2080 |
| 109 | kDa | 2081 | 2082 | 2083 | 2084 | 2085 | 2086 | 2087 | 2088 | 2089 | 2090 |
| 110 | kDa | 2091 | 2092 | 2093 | 2094 | 2095 | 2096 | 2097 | 2098 | 2099 | 2100 |
| 111 | kDa | 2101 | 2102 | 2103 | 2104 | 2105 | 2106 | 2107 | 2108 | 2109 | 2110 |
| 112 | kDa | 2111 | 2112 | 2113 | 2114 | 2115 | 2116 | 2117 | 2118 | 2119 | 2120 |
| 113 | kDa | 2121 | 2122 | 2123 | 2124 | 2125 | 2126 | 2127 | 2128 | 2129 | 2130 |
| 114 | kDa | 2131 | 2132 | 2133 | 2134 | 2135 | 2136 | 2137 | 2138 | 2139 | 2140 |
| 115 | kDa | 2141 | 2142 | 2143 | 2144 | 2145 | 2146 | 2147 | 2148 | 2149 | 2150 |
| 116 | kDa | 2151 | 2152 | 2153 | 2154 | 2155 | 2156 | 2157 | 2158 | 2159 | 2160 |
| 117 | kDa | 2161 | 2162 | 2163 | 2164 | 2165 | 2166 | 2167 | 2168 | 2169 | 2170 |
| 118 | kDa | 2171 | 2172 | 2173 | 2174 | 2175 | 2176 | 2177 | 2178 | 2179 | 2180 |
| 119 | kDa | 2181 | 2182 | 2183 | 2184 | 2185 | 2186 | 2187 | 2188 | 2189 | 2190 |
| 120 | kDa | 2191 | 2192 | 2193 | 2194 | 2195 | 2196 | 2197 | 2198 | 2199 | 2200 |
| 121 | kDa | 2201 | 2202 | 2203 | 2204 | 2205 | 2206 | 2207 | 2208 | 2209 | 2210 |
| 122 | kDa | 2211 | 2212 | 2213 | 2214 | 2215 | 2216 | 2217 | 2218 | 2219 | 2220 |
| 123 | kDa | 2221 | 2222 | 2223 | 2224 | 2225 | 2226 | 2227 | 2228 | 2229 | 2230 |
| 124 | kDa | 2231 | 2232 | 2233 | 2234 | 2235 | 2236 | 2237 | 2238 | 2239 | 2240 |
| 125 | kDa | 2241 | 2242 | 2243 | 2244 | 2245 | 2246 | 2247 | 2248 | 2249 | 2250 |
| 126 | kDa | 2251 | 2252 | 2253 | 2254 | 2255 | 2256 | 2257 | 2258 | 2259 | 2260 |
| 127 | kDa | 2261 | 2262 | 2263 | 2264 | 2265 | 2266 | 2267 | 2268 | 2269 | 2270 |
| 128 | kDa | 2271 | 2272 | 2273 | 2274 | 2275 | 2276 | 2277 | 2278 | 2279 | 2280 |
| 129 | kDa | 2281 | 2282 | 2283 | 2284 | 2285 | 2286 | 2287 | 2288 | 2289 | 2290 |
| 130 | kDa | 2291 | 2292 | 2293 | 2294 | 2295 | 2296 | 2297 | 2298 | 2299 | 2300 |
| 131 | kDa | 2301 | 2302 | 2303 | 2304 | 2305 | 2306 | 2307 | 2308 | 2309 | 2310 |
| 132 | kDa | 2311 | 2312 | 2313 | 2314 | 2315 | 2316 | 2317 | 2318 | 2319 | 2320 |
| 133 | kDa | 2321 | 2322 | 2323 | 2324 | 2325 | 2326 | 2327 | 2328 | 2329 | 2330 |
| 134 | kDa | 2331 | 2332 | 2333 | 2334 | 2335 | 2336 | 2337 | 2338 | 2339 | 2340 |
| 135 | kDa | 2341 | 2342 | 2343 | 2344 | 2345 | 2346 | 2347 | 2348 | 2349 | 2350 |
| 136 | kDa | 2351 | 2352 | 2353 | 2354 | 2355 | 2356 | 2357 | 2358 | 2359 | 2360 |
| 137 | kDa | 2361 | 2362 | 2363 | 2364 | 2365 | 2366 | 2367 | 2368 | 2369 | 2370 |
| 138 | kDa | 2371 | 2372 | 2373 | 2374 | 2375 | 2376 | 2377 | 2378 | 2379 | 2380 |
| 139 | kDa | 2381 | 2382 | 2383 | 2384 | 2385 | 2386 | 2387 | 2388 | 2389 | 2390 |
| 140 | kDa | 2391 | 2392 | 2393 | 2394 | 2395 | 2396 | 2397 | 2398 | 2399 | 2400 |
| 141 | kDa | 2401 | 2402 | 2403 | 2404 | 2405 | 2406 | 2407 | 2408 | 2409 | 2410 |
| 142 | kDa | 2411 | 2412 | 2413 | 2414 | 2415 | 2416 | 2417 | 2418 | 2419 | 2420 |
| 143 | kDa | 2421 | 2422 | 2423 | 2424 | 2425 | 2426 | 2427 | 2428 | 2429 | 2430 |
| 144 | kDa | 2431 | 2432 | 2433 | 2434 | 2435 | 2436 | 2437 | 2438 | 2439 | 2440 |
| 145 | kDa | 2441 | 2442 | 2443 | 2444 | 2445 | 2446 | 2447 | 2448 | 2449 | 2450 |

As used herein, “low molecular weight,” “low MW,” or “low-MW” SPF may include SPF having a weight average molecular weight, or average weight average molecular weight selected from between about 5 kDa to about 38 kDa, about 14 kDa to about 30 kDa, or about 6 kDa to about 17 kDa. In some embodiments, a target low molecular weight for certain SPF may be weight average molecular weight of about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, or about 38 kDa.

As used herein, “medium molecular weight,” “medium MW,” or “mid-MW” SPF may include SPF having a weight average molecular weight, or average weight average molecular weight selected from between about 31 kDa to about 55 kDa, or about 39 kDa to about 54 kDa. In some embodiments, a target medium molecular weight for certain SPF may be weight average molecular weight of about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, about 51 kDa, about 52 kDa, about 53 kDa, about 54 kDa, or about 55 kDa.

As used herein, “high molecular weight,” “high MW,” or “high-MW” SPF may include SPF having a weight average molecular weight, or average weight average molecular weight selected from between about 55 kDa to about 150 kDa. In some embodiments, a target high molecular weight for certain SPF may be about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, about 69 kDa, about 70 kDa, about 71 kDa, about 72 kDa, about 73 kDa, about 74 kDa, about 75 kDa, about 76 kDa, about 77 kDa, about 78 kDa, about 79 kDa, or about 80 kDa.

In some embodiments, the molecular weights described herein (e.g., low molecular weight silk, medium molecular weight silk, high molecular weight silk) may be converted to the approximate number of amino acids contained within the respective SPF, as would be understood by a person having ordinary skill in the art. For example, the average weight of an amino acid may be about 110 daltons (i.e., 110 g/mol). Therefore, in some embodiments, dividing the molecular weight of a linear protein by 110 daltons may be used to approximate the number of amino acid residues contained therein.

In an embodiment, SPF in a composition of the present disclosure have a polydispersity selected from between 1 to about 5.0, including, without limitation, a polydispersity of 1. In an embodiment, SPF in a composition of the present disclosure have a polydispersity selected from between about 1.5 to about 3.0. In an embodiment, SPF in a composition of the present disclosure have a polydispersity selected from between 1 to about 1.5, including, without limitation, a polydispersity of 1. In an embodiment, SPF in a composition of the present disclosure have a polydispersity selected from between about 1.5 to about 2.0. In an embodiment, SPF in a composition of the present disclosure have a polydispersity selected from between about 2.0 to about 2.5. In an embodiment, SPF in a composition of the present disclosure have a polydispersity selected from between about 2.5 to about 3.0. In an embodiment, SPF in a composition of the present disclosure have a polydispersity selected from between about 3.0 to about 3.5. In an embodiment, SPF in a composition of the present disclosure have a polydispersity selected from between about 3.5 to about 4.0. In an embodiment, SPF in a composition of the present disclosure have a polydispersity selected from between about 4.0 to about 4.5. In an embodiment, SPF in a composition of the present disclosure have a polydispersity selected from between about 4.5 to about 5.0.

In an embodiment, SPF in a composition of the present disclosure have a polydispersity of 1. In an embodiment, SPF in a composition of the present disclosure have a polydispersity of about 1.1. In an embodiment, SPF in a composition of the present disclosure have a polydispersity of about 1.2. In an embodiment, SPF in a composition of the present disclosure have a polydispersity of about 1.3. In an embodiment, SPF in a composition of the present disclosure have a polydispersity of about 1.4. In an embodiment, SPF in a composition of the present disclosure have a polydispersity of about 1.5. In an embodiment, SPF in a composition of the present disclosure have a polydispersity of about 1.6. In an embodiment, SPF in a composition of the present disclosure have a polydispersity of about 1.7. In an embodiment, SPF in a composition of the present disclosure have a polydispersity of about 1.8. In an embodiment, SPF in a composition of the present disclosure have a polydispersity of about 1.9. In an embodiment, SPF in a composition of the present disclosure have a polydispersity of about 2.0. In an embodiment, SPF in a composition of the present disclosure have a polydispersity of about 2.1. In an embodiment, SPF in a composition of the present disclosure have a polydispersity of about 2.2. In an embodiment, SPF in a composition of the present disclosure have a polydispersity of about 2.3. In an embodiment, SPF in a composition of the present disclosure have a polydispersity of about 2.4. In an embodiment, SPF in a composition of the present disclosure have a polydispersity of about 2.5. In an embodiment, SPF in a composition of the present disclosure have a polydispersity of about 2.6. In an embodiment, SPF in a composition of the present disclosure have a polydispersity of about 2.7. In an embodiment, SPF in a composition of the present disclosure have a polydispersity of about 2.8. In an embodiment, SPF in a composition of the present disclosure have a polydispersity of about 2.9. In an embodiment, SPF in a composition of the present disclosure have a polydispersity of about 3.0. In an embodiment, SPF in a composition of the present disclosure have a polydispersity of about 3.1. In an embodiment, SPF in a composition of the present disclosure have a polydispersity of about 3.2. In an embodiment, SPF in a composition of the present disclosure have a polydispersity of about 3.3. In an embodiment, SPF in a composition of the present disclosure have a polydispersity of about 3.4. In an embodiment, SPF in a composition of the present disclosure have a polydispersity of about 3.5. In an embodiment, SPF in a composition of the present disclosure have a polydispersity of about 3.6. In an embodiment, SPF in a composition of the present disclosure have a polydispersity of about 3.7. In an embodiment, SPF in a composition of the present disclosure have a polydispersity of about 3.8. In an embodiment, SPF in a composition of the present disclosure have a polydispersity of about 3.9. In an embodiment, SPF in a composition of the present disclosure have a polydispersity of about 4.0. In an embodiment, SPF in a composition of the present disclosure have a polydispersity of about 4.1. In an embodiment, SPF in a composition of the present disclosure have a polydispersity of about 4.2. In an embodiment, SPF in a composition of the present disclosure have a polydispersity of about 4.3. In an embodiment, SPF in a composition of the present disclosure have a polydispersity of about 4.4. In an embodiment, SPF in a composition of the present disclosure have a polydispersity of about 4.5. In an embodiment, SPF in a composition of the present disclosure have a polydispersity of about 4.6. In an embodiment, SPF in a composition of the present disclosure have a polydispersity of about 4.7. In an embodiment, SPF in a composition of the present disclosure have a polydispersity of about 4.8. In an embodiment, SPF in a composition of the present disclosure have a polydispersity of about 4.9. In an embodiment, SPF in a composition of the present disclosure have a polydispersity of about 5.0.

In some embodiments, in compositions described herein having combinations of low, medium, and/or high molecular weight SPF, such low, medium, and/or high molecular weight SPF may have the same or different polydispersities.

### Silk Fibroin Fragments

Methods of making silk fibroin or silk fibroin protein fragments and their applications in various fields are known and are described for example in U.S. Pat. Nos. 9,187,538, 9,511,012, 9,517,191, 9,522,107, 9,522,108, 9,545,369, and 10,166,177, 10,287,728 and 10,301,768, all of which are incorporated herein in their entireties. Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain). Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

Provided herein are methods for producing pure and highly scalable silk fibroin-protein fragment mixture solutions that may be used across multiple industries for a variety of applications. Without wishing to be bound by any particular theory, it is believed that these methods are equally applicable to fragmentation of any SPF described herein, including without limitation recombinant silk proteins, and fragmentation of silk-like or fibroin-like proteins.

As used herein, the term “fibroin” includes silkworm fibroin and insect or spider silk protein. In an embodiment, fibroin is obtained from Bombyx mori. Raw silk from Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. Conversion of these insoluble silk fibroin fibrils into water-soluble silk fibroin protein fragments requires the addition of a concentrated neutral salt (e.g., 8-10 M lithium bromide), which interferes with inter- and intramolecular ionic and hydrogen bonding that would otherwise render the fibroin protein insoluble in water. Methods of making silk fibroin protein fragments, and/or compositions thereof, are known and are described for example in U.S. Pat. Nos. 9,187,538, 9,511,012, 9,517,191, 9,522,107, 9,522,108, 9,545,369, and 10,166,177.

The raw silk cocoons from the silkworm Bombyx mori was cut into pieces. The pieces silk cocoons were processed in an aqueous solution of Na₂CO₃ at about 100° C. for about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L× the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at about 100° C. for about 60 minutes to achieve complete dissolution of the native silk protein. The resulting silk fibroin solution was filtered and dialyzed using Tangential Flow Filtration (TFF) and a 10 kDa membrane against deionized water for 72 hours. The resulting silk fibroin aqueous solution has a concentration of about 8.5 wt. %. Then, 8.5% silk solution was diluted with water to result in a 1.0% w/v silk solution. TFF can then be used to further concentrate the pure silk solution to a concentration of 20.0% w/w silk to water.

Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

In some embodiments, the silk solutions are prepared under various preparation condition parameters such as: 90° C. 30 min, 90° C. 60 min, 100° C. 30 min, and 100° C. 60 min. Briefly, 9.3 M LiBr was prepared and allowed to sit at room temperature for at least 30 minutes. 5 mL of LiBr solution was added to 1.25 g of silk and placed in the 60° C. oven. Samples from each set were removed at 4, 6, 8, 12, 24, 168 and 192 hours.

In some embodiments, the silk solutions are prepared under various preparation condition parameters such as: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the 60° C. oven. Samples from each set were removed at 1, 4 and 6 hours.

In some embodiments, the silk solutions are prepared under various preparation condition parameters such as: Four different silk extraction combinations were used: 90° C. 30 min, 90° C. 60 min, 100° C. 30 min, and 100° C. 60 min. Briefly, 9.3 M LiBr solution was heated to one of four temperatures: 60° C., 80° C., 100° C. or boiling. 5 mL of hot LiBr solution was added to 1.25 g of silk and placed in the oven at the same temperature of the LiBr. Samples from each set were removed at 1, 4 and 6 hours. 1 mL of each sample was added to 7.5 mL of 9.3 M LiBr and refrigerated for viscosity testing.

In some embodiments, SPF are obtained by dissolving raw unscoured, partially scoured, or scoured silkworm fibers with a neutral lithium bromide salt. The raw silkworm silks are processed under selected temperature and other conditions in order to remove any sericin and achieve the desired weight average molecular weight (Mw) and polydispersity (PD) of the fragment mixture. Selection of process parameters may be altered to achieve distinct final silk protein fragment characteristics depending upon the intended use. The resulting final fragment solution is silk fibroin protein fragments and water with parts per million (ppm) to non-detectable levels of process contaminants, levels acceptable in the pharmaceutical, medical and consumer eye care markets. The concentration, size and polydispersity of SPF may further be altered depending upon the desired use and performance requirements.

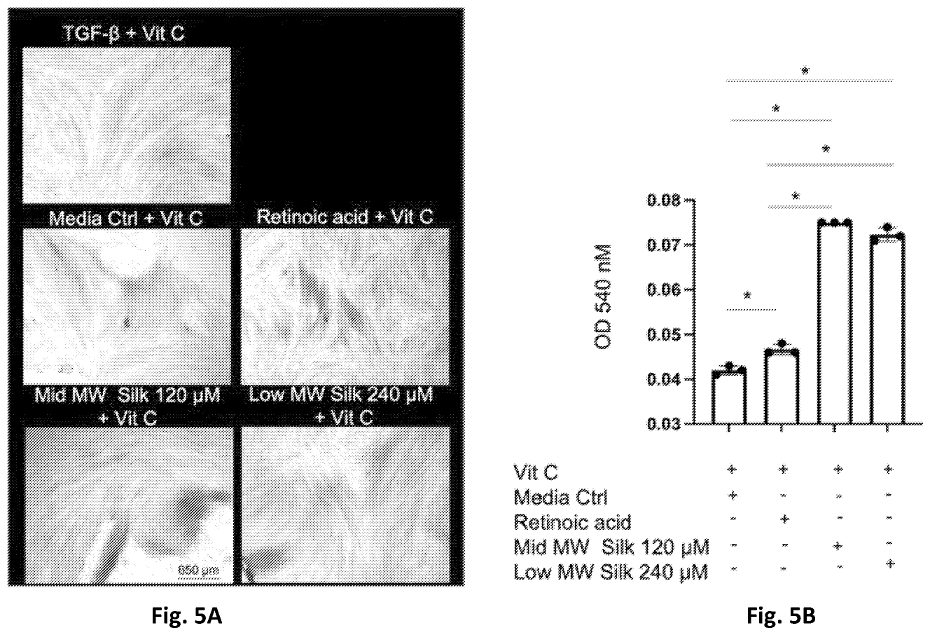

FIG. 5 is a flow chart showing various embodiments for producing pure silk fibroin protein fragments (SPFs) of the present disclosure. It should be understood that not all of the steps illustrated are necessarily required to fabricate all silk solutions of the present disclosure. As illustrated in FIG. 5, step A, cocoons (heat-treated or non-heat-treated), silk fibers, silk powder, spider silk or recombinant spider silk can be used as the silk source. If starting from raw silk cocoons from Bombyx mori, the cocoons can be cut into small pieces, for example pieces of approximately equal size, step B1. The raw silk is then extracted and rinsed to remove any sericin, step C1a. This results in substantially sericin free raw silk. In an embodiment, water is heated to a temperature between 84° C. and 100° C. (ideally boiling) and then Na₂CO₃ (sodium carbonate) is added to the boiling water until the Na₂CO₃ is completely dissolved. The raw silk is added to the boiling water/Na₂CO₃ (100° C.) and submerged for approximately 15-90 minutes, where boiling for a longer time results in smaller silk protein fragments. In an embodiment, the water volume equals about 0.4× raw silk weight and the Na₂CO₃ volume equals about 0.848× raw silk weight. In an embodiment, the water volume equals 0.1× raw silk weight and the Na₂CO₃ volume is maintained at 2.12 g/L.

Subsequently, the water dissolved Na₂CO₃ solution is drained and excess water/Na₂CO₃ is removed from the silk fibroin fibers (e.g., ring out the fibroin extract by hand, spin cycle using a machine, etc.). The resulting silk fibroin extract is rinsed with warm to hot water to remove any remaining adsorbed sericin or contaminate, typically at a temperature range of about 40° C. to about 80° C., changing the volume of water at least once (repeated for as many times as required). The resulting silk fibroin extract is a substantially sericin-depleted silk fibroin. In an embodiment, the resulting silk fibroin extract is rinsed with water at a temperature of about 60° C. In an embodiment, the volume of rinse water for each cycle equals 0.1 L to 0.2 L× raw silk weight. It may be advantageous to agitate, turn or circulate the rinse water to maximize the rinse effect. After rinsing, excess water is removed from the extracted silk fibroin fibers (e.g., ring out fibroin extract by hand or using a machine). Alternatively, methods known to one skilled in the art such as pressure, temperature, or other reagents or combinations thereof may be used for the purpose of sericin extraction. Alternatively, the silk gland (100% sericin free silk protein) can be removed directly from a worm. This would result in liquid silk protein, without any alteration of the protein structure, free of sericin.

The extracted fibroin fibers are then allowed to dry completely. Once dry, the extracted silk fibroin is dissolved using a solvent added to the silk fibroin at a temperature between ambient and boiling, step C1b. In an embodiment, the solvent is a solution of Lithium bromide (LiBr) (boiling for LiBr is 140° C.). Alternatively, the extracted fibroin fibers are not dried but wet and placed in the solvent; solvent concentration can then be varied to achieve similar concentrations as to when adding dried silk to the solvent. The final concentration of LiBr solvent can range from 0.1 M to 9.3 M. Complete dissolution of the extracted fibroin fibers can be achieved by varying the treatment time and temperature along with the concentration of dissolving solvent. Other solvents may be used including, but not limited to, phosphate phosphoric acid, calcium nitrate, calcium chloride solution or other concentrated aqueous solutions of inorganic salts. To ensure complete dissolution, the silk fibers should be fully immersed within the already heated solvent solution and then maintained at a temperature ranging from about 60° C. to about 140° C. for 1-168 hrs. In an embodiment, the silk fibers should be fully immersed within the solvent solution and then placed into a dry oven at a temperature of about 100° C. for about 1 hour.

The temperature at which the silk fibroin extract is added to the LiBr solution (or vice versa) has an effect on the time required to completely dissolve the fibroin and on the resulting molecular weight and polydispersity of the final SPF mixture solution. In an embodiment, silk solvent solution concentration is less than or equal to 20% w/v. In addition, agitation during introduction or dissolution may be used to facilitate dissolution at varying temperatures and concentrations. The temperature of the LiBr solution will provide control over the silk protein fragment mixture molecular weight and polydispersity created. In an embodiment, a higher temperature will more quickly dissolve the silk offering enhanced process scalability and mass production of silk solution. In an embodiment, using a LiBr solution heated to a temperature from 80° C. to 140° C. reduces the time required in an oven in order to achieve full dissolution. Varying time and temperature at or above 60° C. of the dissolution solvent will alter and control the MW and polydispersity of the SPF mixture solutions formed from the original molecular weight of the native silk fibroin protein.

Alternatively, whole cocoons may be placed directly into a solvent, such as LiBr, bypassing extraction, step B2. This requires subsequent filtration of silk worm particles from the silk and solvent solution and sericin removal using methods know in the art for separating hydrophobic and hydrophilic proteins such as a column separation and/or chromatography, ion exchange, chemical precipitation with salt and/or pH, and or enzymatic digestion and filtration or extraction, all methods are common examples and without limitation for standard protein separation methods, step C2. Non-heat treated cocoons with the silkworm removed, may alternatively be placed into a solvent such as LiBr, bypassing extraction. The methods described above may be used for sericin separation, with the advantage that non-heat treated cocoons will contain significantly less worm debris.

Dialysis may be used to remove the dissolution solvent from the resulting dissolved fibroin protein fragment solution by dialyzing the solution against a volume of water, step E1. Pre-filtration prior to dialysis is helpful to remove any debris (i.e., silkworm remnants) from the silk and LiBr solution, step D. In one example, a 3 μm or 5 μm filter is used with a flow-rate of 200-300 mL/min to filter a 0.1% to 1.0% silk-LiBr solution prior to dialysis and potential concentration if desired. A method disclosed herein, as described above, is to use time and/or temperature to decrease the concentration from 9.3 M LiBr to a range from 0.1 M to 9.3 M to facilitate filtration and downstream dialysis, particularly when considering creating a scalable process method. Alternatively, without the use of additional time or temperate, a 9.3 M LiBr-silk protein fragment solution may be diluted with water to facilitate debris filtration and dialysis. The result of dissolution at the desired time and temperate filtration is a translucent particle-free room temperature shelf-stable silk protein fragment-LiBr solution of a known MW and polydispersity. It is advantageous to change the dialysis water regularly until the solvent has been removed (e.g., change water after 1 hour, 4 hours, and then every 12 hours for a total of 6 water changes). The total number of water volume changes may be varied based on the resulting concentration of solvent used for silk protein dissolution and fragmentation. After dialysis, the final silk solution maybe further filtered to remove any remaining debris (i.e., silk worm remnants).

Alternatively, Tangential Flow Filtration (TFF), which is a rapid and efficient method for the separation and purification of biomolecules, may be used to remove the solvent from the resulting dissolved fibroin solution, step E2. TFF offers a highly pure aqueous silk protein fragment solution and enables scalability of the process in order to produce large volumes of the solution in a controlled and repeatable manner. The silk and LiBr solution may be diluted prior to TFF (20% down to 0.1% silk in either water or LiBr). Pre-filtration as described above prior to TFF processing may maintain filter efficiency and potentially avoids the creation of silk gel boundary layers on the filter's surface as the result of the presence of debris particles. Pre-filtration prior to TFF is also helpful to remove any remaining debris (i.e., silk worm remnants) from the silk and LiBr solution that may cause spontaneous or long-term gelation of the resulting water only solution, step D. TFF, recirculating or single pass, may be used for the creation of water-silk protein fragment solutions ranging from 0.1% silk to 30.0% silk (more preferably, 0.1%-6.0% silk). Different cutoff size TFF membranes may be required based upon the desired concentration, molecular weight and polydispersity of the silk protein fragment mixture in solution. Membranes ranging from 1-100 kDa may be necessary for varying molecular weight silk solutions created for example by varying the length of extraction boil time or the time and temperate in dissolution solvent (e.g., LiBr). In an embodiment, a TFF 5 or 10 kDa membrane is used to purify the silk protein fragment mixture solution and to create the final desired silk-to-water ratio. As well, TFF single pass, TFF, and other methods known in the art, such as a falling film evaporator, may be used to concentrate the solution following removal of the dissolution solvent (e.g., LiBr) (with resulting desired concentration ranging from 0.1% to 30% silk). This can be used as an alternative to standard HFIP concentration methods known in the art to create a water-based solution. A larger pore membrane could also be utilized to filter out small silk protein fragments and to create a solution of higher molecular weight silk with and/or without tighter polydispersity values.

An assay for LiBr and Na₂CO₃ detection can be performed using an HPLC system equipped with evaporative light scattering detector (ELSD). The calculation was performed by linear regression of the resulting peak areas for the analyte plotted against concentration. More than one sample of a number of formulations of the present disclosure was used for sample preparation and analysis. Generally, four samples of different formulations were weighed directly in a 10 mL volumetric flask. The samples were suspended in 5 mL of 20 mM ammonium formate (pH 3.0) and kept at 2-8° C. for 2 hours with occasional shaking to extract analytes from the film. After 2 hours the solution was diluted with 20 mM ammonium formate (pH 3.0). The sample solution from the volumetric flask was transferred into HPLC vials and injected into the HPLC-ELSD system for the estimation of sodium carbonate and lithium bromide.

The analytical method developed for the quantitation of Na₂CO₃ and LiBr in silk protein formulations was found to be linear in the range 10-165 μg/mL, with RSD for injection precision as 2% and 1% for area and 0.38% and 0.19% for retention time for sodium carbonate and lithium bromide respectively. The analytical method can be applied for the quantitative determination of sodium carbonate and lithium bromide in silk protein formulations.

FIG. 6 is a flow chart showing various parameters that can be modified during the process of producing a silk protein fragment solution of the present disclosure during the extraction and the dissolution steps. Select method parameters may be altered to achieve distinct final solution characteristics depending upon the intended use, e.g., molecular weight and polydispersity. It should be understood that not all of the steps illustrated are necessarily required to fabricate all silk solutions of the present disclosure.

In an embodiment, silk protein fragment solutions useful for a wide variety of applications are prepared according to the following steps: forming pieces of silk cocoons from the Bombyx mori silkworm; extracting the pieces at about 100° C. in a Na₂CO₃ water solution for about 60 minutes, wherein a volume of the water equals about 0.4× raw silk weight and the amount of Na₂CO₃ is about 0.848× the weight of the pieces to form a silk fibroin extract; triple rinsing the silk fibroin extract at about 60° C. for about 20 minutes per rinse in a volume of rinse water, wherein the rinse water for each cycle equals about 0.2 L× the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also, without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

The extraction step could be completed in a larger vessel, for example an industrial washing machine where temperatures at or in between 60° C. to 100° C. can be maintained. The rinsing step could also be completed in the industrial washing machine, eliminating the manual rinse cycles. Dissolution of the silk in LiBr solution could occur in a vessel other than a convection oven, for example a stirred tank reactor. Dialyzing the silk through a series of water changes is a manual and time intensive process, which could be accelerated by changing certain parameters, for example diluting the silk solution prior to dialysis. The dialysis process could be scaled for manufacturing by using semi-automated equipment, for example a tangential flow filtration system.

Varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions. While almost all parameters resulted in a viable silk solution, methods that allow complete dissolution to be achieved in fewer than 4 to 6 hours are preferred for process scalability.

In an embodiment, solutions of silk fibroin protein fragments having a weight average selected from between about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at most 1 hour; removing the lithium bromide from the silk fibroin extract; and producing an aqueous solution of silk protein fragments, the aqueous solution comprising: fragments having a weight average molecular weight selected from between about 6 kDa to about 17 kDa, and a polydispersity of between 1 and about 5, or between about 1.5 and about 3.0. The method may further comprise drying the silk fibroin extract prior to the dissolving step.

The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. The aqueous solution of silk fibroin protein fragments may be lyophilized. In some embodiments, the silk fibroin protein fragment solution may be further processed into various forms including gel, powder, and nanofiber.

In an embodiment, solutions of silk fibroin protein fragments having a weight average molecular weight selected from between about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin protein fragments comprises fragments having a weight average molecular weight selected from between about 17 kDa to about 39 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay.

In some embodiments, a method for preparing an aqueous solution of silk fibroin protein fragments having an average weight average molecular weight selected from between about 6 kDa to about 17 kDa includes the steps of: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having an average weight average molecular weight selected from between about 6 kDa to about 17 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In some embodiments, a method for preparing an aqueous solution of silk fibroin protein fragments having an average weight average molecular weight selected from between about 17 kDa to about 39 kDa includes the steps of: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of pure silk fibroin protein fragments, wherein the aqueous solution of pure silk fibroin protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of pure silk fibroin protein fragments comprises fragments having an average weight average molecular weight selected from between about 17 kDa to about 39 kDa, and a polydispersity of between 1 and about 5, or between about 1.5 and about 3.0. The method may further comprise drying the silk fibroin extract prior to the dissolving step. The aqueous solution of pure silk fibroin protein fragments may comprise lithium bromide residuals of less than 300 ppm as measured using a high-performance liquid chromatography lithium bromide assay. The aqueous solution of pure silk fibroin protein fragments may comprise sodium carbonate residuals of less than 100 ppm as measured using a high-performance liquid chromatography sodium carbonate assay. The method may further comprise adding a therapeutic agent to the aqueous solution of pure silk fibroin protein fragments. The method may further comprise adding a molecule selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. The method may further comprise adding at least one of zinc oxide or titanium dioxide. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2% and a vitamin content of at least 20%.

In an embodiment, solutions of silk fibroin protein fragments having a weight average molecular weight selected from between about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at most 1 hour; removing the lithium bromide from the silk fibroin extract; and producing an aqueous solution of silk fibroin protein fragments, wherein the aqueous solution of silk fibroin protein fragments comprises lithium bromide residuals of between about 10 ppm and about 300 ppm, sodium carbonate residuals of between about 10 ppm and about 100 ppm, fragments having a weight average molecular weight selected from between about 39 kDa to about 80 kDa, and a polydispersity of between 1 and about 5, or between about 1.5 and about 3.0. The method may further comprise drying the silk fibroin extract prior to the dissolving step. The aqueous solution of silk fibroin protein fragments may comprise lithium bromide residuals of less than 300 ppm as measured using a high-performance liquid chromatography lithium bromide assay. The aqueous solution of silk fibroin protein fragments may comprise sodium carbonate residuals of less than 100 ppm as measured using a high-performance liquid chromatography sodium carbonate assay. In some embodiments, the method may further comprise adding an active agent (e.g., therapeutic agent) to the aqueous solution of pure silk fibroin protein fragments. The method may further comprise adding an active agent selected from one of an antioxidant or an enzyme to the aqueous solution of pure silk fibroin protein fragments. The method may further comprise adding a vitamin to the aqueous solution of pure silk fibroin protein fragments. The vitamin may be vitamin C or a derivative thereof. The aqueous solution of pure silk fibroin protein fragments may be lyophilized. The method may further comprise adding an alpha-hydroxy acid to the aqueous solution of pure silk fibroin protein fragments. The alpha hydroxy acid may be selected from the group consisting of glycolic acid, lactic acid, tartaric acid and citric acid. The method may further comprise adding hyaluronic acid or its salt form at a concentration of about 0.5% to about 10.0% to the aqueous solution of pure silk fibroin protein fragments. A film may be fabricated from the aqueous solution of pure silk fibroin protein fragments produced by this method. The film may comprise from about 1.0 wt. % to about 50.0 wt. % of vitamin C or a derivative thereof. The film may have a water content ranging from about 2.0 wt. % to about 20.0 wt. %. The film may comprise from about 30.0 wt. % to about 99.5 wt. % of pure silk fibroin protein fragments. A gel may be fabricated from the aqueous solution of pure silk fibroin protein fragments produced by this method. The gel may comprise from about 0.5 wt. % to about 20.0 wt. % of vitamin C or a derivative thereof. The gel may have a silk content of at least 2 wt. % and a vitamin content of at least 20 wt. %.

Molecular weight of the silk protein fragments may be controlled based upon the specific parameters utilized during the extraction step, including extraction time and temperature; specific parameters utilized during the dissolution step, including the LiBr temperature at the time of submersion of the silk in to the lithium bromide and time that the solution is maintained at specific temperatures; and specific parameters utilized during the filtration step. By controlling process parameters using the disclosed methods, it is possible to create silk fibroin protein fragment solutions with polydispersity equal to or lower than 2.5 at a variety of different molecular weight selected from between 5 kDa to 200 kDa, or between 10 kDa and 80 kDa. By altering process parameters to achieve silk solutions with different molecular weights, a range of fragment mixture end products, with desired polydispersity of equal to or less than 2.5 may be targeted based upon the desired performance requirements. For example, a higher molecular weight silk film containing an ophthalmic drug may have a controlled slow release rate compared to a lower molecular weight film making it ideal for a delivery vehicle in eye care products. Additionally, the silk fibroin protein fragment solutions with a polydispersity of greater than 2.5 can be achieved. Further, two solutions with different average molecular weights and polydispersity can be mixed to create combination solutions. Alternatively, a liquid silk gland (100% sericin free silk protein) that has been removed directly from a worm could be used in combination with any of the silk fibroin protein fragment solutions of the present disclosure. Molecular weight of the pure silk fibroin protein fragment composition was determined using High Pressure Liquid Chromatography (HPLC) with a Refractive Index Detector (RID). Polydispersity was calculated using Cirrus GPC Online GPC/SEC Software Version 3.3 (Agilent).

Differences in the processing parameters can result in regenerated silk fibroins that vary in molecular weight, and peptide chain size distribution (polydispersity, PD). This, in turn, influences the regenerated silk fibroin performance, including mechanical strength, water solubility etc.

Parameters were varied during the processing of raw silk cocoons into the silk solution. Varying these parameters affected the MW of the resulting silk solution. Parameters manipulated included (i) time and temperature of extraction, (ii) temperature of LiBr, (iii) temperature of dissolution oven, and (iv) dissolution time. Experiments were carried out to determine the effect of varying the extraction time. Tables 1-7 summarize the results. Below is a summary:
A sericin extraction time of 30 minutes resulted in larger molecular weight than a sericin extraction time of 60 minutes
Molecular weight decreases with time in the oven
140° C. LiBr and oven resulted in the low end of the confidence interval to be below a molecular weight of 9500 Da
30 min extraction at the 1 hour and 4 hour time points have undigested silk
30 min extraction at the 1 hour time point resulted in a significantly high molecular weight with the low end of the confidence interval being 35,000 Da
The range of molecular weight reached for the high end of the confidence interval was 18000 to 216000 Da (important for offering solutions with specified upper limit).

TABLE 1
The effect of extraction time (30 min vs 60 min) on molecular
weight of silk processed under the conditions of 100°
C. Extraction Temperature, 100° C. Lithium Bromide (LiBr)
and 100° C. Oven Dissolution (Oven/Dissolution Time was varied).

| Boil | Oven | Average | Std | Confidence | | |
|---|---|---|---|---|---|---|
| Time | Time | Mw | dev | Interval | | PD |
| 30 | 1 | 57247 | 12780 | 35093 | 93387 | 1.63 |
| 60 | 1 | 31520 | 1387 | 11633 | 85407 | 2.71 |
| 30 | 4 | 40973 | 2632 | 14268 | 117658 | 2.87 |
| 60 | 4 | 25082 | 1248 | 10520 | 59803 | 2.38 |
| 30 | 6 | 25604 | 1405 | 10252 | 63943 | 2.50 |
| 60 | 6 | 20980 | 1262 | 10073 | 43695 | 2.08 |

TABLE 2
The effect of extraction time (30 min vs 60 min) on
molecular weight of silk processed under the conditions
of 100° C. Extraction Temperature, boiling Lithium
Bromide (LiBr) and 60° C. Oven Dissolution for 4 hr.

| | Boil | Average | Std | Confidence | | |
|---|---|---|---|---|---|---|
| Sample | Time | Mw | dev | Interval | | PD |
| 30 min, 4 hr | 30 | 49656 | 4580 | 17306 | 142478 | 2.87 |
| 60 min, 4 hr | 60 | 30042 | 1536 | 11183 | 80705 | 2.69 |

TABLE 3
The effect of extraction time (30 min vs 60 min) on molecular
weight of silk processed under the conditions of 100°
C. Extraction Temperature, 60° C. Lithium Bromide (LiBr)
and 60° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 1 hr | 30 | 1 | 58436 | | 22201 | 153809 | 2.63 |
| 60 min, 1 hr | 60 | 1 | 31700 | | 11931 | 84224 | 2.66 |
| 30 min, 4 hr | 30 | 4 | 61956.5 | 13337 | 21463 | 178847 | 2.89 |
| 60 min, 4 hr | 60 | 4 | 25578.5 | 2446 | 9979 | 65564 | 2.56 |

TABLE 4
The effect of extraction time (30 min vs 60 min) on
molecular weight of silk processed under the conditions
of 100° C. Extraction Temperature, 80° C.
Lithium Bromide (LiBr) and 80° C. Oven Dissolution for 6 hr.

| | Boil | Average | Std | Confidence | | |
|---|---|---|---|---|---|---|
| Sample | Time | Mw | dev | Interval | | PD |
| 30 min, 6 hr | 30 | 63510 | | 18693 | 215775 | 3.40 |
| 60 min, 6 hr | 60 | 25164 | 238 | 9637 | 65706 | 2.61 |

TABLE 5
The effect of extraction time (30 min vs 60 min) on molecular
weight of silk processed under the conditions of 100°
C. Extraction Temperature, 80° C. Lithium Bromide (LiBr)
and 60° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 59202 | 14028 | 19073 | 183760 | 3.10 |
| 60 min, 4 hr | 60 | 4 | 26312.5 | 637 | 10266 | 67442 | 2.56 |
| 30 min, 6 hr | 30 | 6 | 46824 | | 18076 | 121293 | 2.59 |
| 60 min, 6 hr | 60 | 6 | 26353 | | 10168 | 68302 | 2.59 |

TABLE 6
The effect of extraction time (30 min vs 60 min) on molecular
weight of silk processed under the conditions of 100°
C. Extraction Temperature, 140° C. Lithium Bromide (LiBr)
and 140° C. Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 30 min, 4 hr | 30 | 4 | 9024.5 | 1102 | 4493 | 18127 | 2.00865 |
| 60 min, 4 hr | 60 | 4 | 15548 | | 6954 | 34762 | 2.2358 |
| 30 min, 6 hr | 30 | 6 | 13021 | | 5987 | 28319 | 2.1749 |
| 60 min, 6 hr | 60 | 6 | 10888 | | 5364 | 22100 | 2.0298 |

Experiments were carried out to determine the effect of varying the extraction temperature. Table 7 summarizes the results. Below is a summary:
Sericin extraction at 90° C. resulted in higher MW than sericin extraction at 100° C. extraction
Both 90° C. and 100° C. show decreasing MW over time in the oven.

TABLE 7
The effect of extraction temperature (90° C. vs.
100° C.) on molecular weight of silk processed
under the conditions of 60 min. Extraction Temperature,
100° C. Lithium Bromide (LiBr) and 100° C.
Oven Dissolution (Oven/Dissolution Time was varied).

| | Boil | Oven | Average | Std | Confidence | | |
|---|---|---|---|---|---|---|---|
| Sample | Time | Time | Mw | dev | Interval | | PD |
| 90° C., 4 hr | 60 | 4 | 37308 | 4204 | 13368 | 104119 | 2.79 |
| 100° C., 4 hr | 60 | 4 | 25082 | 1248 | 10520 | 59804 | 2.38 |
| 90° C., 6 hr | 60 | 6 | 34224 | 1135 | 12717 | 92100 | 2.69 |
| 100° C., 6 hr | 60 | 6 | 20980 | 1262 | 10073 | 43694 | 2.08 |

Experiments were carried out to determine the effect of varying the Lithium Bromide (LiBr) temperature when added to silk. Tables 8-9 summarize the results. Below is a summary:
No impact on molecular weight or confidence interval (all CI˜10500-6500 Da)
Studies illustrated that the temperature of LiBr-silk dissolution, as LiBr is added and begins dissolving, rapidly drops below the original LiBr temperature due to the majority of the mass being silk at room temperature

TABLE 8
The effect of Lithium Bromide (LiBr) temperature on molecular
weight of silk processed under the conditions of 60 min.
Extraction Time., 100° C. Extraction Temperature and
60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. | 60 | 1 | 31700 | | 11931 | 84223 | 2.66 |
| LiBr, | | | | | | | |
| 1 hr | | | | | | | |
| 100° C. | 100 | 1 | 27907 | 200 | 10735 | 72552 | 2.60 |
| LiBr, | | | | | | | |
| 1 hr | | | | | | | |
| RT LiBr, | RT | 4 | 29217 | 1082 | 10789 | 79119 | 2.71 |
| 4 hr | | | | | | | |
| 60° C. | 60 | 4 | 25578 | 2445 | 9978 | 65564 | 2.56 |
| LiBr, | | | | | | | |
| 4 hr | | | | | | | |
| 80° C. | 80 | 4 | 26312 | 637 | 10265 | 67441 | 2.56 |
| LiBr, | | | | | | | |
| 4 hr | | | | | | | |
| 100° C. | 100 | 4 | 27681 | 1729 | 11279 | 67931 | 2.45 |
| LiBr, | | | | | | | |
| 4 hr | | | | | | | |
| Boil LiBr, | Boil | 4 | 30042 | 1535 | 11183 | 80704 | 2.69 |
| 4 hr | | | | | | | |
| RT LiBr, | RT | 6 | 26543 | 1893 | 10783 | 65332 | 2.46 |
| 6 hr | | | | | | | |
| 80° C. | 80 | 6 | 26353 | | 10167 | 68301 | 2.59 |
| LiBr, | | | | | | | |
| 6 hr | | | | | | | |
| 100° C. | 100 | 6 | 27150 | 916 | 11020 | 66889 | 2.46 |
| LiBr, | | | | | | | |
| 6 hr | | | | | | | |

TABLE 9
The effect of Lithium Bromide (LiBr) temperature on molecular
weight of silk processed under the conditions of 30 min.
Extraction Time, 100° C. Extraction Temperature and
60° C. Oven Dissolution (Oven/Dissolution Time was varied).

| | LiBr | | | | | | |
|---|---|---|---|---|---|---|---|
| | Temp | Oven | Average | Std | Confidence | | |
| Sample | (° C.) | Time | Mw | dev | Interval | | PD |
| 60° C. | 60 | 4 | 61956 | 13336 | 21463 | 178847 | 2.89 |
| LiBr, | | | | | | | |
| 4 hr | | | | | | | |
| 80° C. | 80 | 4 | 59202 | 14027 | 19073 | 183760 | 3.10 |
| LiBr, | | | | | | | |
| 4 hr | | | | | | | |
| 100° C. | 100 | 4 | 47853 | | 19757 | 115899 | 2.42 |
| LiBr, | | | | | | | |
| 4 hr | | | | | | | |
| 80° C. | 80 | 6 | 46824 | | 18075 | 121292 | 2.59 |
| LiBr, | | | | | | | |
| 6 hr | | | | | | | |
| 100° C. | 100 | 6 | 55421 | 8991 | 19152 | 160366 | 2.89 |
| LiBr, | | | | | | | |
| 6 hr | | | | | | | |

Experiments were carried out to determine the effect of v oven/dissolution temperature. Tables 10-14 summarize the results. Below is a summary:
Oven temperature has less of an effect on 60 min extracted silk than 30 min extracted silk. Without wishing to be bound by theory, it is believed that the 30 min silk is less degraded during extraction and therefore the oven temperature has more of an effect on the larger MW, less degraded portion of the silk.
For 60° C. vs. 140° C. oven the 30 min extracted silk showed a very significant effect of lower MW at higher oven temp, while 60 min extracted silk had an effect but much less
The 140° C. oven resulted in a low end in the confidence interval at ˜6000 Da.

TABLE 10
The effect of oven/dissolution temperature on molecular weight
of silk processed under the conditions of 100° C. Extraction
Temperature, 30 min. Extraction Time, and 100° C. Lithium
Bromide (LiBr) (Oven/Dissolution Time was varied).

| Boil | Oven | Oven | Average | Std | Confidence | | |
|---|---|---|---|---|---|---|---|
| Time | Temp (° C.) | Time | Mw | dev | Interval | | PD |
| 30 | 60 | 4 | 47853 | | 19758 | 115900 | 2.42 |
| 30 | 100 | 4 | 40973 | 2632 | 14268 | 117658 | 2.87 |
| 30 | 60 | 6 | 55421 | 8992 | 19153 | 160366 | 2.89 |
| 30 | 100 | 6 | 25604 | 1405 | 10252 | 63943 | 2.50 |

TABLE 11
The effect of oven/dissolution temperature on molecular weight
of silk processed under the conditions of 100° C. Extraction
Temperature, 60 min. Extraction Time, and 100° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 60 | 60 | 1 | 27908 | 200 | 10735 | 72552 | 2.60 |
| 60 | 100 | 1 | 31520 | 1387 | 11633 | 85407 | 2.71 |
| 60 | 60 | 4 | 27681 | 1730 | 11279 | 72552 | 2.62 |
| 60 | 100 | 4 | 25082 | 1248 | 10520 | 59803 | 2.38 |
| 60 | 60 | 6 | 27150 | 916 | 11020 | 66889 | 2.46 |
| 60 | 100 | 6 | 20980 | 1262 | 10073 | 43695 | 2.08 |

TABLE 12
The effect of oven/dissolution temperature on molecular weight
of silk processed under the conditions of 100° C. Extraction
Temperature, 60 min. Extraction Time, and 140° C. Lithium
Bromide (LiBr) (Oven/Dissolution Time was varied).

| Boil | Oven | | | | | | |
|---|---|---|---|---|---|---|---|
| Time | Temp(° | Oven | Average | Std | Confidence | | |
| (minutes) | C.) | Time | Mw | dev | Interval | | PD |
| 60 | 60 | 4 | 30042 | 1536 | 11183 | 80705 | 2.69 |
| 60 | 140 | 4 | 15548 | | 7255 | 33322 | 2.14 |

TABLE 13
The effect of oven/dissolution temperature on molecular weight
of silk processed under the conditions of 100° C. Extraction
Temperature, 30 min. Extraction Time, and 140° C. Lithium
Bromide (LiBr) (Oven/Dissolution Time was varied).

| Boil | Oven | | | | | | |
|---|---|---|---|---|---|---|---|
| Time | Temp | Oven | Average | Std | Confidence | | |
| (minutes) | (° C.) | Time | Mw | dev | Interval | | PD |
| 30 | 60 | 4 | 49656 | 4580 | 17306 | 142478 | 2.87 |
| 30 | 140 | 4 | 9025 | 1102 | 4493 | 18127 | 2.01 |
| 30 | 60 | 6 | 59383 | 11640 | 17641 | 199889 | 3.37 |
| 30 | 140 | 6 | 13021 | | 5987 | 28319 | 2.17 |

TABLE 14
The effect of oven/dissolution temperature on molecular weight
of silk processed under the conditions of 100° C. Extraction
Temperature, 60 min. Extraction Time, and 80° C. Lithium
Bromide (LiBr) (Oven/Dissolution Time was varied).

| Boil | Oven | | | | | | |
|---|---|---|---|---|---|---|---|
| Time | Temp(° | Oven | Average | Std | Confidence | | |
| (minutes) | C.) | Time | Mw | dev | Interval | | PD |
| 60 | 60 | 4 | 26313 | 637 | 10266 | 67442 | 2.56 |
| 60 | 80 | 4 | 30308 | 4293 | 12279 | 74806 | 2.47 |
| 60 | 60 | 6 | 26353 | | 10168 | 68302 | 2.59 |
| 60 | 80 | 6 | 25164 | 238 | 9637 | 65706 | 2.61 |

The raw silk cocoons from the silkworm Bombyx mori was cut into pieces. The pieces of raw silk cocoons were boiled in an aqueous solution of Na₂CO₃ (about 100° C.) for a period of time between about 30 minutes to about 60 minutes to remove sericin (degumming). The volume of the water used equals about 0.4× raw silk weight and the amount of Na₂CO₃ is about 0.848× the weight of the raw silk cocoon pieces. The resulting degummed silk cocoon pieces were rinsed with deionized water three times at about 60° C. (20 minutes per rinse). The volume of rinse water for each cycle was 0.2 L× the weight of the raw silk cocoon pieces. The excess water from the degummed silk cocoon pieces was removed. After the DI water washing step, the wet degummed silk cocoon pieces were dried at room temperature. The degummed silk cocoon pieces were mixed with a LiBr solution, and the mixture was heated to about 100° C. The warmed mixture was placed in a dry oven and was heated at a temperature ranging from about 60° C. to about 140° C. for about 60 minutes to achieve complete dissolution of the native silk protein. The resulting solution was allowed to cool to room temperature and then was dialyzed to remove LiBr salts using a 3,500 Da MWCO membrane. Multiple exchanges were performed in Di water until Br⁻ ions were less than 1 ppm as determined in the hydrolyzed fibroin solution read on an Oakton Bromide (Br) double-junction ion-selective electrode.

The resulting silk fibroin aqueous solution has a concentration of about 8.0% w/v containing pure silk fibroin protein fragments having an average weight average molecular weight selected from between about 6 kDa to about 16 kDa, about 17 kDa to about 39 kDa, and about 39 kDa to about 80 kDa and a polydispersity of between about 1.5 and about 3.0. The 8.0% w/v was diluted with DI water to provide a 1.0% w/v, 2.0% w/v, 3.0% w/v, 4.0% w/v, 5.0% w/v by the coating solution.

A variety of % silk concentrations have been produced through the use of Tangential Flow Filtration (TFF). In all cases a 1% silk solution was used as the input feed. A range of 750-18,000 mL of 1% silk solution was used as the starting volume. Solution is diafiltered in the TFF to remove lithium bromide. Once below a specified level of residual LiBr, solution undergoes ultrafiltration to increase the concentration through removal of water. See examples below.

Six (6) silk solutions were utilized in standard silk structures with the following results:

Solution #1 is a silk concentration of 5.9 wt. %, average MW of 19.8 kDa and 2.2 PDI (made with a 60 min boil extraction, 100° C. LiBr dissolution for 1 hour).

Solution #2 is a silk concentration of 6.4 wt. % (made with a 30 min boil extraction, 60° C. LiBr dissolution for 4 hrs).

Solution #3 is a silk concentration of 6.17 wt. % (made with a 30 min boil extraction 100° C. LiBr dissolution for 1 hour).

Solution #4 is a silk concentration of 7.30 wt. %: A 7.30% silk solution was produced beginning with 30 minute extraction batches of 100 g silk cocoons per batch. Extracted silk fibers were then dissolved using 100° C. 9.3 M LiBr in a 100° C. oven for 1 hour. 100 g of silk fibers were dissolved per batch to create 20% silk in LiBr. Dissolved silk in LiBr was then diluted to 1% silk and filtered through a 5 μm filter to remove large debris. 15,500 mL of 1%, filtered silk solution was used as the starting volume/diafiltration volume for TFF. Once LiBr was removed, the solution was ultrafiltered to a volume around 1300 mL. 1262 mL of 7.30% silk was then collected. Water was added to the feed to help remove the remaining solution and 547 mL of 3.91% silk was then collected.

Solution #5 is a silk concentration of 6.44 wt. %: A 6.44 wt. % silk solution was produced beginning with 60 minute extraction batches of a mix of 25, 33, 50, 75 and 100 g silk cocoons per batch. Extracted silk fibers were then dissolved using 100° C. 9.3 M LiBr in a 100° C. oven for 1 hour. 35, 42, 50 and 71 g per batch of silk fibers were dissolved to create 20% silk in LiBr and combined. Dissolved silk in LiBr was then diluted to 1% silk and filtered through a 5 μm filter to remove large debris. 17,000 mL of 1%, filtered silk solution was used as the starting volume/diafiltration volume for TFF. Once LiBr was removed, the solution was ultrafiltered to a volume around 3000 mL. 1490 mL of 6.44% silk was then collected. Water was added to the feed to help remove the remaining solution and 1454 mL of 4.88% silk was then collected.

Solution #6 is a silk concentration of 2.70 wt. %: A 2.70% silk solution was produced beginning with 60-minute extraction batches of 25 g silk cocoons per batch. Extracted silk fibers were then dissolved using 100° C. 9.3 M LiBr in a 100° C. oven for 1 hour. 35.48 g of silk fibers were dissolved per batch to create 20% silk in LiBr. Dissolved silk in LiBr was then diluted to 1% silk and filtered through a 5 μm filter to remove large debris. 1000 mL of 1%, filtered silk solution was used as the starting volume/diafiltration volume for TFF. Once LiBr was removed, the solution was ultrafiltered to a volume around 300 mL. 312 mL of 2.7% silk was then collected.

The preparation of silk fibroin solutions with higher molecular weights is given in Table 15.

TABLE 15
Preparation and properties of silk fibroin solutions.

| | | | | | Average | |
|---|---|---|---|---|---|---|
| | | | | | weight | |
| | | | | | average | |
| | Extraction | Extraction | LiBr | Oven/ | molecular | Average |
| Sample | Time | Temp | Temp | Sol'n | weight | polydis- |
| Name | (mins) | (° C.) | (° C.) | Temp | (kDa) | persity |
| Group A | 60 | 100 | 100 | 100° C. | 34.7 | 2.94 |
| TFF | | | | oven | | |
| Group A | 60 | 100 | 100 | 100° C. | 44.7 | 3.17 |
| DIS | | | | oven | | |
| Group B | 60 | 100 | 100 | 100° C. | 41.6 | 3.07 |
| TFF | | | | sol'n | | |
| Group B | 60 | 100 | 100 | 100° C. | 44.0 | 3.12 |
| DIS | | | | sol'n | | |
| Group D | 30 | 90 | 60 | 60° C. | 129.7 | 2.56 |
| DIS | | | | sol'n | | |
| Group D | 30 | 90 | 60 | 60° C. | 144.2 | 2.73 |
| FIL | | | | sol'n | | |
| Group E | 15 | 100 | RT | 60° C. | 108.8 | 2.78 |
| DIS | | | | sol'n | | |
| Group E | 15 | 100 | RT | 60° C. | 94.8 | 2.62 |
| FIL | | | | sol'n | | |

Silk aqueous coating composition for application to fabrics are given in Tables 16 and 17 below.

TABLE 16
Silk Solution Characteristics

| | | | | | |
|---|---|---|---|---|---|
| | Molecular Weight: | 57 kDa | | | |
| | Polydispersity: | 1.6 | | | |
| | % Silk | 5.0% | 3.0% | 1.0% | 0.5% |
| Process | | | | | |
| Parameters | | | | | |
| | Extraction | | | | |
| | Boil Time: | 30 minutes | | | |
| | Boil Temperature: | 100° C. | | | |
| | Rinse Temperature: | 60° C. | | | |
| | Dissolution | | | | |
| | LiBr Temperature: | 100 | | | |
| | Oven Temperature: | 100° C. | | | |
| | Oven Time: | 60 minutes | | | |

TABLE 17
Silk Solution Characteristics

| | | | | | |
|---|---|---|---|---|---|
| | Molecular Weight: | 25 kDa | | | |
| | Polydispersity: | 2.4 | | | |
| | % Silk | 5.0% | 3.0% | 1.0% | 0.5% |
| Process | | | | | |
| Parameters | | | | | |
| | Extraction | | | | |
| | Boil Time: | 60 minutes | | | |
| | Boil Temperature: | 100° C. | | | |
| | Rinse Temperature: | 60° C. | | | |
| | Dissolution | | | | |
| | LiBr Temperature: | 100° C. | | | |
| | Oven Temperature: | 100° C. | | | |
| | Oven Time: | 60 minutes | | | |

Three (3) silk solutions were utilized in film making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

Films were made in accordance with Rockwood et al. (Nature Protocols; Vol. 6; No. 10; published on-line Sep. 22, 2011; doi:10.1038/nprot.2011.379). 4 mL of 1% or 2% (wt/vol) aqueous silk solution was added into 100 mm Petri dish (Volume of silk can be varied for thicker or thinner films and is not critical) and allowed to dry overnight uncovered. The bottom of a vacuum desiccator was filled with water. Dry films were placed in the desiccator and vacuum applied, allowing the films to water anneal for 4 hours prior to removal from the dish. Films cast from solution #1 did not result in a structurally continuous film; the film was cracked in several pieces. These pieces of film dissolved in water in spite of the water annealing treatment.

Silk solutions of various molecular weights and/or combinations of molecular weights can be optimized for gel applications. The following provides an example of this process but it not intended to be limiting in application or formulation. Three (3) silk solutions were utilized in gel making with the following results:

Solution #1 is a silk concentration of 5.9%, average MW of 19.8 kDa and 2.2 PD (made with a 60 min boil extraction, 100° C. LiBr dissolution for 1 hr).

Solution #2 is a silk concentration of 6.4% (made with a 30 min boil extraction, 60° C. LiBr dissolution for 4 hrs).

Solution #3 is a silk concentration of 6.17% (made with a 30 min boil extraction, 100° C. LiBr dissolution for 1 hour).

“Egel” is an electrogelation process as described in Rockwood of al. Briefly, 10 ml of aqueous silk solution is added to a 50 ml conical tube and a pair of platinum wire electrodes immersed into the silk solution. A 20 volt potential was applied to the platinum electrodes for 5 minutes, the power supply turned off and the gel collected. Solution #1 did not form an EGEL over the 5 minutes of applied electric current.

Solutions #2 and #3 were gelled in accordance with the published horseradish peroxidase (HRP) protocol. Behavior seemed typical of published solutions.

Materials and Methods: the following equipment and material are used in determination of Silk Molecular weight: Agilent 1100 with chemstation software ver. 10.01; Refractive Index Detector (RID); analytical balance; volumetric flasks (1000 mL, 10 mL and 5 mL); HPLC grade water; ACS grade sodium chloride; ACS grade sodium phosphate dibasic heptahydrate; phosphoric acid; dextran MW Standards-Nominal Molecular Weights of 5 kDa, 11.6 kDa, 23.8 kDa, 48.6 kDa, and 148 kDa; 50 mL PET or polypropylene disposable centrifuge tubes; graduated pipettes; amber glass HPLC vials with Teflon caps; Phenomenex PolySep GFC P-4000 column (size: 7.8 mm×300 mm).

### Procedural Steps:

A) Preparation of 1 L Mobile Phase (0.1 M Sodium Chloride solution in 0.0125 M Sodium phosphate buffer)

Take a 250 mL clean and dry beaker, place it on the balance and tare the weight. Add about 3.3509 g of sodium phosphate dibasic heptahydrate to the beaker. Note down the exact weight of sodium phosphate dibasic weighed. Dissolve the weighed sodium phosphate by adding 100 mL of HPLC water into the beaker. Take care not to spill any of the content of the beaker. Transfer the solution carefully into a clean and dry 1000 mL volumetric flask. Rinse the beaker and transfer the rinse into the volumetric flask. Repeat the rinse 4-5 times. In a separate clean and dry 250 mL beaker weigh exactly about 5.8440 g of sodium chloride. Dissolve the weighed sodium chloride in 50 mL of water and transfer the solution to the sodium phosphate solution in the volumetric flask. Rinse the beaker and transfer the rinse into the volumetric flask. Adjust the pH of the solution to 7.0±0.2 with phosphoric acid. Make up the volume in volumetric flask with HPLC water to 1000 mL and shake it vigorously to homogeneously mix the solution. Filter the solution through 0.45 μm polyamide membrane filter. Transfer the solution to a clean and dry solvent bottle and label the bottle. The volume of the solution can be varied to the requirement by correspondingly varying the amount of sodium phosphate dibasic heptahydrate and sodium chloride.
B) Preparation of Dextran Molecular Weight Standard solutions

At least five different molecular weight standards are used for each batch of samples that are run so that the expected value of the sample to be tested is bracketed by the value of the standard used. Label six 20 mL scintillation glass vials respective to the molecular weight standards. Weigh accurately about 5 mg of each of dextran molecular weight standards and record the weights. Dissolve the dextran molecular weight standards in 5 mL of mobile phase to make a 1 mg/mL standard solution.
C) Preparation of Sample solutions

When preparing sample solutions, if there are limitations on how much sample is available, the preparations may be scaled as long as the ratios are maintained. Depending on sample type and silk protein content in sample weigh enough sample in a 50 mL disposable centrifuge tube on an analytical balance to make a 1 mg/mL sample solution for analysis. Dissolve the sample in equivalent volume of mobile phase make a 1 mg/mL solution. Tightly cap the tubes and mix the samples (in solution). Leave the sample solution for 30 minutes at room temperature. Gently mix the sample solution again for 1 minute and centrifuge at 4000 RPM for 10 minutes.
D) HPLC analysis of the samples

Transfer 1.0 mL of all the standards and sample solutions into individual HPLC vials. Inject the molecular weight standards (one injection each) and each sample in duplicate. Analyze all the standards and sample solutions using the following HPLC conditions:

| | |
|---|---|
| Column | PolySep GFC P-4000 (7.8 × 300 mm) |
| Column Temperature | 25° C. |
| Detector | Refractive Index Detector |
| | (Temperature @ 35° C.) |
| Injection Volume | 25.0 μL |
| Mobile Phase | 0.1M Sodium Chloride solution in |
| | 0.0125M sodium phosphate |
| | buffer |
| Flow Rate | 1.0 mL/min |
| Run Time | 20.0 min |

E) Data analysis and calculations—Calculation of Average Molecular Weight using Cirrus Software

Upload the chromatography data files of the standards and the analytical samples into Cirrus SEC data collection and molecular weight analysis software. Calculate the weight average molecular weight (Mw), number average molecular weight (Mn), peak average molecular weight (Mp), and polydispersity for each injection of the sample.

### Spider Silk Fragments

Spider silks are natural polymers that consist of three domains: a repetitive middle core domain that dominates the protein chain, and non-repetitive N-terminal and C-terminal domains. The large core domain is organized in a block copolymer-like arrangement, in which two basic sequences, crystalline [poly(A) or poly(GA)] and less crystalline (GGX or GPGXX) polypeptides alternate. Dragline silk is the protein complex composed of major ampullate dragline silk protein 1 (MaSp1) and major ampullate dragline silk protein 2 (MaSp2). Both silks are approximately 3500 amino acid long. MaSp1 can be found in the fibre core and the periphery, whereas MaSp2 forms clusters in certain core areas. The large central domains of MaSp1 and MaSp2 are organized in block copolymer-like arrangements, in which two basic sequences, crystalline [poly(A) or poly(GA)] and less crystalline (GGX or GPGXX) polypeptides alternate in core domain. Specific secondary structures have been assigned to poly(A)/(GA), GGX and GPGXX motifs including β-sheet, α-helix and β-spiral respectively. The primary sequence, composition and secondary structural elements of the repetitive core domain are responsible for mechanical properties of spider silks; whereas, non-repetitive N- and C-terminal domains are essential for the storage of liquid silk dope in a lumen and fiber formation in a spinning duct.

The main difference between MaSp1 and MaSp2 is the presence of proline (P) residues accounting for 15% of the total amino acid content in MaSp2, whereas MaSp1 is proline-free. By calculating the number of proline residues in N. clavipes dragline silk, it is possible to estimate the presence of the two proteins in fibers; 81% MaSp1 and 19% MaSp2. Different spiders have different ratios of MaSp1 and MaSp2. For example, a dragline silk fiber from the orb weaver Argiope aurantia contains 41% MaSp1 and 59% MaSp2. Such changes in the ratios of major ampullate silks can dictate the performance of the silk fiber.

At least seven different types of silk proteins are known for one orb-weaver species of spider. Silks differ in primary sequence, physical properties and functions. For example, dragline silks used to build frames, radii and lifelines are known for outstanding mechanical properties including strength, toughness and elasticity. On an equal weight basis, spider silk has a higher toughness than steel and Kevlar. Flageliform silk found in capture spirals has extensibility of up to 500%. Minor ampullate silk, which is found in auxiliary spirals of the orb-web and in prey wrapping, possesses high toughness and strength almost similar to major ampullate silks, but does not supercontract in water.

Spider silks are known for their high tensile strength and toughness. The recombinant silk proteins also confer advantageous properties to cosmetic or dermatological compositions, in particular to be able to improve the hydrating or softening action, good film forming property and low surface density. Diverse and unique biomechanical properties together with biocompatibility and a slow rate of degradation make spider silks excellent candidates as biomaterials for tissue engineering, guided tissue repair and drug delivery, for cosmetic products (e.g., nail and hair strengthener, skin care products), and industrial materials (e.g. nanowires, nanofibers, surface coatings).

In an embodiment, a silk protein may include a polypeptide derived from natural spider silk proteins. The polypeptide is not limited particularly as long as it is derived from natural spider silk proteins, and examples of the polypeptide include natural spider silk proteins and recombinant spider silk proteins such as variants, analogs, derivatives or the like of the natural spider silk proteins. In terms of excellent tenacity, the polypeptide may be derived from major dragline silk proteins produced in major ampullate glands of spiders. Examples of the major dragline silk proteins include major ampullate spidroin MaSp1 and MaSp2 from Nephila clavipes, and ADF3 and ADF4 from Araneus diadematus, etc. Examples of the polypeptide derived from major dragline silk proteins include variants, analogs, derivatives or the like of the major dragline silk proteins. Further, the polypeptide may be derived from flagelliform silk proteins produced in flagelliform glands of spiders. Examples of the flagelliform silk proteins include flagelliform silk proteins derived from Nephila clavipes, etc.

Examples of the polypeptide derived from major dragline silk proteins include a polypeptide containing two or more units of an amino acid sequence represented by the formula 1: REP1-REP2 (1), preferably a polypeptide containing five or more units thereof, and more preferably a polypeptide containing ten or more units thereof. Alternatively, the polypeptide derived from major dragline silk proteins may be a polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453. In the polypeptide derived from major dragline silk proteins, units of the amino acid sequence represented by the formula 1: REP1-REP2 (1) may be the same or may be different from each other. In the case of producing a recombinant protein using a microbe such as Escherichia coli as a host, the molecular weight of the polypeptide derived from major dragline silk proteins is 500 kDa or less, or 300 kDa or less, or 200 kDa or less, in terms of productivity.

In the formula (1), the REP1 indicates polyalanine. In the REP1, the number of alanine residues arranged in succession is preferably 2 or more, more preferably 3 or more, further preferably 4 or more, and particularly preferably 5 or more. Further, in the REPI, the number of alanine residues arranged in succession is preferably 20 or less, more preferably 16 or less, further preferably 12 or less, and particularly preferably 10 or less. In the formula (1), the REP2 is an amino acid sequence composed of 10 to 200 amino acid residues. The total number of glycine, serine, glutamine and alanine residues contained in the amino acid sequence is 40% or more, preferably 60% or more, and more preferably 70% or more with respect to the total number of amino acid residues contained therein.

In the major dragline silk, the REP1 corresponds to a crystal region in a fiber where a crystal β sheet is formed, and the REP2 corresponds to an amorphous region in a fiber where most of the parts lack regular configurations and that has more flexibility. Further, the [REP1-REP2] corresponds to a repetitious region (repetitive sequence) composed of the crystal region and the amorphous region, which is a characteristic sequence of dragline silk proteins.

### Recombinant Silk Fragments

In some embodiments, the recombinant silk protein refers to recombinant spider silk polypeptides, recombinant insect silk polypeptides, or recombinant mussel silk polypeptides. In some embodiments, the recombinant silk protein fragment disclosed herein include recombinant spider silk polypeptides of Araneidae or Araneoids, or recombinant insect silk polypeptides of Bombyx mori. In some embodiments, the recombinant silk protein fragment disclosed herein include recombinant spider silk polypeptides of Araneidae or Araneoids. In some embodiments, the recombinant silk protein fragment disclosed herein include block copolymer having repetitive units derived from natural spider silk polypeptides of Araneidae or Araneoids. In some embodiments, the recombinant silk protein fragment disclosed herein include block copolymer having synthetic repetitive units derived from spider silk polypeptides of Araneidae or Araneoids and non-repetitive units derived from natural repetitive units of spider silk polypeptides of Araneidae or Araneoids.

Recent advances in genetic engineering have provided a route to produce various types of recombinant silk proteins. Recombinant DNA technology has been used to provide a more practical source of silk proteins. As used herein “recombinant silk protein” refers to synthetic proteins produced heterologously in prokaryotic or eukaryotic expression systems using genetic engineering methods.

Various methods for synthesizing recombinant silk peptides are known and have been described by Ausubel et al., Current Protocols in Molecular Biology § 8 (John Wiley & Sons 1987, (1990)), incorporated herein by reference. A gram-negative, rod-shaped bacterium E. coli is a well-established host for industrial scale production of proteins. Therefore, the majority of recombinant silks have been produced in E. coli. E. coli which is easy to manipulate, has a short generation time, is relatively low cost and can be scaled up for larger amounts protein production.

The recombinant silk proteins can be produced by transformed prokaryotic or eukaryotic systems containing the cDNA coding for a silk protein, for a fragment of this protein or for an analog of such a protein. The recombinant DNA approach enables the production of recombinant silks with programmed sequences, secondary structures, architectures and precise molecular weight. There are four main steps in the process: (i) design and assembly of synthetic silk-like genes into genetic ‘cassettes’, (ii) insertion of this segment into a DNA recombinant vector, (iii) transformation of this recombinant DNA molecule into a host cell and (iv) expression and purification of the selected clones.

The term “recombinant vectors”, as used herein, includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, or plant) or in in vitro expression systems. Cloning vectors are generally used to engineer and amplify a certain desired DNA fragment and may lack functional sequences needed for expression of the desired DNA fragments.

The prokaryotic systems include Gram-negative bacteria or Gram-positive bacteria. The prokaryotic expression vectors can include an origin of replication which can be recognized by the host organism, a homologous or heterologous promoter which is functional in the said host, the DNA sequence coding for the spider silk protein, for a fragment of this protein or for an analogous protein. Nonlimiting examples of prokaryotic expression organisms are Escherichia coli, Bacillus subtilis, Bacillus megaterium, Corynebacterium glutamicum, Anabaena, Caulobacter, Gluconobacter, Rhodobacter, Pseudomonas, Para coccus, Bacillus (e.g. Bacillus subtilis) Brevibacterium, Corynebacterium, Rhizobium (Sinorhizobium), Flavobacterium, Klebsiella, Enterobacter, Lactobacillus, Lactococcus, Methylobacterium, Propionibacterium, Staphylococcus or Streptomyces cells.

The eukaryotic systems include yeasts and insect, mammalian or plant cells. In this case, the expression vectors can include a yeast plasmid origin of replication or an autonomous replication sequence, a promoter, a DNA sequence coding for a spider silk protein, for a fragment or for an analogous protein, a polyadenylation sequence, a transcription termination site and, lastly, a selection gene. Nonlimiting examples of eukaryotic expression organisms include yeasts, such as Saccharomyces cerevisiae, Pichia pastoris, basidiosporogenous, ascosporogenous, filamentous fungi, such as Aspergillus niger, Aspergillus oryzae, Aspergillus nidulans, Trichoderma reesei, Acremonium chrysogenum, Candida, Hansenula, Kluyveromyces, Saccharomyces (e.g. Saccharomyces cerevisiae), Schizosaccharomyces, Pichia (e.g. Pichia pastoris) or Yarrowia cells etc., mammalian cells, such as HeLa cells, COS cells, CHO cells etc., insect cells, such as Sf9 cells, MEL cells, etc., “insect host cells” such as Spodoptera frugiperda or Trichoplusia ni cells. SF9 cells, SF-21 cells or High-Five cells, wherein SF-9 and SF-21 are ovarian cells from Spodoptera frugiperda, and High-Five cells are egg cells from Trichoplusia ni., “plant host cells”, such as tobacco, potato or pea cells.

A variety of heterologous host systems have been explored to produce different types of recombinant silks. Recombinant partial spidroins as well as engineered silks have been cloned and expressed in bacteria (Escherichia coli), yeast (Pichia pastoris), insects (silkworm larvae), plants (tobacco, soybean, potato, Arabidopsis), mammalian cell lines (BHT/hamster) and transgenic animals (mice, goats). Most of the silk proteins are produced with an N- or C-terminal His-tags to make purification simple and produce enough amounts of the protein.

In some embodiments, the host suitable for expressing the recombinant spider silk protein using heterogeneous system may include transgenic animals and plants. In some embodiments, the host suitable for expressing the recombinant spider silk protein using heterogeneous system comprises bacteria, yeasts, mammalian cell lines. In some embodiments, the host suitable for expressing the recombinant spider silk protein using heterogeneous system comprises E. coli. In some embodiments, the host suitable for expressing the recombinant spider silk protein using heterogeneous system comprises transgenic B. mori silkworm generated using genome editing technologies (e.g. CRISPR).

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences.

In some embodiments, “recombinant silk protein” refers to recombinant silkworm silk protein or fragments thereof. The recombinant production of silk fibroin and silk sericin has been reported. A variety of hosts are used for the production including E. coli, Saccharomyces cerevisiae, Pseudomonas sp., Rhodopseudomonas sp., Bacillus sp., and Strepomyces. See EP 0230702, which is incorporate by reference herein by its entirety.

Provided herein also include design and biological-synthesis of silk fibroin protein-like multiblock polymer comprising GAGAGX hexapeptide (X is A, Y, V or S) derived from the repetitive domain of B. mori silk heavy chain (H chain) In some embodiments, this disclosure provides silk protein-like multiblock polymers derived from the repetitive domain of B. mori silk heavy chain (H chain) comprising the GAGAGS hexapeptide repeating units. The GAGAGS hexapeptide is the core unit of H-chain and plays an important role in the formation of crystalline domains. The silk protein-like multiblock polymers containing the GAGAGS hexapeptide repeating units spontaneously aggregate into β-sheet structures, similar to natural silk fibroin protein, where in the silk protein-like multiblock polymers having any weight average molecular weight described herein.

In some embodiments, this disclosure provides silk-peptide like multiblock copolymers composed of the GAGAGS hexapeptide repetitive fragment derived from H chain of B. mori silk heavy chain and mammalian elastin VPGVG motif produced by E. coli. In some embodiments, this disclosure provides fusion silk fibroin proteins composed of the GAGAGS hexapeptide repetitive fragment derived from H chain of B. mori silk heavy chain and GVGVP produced by E. coli, where in the silk protein-like multiblock polymers having any weight average molecular weight described herein.

In some embodiments, this disclosure provides B. mori silkworm recombinant proteins composed of the (GAGAGS)₁₆ repetitive fragment. In some embodiments, this disclosure provides recombinant proteins composed of the (GAGAGS)₁₆ repetitive fragment and the non-repetitive (GAGAGS)₁₆—F—COOH, (GAGAGS)₁₆—F—F—COOH, (GAGAGS)₁₆—F—F—F—COOH, (GAGAGS)₁₆—F—F—F—F—COOH, (GAGAGS)₁₆—F—F—F—F—F—F—F—F—COOH, (GAGAGS)₁₆—F—F—F—F—F—F—F—F—F—F—F—F—COOH produced by E. coli, where F has the following amino acid sequence SGFGPVANGGSGEASSESDFGSSGFGPVANASSGEASSESDFAG, and where in the silk protein-like multiblock polymers having any weight average molecular weight described herein.

In some embodiments, “recombinant silk protein” refers to recombinant spider silk protein or fragments thereof. The productions of recombinant spider silk proteins based on a partial cDNA clone have been reported. The recombinant spider silk proteins produced as such comprise a portion of the repetitive sequence derived from a dragline spider silk protein, Spidroin 1, from the spider Nephila clavipes. see Xu et al. (Proc. Natl. Acad. Sci. U.S.A., 87:7120-7124 (1990). cDNA clone encoding a portion of the repeating sequence of a second fibroin protein, Spidroin 2, from dragline silk of Nephila clavipes and the recombinant synthesis thereof is described in J. Biol. Chem., 1992, volume 267, pp. 19320-19324. The recombinant synthesis of spider silk proteins including protein fragments and variants of Nephila clavipes from transformed E. coli is described in U.S. Pat. Nos. 5,728,810 and 5,989,894. cDNA clones encoding minor ampullate spider silk proteins and the expression thereof is described in U.S. Pat. Nos. 5,733,771 and 5,756,677. cDNA clone encoding the flagelliform silk protein from an orb-web spinning spider is described in U.S. Pat. No. 5,994,099. U.S. Pat. No. 6,268,169 describes the recombinant synthesis of spider silk like proteins derived from the repeating peptide sequence found in the natural spider dragline of Nephila clavipes by E. coli, Bacillus subtilis, and Pichia pastoris recombinant expression systems. WO 03/020916 describes the cDNA clone encoding and recombinant production of spider spider silk proteins having repeative sequences derived from the major ampullate glands of Nephila madagascariensis, Nephila senegalensis, Tetragnatha kauaiensis, Tetragnatha versicolor, Argiope aurantia, Argiope trifasciata, Gasteracantha mammosa, and Latrodectus geometricus, the flagelliform glands of Argiope trifasciata, the ampullate glands of Dolomedes tenebrosus, two sets of silk glands from Plectreurys tristis, and the silk glands of the mygalomorph Euagrus chisoseus. Each of the above reference is incorporated herein by reference in its entirety.

In some embodiments, the recombinant spider silk protein is a hybrid protein of a spider silk protein and an insect silk protein, a spider silk protein and collagen, a spider silk protein and resilin, or a spider silk protein and keratin. The spider silk repetitive unit comprises or consists of an amino acid sequence of a region that comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide.

In some embodiments, the recombinant spider silk protein in this disclosure comprises synthetic spider silk proteins derived from repetitive units of natural spider silk proteins, consensus sequence, and optionally one or more natural non-repetitive spider silk protein sequences. The repeated units of natural spider silk polypeptide may include dragline spider silk polypeptides or flagelliform spider silk polypeptides of Araneidae or Araneoids.

As used herein, the spider silk “repetitive unit” comprises or consists of at least one peptide motif that repetitively occurs within a naturally occurring major ampullate gland polypeptide, such as a dragline spider silk polypeptide, a minor ampullate gland polypeptide, a flagelliform polypeptide, an aggregate spider silk polypeptide, an aciniform spider silk polypeptide or a pyriform spider silk polypeptide. A “repetitive unit” refers to a region which corresponds in amino acid sequence to a region that comprises or consists of at least one peptide motif (e.g. AAAAAA) or GPGQQ) that repetitively occurs within a naturally occurring silk polypeptide (e.g. MaSpI, ADF-3, ADF-4, or Flag) (i.e. identical amino acid sequence) or to an amino acid sequence substantially similar thereto (i.e. variational amino acid sequence). A “repetitive unit” having an amino acid sequence which is “substantially similar” to a corresponding amino acid sequence within a naturally occurring silk polypeptide (i.e. wild-type repetitive unit) is also similar with respect to its properties, e.g. a silk protein comprising the “substantially similar repetitive unit” is still insoluble and retains its insolubility. A “repetitive unit” having an amino acid sequence which is “identical” to the amino acid sequence of a naturally occurring silk polypeptide, for example, can be a portion of a silk polypeptide corresponding to one or more peptide motifs of MaSpI, MaSpII, ADF-3 and/or ADF-4. A “repetitive unit” having an amino acid sequence which is “substantially similar” to the amino acid sequence of a naturally occurring silk polypeptide, for example, can be a portion of a silk polypeptide corresponding to one or more peptide motifs of MaSpI, MaSpII, ADF-3 and/or ADF-4, but having one or more amino acid substitution at specific amino acid positions.

As used herein, the term “consensus peptide sequence” refers to an amino acid sequence which contains amino acids which frequently occur in a certain position (e.g., “G”) and wherein, other amino acids which are not further determined are replaced by the place holder “X”. In some embodiments, the consensus sequence is at least one of (i) GPGXX, wherein X is an amino acid selected from A, S, G, Y, P and Q; (ii) GGX, wherein X is an amino acid selected from Y, P, R, S, A, T, N and Q, preferably Y, P and Q; (iii) Aₓ, wherein x is an integer from 5 to 10.

The consensus peptide sequences GPGXX and GGX, i.e., glycine rich motifs, provide flexibility to the silk polypeptide and thus, to the thread formed from the silk protein containing said motifs. In detail, the iterated GPGXX motif forms turn spiral structures, which imparts elasticity to the silk polypeptide. Major ampullate and flagelliform silks both have a GPGXX motif. The iterated GGX motif is associated with a helical structure having three amino acids per turn and is found in most spider silks. The GGX motif may provide additional elastic properties to the silk. The iterated polyalanine Ax (peptide) motif forms a crystalline β-sheet structure that provides strength to the silk polypeptide, as described for example in WO 03/057727.

In some embodiments, the recombinant spider silk protein in this disclosure comprises two identical repetitive units each comprising at least one, preferably one, amino acid sequence selected from the group consisting of: GGRPSDTYG and GGRPSSSYG derived from Resilin. Resilin is an elastomeric protein found in most arthropods that provides low stiffness and high strength.

As used herein, “non-repetitive units” refers to an amino acid sequence which is “substantially similar” to a corresponding non-repetitive (carboxy terminal) amino acid sequence within a naturally occurring dragline polypeptide (i.e. wild-type non-repetitive (carboxy terminal) unit), preferably within ADF-3 (SEQ ID NO:1), ADF-4 (SEQ ID NO:2), NR3 (SEQ ID NO:41), NR4 (SEQ ID NO:42), ADF-4 of the spider Araneus diadematus as described in U.S. Pat. No. 8,367,803, C16 peptide (spider silk protein eADF4, molecular weight of 47.7 kDa, AMSilk) comprising the 16 repeats of the sequence GSSAAAAAAAASGPGGYGPENQGPSGPGGYGPGGP, an amino acid sequence adapted from the natural sequence of ADF4 from A. diadematus. Non-repetitive ADF-4 and variants thereof display efficient assembly behavior.

Among the synthetic spider silk proteins, the recombinant silk protein in this disclosure comprises in some embodiments the C16-protein having the polypeptide sequence SEQ ID NO: 1 as described in U.S. Pat. No. 8,288,512. Besides the polypeptide sequence shown in SEQ ID NO:1, particularly functional equivalents, functional derivatives and salts of this sequence are also included.

As used herein, “functional equivalents” refers to mutant which, in at least one sequence position of the abovementioned amino acid sequences, have an amino acid other than that specifically mentioned.

In some embodiments, the recombinant spider silk protein in this disclosure comprises, in an effective amount, at least one natural or recombinant silk protein including spider silk protein, corresponding to Spidroin major 1 described by Xu et al., PNAS, USA, 87, 7120, (1990), Spidroin major 2 described by Hinman and Lewis, J. Biol. Chem., 267, 19320, (1922), recombinant spider silk protein as described in U.S. Patent Application No. 2016/0222174 and U.S. Pat. Nos. 9,051,453, 9,617,315, 9,689,089, 8,173,772, 8,642,734, 8,367,803 8,097,583, 8,030,024, 7,754,851, 7,148,039, 7,060,260, or alternatively the minor Spidroins described in patent application WO 95/25165. Each of the above-cited references is incorporated herein by reference in its entirety. Additional recombinant spider silk proteins suitable for the recombinant RSPF of this disclosure include ADF3 and ADF4 from the “Major Ampullate” gland of Araneus diadematus.

Recombinant silk is also described in other patents and patent applications, incorporated by reference herein: US 2004590196, U.S. Pat. No. 7,754,851, US 2007654470, U.S. Pat. No. 7,951,908, US 2010785960, U.S. Pat. No. 8,034,897, US 20090263430, US 2008226854, US 20090123967, US 2005712095, US 2007991037, US 20090162896, US 200885266, U.S. Pat. No. 8,372,436, US 2007989907, US 2009267596, US 2010319542, US 2009265344, US 2012684607, US 2004583227, U.S. Pat. No. 8,030,024, US 2006643569, U.S. Pat. No. 7,868,146, US 2007991916, U.S. Pat. No. 8,097,583, US 2006643200, U.S. Pat. Nos. 8,729,238, 8,877,903, US 20190062557, US 20160280960, US 20110201783, US 2008991916, US 2011986662, US 2012697729, US 20150328363, U.S. Pat. No. 9,034,816, US 20130172478, U.S. Pat. No. 9,217,017, US 20170202995, U.S. Pat. No. 8,721,991, US 2008227498, U.S. Pat. Nos. 9,233,067, 8,288,512, US 2008161364, U.S. Pat. No. 7,148,039, U.S. Ser. No. 19/992,47806, US 2001861597, US 2004887100, U.S. Pat. Nos. 9,481,719, 8,765,688, US 200880705, US 2010809102, U.S. Pat. No. 8,367,803, US 2010664902, U.S. Pat. No. 7,569,660, U.S. Ser. No. 19/991,38833, US 2000591632, US 20120065126, US 20100278882, US 2008161352, US 20100015070, US 2009513709, US 20090194317, US 2004559286, US 200589551, US 2008187824, US 20050266242, US 20050227322, and US 20044418.

Recombinant silk is also described in other patents and patent applications, incorporated by reference herein: US 20190062557, US 20150284565, US 20130225476, US 20130172478, US 20130136779, US 20130109762, US 20120252294, US 20110230911, US 20110201783, US 20100298877, U.S. Pat. Nos. 10,478,520, 10,253,213, 10,072,152, 9,233,067, 9,217,017, 9,034,816, 8,877,903, 8,729,238, 8,721,991, 8,097,583, 8,034,897, 8,030,024, 7,951,908, 7,868,146, and 7,754,851.

In some embodiments, the recombinant spider silk protein in this disclosure comprises or consists of 2 to 80 repetitive units, each independently selected from GPGXX, GGX and Aₓ as defined herein.

In some embodiments, the recombinant spider silk protein in this disclosure comprises or consists of repetitive units each independently selected from selected from the group consisting of GPGAS, GPGSG, GPGGY, GPGGP, GPGGA, GPGQQ, GPGGG, GPGQG, GPGGS, GGY, GGP, GGA, GGR, GGS, GGT, GGN, GGQ, AAAAA, AAAAAA, AAAAAAA, AAAAAAAA, AAAAAAAAA, AAAAAAAAAA, GGRPSDTYG and GGRPSSSYG, (i) GPYGPGASAAAAAAGGYGPGSGQQ, (ii) GSSAAAAAAAASGPGGYGPENQGPSGPGGYGPGGP, (iii) GPGQQGPGQQGPGQQGPGQQ: (iv) GPGGAGGPYGPGGAGGPYGPGGAGGPY, (v) GGTTIIEDLDITIDGADGPITISEELTI, (vi) PGSSAAAAAAAASGPGQGQGQGQGQGGRPSDTYG, (vii) SAAAAAAAAGPGGGNGGRPSDTYGAPGGGNGGRPSSSYG, (viii) GGAGGAGGAGGSGGAGGS (SEQ ID NO: 27), (ix) GPGGAGPGGYGPGGSGPGGYGPGGSGPGGY, (x) GPYGPGASAAAAAAGGYGPGCGQQ, (xi) GPYGPGASAAAAAAGGYGPGKGQQ, (xii) GSSAAAAAAAASGPGGYGPENQGPCGPGGYGPGGP, (xiii) GSSAAAAAAAASGPGGYGPKNQGPSGPGGYGPGGP, (xiv) GSSAAAAAAAASGPGGYGPKNQGPSGPGGYGPGGP, or variants thereof as described in U.S. Pat. No. 8,877,903, for example, a synthetic spider peptide having sequential order of GPGAS, GGY, GPGSG in the peptide chain, or sequential order of AAAAAAAA, GPGGY, GPGGP in the peptide chain, sequential order of AAAAAAAA, GPGQG, GGR in the peptide chain.

In some embodiments, this disclosure provides silk protein-like multiblock peptides that imitate the repeating units of amino acids derived from natural spider silk proteins such as Spidroin major 1 domain, Spidroin major 2 domain or Spidroin minor 1 domain and the profile of variation between the repeating units without modifying their three-dimensional conformation, wherein these silk protein-like multiblock peptides comprise a repeating unit of amino acids corresponding to one of the sequences (I), (II), (III) and/or (IV) below.

[(XGG)_{w}(XGA)(GXG)ₓ(AGA)_{y}(G)_{z}AG]ₚ Formula (I) in which: X corresponds to tyrosine or to glutamine, w is an integer equal to 2 or 3, x is an integer from 1 to 3, y is an integer from 5 to 7, z is an integer equal to 1 or 2, and p is an integer and having any weight average molecular weight described herein, and/or

[(GPG₂YGPGQ₂)ₐ(X′)₂S(A)_{b}]ₚ Formula (II) in which: X′ corresponds to the amino acid sequence GPS or GPG, a is equal to 2 or 3, b is an integer from 7 to 10, and p is an integer and having any weight average molecular weight described herein, and/or

[(GR)(GA)ₗ(A)ₘ(GGX)ₙ(GA)ᵢ(A)ₘ]ₚ Formula (III) and/or [(GGX)ₙ(GA)ₘ(A)ₗ]ₚ Formula (IV) in which: X″ corresponds to tyrosine, glutamine or alanine, 1 is an integer from 1 to 6, m is an integer from 0 to 4, n is an integer from 1 to 4, and p is an integer.

In some embodiments, the recombinant spider silk protein or an analog of a spider silk protein comprising an amino acid repeating unit of sequence (V):

[(Xaa Gly Gly)_{w}(Xaa Gly Ala)(Gly Xaa Gly)ₓ(Ala Gly Ala)_{y}(Gly)_{z}Ala Gly]ₚ Formula (V), wherein Xaa is tyrosine or glutamine, w is an integer equal to 2 or 3, x is an integer from 1 to 3, y is an integer from 5 to 7, z is an integer equal to 1 or 2, and p is an integer.

In some embodiments, the recombinant spider silk protein in this disclosure is selected from the group consisting of ADF-3 or variants thereof, ADF-4 or variants thereof, MaSpI (SEQ ID NO: 43) or variants thereof, MaSpII (SEQ ID NO: 44) or variants thereof as described in U.S. Pat. No. 8,367,803.

In some embodiments, this disclosure provides water soluble recombinant spider silk proteins produced in mammalian cells. The solubility of the spider silk proteins produced in mammalian cells was attributed to the presence of the COOH-terminus in these proteins, which makes them more hydrophilic. These COOH-terminal amino acids are absent in spider silk proteins expressed in microbial hosts.

In some embodiments, the recombinant spider silk protein in this disclosure comprises water soluble recombinant spider silk protein C16 modified with an amino or carboxyl terminal selected from the amino acid sequences consisting of: GCGGGGGG, GKGGGGGG, GCGGSGGGGSGGGG, GKGGGGGGSGGGG, and GCGGGGGGSGGGG. In some embodiments, the recombinant spider silk protein in this disclosure comprises C₁₆NR4, C₃₂NR4, C₁₆, C₃₂, NR4C₁₆NR4, NR4C₃₂NR4, NR3C₁₆NR3, or NR3C₃₂NR3 such that the molecular weight of the protein ranges as described herein.

In some embodiments, the recombinant spider silk protein in this disclosure comprises recombinant spider silk protein having a synthetic repetitive peptide segments and an amino acid sequence adapted from the natural sequence of ADF4 from A. diadematus as described in U.S. Pat. No. 8,877,903. In some embodiments, the RSPF in this disclosure comprises the recombinant spider silk proteins having repeating peptide units derived from natural spider silk proteins such as Spidroin major 1 domain, Spidroin major 2 domain or Spidroin minor 1 domain, wherein the repeating peptide sequence is GSSAAAAAAAASGPGQGQGQGQGQGGRPSDTYG or SAAAAAAAAGPGGGNGGRPSDTYGAPGGGNGGRPSSSYG, as described in U.S. Pat. No. 8,367,803.

In some embodiments, this disclosure provides recombinant spider proteins composed of the GPGGAGPGGYGPGGSGPGGYGPGGSGPGGY repetitive fragment and having a molecular weight as described herein.

As used herein, the term “recombinant silk” refers to recombinant spider and/or silkworm silk protein or fragments thereof. In an embodiment, the spider silk protein is selected from the group consisting of swathing silk (Achniform gland silk), egg sac silk (Cylindriform gland silk), egg case silk (Tubuliform silk), non-sticky dragline silk (Ampullate gland silk), attaching thread silk (Pyriform gland silk), sticky silk core fibers (Flagelliform gland silk), and sticky silk outer fibers (Aggregate gland silk). For example, recombinant spider silk protein, as described herein, includes the proteins described in U.S. Patent Application No. 2016/0222174 and U.S. Pat. Nos. 9,051,453, 9,617,315, 9,689,089, 8,173,772, and 8,642,734.

Some organisms make multiple silk fibers with unique sequences, structural elements, and mechanical properties. For example, orb weaving spiders have six unique types of glands that produce different silk polypeptide sequences that are polymerized into fibers tailored to fit an environmental or lifecycle niche. The fibers are named for the gland they originate from and the polypeptides are labeled with the gland abbreviation (e.g. “Ma”) and “Sp” for spidroin (short for spider fibroin). In orb weavers, these types include Major Ampullate (MaSp, also called dragline), Minor Ampullate (MiSp), Flagelliform (Flag), Aciniform (AcSp), Tubuliform (TuSp), and Pyriform (PySp). This combination of polypeptide sequences across fiber types, domains, and variation amongst different genus and species of organisms leads to a vast array of potential properties that can be harnessed by commercial production of the recombinant fibers. To date, the vast majority of the work with recombinant silks has focused on the Major Ampullate Spidroins (MaSp).

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSpI and MaSp2. MaSpI silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1 of U.S. Published Application No. 2016/0222174, the entirety of which is incorporated herein, lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements.

The construction of certain spider silk block copolymer polypeptides from the blocks and/or macro-repeat domains, according to certain embodiments of the disclosure, is illustrated in U.S. Published Patent Application No. 2016/0222174.

The recombinant block copolymer polypeptides based on spider silk sequences produced by gene expression in a recombinant prokaryotic or eukaryotic system can be purified according to methods known in the art. In a preferred embodiment, a commercially available expression/secretion system can be used, whereby the recombinant polypeptide is expressed and thereafter secreted from the host cell, to be easily purified from the surrounding medium. If expression/secretion vectors are not used, an alternative approach involves purifying the recombinant block copolymer polypeptide from cell lysates (remains of cells following disruption of cellular integrity) derived from prokaryotic or eukaryotic cells in which a polypeptide was expressed. Methods for generation of such cell lysates are known to those of skill in the art. In some embodiments, recombinant block copolymer polypeptides are isolated from cell culture supernatant.

Recombinant block copolymer polypeptide may be purified by affinity separation, such as by immunological interaction with antibodies that bind specifically to the recombinant polypeptide or nickel columns for isolation of recombinant polypeptides tagged with 6-8 histidine residues at their N-terminus or C-terminus Alternative tags may comprise the FLAG epitope or the hemagglutinin epitope. Such methods are commonly used by skilled practitioners.

A solution of such polypeptides (i.e., recombinant silk protein) may then be prepared and used as described herein.

In another embodiment, recombinant silk protein may be prepared according to the methods described in U.S. Pat. No. 8,642,734, the entirety of which is incorporated herein, and used as described herein.

In an embodiment, a recombinant spider silk protein is provided. The spider silk protein typically consists of from 170 to 760 amino acid residues, such as from 170 to 600 amino acid residues, preferably from 280 to 600 amino acid residues, such as from 300 to 400 amino acid residues, more preferably from 340 to 380 amino acid residues. The small size is advantageous because longer spider silk proteins tend to form amorphous aggregates, which require use of harsh solvents for solubilization and polymerization. The recombinant spider silk protein may contain more than 760 residues, in particular in cases where the spider silk protein contains more than two fragments derived from the N-terminal part of a spider silk protein, The spider silk protein comprises an N-terminal fragment consisting of at least one fragment (NT) derived from the corresponding part of a spider silk protein, and a repetitive fragment (REP) derived from the corresponding internal fragment of a spider silk protein. Optionally, the spider silk protein comprises a C-terminal fragment (CT) derived from the corresponding fragment of a spider silk protein. The spider silk protein comprises typically a single fragment (NT) derived from the N-terminal part of a spider silk protein, but in preferred embodiments, the N-terminal fragment include at least two, such as two fragments (NT) derived from the N-terminal part of a spider silk protein. Thus, the spidroin can schematically be represented by the formula NTₘ-REP, and alternatively NTₘ-REP-CT, where m is an integer that is 1 or higher, such as 2 or higher, preferably in the ranges of 1-2, 1-4, 1-6, 2-4 or 2-6. Preferred spidroins can schematically be represented by the formulas NT₂-REP or NT-REP, and alternatively NT₂-REP-CT or NT-REP-CT. The protein fragments are covalently coupled, typically via a peptide bond. In one embodiment, the spider silk protein consists of the NT fragment(s) coupled to the REP fragment, which REP fragment is optionally coupled to the CT fragment.

In one embodiment, the first step of the method of producing polymers of an isolated spider silk protein involves expression of a polynucleic acid molecule which encodes the spider silk protein in a suitable host, such as Escherichia coli. The thus obtained protein is isolated using standard procedures. Optionally, lipopolysaccharides and other pyrogens are actively removed at this stage.

In the second step of the method of producing polymers of an isolated spider silk protein, a solution of the spider silk protein in a liquid medium is provided. By the terms “soluble” and “in solution” is meant that the protein is not visibly aggregated and does not precipitate from the solvent at 60,000×g. The liquid medium can be any suitable medium, such as an aqueous medium, preferably a physiological medium, typically a buffered aqueous medium, such as a 10-50 mM Tris-HCl buffer or phosphate buffer. The liquid medium has a pH of 6.4 or higher and/or an ion composition that prevents polymerization of the spider silk protein. That is, the liquid medium has either a pH of 6.4 or higher or an ion composition that prevents polymerization of the spider silk protein, or both.

Ion compositions that prevent polymerization of the spider silk protein can readily be prepared by the skilled person utilizing the methods disclosed herein. A preferred ion composition that prevents polymerization of the spider silk protein has an ionic strength of more than 300 mM. Specific examples of ion compositions that prevent polymerization of the spider silk protein include above 300 mM NaCl, 100 mM phosphate and combinations of these ions having desired preventive effect on the polymerization of the spider silk protein, e.g., a combination of 10 mM phosphate and 300 mM NaCl.

The presence of an NT fragment improves the stability of the solution and prevents polymer formation under these conditions. This can be advantageous when immediate polymerization may be undesirable, e.g. during protein purification, in preparation of large batches, or when other conditions need to be optimized. It is preferred that the pH of the liquid medium is adjusted to 6.7 or higher, such as 7.0 or higher, or even 8.0 or higher, such as up to 10.5, to achieve high solubility of the spider silk protein. It can also be advantageous that the pH of the liquid medium is adjusted to the range of 6.4-6.8, which provides sufficient solubility of the spider silk protein but facilitates subsequent pH adjustment to 6.3 or lower.

In the third step, the properties of the liquid medium are adjusted to a pH of 6.3 or lower and ion composition that allows polymerization. That is, if the liquid medium wherein the spider silk protein is dissolved has a pH of 6.4 or higher, the pH is decreased to 6.3 or lower. The skilled person is well aware of various ways of achieving this, typically involving addition of a strong or weak acid. If the liquid medium wherein the spider silk protein is dissolved has an ion composition that prevents polymerization, the ion composition is changed so as to allow polymerization. The skilled person is well aware of various ways of achieving this, e.g. dilution, dialysis or gel filtration. If required, this step involves both decreasing the pH of the liquid medium to 6.3 or lower and changing the ion composition so as to allow polymerization. It is preferred that the pH of the liquid medium is adjusted to 6.2 or lower, such as 6.0 or lower. In particular, it may be advantageous from a practical point of view to limit the pH drop from 6.4 or 6.4-6.8 in the preceding step to 6.3 or 6.0-6.3, e.g., 6.2 in this step. In a preferred embodiment, the pH of the liquid medium of this step is 3 or higher, such as 4.2 or higher. The resulting pH range, e.g., 4.2-6.3 promotes rapid polymerization,

In the fourth step, the spider silk protein is allowed to polymerize in the liquid medium having pH of 6.3 or lower and an ion composition that allows polymerization of the spider silk protein. Although the presence of the NT fragment improves solubility of the spider silk protein at a pH of 6.4 or higher and/or an ion composition that prevents polymerization of the spider silk protein, it accelerates polymer formation at a pH of 6.3 or lower when the ion composition allows polymerization of the spider silk protein. The resulting polymers are preferably solid and macroscopic, and they are formed in the liquid medium having a pH of 6.3 or lower and an ion composition that allows polymerization of the spider silk protein. In a preferred embodiment, the pH of the liquid medium of this step is 3 or higher, such as 4.2 or higher. The resulting pH range, e.g., 4.2-6.3 promotes rapid polymerization, Resulting polymer may be provided at the molecular weights described herein and prepared as a solution form that may be used as necessary for article coatings.

Ion compositions that allow polymerization of the spider silk protein can readily be prepared by the skilled person utilizing the methods disclosed herein. A preferred ion composition that allows polymerization of the spider silk protein has an ionic strength of less than 300 mM. Specific examples of ion compositions that allow polymerization of the spider silk protein include 150 mM NaCl, 10 mM phosphate, 20 mM phosphate and combinations of these ions lacking preventive effect on the polymerization of the spider silk protein, e.g. a combination of 10 mM phosphate or 20 mM phosphate and 150 mM NaCl. It is preferred that the ionic strength of this liquid medium is adjusted to the range of 1-250 mM.

Without desiring to be limited to any specific theory, it is envisaged that the NT fragments have oppositely charged poles, and that environmental changes in pH affects the charge balance on the surface of the protein followed by polymerization, whereas salt inhibits the same event.

At neutral pH, the energetic cost of burying the excess negative charge of the acidic pole may be expected to prevent polymerization. However, as the dimer approaches its isoelectric point at lower pH, attractive electrostatic forces will eventually become dominant, explaining the observed salt and pH-dependent polymerization behavior of NT and NT-containing minispidroins. It is proposed that, in some embodiments, pH-induced NT polymerization, and increased efficiency of fiber assembly of NT-minispidroins, are due to surface electrostatic potential changes, and that clustering of acidic residues at one pole of NT shifts its charge balance such that the polymerization transition occurs at pH values of 6.3 or lower.

In a fifth step, the resulting, preferably solid spider silk protein polymers are isolated from said liquid medium. Optionally, this step involves actively removing lipopolysaccharides and other pyrogens from the spidroin polymers.

Without desiring to be limited to any specific theory, it has been observed that formation of spidroin polymers progresses via formation of water-soluble spidroin dimers. The present disclosure thus also provides a method of producing dimers of an isolated spider silk protein, wherein the first two method steps are as described above. The spider silk proteins are present as dimers in a liquid medium at a pH of 6.4 or higher and/or an ion composition that prevents polymerization of said spider silk protein. The third step involves isolating the dimers obtained in the second step, and optionally removal of lipopolysaccharides and other pyrogens. In a preferred embodiment, the spider silk protein polymer of the disclosure consists of polymerized protein dimers. The present disclosure thus provides a novel use of a spider silk protein, preferably those disclosed herein, for producing dimers of the spider silk protein.

According to another aspect, the disclosure provides a polymer of a spider silk protein as disclosed herein. In an embodiment, the polymer of this protein is obtainable by any one of the methods therefor according to the disclosure. Thus, the disclosure provides various uses of recombinant spider silk protein, preferably those disclosed herein, for producing polymers of the spider silk protein as recombinant silk based coatings. According to one embodiment, the present disclosure provides a novel use of a dimer of a spider silk protein, preferably those disclosed herein, for producing polymers of the isolated spider silk protein as recombinant silk based coatings. In these uses, it is preferred that the polymers are produced in a liquid medium having a pH of 6.3 or lower and an ion composition that allows polymerization of said spider silk protein. In an embodiment, the pH of the liquid medium is 3 or higher, such as 4.2 or higher. The resulting pH range, e.g., 4.2-6.3 promotes rapid polymerization,

Using the method(s) of the present disclosure, it is possible to control the polymerization process, and this allows for optimization of parameters for obtaining silk polymers with desirable properties and shapes.

In an embodiment, the recombinant silk proteins described herein, include those described in U.S. Pat. No. 8,642,734, the entirety of which is incorporated by reference.

In another embodiment, the recombinant silk proteins described herein may be prepared according to the methods described in U.S. Pat. No. 9,051,453, the entirety of which is incorporated herein by reference.

An amino acid sequence represented by SEQ ID NO: 1 of U.S. Pat. No. 9,051,453 is identical to an amino acid sequence that is composed of 50 amino acid residues of an amino acid sequence of ADF3 at the C-terminal (NCBI Accession No.: AAC47010, GI: 1263287). An amino acid sequence represented by SEQ ID NO: 2 of U.S. Pat. No. 9,051,453 is identical to an amino acid sequence represented by SEQ ID NO: 1 of U.S. Pat. No. 9,051,453 from which 20 residues have been removed from the C-terminal. An amino acid sequence represented by SEQ ID NO: 3 of U.S. Pat. No. 9,051,453 is identical to an amino acid sequence represented by SEQ ID NO: 1 from which 29 residues have been removed from the C-terminal.

An example of the polypeptide that contains units of the amino acid sequence represented by the formula 1: REP1-REP2 (1) and that has, at a C-terminal, an amino acid sequence represented by any of SEQ ID NOS: 1 to 3 or an amino acid sequence having a homology of 90% or more with the amino acid sequence represented by any of SEQ ID NOS: 1 to 3 of U.S. Pat. No. 9,051,453 is a polypeptide having an amino acid sequence represented by SEQ ID NO: 8 of U.S. Pat. No. 9,051,453. The polypeptide having the amino acid sequence represented by SEQ ID NO: 8 of U.S. Pat. No. 9,051,453 is obtained by the following mutation: in an amino acid sequence of ADF3 (NCBI Accession No.: AAC47010, GI: 1263287) to the N-terminal of which has been added an amino acid sequence (SEQ ID NO: 5 of U.S. Pat. No. 9,051,453) composed of a start codon, His 10 tags and an HRV3C Protease (Human rhinovirus 3C Protease) recognition site, 1^{st} to 13^{th} repetitive regions are about doubled and the translation ends at the 11₅₄′ amino acid residue. In the polypeptide having the amino acid sequence represented by SEQ ID NO: 8 of U.S. Pat. No. 9,051,453, the C-terminal sequence is identical to the amino acid sequence represented by SEQ ID NO: 3.

Further, the polypeptide that contains units of the amino acid sequence represented by the formula 1: REP1-REP2 (1) and that has, at a C-terminal, an amino acid sequence represented by any of SEQ ID NOS: 1 to 3 of U.S. Pat. No. 9,051,453 or an amino acid sequence having a homology of 90% or more with the amino acid sequence represented by any of SEQ ID NOS: 1 to 3 of U.S. Pat. No. 9,051,453 may be a protein that has an amino acid sequence represented by SEQ ID NO: 8 of U.S. Pat. No. 9,051,453 in which one or a plurality of amino acids have been substituted, deleted, inserted and/or added and that has a repetitious region composed of a crystal region and an amorphous region.

Further, an example of the polypeptide containing two or more units of the amino acid sequence represented by the formula 1: REP1-REP2 (1) is a recombinant protein derived from ADF4 having an amino acid sequence represented by SEQ ID NO: 15 of U.S. Pat. No. 9,051,453. The amino acid sequence represented by SEQ ID NO: 15 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial amino acid sequence of ADF4 obtained from the NCBI database (NCBI Accession No.: AAC47011, GI: 1263289). Further, the polypeptide containing two or more units of the amino acid sequence represented by the formula 1: REP1-REP2 (1) may be a polypeptide that has an amino acid sequence represented by SEQ ID NO: 15 of U.S. Pat. No. 9,051,453 in which one or a plurality of amino acids have been substituted, deleted, inserted and/or added and that has a repetitious region composed of a crystal region and an amorphous region. Further, an example of the polypeptide containing two or more units of the amino acid sequence represented by the formula 1: REP1-REP2 (1) is a recombinant protein derived from MaSp2 that has an amino acid sequence represented by SEQ ID NO: 17 of U.S. Pat. No. 9,051,453. The amino acid sequence represented by SEQ ID NO: 17 of U.S. Pat. No. 9,051,453 is an amino acid sequence obtained by adding the amino acid sequence (SEQ ID NO: 5 of U.S. Pat. No. 9,051,453) composed of a start codon, His 10 tags and an HRV3C Protease (Human rhinovirus 3C Protease) recognition site, to the N-terminal of a partial sequence of MaSp2 obtained from the NCBI web database (NCBI Accession No.: AAT75313, GI: 50363147). Furthermore, the polypeptide containing two or more units of the amino acid sequence represented by the formula 1: REP1-REP2 (1) may be a polypeptide that has an amino acid sequence represented by SEQ ID NO: 17 of U.S. Pat. No. 9,051,453 in which one or a plurality of amino acids have been substituted, deleted, inserted and/or added and that has a repetitious region composed of a crystal region and an amorphous region.

Examples of the polypeptide derived from flagelliform silk proteins include a polypeptide containing 10 or more units of an amino acid sequence represented by the formula 2: REP3 (2), preferably a polypeptide containing 20 or more units thereof, and more preferably a polypeptide containing 30 or more units thereof. In the case of producing a recombinant protein using a microbe such as Escherichia coli as a host, the molecular weight of the polypeptide derived from flagelliform silk proteins is preferably 500 kDa or less, more preferably 300 kDa or less, and further preferably 200 kDa or less, in terms of productivity.

In the formula (2), the REP 3 indicates an amino acid sequence composed of Gly-Pro-Gly-Gly-X, where X indicates an amino acid selected from the group consisting of Ala, Ser, Tyr and Val.

A major characteristic of the spider silk is that the flagelliform silk does not have a crystal region, but has a repetitious region composed of an amorphous region. Since the major dragline silk and the like have a repetitious region composed of a crystal region and an amorphous region, they are expected to have both high stress and stretchability. Meanwhile, as to the flagelliform silk, although the stress is inferior to that of the major dragline silk, the stretchability is high. The reason for this is considered to be that most of the flagelliform silk is composed of amorphous regions.

An example of the polypeptide containing 10 or more units of the amino acid sequence represented by the formula 2: REP3 (2) is a recombinant protein derived from flagelliform silk proteins having an amino acid sequence represented by SEQ ID NO: 19 of U.S. Pat. No. 9,051,453. The amino acid sequence represented by SEQ ID NO: 19 of U.S. Pat. No. 9,051,453 is an amino acid sequence obtained by combining a partial sequence of flagelliform silk protein of Nephila clavipes obtained from the NCBI database (NCBI Accession No.: AAF36090, GI: 7106224), specifically, an amino acid sequence thereof from the 1220^{th} residue to the 1659^{th} residue from the N-terminal that corresponds to repetitive sections and motifs (referred to as a PR1 sequence), with a partial sequence of flagelliform silk protein of Nephila clavipes obtained from the NCBI database (NCBI Accession No.: AAC38847, GI: 2833649), specifically, a C-terminal amino acid sequence thereof from the 816^{th} residue to the 907^{th} residue from the C-terminal, and thereafter adding the amino acid sequence (SEQ ID NO: 5 of U.S. Pat. No. 9,051,453) composed of a start codon, His 10 tags and an HRV3C Protease recognition site, to the N-terminal of the combined sequence. Further, the polypeptide containing 10 or more units of the amino acid sequence represented by the formula 2: REP3 (2) may be a polypeptide that has an amino acid sequence represented by SEQ ID NO: 19 of U.S. Pat. No. 9,051,453 in which one or a plurality of amino acids have been substituted, deleted, inserted and/or added and that has a repetitious region composed of an amorphous region.

The polypeptide can be produced using a host that has been transformed by an expression vector containing a gene encoding a polypeptide. A method for producing a gene is not limited particularly, and it may be produced by amplifying a gene encoding a natural spider silk protein from a cell derived from spiders by a polymerase chain reaction (PCR), etc., and cloning it, or may be synthesized chemically. Also, a method for chemically synthesizing a gene is not limited particularly, and it can be synthesized as follows, for example: based on information of amino acid sequences of natural spider silk proteins obtained from the NCBI web database, etc., oligonucleotides that have been synthesized automatically with AKTA oligopilot plus 10/100 (GE Healthcare Japan Corporation) are linked by PCR, etc. At this time, in order to facilitate the purification and observation of protein, it is possible to synthesize a gene that encodes a protein having an amino acid sequence of the above-described amino acid sequence to the N-terminal of which has been added an amino acid sequence composed of a start codon and His 10 tags.

Examples of the expression vector include a plasmid, a phage, a virus, and the like that can express protein based on a DNA sequence. The plasmid-type expression vector is not limited particularly as long as it allows a target gene to be expressed in a host cell and it can amplify itself. For example, in the case of using Escherichia coli Rosetta (DE3) as a host, a pET22b(+) plasmid vector, a pCold plasmid vector, and the like can be used. Among these, in terms of productivity of protein, it is preferable to use the pET22b(+) plasmid vector. Examples of the host include animal cells, plant cells, microbes, etc.

The polypeptide used in the present disclosure is preferably a polypeptide derived from ADF3, which is one of two principal dragline silk proteins of Araneus diadematus. This polypeptide has advantages of basically having high strength-elongation and toughness and of being synthesized easily.

Accordingly, the recombinant silk protein (e.g., the recombinant spider silk-based protein) used in accordance with the embodiments, articles, and/or methods described herein, may include one or more recombinant silk proteins described above or recited in U.S. Pat. Nos. 8,173,772, 8,278,416, 8,618,255, 8,642,734, 8,691,581, 8,729,235, 9,115,204, 9,157,070, 9,309,299, 9,644,012, 9,708,376, 9,051,453, 9,617,315, 9,968,682, 9,689,089, 9,732,125, 9,856,308, 9,926,348, 10,065,997, 10,316,069, and 10,329,332; and U.S. Patent Publication Nos. 2009/0226969, 2011/0281273, 2012/0041177, 2013/0065278, 2013/0115698, 2013/0316376, 2014/0058066, 2014/0079674, 2014/0245923, 2015/0087046, 2015/0119554, 2015/0141618, 2015/0291673, 2015/0291674, 2015/0239587, 2015/0344542, 2015/0361144, 2015/0374833, 2015/0376247, 2016/0024464, 2017/0066804, 2017/0066805, 2015/0293076, 2016/0222174, 2017/0283474, 2017/0088675, 2019/0135880, 2015/0329587, 2019/0040109, 2019/0135881, 2019/0177363, 2019/0225646, 2019/0233481, 2019/0031842, 2018/0355120, 2019/0186050, 2019/0002644, 2020/0031887, 2018/0273590, 20191/094403, 2019/0031843, 2018/0251501, 2017/0066805, 2018/0127553, 2019/0329526, 2020/0031886, 2018/0080147, 2019/0352349, 2020/0043085, 2019/0144819, 2019/0228449, 2019/0340666, 2020/0000091, 2019/0194710, 2019/0151505, 2018/0265555, 2019/0352330, 2019/0248847, and 2019/0378191, the entirety of which are incorporated herein by reference.

### Silk Fibroin-Like Protein Fragments

The recombinant silk protein in this disclosure comprises synthetic proteins which are based on repeat units of natural silk proteins. Besides the synthetic repetitive silk protein sequences, these can additionally comprise one or more natural nonrepetitive silk protein sequences. As used herein, “silk fibroin-like protein fragments” refer to protein fragments having a molecular weight and polydispersity as defined herein, and a certain degree of homology to a protein selected from native silk protein, fibroin heavy chain, fibroin light chain, or any protein comprising one or more GAGAGS hexa amino acid repeating units. In some embodiments, a degree of homology is selected from about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, or less than 75%.

As described herein, a protein such as native silk protein, fibroin heavy chain, fibroin light chain, or any protein comprising one or more GAGAGS hexa amino acid repeating units includes between about 9% and about 45% glycine, or about 9% glycine, or about 10% glycine, about 43% glycine, about 44% glycine, about 45% glycine, or about 46% glycine. As described herein, a protein such as native silk protein, fibroin heavy chain, fibroin light chain, or any protein comprising one or more GAGAGS hexa amino acid repeating units includes between about 13% and about 30% alanine, or about 13% alanine, or about 28% alanine, or about 29% alanine, or about 30% alanine, or about 31% alanine. As described herein, a protein such as native silk protein, fibroin heavy chain, fibroin light chain, or any protein comprising one or more GAGAGS hexa amino acid repeating units includes between 9% and about 12% serine, or about 9% serine, or about 10% serine, or about 11% serine, or about 12% serine.

In some embodiments, a silk fibroin-like protein described herein includes about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, or about 55% glycine. In some embodiments, a silk fibroin-like protein described herein includes about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, or about 39% alanine. In some embodiments, a silk fibroin-like protein described herein includes about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, or about 22% serine. In some embodiments, a silk fibroin-like protein described herein may include independently any amino acid known to be included in natural fibroin. In some embodiments, a silk fibroin-like protein described herein may exclude independently any amino acid known to be included in natural fibroin. In some embodiments, on average 2 out of 6 amino acids, 3 out of 6 amino acids, or 4 out of 6 amino acids in a silk fibroin-like protein described herein is glycine. In some embodiments, on average 1 out of 6 amino acids, 2 out of 6 amino acids, or 3 out of 6 amino acids in a silk fibroin-like protein described herein is alanine. In some embodiments, on average none out of 6 amino acids, 1 out of 6 amino acids, or 2 out of 6 amino acids in a silk fibroin-like protein described herein is serine.

### Other Properties of SPF

Compositions of the present disclosure are “biocompatible” or otherwise exhibit “biocompatibility” meaning that the compositions are compatible with living tissue or a living system by not being toxic, injurious, or physiologically reactive and not causing immunological rejection or an inflammatory response. Such biocompatibility can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely. For example, in some embodiments, the coatings described herein are biocompatible coatings.

In some embodiments, compositions described herein, which may be biocompatible compositions (e.g., biocompatible coatings that include silk), may be evaluated and comply with International Standard ISO 10993-1, titled the “Biological evaluation of medical devices—Part 1: Evaluation and testing within a risk management process.” In some embodiments, compositions described herein, which may be biocompatible compositions, may be evaluated under ISO 106993-1 for one or more of cytotoxicity, sensitization, hemocompatibility, pyrogenicity, implantation, genotoxicity, carcinogenicity, reproductive and developmental toxicity, and degradation.

Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

In an embodiment, the stability of a composition of the present disclosure is about 1 day. In an embodiment, the stability of a composition of the present disclosure is about 2 days. In an embodiment, the stability of a composition of the present disclosure is about 3 days. In an embodiment, the stability of a composition of the present disclosure is about 4 days. In an embodiment, the stability of a composition of the present disclosure is about 5 days. In an embodiment, the stability of a composition of the present disclosure is about 6 days. In an embodiment, the stability of a composition of the present disclosure is about 7 days. In an embodiment, the stability of a composition of the present disclosure is about 8 days. In an embodiment, the stability of a composition of the present disclosure is about 9 days. In an embodiment, the stability of a composition of the present disclosure is about 10 days.

In an embodiment, the stability of a composition of the present disclosure is about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a SPF composition of the present disclosure is not soluble in an aqueous solution due to the crystallinity of the protein. In an embodiment, a SPF composition of the present disclosure is soluble in an aqueous solution. In an embodiment, the SPF of a composition of the present disclosure include a crystalline portion of about two-thirds and an amorphous region of about one-third. In an embodiment, the SPF of a composition of the present disclosure include a crystalline portion of about one-half and an amorphous region of about one-half. In an embodiment, the SPF of a composition of the present disclosure include a 99% crystalline portion and a 1% amorphous region. In an embodiment, the SPF of a composition of the present disclosure include a 95% crystalline portion and a 5% amorphous region. In an embodiment, the SPF of a composition of the present disclosure include a 90% crystalline portion and a 10% amorphous region. In an embodiment, the SPF of a composition of the present disclosure include a 85% crystalline portion and a 15% amorphous region. In an embodiment, the SPF of a composition of the present disclosure include a 80% crystalline portion and a 20% amorphous region. In an embodiment, the SPF of a composition of the present disclosure include a 75% crystalline portion and a 25% amorphous region. In an embodiment, the SPF of a composition of the present disclosure include a 70% crystalline portion and a 30% amorphous region. In an embodiment, the SPF of a composition of the present disclosure include a 65% crystalline portion and a 35% amorphous region. In an embodiment, the SPF of a composition of the present disclosure include a 60% crystalline portion and a 40% amorphous region. In an embodiment, the SPF of a composition of the present disclosure include a 50% crystalline portion and a 50% amorphous region. In an embodiment, the SPF of a composition of the present disclosure include a 40% crystalline portion and a 60% amorphous region. In an embodiment, the SPF of a composition of the present disclosure include a 35% crystalline portion and a 65% amorphous region. In an embodiment, the SPF of a composition of the present disclosure include a 30% crystalline portion and a 70% amorphous region. In an embodiment, the SPF of a composition of the present disclosure include a 25% crystalline portion and a 75% amorphous region. In an embodiment, the SPF of a composition of the present disclosure include a 20% crystalline portion and a 80% amorphous region. In an embodiment, the SPF of a composition of the present disclosure include a 15% crystalline portion and a 85% amorphous region. In an embodiment, the SPF of a composition of the present disclosure include a 10% crystalline portion and a 90% amorphous region. In an embodiment, the SPF of a composition of the present disclosure include a 5% crystalline portion and a 90% amorphous region. In an embodiment, the SPF of a composition of the present disclosure include a 1% crystalline portion and a 99% amorphous region.

As used herein, the term “substantially free of inorganic residuals” means that the composition exhibits residuals of 0.1% (w/w) or less. In an embodiment, substantially free of inorganic residuals refers to a composition that exhibits residuals of 0.05% (w/w) or less. In an embodiment, substantially free of inorganic residuals refers to a composition that exhibits residuals of 0.01% (w/w) or less. In an embodiment, the amount of inorganic residuals is between 0 ppm (“non-detectable” or “ND”) and 1000 ppm. In an embodiment, the amount of inorganic residuals is ND to about 500 ppm. In an embodiment, the amount of inorganic residuals is ND to about 400 ppm. In an embodiment, the amount of inorganic residuals is ND to about 300 ppm. In an embodiment, the amount of inorganic residuals is ND to about 200 ppm. In an embodiment, the amount of inorganic residuals is ND to about 100 ppm. In an embodiment, the amount of inorganic residuals is between 10 ppm and 1000 ppm.

As used herein, the term “substantially free of organic residuals” means that the composition exhibits residuals of 0.1% (w/w) or less, in an embodiment, substantially free of organic residuals refers to a composition that exhibits residuals of 0.05% (w/w) or less. In an embodiment, substantially free of organic residuals refers to a composition that exhibits residuals of 0.01% (w/w) or less. In an embodiment, the amount of organic residuals is between 0 ppm (“non-detectable” or “ND”) and 1000 ppm. In an embodiment, the amount of organic residuals is ND to about 500 ppm. In an embodiment, the amount of organic residuals is ND to about 400 ppm. In an embodiment, the amount of organic residuals is ND to about 300 ppm. In an embodiment, the amount of organic residuals is ND to about 200 ppm. In an embodiment, the amount of organic residuals is ND to about 100 ppm. In an embodiment, the amount of organic residuals is between 10 ppm and 1000 ppm.

Compositions of the present disclosure exhibit “biocompatibility” meaning that the compositions are compatible with living tissue or a living system by not being toxic, injurious, or physiologically reactive and not causing immunological rejection. Such biocompatibility can be evidenced by participants topically applying compositions of the present disclosure on their skin for an extended period of time. In an embodiment, the extended period of time is about 3 days. In an embodiment, the extended period of time is about 7 days, in an embodiment, the extended period of time is about 14 days, in an embodiment, the extended period of time is about 21 days. In an embodiment, the extended period of time is about 30 days. In an embodiment, the extended period of time is selected from the group consisting of about I month, about 2 months, about 3 months, about 4 months, about 5 months, about 6 months, about 7 months, about 8 months, about 9 months, about 10 months, about 11 months, about 12 months, and indefinitely. Compositions of the present disclosure are “hypoallergenic” meaning that they are relatively unlikely to cause an allergic reaction. Such hypoallergenicity can be evidenced by participants topically applying compositions of the present disclosure on their skin for an extended period of time. In an embodiment, the extended period of time is about 3 days. In an embodiment, the extended period of time is about 7 days. In an embodiment, the extended period of time is about 14 days. In an embodiment, the extended period of time is about 21 days. In an embodiment, the extended period of time is about 30 days. In an embodiment, the extended period of time is selected from the group consisting of about 1 month, about 2 months, about 3 months, about 4 months, about 5 months, about 6 months, about 7 months, about 8 months, about 9 months, about 10 months, about 11 months, about 12 months, and indefinitely.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent SPF in the solution is less than 30.0 wt. %. In an embodiment, the percent SPF in the solution is less than 25.0 wt. %. In an embodiment, the percent SPF in the solution is less than 20.0 wt. %. In an embodiment, the percent SPF in the solution is less than 19.0 wt. %. In an embodiment, the percent SPF in the solution is less than 18.0 wt. %. In an embodiment, the percent SPF in the solution is less than 17.0 wt. %. In an embodiment, the percent SPF in the solution is less than 16.0 wt. %. In an embodiment, the percent SPF in the solution is less than 15.0 wt. %. In an embodiment, the percent SPF in the solution is less than 14.0 wt. %. In an embodiment, the percent SPF in the solution is less than 13.0 wt. %. In an embodiment, the percent SPF in the solution is less than 12.0 wt. %. In an embodiment, the percent SPF in the solution is less than 11.0 wt. %. In an embodiment, the percent SPF in the solution is less than 10.0 wt. %. In an embodiment, the percent SPF in the solution is less than 9.0 wt. %. In an embodiment, the percent SPF in the solution is less than 8.0 wt. %. In an embodiment, the percent SPF in the solution is less than 7.0 wt. %. In an embodiment, the percent SPF in the solution is less than 6.0 wt. %. In an embodiment, the percent SPF in the solution is less than 5.0 wt. %. In an embodiment, the percent SPF in the solution is less than 4.0 wt. %. In an embodiment, the percent SPF in the solution is less than 3.0 wt. %. In an embodiment, the percent SPF in the solution is less than 2.0 wt. %. In an embodiment, the percent SPF in the solution is less than 1.0 wt. %. In an embodiment, the percent SPF in the solution is less than 0.9 wt. %. In an embodiment, the percent SPF in the solution is less than 0.8 wt. %. In an embodiment, the percent SPF in the solution is less than 0.7 wt. %. In an embodiment, the percent SPF in the solution is less than 0.6 wt. %. In an embodiment, the percent SPF in the solution is less than 0.5 wt. %. In an embodiment, the percent SPF in the solution is less than 0.4 wt. %. In an embodiment, the percent SPF in the solution is less than 0.3 wt. %. In an embodiment, the percent SPF in the solution is less than 0.2 wt. %. In an embodiment, the percent SPF in the solution is less than 0.1 wt. %.

In an embodiment, the percent SPF in the solution is greater than 0.1 wt. %. In an embodiment, the percent SPF in the solution is greater than 0.2 wt. %. In an embodiment, the percent SPF in the solution is greater than 0.3 wt. %. In an embodiment, the percent SPF in the solution is greater than 0.4 wt. %. In an embodiment, the percent SPF in the solution is greater than 0.5 wt. %. In an embodiment, the percent SPF in the solution is greater than 0.6 wt. %. In an embodiment, the percent SPF in the solution is greater than 0.7 wt. %. In an embodiment, the percent SPF in the solution is greater than 0.8 wt. %. In an embodiment, the percent SPF in the solution is greater than 0.9 wt. %. In an embodiment, the percent SPF in the solution is greater than 1.0 wt. %. In an embodiment, the percent SPF in the solution is greater than 2.0 wt. %. In an embodiment, the percent SPF in the solution is greater than 3.0 wt. %. In an embodiment, the percent SPF in the solution is greater than 4.0 wt. %. In an embodiment, the percent SPF in the solution is greater than 5.0 wt. %. In an embodiment, the percent SPF in the solution is greater than 6.0 wt. %. In an embodiment, the percent SPF in the solution is greater than 7.0 wt. %. In an embodiment, the percent SPF in the solution is greater than 8.0 wt. %. In an embodiment, the percent SPF in the solution is greater than 9.0 wt. %. In an embodiment, the percent SPF in the solution is greater than 10.0 wt. %. In an embodiment, the percent SPF in the solution is greater than 11.0 wt. %. In an embodiment, the percent SPF in the solution is greater than 12.0 wt. %. In an embodiment, the percent SPF in the solution is greater than 13.0 wt. %. In an embodiment, the percent SPF in the solution is greater than 14.0 wt. %. In an embodiment, the percent SPF in the solution is greater than 15.0 wt. %. In an embodiment, the percent SPF in the solution is greater than 16.0 wt. %. In an embodiment, the percent SPF in the solution is greater than 17.0 wt. %. In an embodiment, the percent SPF in the solution is greater than 18.0 wt. %. In an embodiment, the percent SPF in the solution is greater than 19.0 wt. %. In an embodiment, the percent SPF in the solution is greater than 20.0 wt. %. In an embodiment, the percent SPF in the solution is greater than 25.0 wt. %.

In an embodiment, the percent SPF in the solution ranges from about 0.1 wt. % to about 30.0 wt. %. In an embodiment, the percent SPF in the solution ranges from about 0.1 wt. % to about 25.0 wt. %. In an embodiment, the percent SPF in the solution ranges from about 0.1 wt. % to about 20.0 wt. %. In an embodiment, the percent SPF in the solution ranges from about 0.1 wt. % to about 15.0 wt. %. In an embodiment, the percent SPF in the solution ranges from about 0.1 wt. % to about 10.0 wt. %. In an embodiment, the percent SPF in the solution ranges from about 0.1 wt. % to about 9.0 wt. %. In an embodiment, the percent SPF in the solution ranges from about 0.1 wt. % to about 8.0 wt. %. In an embodiment, the percent SPF in the solution ranges from about 0.1 wt. % to about 7.0 wt. %. In an embodiment, the percent SPF in the solution ranges from about 0.1 wt. % to about 6.5 wt. %. In an embodiment, the percent SPF in the solution ranges from about 0.1 wt. % to about 6.0 wt. %. In an embodiment, the percent SPF in the solution ranges from about 0.1 wt. % to about 5.5 wt. %. In an embodiment, the percent SPF in the solution ranges from about 0.1 wt. % to about 5.0 wt. %. In an embodiment, the percent SPF in the solution ranges from about 0.1 wt. % to about 4.5 wt. %. In an embodiment, the percent SPF in the solution ranges from about 0.1 wt. % to about 4.0 wt. %. In an embodiment, the percent SPF in the solution ranges from about 0.1 wt. % to about 3.5 wt. %. In an embodiment, the percent SPF in the solution ranges from about 0.1 wt. % to about 3.0 wt. %. In an embodiment, the percent SPF in the solution ranges from about 0.1 wt. % to about 2.5 wt. %. In an embodiment, the percent SPF in the solution ranges from about 0.1 wt. % to about 2.0 wt. %. In an embodiment, the percent SPF in the solution ranges from about 0.1 wt. % to about 2.4 wt. %. In an embodiment, the percent SPF in the solution ranges from about 0.5 wt. % to about 5.0 wt. %. In an embodiment, the percent SPF in the solution ranges from about 0.5 wt. % to about 4.5 wt. %. In an embodiment, the percent SPF in the solution ranges from about 0.5 wt. % to about 4.0 wt. %. In an embodiment, the percent SPF in the solution ranges from about 0.5 wt. % to about 3.5 wt. %. In an embodiment, the percent SPF in the solution ranges from about 0.5 wt. % to about 3.0 wt. %. In an embodiment, the percent SPF in the solution ranges from about 0.5 wt. % to about 2.5 wt. %. In an embodiment, the percent SPF in the solution ranges from about 1.0 wt. % to about 4.0 wt. %. In an embodiment, the percent SPF in the solution ranges from about 1.0 wt. % to about 3.5 wt. %. In an embodiment, the percent SPF in the solution ranges from about 1.0 wt. % to about 3.0 wt. %. In an embodiment, the percent SPF in the solution ranges from about 1.0 wt. % to about 2.5 wt. %. In an embodiment, the percent SPF in the solution ranges from about 1.0 wt. % to about 2.4 wt. %. In an embodiment, the percent SPF in the solution ranges from about 1.0 wt. % to about 2.0 wt. %.

In an embodiment, the percent SPF in the solution ranges from about 20.0 wt. % to about 30.0 wt. %. In an embodiment, the percent SPF in the solution ranges from about 0.1 wt. % to about 10.0 wt. %. In an embodiment, the percent SPF in the solution ranges from about 1.0 wt. % to about 10.0 wt. %. In an embodiment, the percent SPF in the solution ranges from about 2 wt. % to about 10.0 wt. %. In an embodiment, the percent SPF in the solution ranges from about 0.1 wt. % to about 6.0 wt. %. In an embodiment, the percent SPF in the solution ranges from about 6.0 wt. % to about 10.0 wt. %. In an embodiment, the percent SPF in the solution ranges from about 6.0 wt. % to about 8.0 wt. %. In an embodiment, the percent SPF in the solution ranges from about 6.0 wt. % to about 9.0 wt. %. In an embodiment, the percent SPF in the solution ranges from about 10.0 wt. % to about 20.0 wt. %. In an embodiment, the percent SPF in the solution ranges from about 11.0 wt. % to about 19.0 wt. %. In an embodiment, the percent SPF in the solution ranges from about 12.0 wt. % to about 18.0 wt. %. In an embodiment, the percent SPF in the solution ranges from about 13.0 wt. % to about 17.0 wt. %. In an embodiment, the percent SPF in the solution ranges from about 14.0 wt. % to about 16.0 wt. %. In an embodiment, the percent SPF in the solution is about 1.0 wt. %. In an embodiment, the percent SPF in the solution is about 1.5 wt. %. In an embodiment, the percent SPF in the solution is about 2.0 wt. %. In an embodiment, the percent SPF in the solution is about 2.4 wt. %. In an embodiment, the percent SPF in the solution is 3.0 wt. %. In an embodiment, the percent SPF in the solution is 3.5 wt. %. In an embodiment, the percent SPF in the solution is about 4.0 wt. %. In an embodiment, the percent SPF in the solution is about 4.5 wt. %. In an embodiment, the percent SPF in the solution is about 5.0 wt. %. In an embodiment, the percent SPF in the solution is about 5.5 wt. %. In an embodiment the percent SPF in the solution is about 6.0 wt. %. In an embodiment, the percent SPF in the solution is about 6.5 wt. %. In an embodiment, the percent SPF in the solution is about 7.0 wt. %. In an embodiment, the percent SPF in the solution is about 7.5 wt. %. In an embodiment, the percent SPF in the solution is about 8.0 wt. %. In an embodiment, the percent SPF in the solution is about 8.5 wt. %. In an embodiment, the percent SPF in the solution is about 9.0 wt. %. In an embodiment, the percent SPF in the solution is about 9.5 wt. %. In an embodiment, the percent SPF in the solution is about 10.0 wt. %.

In an embodiment, the percent sericin in the solution is non-detectable to 25.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 25.0 wt. %.

In some embodiments, the silk fibroin protein fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent SPF, and number of shipments and shipment conditions. Additionally, pH may be altered to extend shelf life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure having SPF has non-detectable levels of LiBr residuals. In an embodiment, the amount of the LiBr residuals in a composition of the present disclosure is between 10 ppm and 1000 ppm. In an embodiment, the amount of the LiBr residuals in a composition of the present disclosure is between 10 ppm and 300 ppm. In an embodiment, the amount of the LiBr residuals in a composition of the present disclosure is less than 25 ppm. In an embodiment, the amount of the Li Br residuals in a composition of the present disclosure is less than 50 ppm. In an embodiment, the amount of the LiBr residuals in a composition of the present disclosure is less than 75 ppm. In an embodiment, the amount of the LiBr residuals in a composition of the present disclosure is less than 100 ppm. In an embodiment, the amount of the LiBr residuals in a composition of the present disclosure is less than 200 ppm. In an embodiment, the amount of the LiBr residuals in a composition of the present disclosure is less than 300 ppm. In an embodiment, the amount of the LiBr residuals in a composition of the present disclosure is less than 400 ppm. In an embodiment, the amount of the LiBr residuals in a composition of the present disclosure is less than 500 ppm. In an embodiment, the amount of the LiBr residuals in a composition of the present disclosure is less than 600 ppm. In an embodiment, the amount of the LiBr residuals in a composition of the present disclosure is less than 700 ppm. In an embodiment, the amount of the LiBr residuals in a composition of the present disclosure is less than 800 ppm. In an embodiment, the amount of the LiBr residuals in a composition of the present disclosure is less than 900 ppm. In an embodiment, the amount of the LiBr residuals in a composition of the present disclosure is less than 1000 ppm. In an embodiment, the amount of the LiBr residuals in a composition of the present disclosure is non-detectable to 500 ppm. In an embodiment, the amount of the LiBr residuals in a composition of the present disclosure is non-detectable to 450 ppm. In an embodiment, the amount of the LiBr residue in a composition of the present disclosure is non-detectable to 400 ppm. In an embodiment, the amount of the LiBr residuals in a composition of the present disclosure is non-detectable to 350 ppm. In an embodiment, the amount of the LiBr residuals in a composition of the present disclosure is non-detectable to 300 ppm. In an embodiment, the amount of the LiBr residuals in a composition of the present disclosure is non-detectable to 250 ppm. In an embodiment, the amount of the LiBr residuals in a composition of the present disclosure is non-detectable to 200 ppm. In an embodiment, the amount of the LiBr residuals in a composition of the present disclosure is non-detectable to 150 ppm. In an embodiment, the amount of the LiBr residuals in a composition of the present disclosure is non-detectable to 100 ppm. In an embodiment, the amount of the LiBr residuals in a composition of the present disclosure is 100 ppm to 200 ppm. In an embodiment, the amount of the LiBr residuals in a composition of the present disclosure is 200 ppm to 300 ppm. In an embodiment, the amount of the LiBr residuals in a composition of the present disclosure is 300 ppm to 400 ppm. In an embodiment, the amount of the LiBr residuals in a composition of the present disclosure is 400 ppm to 500 ppm.

In an embodiment, a composition of the present disclosure having SPF, has non-detectable levels of Na₂CO₃ residuals. In an embodiment, the amount of the Na₂CO₃ residuals in a composition of the present disclosure is less than 100 ppm. In an embodiment, the amount of the Na₂CO₃ residuals in a composition of the present disclosure is less than 200 ppm. In an embodiment, the amount of the Na₂CO₃ residuals in a composition of the present disclosure is less than 300 ppm. In an embodiment, the amount of the Na₂CO₃ residuals in a composition of the present disclosure is less than 400 ppm. In an embodiment, the amount of the Na₂CO₃ residuals in a composition of the present disclosure is less than 500 ppm. In an embodiment, the amount of the Na₂CO₃ residuals in a composition of the present disclosure is less than 600 ppm. In an embodiment, the amount of the Na₂CO₃ residuals in a composition of the present disclosure is less than 700 ppm. In an embodiment, the amount of the Na₂CO₃ residuals in a composition of the present disclosure is less than 800 ppm. In an embodiment, the amount of the Na₂CO₃ residuals in a composition of the present disclosure is less than 900 ppm. In an embodiment, the amount of the Na₂CO₃ residuals in a composition of the present disclosure is less than 1000 ppm. In an embodiment, the amount of the Na₂CO₃ residuals in a composition of the present disclosure is non-detectable to 500 ppm. In an embodiment, the amount of the Na₂CO₃ residuals in a composition of the present disclosure is non-detectable to 450 ppm. In an embodiment, the amount of the Na₂CO₃ residuals in a composition of the present disclosure is non-detectable to 400 ppm. In an embodiment, the amount of the Na₂CO₃ residuals in a composition of the present disclosure is non-detectable to 350 ppm. In an embodiment, the amount of the Na₂CO₃ residuals in a composition of the present disclosure is non-detectable to 300 ppm. In an embodiment, the amount of the Na₂CO₃ residuals in a composition of the present disclosure is non-detectable to 250 ppm. In an embodiment, the amount of the Na₂CO₃ residuals in a composition of the present disclosure is non-detectable to 200 ppm. In an embodiment, the amount of the Na₂CO₃ residuals in a composition of the present disclosure is non-detectable to 150 ppm. In an embodiment, the amount of the Na₂CO₃ residuals in a composition of the present disclosure is non-detectable to 100 ppm. In an embodiment, the amount of the Na₂CO₃ residuals in a composition of the present disclosure is 100 ppm to 200 ppm. In an embodiment, the amount of the Na₂CO₃ residuals in a composition of the present disclosure is 200 ppm to 300 ppm. In an embodiment, the amount of the Na₂CO₃ residuals in a composition of the present disclosure is 300 ppm to 400 ppm. In an embodiment, the amount of the Na₂CO₃ residuals in a composition of the present disclosure is 400 ppm to 500 ppm.

A unique feature of the SPF compositions of the present disclosure are shelf stability (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally, pH may be altered to extend shelf-life and/or support shipping conditions by preventing premature folding and aggregation of the silk. In an embodiment, a SPF solution composition of the present disclosure has a shelf stability for up to 2 weeks at room temperature (RT). In an embodiment, a SPF solution composition of the present disclosure has a shelf stability for up to 4 weeks at RT. In an embodiment, a SPF solution composition of the present disclosure has a shelf stability for up to 6 weeks at RT. In an embodiment, a SPF solution composition of the present disclosure has a shelf stability for up to 8 weeks at RT. In an embodiment, a SPF solution composition of the present disclosure has a shelf stability for up to 10 weeks at RT. In an embodiment, a SPF solution composition of the present disclosure has a shelf stability for up to 12 weeks at RT. In an embodiment, a SPF solution composition of the present disclosure has a shelf stability ranging from about 4 weeks to about 52 weeks at RT.

Table 18 below shows shelf stability test results for embodiments of SPF compositions of the present disclosure.

TABLE 18
Shelf Stability of SPF Compositions of the Present Disclosure

| % Silk | Temperature | Time to Gelation |
|---|---|---|
| 2 | RT | 4 weeks |
| 2 | 4° C. | >9 weeks |
| 4 | RT | 4 weeks |
| 4 | 4° C. | >9 weeks |
| 6 | RT | 2 weeks |
| 6 | 4° C. | >9 weeks |

In some embodiments, the water solubility of the silk film derived from silk fibroin protein fragments as described herein can be modified by solvent annealing (water annealing or methanol annealing), chemical crosslinking, enzyme crosslinking and heat treatment.

In some embodiments, the process of annealing may involve inducing beta-sheet formation in the silk fibroin protein fragment solutions used as a coating material. Techniques of annealing (e.g., increase crystallinity) or otherwise promoting “molecular packing” of silk fibroin-protein based fragments have been described. In some embodiments, the amorphous silk film is annealed to introduce beta-sheet in the presence of a solvent selected from the group of water or organic solvent. In some embodiments, the amorphous silk film is annealed to introduce beta-sheet in the presence of water (water annealing process). In some embodiments, the amorphous silk fibroin protein fragment film is annealed to introduce beta-sheet in the presence of methanol. In some embodiments, annealing (e.g., the beta sheet formation) is induced by addition of an organic solvent. Suitable organic solvents include, but are not limited to methanol, ethanol, acetone, isopropanol, or combination thereof.

In some embodiments, annealing is carried out by so-called “water-annealing” or “water vapor annealing” in which water vapor is used as an intermediate plasticizing agent or catalyst to promote the packing of beta-sheets. In some embodiments, the process of water annealing may be performed under vacuum. Suitable such methods have been described in Jin H-J et al. (2005), Water-stable Silk Films with Reduced Beta-Sheet Content, Advanced Functional Materials, 15: 1241-1247; Xiao H. et al. (2011), Regulation of Silk Material Structure by Temperature-Controlled Water Vapor Annealing, Biomacromolecules, 12(5): 1686-1696.

The important feature of the water annealing process is to drive the formation of crystalline beta-sheet in the silk fibroin protein fragment peptide chain to allow the silk fibroin self-assembling into a continuous film. In some embodiments, the crystallinity of the silk fibroin protein fragment film is controlled by controlling the temperature of water vapor and duration of the annealing. In some embodiments, the annealing is performed at a temperature ranging from about 65° C. to about 110° C. In some embodiments, the temperature of the water is maintained at about 80° C. In some embodiments, annealing is performed at a temperature selected from the group of about 65° C., about 70° C., about 75° C., about 80° C., about 85° C., about 90° C., about 95° C., about 100° C., about 105° C., and about 110° C.

In some embodiments, the annealing process lasts a period of time selected from the group of about 1 minute to about 40 minutes, about 1 minute to about 50 minutes, about 1 minute to about 60 minutes, about 1 minute to about 70 minutes, about 1 minute to about 80 minutes, about 1 minute to about 90 minutes, about 1 minute to about 100 minutes, about 1 minute to about 110 minutes, about 1 minute to about 120 minutes, about 1 minute to about 130 minutes, about 5 minutes to about 40 minutes, about 5 minutes to about 50 minutes, about 5 minutes to about 60 minutes, about 5 minutes to about 70 minutes, about 5 minutes to about 80 minutes, about 5 minutes to about 90 minutes, about 5 minutes to about 100 minutes, about 5 minutes to about 110 minutes, about 5 minutes to about 120 minutes, about 5 minutes to about 130 minutes, about 10 minutes to about 40 minutes, about 10 minutes to about 50 minutes, about 10 minutes to about 60 minutes, about 10 minutes to about 70 minutes, about 10 minutes to about 80 minutes, about 10 minutes to about 90 minutes, about 10 minutes to about 100 minutes, about 10 minutes to about 110 minutes, about 10 minutes to about 120 minutes, about 10 minutes to about 130 minutes, about 15 minutes to about 40 minutes, about 15 minutes to about 50 minutes, about 15 minutes to about 60 minutes, about 15 minutes to about 70 minutes, about 15 minutes to about 80 minutes, about 15 minutes to about 90 minutes, about 15 minutes to about 100 minutes, about 15 minutes to about 110 minutes, about 15 minutes to about 120 minutes, about 15 minutes to about 130 minutes, about 20 minutes to about 40 minutes, about 20 minutes to about 50 minutes, about 20 minutes to about 60 minutes, about 20 minutes to about 70 minutes, about 20 minutes to about 80 minutes, about 20 minutes to about 90 minutes, about 20 minutes to about 100 minutes, about 20 minutes to about 110 minutes, about 20 minutes to about 120 minutes, about 20 minutes to about 130 minutes, about 25 minutes to about 40 minutes, about 25 minutes to about 50 minutes, about 25 minutes to about 60 minutes, about 25 minutes to about 70 minutes, about 25 minutes to about 80 minutes, about 25 minutes to about 90 minutes, about 25 minutes to about 100 minutes, about 25 minutes to about 110 minutes, about 25 minutes to about 120 minutes, about 25 minutes to about 130 minutes, about 30 minutes to about 40 minutes, about 30 minutes to about 50 minutes, about 30 minutes to about 60 minutes, about 30 minutes to about 70 minutes, about 30 minutes to about 80 minutes, about 30 minutes to about 90 minutes, about 30 minutes to about 100 minutes, about 30 minutes to about 110 minutes, about 30 minutes to about 120 minutes, about 30 minutes to about 130 minutes, about 35 minutes to about 40 minutes, about 35 minutes to about 50 minutes, about 35 minutes to about 60 minutes, about 35 minutes to about 70 minutes, about 35 minutes to about 80 minutes, about 35 minutes to about 90 minutes, about 35 minutes to about 100 minutes, about 35 minutes to about 110 minutes, about 35 minutes to about 120 minutes, about 35 minutes to about 130 minutes, about 40 minutes to about 50 minutes, about 40 minutes to about 60 minutes, about 40 minutes to about 70 minutes, about 40 minutes to about 80 minutes, about 40 minutes to about 90 minutes, about 40 minutes to about 100 minutes, about 40 minutes to about 110 minutes, about 40 minutes to about 120 minutes, about 40 minutes to about 130 minutes, about 45 minutes to about 50 minutes, about 45 minutes to about 60 minutes, about 45 minutes to about 70 minutes, about 45 minutes to about 80 minutes, about 45 minutes to about 90 minutes, about 45 minutes to about 100 minutes, about 45 minutes to about 110 minutes, about 45 minutes to about 120 minutes, and about 45 minutes to about 130 minutes. In some embodiments, the annealing process lasts a period of time ranging from about 1 minute to about 60 minutes. In some embodiments, the annealing process lasts a period of time ranging from about 45 minutes to about 60 minutes. The longer water annealing post-processing corresponded an increased crystallinity of silk fibroin protein fragments.

In some embodiments, the annealed silk fibroin protein fragment film is immersing the wet silk fibroin protein fragment film in 100% methanol for 60 minutes at room temperature. The methanol annealing changed the composition of silk fibroin protein fragment film from predominantly amorphous random coil to crystalline antiparallel beta-sheet structure.

In some embodiments, the SPF as described herein can be used to prepare SPF microparticles by precipitation with methanol. Alternative flash drying, fluid-bed drying, spray drying or vacuum drying can be applied to remove water from the silk solution. The SPF powder can then be stored and handled without refrigeration or other special handling procedures. In some embodiments, the SPF powders comprise low molecular weight silk fibroin protein fragments. In some embodiments, the SPF powders comprise mid-molecular weight silk fibroin protein fragments. In some embodiments, the SPF powders comprise a mixture of low molecular weight silk fibroin protein fragments and mid-molecular weight silk fibroin protein fragment.

### Silk Protein Fragments in Collagen Boosting Compositions and Methods Thereof

The disclosure provides a method of treatment or prevention of a disorder, disease, or condition alleviated by stimulating or modulating collagen expression in a subject in need thereof, comprising administering to the subject a composition comprising silk fibroin fragments, or without limitation any other silk protein fragments described herein, having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 14 kDa and about 30 kDa, between about 17 kDa and about 39 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5. Any other molecular weight, molecular weight range, and polydispersity of silk fibroin fragments, or without limitation any other silk protein fragments, described herein, can be used in the methods and compositions of the disclosure.

In some embodiments, the composition further comprises 0 to 500 ppm lithium bromide. In some embodiments, the composition further comprises 0 to 500 ppm sodium carbonate. In some embodiments, the silk fibroin fragments, or without limitation any other silk protein fragments described herein, have a polydispersity between 1 and about 1.5. In some embodiments, the silk fibroin fragments, or without limitation any other silk protein fragments described herein, have a polydispersity between about 1.5 and about 2.0. In some embodiments, the silk fibroin fragments, or without limitation any other silk protein fragments described herein, have a polydispersity between about 1.5 and about 3.0. In some embodiments, the silk fibroin fragments, or without limitation any other silk protein fragments described herein, have a polydispersity between about 2.0 and about 2.5. In some embodiments, the silk fibroin fragments, or without limitation any other silk protein fragments described herein, have a polydispersity between about 2.5 and about 3.0. In some embodiments, the silk fibroin fragments, or without limitation any other silk protein fragments described herein, are present in the composition at about 0.001 wt. % to about 10.0 wt. % relative to the total weight of the composition. In some embodiments, the composition further comprises about 0.001% (w/w) to about 10% (w/w) sericin relative to the total weight of the composition. In some embodiments, the composition further comprises about 0.001% (w/w) to about 10% (w/w) sericin relative to the silk fibroin fragments, or without limitation any other silk protein fragments described herein. In some embodiments, the silk fibroin fragments, or without limitation any other silk protein fragments described herein, do not spontaneously or gradually gelate and do not visibly change in color or turbidity when in an aqueous solution for at least 10 days prior to formulation into the composition. In some embodiments, the silk fibroin fragments, or without limitation any other silk protein fragments described herein, are present in the composition at about 0.01 wt. % to about 10.0 wt. % relative to the total weight of the composition. In some embodiments, the silk fibroin fragments, or without limitation any other silk protein fragments described herein, are present in the composition at about 0.01 wt. % to about 1.0 wt. % relative to the total weight of the composition. In some embodiments, the silk fibroin fragments, or without limitation any other silk protein fragments described herein, are present in the composition at about 1.0 wt. % to about 2.0 wt. % relative to the total weight of the composition. In some embodiments, the silk fibroin fragments, or without limitation any other silk protein fragments described herein, are present in the composition at about 2.0 wt. % to about 3.0 wt. % relative to the total weight of the composition. In some embodiments, the silk fibroin fragments, or without limitation any other silk protein fragments described herein, are present in the composition at about 3.0 wt. % to about 4.0 wt. % relative to the total weight of the composition. In some embodiments, the silk fibroin fragments, or without limitation any other silk protein fragments described herein, are present in the composition at about 4.0 wt. % to about 5.0 wt. % relative to the total weight of the composition. In some embodiments, the silk fibroin fragments, or without limitation any other silk protein fragments described herein, are present in the composition at about 5.0 wt. % to about 6.0 wt. % relative to the total weight of the composition.

In some embodiments, the composition is formulated as an injectable composition or as a topical composition. In some embodiments, the composition is formulated for improving look and feel of skin, including without limitation by boosting collagen (e.g., without limitation, stimulating or modulating collagen expression). In some embodiments, the composition is formulated for boosting collagen on skin. In some embodiments, the composition is formulated for boosting collagen intradermally. In some embodiments, the composition is formulated for boosting collagen on scalp. In some embodiments, the composition is formulated as a liquid solution for boosting collagen. In some embodiments, the composition is formulated as a film for boosting collagen. In some embodiments, the composition is formulated as a solid for boosting collagen. In some embodiments, the composition is formulated as a powder for boosting collagen. In some embodiments, the composition is formulated as a gel for boosting collagen. In some embodiments, the composition is formulated as a silk gel for boosting collagen. In some embodiments, the composition is formulated as a silk/HA gel, with or without lidocaine, for boosting collagen. In some embodiments, the composition is formulated as a soap for boosting collagen. In some embodiments, the composition is formulated as a cream for boosting collagen. In some embodiments, the composition is formulated as a lotion for boosting collagen. In some embodiments, the composition is formulated as a shampoo for boosting collagen. In some embodiments, the composition is formulated as a conditioner for boosting collagen. In some embodiments, the composition is formulated as a nourishing agent for boosting collagen. In some embodiments, the composition is formulated as a mask for boosting collagen. In some embodiments, the composition is formulated as an over the counter product for boosting collagen. In some embodiments, the composition is formulated as a drug for boosting collagen. In some embodiments, the composition is formulated as a therapeutic for boosting collagen. In some embodiments, the composition is formulated as a silk-coated fabric for boosting collagen. In some embodiments, the composition is formulated as a silk-coated non-woven material for boosting collagen.

In some embodiments, the composition further comprises a pharmaceutically acceptable carrier. In some embodiments, the composition further comprises a dermatologically acceptable carrier. In some embodiments, the composition further comprises an injectable acceptable carrier. In some embodiments, the pharmaceutically acceptable carrier comprises one or more of a suspension, an emulsion, a powder, a solution, a dispersion, or an elixir. In some embodiments, the pharmaceutically acceptable carrier comprises or is formulated as one or more of a gel, a jelly, a cream, a lotion, a foam, a slurry, an ointment, an oil, a paste, a suppository, a spray, a semisolid composition, a solid composition, a stick, or a mousse. In some embodiments, the pharmaceutically acceptable carrier comprises one or more of sesame oil, corn oil, cottonseed oil, or peanut oil. In some embodiments, the pharmaceutically acceptable carrier comprises one or more of mannitol or dextrose. In some embodiments, the pharmaceutically acceptable carrier comprises about 0.001% to about 10% (w/v) hyaluronic acid. In some embodiments, the pharmaceutically acceptable carrier comprises about 1% to about 10% (w/v), about 10% to about 25% (w/v), about 25% to about 50% (w/v), or about 50% to about 99.99% (w/v) hyaluronic acid. In some embodiments, HA described herein has a molecular weight of 100,000 daltons or greater, 150,000 daltons or greater, 1 million daltons or greater, or 2 million daltons or greater. In some embodiments, HA described herein has a molecular weight of 100,000 daltons or less, 150,000 daltons or less, 1 million daltons or less, or 2 million daltons or less. In some embodiments, the HA described herein has a high molecular weight (e.g., an HA molecular weight of about 1 MDa to about 4 MDa). In some embodiments, the HA described herein has a low molecular weight (e.g., an HA molecular weight of less than about 1 MDa). In some embodiments, the HA source may be a hyaluronate salt such as, for example, sodium hyaluronate. In some embodiments, the HA is crosslinked. Crosslinked HA can be formulated into a variety of shapes, such as membranes, gels, semi-gels, sponges, or microspheres. In some embodiments, the crosslinked HA is in fluid gel form, i.e., it takes the shape of its container. The viscosity of an HA gel or semi-gel can be altered by the addition of unconjugated HA and/or hyaluronate. Viscosity can also be tuned by varying the degree of SPF-SPF, SPF-HA, and/or HA-HA cross-linking as described herein. In some embodiment, about 4% to about 12% of the HA may be crosslinked as HA-HA or HA-SPF.

In some embodiments, the pharmaceutically acceptable carrier comprises one or more of aliphatic oil, a fatty alcohol, a fatty acid, a glyceride, an acylglycerol, and a phospholipid. In some embodiments, the pharmaceutically acceptable carrier comprises one or more of a monoglyceride, a diglyceride, or a triglyceride. In some embodiments, the pharmaceutically acceptable carrier comprises an aqueous phase. In some embodiments, the pharmaceutically acceptable carrier comprises an oil-in-water emulsion or a water-in-oil emulsion. In some embodiments, the pharmaceutically acceptable carrier comprises one or more of a hydrocarbon oil, a fatty acid, a fatty oil, a fatty acid ester, or a cationic quaternary ammonium salt. In some embodiments, a portion of the pharmaceutically acceptable carrier is modified with a cross-linking agent, a cross-linking precursor, or an activating agent selected from a polyepoxy linker, a diepoxy linker, a polyepoxy-PEG, a diepoxy-PEG, a polyglycidyl-PEG, a diglycidyl-PEG, a poly acrylate PEG, a diacrylate PEG, 1,4-bis(2,3-epoxypropoxy)butane, 1,4-bisglycidyloxybutane, divinyl sulfone (DVS), 1,4-butanediol diglycidyl ether (BDDE), UV light, glutaraldehyde, 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), 1-(2,3-epoxypropyl)-2,3-epoxycyclohexane, a carbodiimide, and any combinations thereof. In some embodiments, the polyepoxy linker is selected from 1,4-butanediol diglycidyl ether (BDDE), ethylene glycol diglycidyl ether (EGDGE), 1,6-hexanediol diglycidyl ether, polyethylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylolpropane polyglycidyl ether, pentaerythritol polyglycidyl ether, and sorbitol polyglycidyl ether.

In some embodiments, the composition further comprises an anesthetic compound. In some embodiments, the compound is selected from benzocaine, chloroprocaine, cocaine, cyclomethycaine, dimethocaine, piperocaine, propoxycaine, procaine, proparacaine, tetracaine, articaine, bupivacaine, cinchocaine, etidocaine, levobupivacaine, lidocaine, mepivacaine, prilocaine, ropivacaine, and trimecaine. In some embodiments, the composition further includes lidocaine. In some embodiments, the concentration of lidocaine in the composition is between about 0.01% and about 1%, including any increment of 0.01%. In some embodiments, the concentration of lidocaine in the composition is about 0.3%.

In certain embodiments, the compositions described herein can include one or more anesthetic agents in an amount effective to ameliorate or mitigate pain or discomfort at a composition injection site. The local anesthetic can be selected from the group of ambucaine, amolanone, amylocalne, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocalne, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

In some embodiments, the compositions described herein may include lidocaine or other anesthetic recited herein at a concentration, by weight, of about 0.01% to about 0.02%, or about 0.03% to about 0.04%, or about 0.05% to about 0.06% to about 0.07%, or about 0.08% to about 0.09%, or about 0.1% to about 0.2%, or about 0.3% to about 0.4%, or about 0.5% to about 0.6%, or about 0.7% to about 0.8%, or about 0.9% to about 1.0%, or about 1% to about 1.5%, or about 1.5% to about 2.0%, or about 2.0% to about 2.5%, or about 2.5% to about 3.0%, or about 3.0% to about 3.5%, or about 3.5% to about 4.0%, or about 4.0% to about 4.5%, or about 4.5% to about 5.0%, or about 5.0% to about 5.5%, or about 5.5% to about 6.0%, or about 6.0% to about 6.5%, or about 6.5% to about 7.0%, or about 7.5% to about 8.0%, or about 8.0% to about 8.5%, or about 8.5% to about 9.0%, or about 9.5% to about 10%.

In some embodiments, the pharmaceutically acceptable carrier comprises or is formulated as a gel. The gel can be either an injectable gel, for example but without limitation, a tissue filler, or a gel for topical administration. Suitable gels are described for example in WO2019005848, incorporated herein by reference. In some embodiments, the gel comprises silk fibroin or silk fibroin fragments, or any other SPF described herein, hyaluronic acid (HA), and polyethylene glycol (PEG) and/or polypropylene glycol (PPG). In some embodiments, a portion of the HA is modified or crosslinked by one or more linker moieties comprising one or more of polyethylene glycol (PEG), polypropylene glycol (PPG), and a secondary alcohol, wherein the linker moieties are attached to the HA at one end of the linker. In some embodiments, a portion of the silk fibroin or silk fibroin fragments, or any other SPF described herein, are modified or crosslinked. In some embodiments, a portion of the silk fibroin or silk fibroin fragments, or any other SPF described herein, are free. In some embodiments, a portion of the silk fibroin or silk fibroin fragments, or any other SPF described herein, are crosslinked to HA. In some embodiments, a portion of the silk fibroin or silk fibroin fragments, or any other SPF described herein, are crosslinked to silk fibroin or silk fibroin fragments, or any other SPF described herein. In some embodiments, the silk fibroin or silk fibroin fragments are substantially devoid of sericin. In some embodiments, the gel has a degree of modification (MoD) of about 5%, about 6%, about 7%, about 8%, about 9%, about 10%, about 11%, about 12%, about 13%, about 14%, or about 15%. In some embodiments, modification or crosslinking is obtained using as crosslinker a diepoxy-PEG, a polyglycidyl-PEG, a diglycidyl-PEG, a diepoxy-PPG, a polyglycidyl-PPG, a diglycidyl-PPG, or any combinations thereof. In some embodiments, modification or cross-linking is obtained using polyethylene glycol diglycidyl ether having a MW of about 200 Da, about 500 Da, 1000 Da, about 2,000 Da, or about 6000 Da. In some embodiments, modification or cross-linking is obtained using polypropylene glycol diglycidyl ether having a MW of about 380 Da, or about 640 Da. In some embodiments, the gel is a hydrogel. In some embodiments, the gel further includes water. In some embodiments, the gel is monophasic. In some embodiments, the total concentration of HA and silk in the gel is about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, or about 30 mg/mL. In some embodiments, the ratio of HA to silk fibroin or silk fibroin fragments in the gel is about 92/8, about 93/7, about 94/6, about 95/5, about 96/4, about 97/3, about 18/12, about 27/3, about 29.4/0.6, about 99/1, about 92.5/7.5, or about 90/10. In some embodiments, the gel is a dermal filler. In some embodiments, the gel is biodegradable. In some embodiments, the gel is injectable. In some embodiments, the gel is injectable through 30 G or 27 G needles. In some embodiments, the gel has a storage modulus (G′) of from about 5 Pa to about 500 Pa. In some embodiments, G′ is measured by means of an oscillatory stress of about 1 Hz, about 5 Hz, or about 10 Hz. In some embodiments, the gel has a complex viscosity from about 1 Pa s to about 10 Pa s. In some embodiments, the complex viscosity is measured by means of an oscillatory stress of about 1 Hz, about 5 Hz, or about 10 Hz. In some embodiments, the gel comprises a glycosaminoglycan selected from the group consisting of hyaluronic acid (HA), carboxymethyl cellulose (CMC), starch, alginate, chondroitin-4-sulfate, chondroitin-6-sulfate, xanthan gum, chitosan, pectin, agar, carrageenan, and guar gum.

In some embodiments, the composition is administered parenterally. In some embodiments, the composition is an injectable composition. In some embodiments, the composition is administered by injection. In some embodiments, the composition is administered by subcutaneous injection, intradermal injection, transdermal injection, or subdermal injection. In some embodiments, the composition is administered by intramuscular injection, intravenous injection, intraperitoneal injection, intraosseous injection, intracardiac injection, intraarticular injection, or intracavernous injection. In some embodiments, the composition is administered by depot injection. In some embodiments, the composition is administered by infiltration injection. In some embodiments, the composition is administered by an indwelling catheter. In some embodiments, the composition, or portions thereof, is biocompatible, biodegradable, bioabsorbable, bioresorbable, or a combination thereof. In some embodiments, the composition provided herein include a fluid component, for example a single fluid or a solution including substantially one or more fluids. In some embodiments, the composition includes water or an aqueous solution. In some embodiments, the composition is injectable, implantable, or deliverable under the skin by any means known in the art such as, for example, following surgical resection of the tissue. In some embodiments, the compositions are dermal fillers. In some embodiments, the compositions are sterile.

In an embodiment, the percent water content, by weight, in the compositions described herein is about 1%, or about 2%, or about 3%, or about 4%, or about 5%, or about 6%, or about 7%, or about 8%, or about 9%, or about 10%, or about 11%, or about 12%, or about 13%, or about 14%, or about 15%, or about 16%, or about 17%, or about 18%, or about 19%, or about 20%, or about 21%, or about 22%, or about 23%, or about 24%, or about 25%, or about 26%, or about 27%, or about 28%, or about 29%, or about 30%, or about 31%, or about 32%, or about 33%, or about 34%, or about 35%, or about 36%, or about 37%, or about 38%, or about 39%, or about 40%, or about 41%, or about 42%, or about 43%, or about 44%, or about 45%, or about 46%, or about 47%, or about 48%, or about 49%, or about 50%, or about 51%, or about 52%, or about 53%, or about 54%, or about 55%, or about 56%, or about 57%, or about 58%, or about 59%, or about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%.

In some embodiments, the composition can be administered in and about soft tissue to add volume, add support, or otherwise treat a soft tissue deficiency, in addition to boosting collagen expression. The compositions described herein can be administered at multiple levels beneath the dermis. As used herein, the term “soft tissue” may refer to those tissues that connect, support, or surround other structures and organs of the body. For example, soft tissues described herein may include, without limitation, skin, dermal tissues, subdermal tissues, cutaneous tissues, subcutaneous tissues, intradural tissue, muscles, tendons, ligaments, fibrous tissues, fat, blood vessels and arteries, nerves, and synovial (intradermal) tissues. In some embodiments, the disclosure provides methods of treating a soft tissue condition of an individual, including administering one or more compositions disclosed herein to a site of the soft tissue condition of the individual, wherein the administration of the composition improves the soft tissue condition, thereby treating the soft tissue condition. In some embodiments, a soft tissue condition is a breast tissue condition, a facial tissue condition, a neck condition, a skin condition, an upper arm condition, a lower arm condition, a hand condition, a shoulder condition, a back condition, a torso including abdominal condition, a buttock condition, an upper leg condition, a lower leg condition including calf condition, a foot condition including plantar fat pad condition, an eye condition, a genital condition, or a condition effecting another body part, region or area.

In some embodiments, the disclosure provides for compositions and methods of treatment involving a dermal region, including without limitation, the region of skin comprising the epidermal-dermal junction and the dermis including the superficial dermis (papillary region) and the deep dermis (reticular region). The skin is composed of three primary layers: the epidermis, which provides waterproofing and serves as a barrier to infection; the dermis, which serves as a location for the appendages of skin; and the hypodermis (subcutaneous adipose layer). The epidermis contains no blood vessels, and is nourished by diffusion from the dermis. The main type of cells which make up the epidermis are keratinocytes, melanocytes, Langerhans cells, and Merkels cells.

In an embodiment, the compositions described herein may be provided in methods of treating one or more conditions in a patient in need thereof. In some embodiments, a therapeutically effective amount of a composition may be delivered into a tissue of a patient in need thereof to treat a condition or other tissue deficiency.

As used herein, the term “treating,”, “treat”, or “treatment” refers to reducing or eliminating in a patient a cosmetic or clinical symptom of a condition, such as a soft tissue condition, or delaying or preventing in an individual the onset of a cosmetic or clinical symptom of a condition.

In some embodiments, the condition treated by the compositions described herein may include a soft tissue condition. Soft tissue conditions include, without limitation, augmentations, reconstructions, diseases, disorders, defects, or imperfections of a body part, region or area. In one aspect, a soft tissue condition treated by the disclosed compositions include, without limitation, a facial augmentation, a facial reconstruction, a facial disease, a facial disorder, a facial defect, or a facial imperfection. In some embodiments, a soft tissue condition treated by the compositions described herein include, without limitation, skin dehydration, a lack of skin elasticity, skin roughness, a lack of skin tautness, a skin stretch line or mark, skin paleness, a dermal divot, a sunken check, a sunken temple, a thin lip, a urethra defect, a skin defect, a breast defect, a retro-orbital defect, a facial fold, or a wrinkle. In some embodiments, a soft tissue condition treated by the compositions described herein include, without limitation, breast imperfection, defect, disease and/or disorder, such as, e.g., a breast augmentation, a breast reconstruction, mastopexy, micromastia, thoracic hypoplasia, Poland's syndrome, defects due to implant complications like capsular contraction and/or rupture; a facial imperfection, defect, disease or disorder, such as, e.g., a facial augmentation, a facial reconstruction, Parry-Romberg syndrome, lupus erythematosus profundus, dermal divots, sunken cheeks, sunken temples, thin lips, nasal imperfections or defects, retro-orbital imperfections or defects, a facial fold, line and/or wrinkle like a glabellar line, a nasolabial line, a perioral line, and/or a marionette line, and/or other contour deformities or imperfections of the face; a neck imperfection, defect, disease or disorder; a skin imperfection, defect, disease and/or disorder; other soft tissue imperfections, defects, diseases and/or disorders, such as, e.g., an augmentation or a reconstruction of the upper arm, lower arm, hand, shoulder, back, torso including abdomen, buttocks, upper leg, lower leg including calves, foot including plantar fat pad, eye, genitals, or other body part, region or area, or a disease or disorder affecting these body parts, regions or areas; urinary incontinence, fecal incontinence, other forms of incontinence; and gastroesophageal reflux disease (GERD).

In some embodiments, the compositions described herein may be delivered to soft tissues including, without limitation skin, dermal tissues, subdermal tissues, cutaneous tissues, subcutaneous tissues, intradural tissue, muscles, tendons, ligaments, fibrous tissues, fat, blood vessels and arteries, nerves, and synovial (intradermal) tissues.

In some embodiments, the compositions described herein can be placed directly in a wound to aid in healing by providing an artificial biodegradable matrix along with cell attachment, migration, and proliferation signals. In some embodiments, the compositions described herein can be coated on a biodegradable mesh or other implanted material, or it can itself be formed into sheets or other structures, or can be maintained in a hydrated form.

In some embodiments, the amount of a composition used with any of the methods as disclosed herein will be determined based on the alteration and/or improvement desired, the reduction and/or elimination of a condition symptom desired, the clinical and/or cosmetic effect desired by the individual and/or physician, and the body part or region being treated. The effectiveness of composition administration may be manifested by one or more of the following clinical and/or cosmetic measures: altered and/or improved soft tissue shape, altered and/or improved soft tissue size, altered and/or improved soft tissue contour, altered and/or improved tissue function, tissue ingrowth support and/or new collagen deposition, sustained engraftment of the composition, improved patient satisfaction and/or quality of life, and decreased use of implantable foreign material. For example, for breast augmentation procedures, effectiveness of the compositions and methods may be manifested by one or more of the following clinical and/or cosmetic measures: increased breast size, altered breast shape, altered breast contour, sustained engraftment, reduction in the risk of capsular contraction, decreased rate of liponecrotic cyst formation, improved patient satisfaction and/or quality of life, and decreased use of breast implant.

In some embodiments, administering the composition decreases expression of one or more metalloproteinases (MMP) in the subject. In some embodiments, stimulating or modulating collagen expression comprises increasing collagen expression.

In some embodiments, collagen expression is increased over a base level by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, collagen expression is increased over a base level by about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 1²²%, about 123%, about 124%, about 125%, about 126%, about 127%, about 128%, about 129%, about 130%, about 131%, about 132%, about 133%, about 134%, about 135%, about 136%, about 137%, about 138%, about 139%, about 140%, about 141%, about 142%, about 143%, about 144%, about 145%, about 146%, about 147%, about 148%, about 149%, about 150%, about 151%, about 152%, about 153%, about 154%, about 155%, about 156%, about 157%, about 158%, about 159%, about 160%, about 161%, about 162%, about 163%, about 164%, about 165%, about 166%, about 167%, about 168%, about 169%, about 170%, about 171%, about 172%, about 173%, about 174%, about 175%, about 176%, about 177%, about 178%, about 179%, about 180%, about 181%, about 182%, about 183%, about 184%, about 185%, about 186%, about 187%, about 188%, about 189%, about 190%, about 191%, about 192%, about 193%, about 194%, about 195%, about 196%, about 197%, about 198%, about 199%, or about 200%.

In some embodiments, collagen expression is increased over a base level by about 225%, about 250%, about 275%, about 300%, about 325%, about 350%, about 375%, about 400%, about 425%, about 450%, about 475%, about 500%, about 525%, about 550%, about 575%, about 600%, about 625%, about 650%, about 675%, about 700%, about 725%, about 750%, about 775%, about 800%, about 825%, about 850%, about 875%, about 900%, about 925%, about 950%, about 975%, or about 1000%.

In some embodiments, administering the composition results in one or more of preventing or reversing wrinkles in the subject, preventing or reversing age spots in the subject, preventing or reversing dry skin in the subject, increasing uneven skin tone in the subject, or improving look and feel of skin. Improving look and feel of skin includes without limitation improving look and feel of damaged skin, but also improving look and feel of skin which is not otherwise visibly damaged. In some embodiments, administering the composition results in one or more of preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject. In some embodiments, the disorder, disease, or condition comprises wrinkles, age spots, dry skin, uneven skin tone, skin sagging, skin aging, reduced skin tensile strength, photodamaged skin, or striae distensae (stretch marks). In some embodiments, the disorder, disease, or condition comprises a skin condition. In some embodiments, the skin condition can be skin dehydration, lack of skin elasticity, skin roughness, lack of skin tautness, a skin stretch line, a skin stretch mark, skin paleness, a dermal divot, a sunken cheek, sunken temple, a thin lip, a retro-orbital defect, a facial fold, or a wrinkle.

In some embodiments, the methods of treatment disclosed comprise an augmentation, a reconstruction, treating a disease, treating a disorder, correcting a defect or imperfection of a body part, region or area. In some embodiments, the methods of treatment disclosed comprise a facial augmentation, a facial reconstruction, treating a facial disease, treating a facial disorder, treating a facial defect, or treating a facial imperfection.

In some embodiments, the methods of treatment provided include one or more of administering a composition of the disclosure before, after, or during a laser treatment, administering a composition of the disclosure before, after, or during a skin peel, administering a composition of the disclosure before, after, or during a radiation treatment. In some embodiments, the methods of treatment provided include one or more of administering a composition of the disclosure to treat a burn, including without limitation any type of burn (e.g., thermic burn, sunburn, fire burn, hot liquid burn, radiation burn, chemical burn, and the like). In some embodiments, the methods of treatment provided include one or more of administering a composition of the disclosure to treat a burn, including without limitation a first-, second-, or third-degree burn. In some embodiments, the methods of treatment provided include one or more of administering a composition of the disclosure for treating a skin condition due to aging.

In some embodiments, the disorder, disease, or condition comprises thyroid hormone-induced myocardial hypertrophy. In some embodiments, the disorder, disease, or condition comprises a tendon rupture, damage, or tear. In some embodiments, the tendon is selected from Teres minor tendons, Infraspinatus tendons, Supraspinatus tendons, Subscapularis tendons, Deltoid tendons, Biceps tendons, Triceps tendons, Brachioradialis tendons, Supinator tendons, Flexor carpi radialis tendons, Flexor carpi ulnaris tendons, Extensor carpi radialis tendons, Extensor carpi radialis brevis tendons, Iliopsoas tendons, Obturator internus tendons, Adductor longus, brevis or magnus tendons, Gluteus maximus or gluteus medius tendons, Quadriceps tendons, patellar tendon, Hamstring tendons, Sartorius tendons, Gastrocnemius tendons, Achilles tendon, Soleus tendons, Tibialis anterior tendons, Peroneus longus tendons, Flexor digitorum longus tendons, Interosseus tendons, Flexor digitorum profundus tendons, Abductor digiti minimi tendons, Opponens pollicis tendons, Flexor pollicis longus tendons, Extensor or abductor pollicis tendons, Flexor hallucis longus tendons, Flexor digitorum brevis tendons, Lumbrical tendons, Abductor hallucis tendons, Flexor digitorum longus tendons, Abductor digiti minimi tendons, Ocular tendons, Levator palpebrae tendons, Masseter tendons, Temporalis tendons, Trapezius tendons, Sternocleidomastoid tendons, Semispinalis capitis or splenius capitis tendons, Mylohyoid or thyrohyoid tendons, Sternohyoid tendons, Rectus abdominis tendons, External oblique tendons, Transversus abdominis tendons, Latissimus dorsi tendons, and Erector spinae tendons. In some embodiments, the disorder, disease, or condition comprises Werner's syndrome. In some embodiments, the disorder, disease, or condition comprises diminished diabetic skin integrity. In some embodiments, the disorder, disease, or condition comprises arthritis. In some embodiments, the disorder, disease, or condition comprises rheumatoid arthritis. In some embodiments, the disorder, disease, or condition comprises tumor progression or tumor growth. In some embodiments, the disorder, disease, or condition comprises diminished cardiac function. In some embodiments, the disorder, disease, or condition comprises Ehlers-Danlos syndrome. In some embodiments, the disorder, disease, or condition comprises abdominal aortic aneurysms. In some embodiments, the disorder, disease, or condition comprises a wound. In some embodiments, the disorder, disease, or condition comprises a skin or connective tissue disease. In some embodiments, the disorder, disease, or condition comprises a cartilage disease. In some embodiments, the disorder, disease, or condition is selected from relapsing polychondritis, Tietze's Syndrome, cellulitis, Ehler's Danlos syndrome, keloids (including acne keloids), mucopolysaddaridosis I, necrobiotic disorders (including granuloma annulare, necrobiosis lipoidica), osteogenesis imperfect, cutis laxa, dermatomyositis, Dupytren's contracture, homocystinuria, lupus erythematosis (including cutaneous, discoid, panniculitis, systemic and nephritis), marfan syndrome, mixed connective tissue disease, mucinosis (including follicular), mucopolysaccaridoses (I, II, UU, IV, IV, and VII), myxedema, scleredemo adultorum and synovial cysts, connective tissue neoplasms, Noonan syndrome, osteopoikilosis, panniculitis, including erythema induratum, nodular nonsuppurative and peritoneal, penile induration, pseudoxanthoma elasticum, rheumatic diseases, including arthritis (rheumatoid, juvenile rheumatoid, Caplan's syndrome, Felty's syndrome, rheumatoid nodule, ankylosing spondylitis, and still's disease), hyperostosis, polymyalgia rheumatics, circumscribed scleroderma, and systemic scleroderma (CREST syndrome). In some embodiments, the disorder, disease, or condition is selected from angiolymphoid hyperplasia with eosinophilia; cicatix (including hypertophic); cutaneous fistula, cuis laxa; dermatitis, including acrodermatitis, atopic dermatitis, contact dermatitis (allergic contact, photoallergic, toxicodendron), irritant dermatitis (phototoxic, diaper rash), occupational dermatitis; exfoliative dermatitis, herpetiformis dermatitis, seborrheic dermatitis, drug eruptions (such as toxic epidermal necrolysis, erythema nodosum, serum sickness) eczema, including dyshidrotic, intertrigo, neurodermatitis, and radiodermatitis; dermatomyositis; erythema, including chronicum migrans, induratum, infectiosum, multiforme (Stevens-Johnson syndrome), and nodosum (Sweet's syndrome); exanthema, including subitum; facial dermatosis, including acneiform eruptions (keloid, rosacea, vulgaris and Favre-Racouchot syndrome); foot dermatosis, including tinea pedis; hand dermatoses; keratoacanthoma; keratosis, including callosities, cholesteatoma (including middle ear), ichthyosis (including congenital ichtyosiform erythroderms, epidermolytic hyperkeratosis, lamellar ichthyosis, ichthyosis vulgaris, X-linked ichthyosis, and Sjogren-Larsson syndrome), keratoderma blennorrhagicum, palmoplantar keratoderms, follicularis keratosis, seborrheic keratosis, parakeratosis and porokeratosis; leg dermatosis, mastocytosis (urticaria pigmentosa), necrobiotic disorders (granuloma annulare and necrobiosis lipoidica), photosensitivity disorders (photoallergic or photoxic dermatitis, hydroa vacciniforme, sundurn, and xeroderma pigmentosum); pigmentation disorders, including argyria, hyperpigmentation, melanosis, aconthosis nigricans, lentigo, Peutz-Jeghers syndrome, hypopigmentation, albinism, pibaldism, vitiligo, incontinentia pigmenti, urticaria pigmentosa, xeroderma pigmentosum, prurigo; pruritis (including ani and vulvae); pyoderma, including ecthyma and pyoderma gangrenosum; sclap dermatoses; sclerodema adultorum; sclerma neonatorum; skin appenage diseases, including hair diseases (alopecia, folliculitis, hirsutism, hypertichosis, Kinky hair syndrome), nail diseases (nail-patella syndrome, ingrown or malformed nails, onychomycosis, paronychia), sebaceous gland diseases (rhinophyma, neoplasms), sweat gland diseases (hidradenitis, hyperhidrosis, hypohidrosis, miliara, Fox-Fordyce disease, neoplasms); genetic skin diseases, including alfinism, cutis laxa, benign familial pemphigis, porphyria, acrodermatitis, ectodermal dysplasia, Ellis-Van Creveld syndrome, focal dermal hypoplasia, Ehlers-Danlos syndrome, epidermolysis bullosa, ichtysosis; infectious skin diseases, including dermatomycoses, blastomycosis, candidiasis, chromoblastomycosis, maduromycosis, paracoccidioidomycosis, sporotrichosis, tinea; bacterial skin diseases, such as cervicofacial actinomycosis, bacilliary angiomatosis, ecthyma, erysipelas, erythema chronicum migrans, erythrasma, granuloma inguinale, hidradenitis suppurativa, maduromycosis, paronychia, pinta, rhinoscleroma, staphylococcal skin infections (furuncolosis, carbuncle, impetigo, scalded skin syndrome), cutaneous syphilis, cutaneous tuberculosis, yaws; parasitic skin diseases, including larva migrans, Leishmaniasis, pediculosis, and scabies; viral skin diseases, including erythema infectiosum, exanthema subitum, herpes simplex, moolusum contagiosum, and warts.

In some embodiments, the amount of a composition used with any of the methods disclosed herein will typically be a therapeutically effective amount. As used herein, the term “therapeutically effective amount” is synonymous with “effective amount”, “therapeutically effective dose”, and/or “effective dose,” and refers to the amount of composition that will elicit the expected biological, cosmetic, or clinical response in a patient in need thereof. As a non-limiting example, an effective amount is an amount sufficient to achieve one or more of the clinical and/or cosmetic measures disclosed herein. The appropriate effective amount to be administered for a particular application of the disclosed methods can be determined by those skilled in the art, using the guidance provided herein. For example, an effective amount can be extrapolated from any and all in vitro and in vivo assays as described herein. One skilled in the art will recognize that the condition of the individual can be monitored throughout the course of therapy and that the effective amount of a composition disclosed herein that is administered can be adjusted accordingly.

In some embodiments, the amount of a composition administered is, without limitation, at least 0.001 g, or at least 0.002 g, or at least 0.003 g, or at least 0.004 g, or at least 0.005 g, or at least 0.006 g, or at least 0.007 g, or at least 0.008 g, or at least 0.009 g, or at least 0.01 g, or at least 0.02 g, or at least 0.03 g, or at least 0.04 g, or at least 0.05 g, or at least 0.06 g, or at least 0.07 g, or at least 0.08 g, or at least 0.09 g, or at least 0.1 g, or at least 0.2 g, or at least 0.3 g, or at least 0.4 g, or at least 0.5 g, or at least 0.6 g, or at least 0.7 g, or at least 0.8 g, or at least 0.9 g, or at least 1 g, or at least 2 g, or at least 3 g, or at least 4 g, or at least 5 g, or at least 6 g, or at least 7 g, or at least 8 g, or at least 9 g, or at least 10 g, or at least 11 g, or at least 12 g, or at least 13 g, or at least 14 g, or at least 15 g, or at least 20 g, or at least 25 g, or at least 30 g, or at least 35 g, or at least 40 g, or at least 45 g, or at least 50 g, or at least 55 g, or at least 60 g, or at least 65 g, or at least 70 g, or at least 75 g, or at least 80 g, or at least 85 g, or at least 90 g, or at least 95 g, or at least 100 g.

In some embodiments, the amount of a composition administered is, without limitation, at most 0.001 g, or at most 0.002 g, or at most 0.003 g, or at most 0.004 g, or at most 0.005 g, or at most 0.006 g, or at most 0.007 g, or at most 0.008 g, or at most 0.009 g, or at most 0.01 g, or at most 0.02 g, or at most 0.03 g, or at most 0.04 g, or at most 0.05 g, or at most 0.06 g, or at most 0.07 g, or at most 0.08 g, or at most 0.09 g, or at most 0.1 g, or at most 0.2 g, or at most 0.3 g, or at most 0.4 g, or at most 0.5 g, or at most 0.6 g, or at most 0.7 g, or at most 0.8 g, or at most 0.9 g, or at most 1 g, or at most 2 g, or at most 3 g, or at most 4 g, or at most 5 g, or at most 6 g, or at most 7 g, or at most 8 g, or at most 9 g, or at most 10 g, or at most 11 g, or at most 12 g, or at most 13 g, or at most 14 g, or at most 15 g, or at most 20 g, or at most 25 g, or at most 30 g, or at most 35 g, or at most 40 g, or at most 45 g, or at most 50 g, or at most 55 g, or at most 60 g, or at most 65 g, or at most 70 g, or at most 75 g, or at most 80 g, or at most 85 g, or at most 90 g, or at most 95 g, or at most 100 g.

In some embodiments, the amount of a composition administered is, without limitation, about 0.001 g, or about 0.002 g, or about 0.003 g, or about 0.004 g, or about 0.005 g, or about 0.006 g, or about 0.007 g, or about 0.008 g, or about 0.009 g, or about 0.01 g, or about 0.02 g, or about 0.03 g, or about 0.04 g, or about 0.05 g, or about 0.06 g, or about 0.07 g, or about 0.08 g, or about 0.09 g, or about 0.1 g, or about 0.2 g, or about 0.3 g, or about 0.4 g, or about 0.5 g, or about 0.6 g, or about 0.7 g, or about 0.8 g, or about 0.9 g, or about 1 g, or about 2 g, or about 3 g, or about 4 g, or about 5 g, or about 6 g, or about 7 g, or about 8 g, or about 9 g, or about 10 g, or about 11 g, or about 12 g, or about 13 g, or about 14 g, or about 15 g, or about 20 g, or about 25 g, or about 30 g, or about 35 g, or about 40 g, or about 45 g, or about 50 g, or about 55 g, or about 60 g, or about 65 g, or about 70 g, or about 75 g, or about 80 g, or about 85 g, or about 90 g, or about 95 g, or about 100 g.

In some embodiments, the amount of a composition administered is, without limitation, 0.001 g to 0.01 g, or 0.01 g to 0.1 g, or 0.1 g to 1 g, or 1 g to 10 g, or 10 g to 20 g, or 20 g to 30 g, or 30 g to 40 g, or 40 g to 50 g, or 50 g to 60 g, or 60 g to 70 g, or 70 g to 80 g, or 80 g to 90 g, or 90 g to 100 g.

In some embodiments, the volume of a composition administered is, without limitation, at least 0.01 mL, or at least 0.02 mL, or at least 0.03 mL, or at least 0.04 mL, or at least 0.05 mL, or at least 0.06 mL, or at least 0.07 mL, or at least 0.08 mL, or at least 0.09 mL, or at least 0.10 mL, or at least 0.15 mL, or at least 0.20 mL, or at least 0.25 mL, or at least 0.30 mL, or at least 0.35 mL, or at least 0.40 mL, or at least 0.45 mL, or at least 0.50 mL, or at least 0.55 mL, or at least 0.60 mL, or at least 0.65 mL, or at least 0.70 mL, or at least 0.75 mL, or at least 0.80 mL, or at least 0.85 mL, or at least 0.90 mL, or at least 0.95 mL, or at least 1 mL, or at least 2 mL, or at least 3 mL, or at least 4 mL, or at least 5 mL, or at least 6 mL, or at least 7 mL, or at least, 8 mL, or at least 9 mL, or at least 10 mL, or at least 15 mL, or at least 20 mL, or at least 25 mL, or at least 30 mL, or at least 35 mL, or at least 40 mL, or at least 45 mL, or at least 50 mL, or at least 55 mL, or at least 60 mL, or at least 65 mL, or at least 70 mL, or at least 75 mL, or at least 80 mL, or at least 85 mL, or at least 90 mL, or at least 95 mL, or at least 100 mL, or at least 110 mL, or at least 120 mL, or at least 130 mL, or at least 140 mL, or at least 150 mL, or at least 160 mL, or at least 170 mL, or at least 180 mL, or at least 190 mL, or at least 200 mL, or at least 210 mL, or at least 220 mL, or at least 230 mL, or at least 240 mL, or at least 250 mL, or at least 260 mL, or at least 270 mL, or at least 280 mL, or at least 290 mL, or at least 300 mL, or at least 325, 350 mL, or at least 375 mL, or at least 400 mL, or at least 425 mL, or at least 450 mL, or at least 475 mL, or at least 500 mL, or at least 525 mL, or at least 550 mL, or at least 575 mL, or at least 600 mL, or at least 625 mL, or at least 650 mL, or at least 675 mL, or at least 700 mL, or at least 725 mL, or at least 750 mL, or at least 775 mL, or at least 800 mL, or at least 825 mL, or at least 850 mL, or at least 875 mL, or at least 900 mL, or at least 925 mL, or at least 950 mL, or at least 975 mL, or at least 1000 mL.

In some embodiments, the volume of a composition administered is, without limitation, at most 0.01 mL, or at most 0.02 mL, or at most 0.03 mL, or at most 0.04 mL, or at most 0.05 mL, or at most 0.06 mL, or at most 0.07 mL, or at most 0.08 mL, or at most 0.09 mL, or at most 0.10 mL, or at most 0.15 mL, or at most 0.20 mL, or at most 0.25 mL, or at most 0.30 mL, or at most 0.35 mL, or at most 0.40 mL, or at most 0.45 mL, or at most 0.50 mL, or at most 0.55 mL, or at most 0.60 mL, or at most 0.65 mL, or at most 0.70 mL, or at most 0.75 mL, or at most 0.80 mL, or at most 0.85 mL, or at most 0.90 mL, or at most 0.95 mL, or at most 1 mL, or at most 2 mL, or at most 3 mL, or at most 4 mL, or at most 5 mL, or at most 6 mL, or at most 7 mL, or at most, 8 mL, or at most 9 mL, or at most 10 mL, or at most 15 mL, or at most 20 mL, or at most 25 mL, or at most 30 mL, or at most 35 mL, or at most 40 mL, or at most 45 mL, or at most 50 mL, or at most 55 mL, or at most 60 mL, or at most 65 mL, or at most 70 mL, or at most 75 mL, or at most 80 mL, or at most 85 mL, or at most 90 mL, or at most 95 mL, or at most 100 mL, or at most 110 mL, or at most 120 mL, or at most 130 mL, or at most 140 mL, or at most 150 mL, or at most 160 mL, or at most 170 mL, or at most 180 mL, or at most 190 mL, or at most 200 mL, or at most 210 mL, or at most 220 mL, or at most 230 mL, or at most 240 mL, or at most 250 mL, or at most 260 mL, or at most 270 mL, or at most 280 mL, or at most 290 mL, or at most 300 mL, or at most 325, 350 mL, or at most 375 mL, or at most 400 mL, or at most 425 mL, or at most 450 mL, or at most 475 mL, or at most 500 mL, or at most 525 mL, or at most 550 mL, or at most 575 mL, or at most 600 mL, or at most 625 mL, or at most 650 mL, or at most 675 mL, or at most 700 mL, or at most 725 mL, or at most 750 mL, or at most 775 mL, or at most 800 mL, or at most 825 mL, or at most 850 mL, or at most 875 mL, or at most 900 mL, or at most 925 mL, or at most 950 mL, or at most 975 mL, or at most 1000 mL.

In some embodiments, the volume of a composition administered is, without limitation, about 0.01 mL, or about 0.02 mL, or about 0.03 mL, or about 0.04 mL, or about 0.05 mL, or about 0.06 mL, or about 0.07 mL, or about 0.08 mL, or about 0.09 mL, or about 0.10 mL, or about 0.15 mL, or about 0.20 mL, or about 0.25 mL, or about 0.30 mL, or about 0.35 mL, or about 0.40 mL, or about 0.45 mL, or about 0.50 mL, or about 0.55 mL, or about 0.60 mL, or about 0.65 mL, or about 0.70 mL, or about 0.75 mL, or about 0.80 mL, or about 0.85 mL, or about 0.90 mL, or about 0.95 mL, or about 1 mL, or about 2 mL, or about 3 mL, or about 4 mL, or about 5 mL, or about 6 mL, or about 7 mL, or about, 8 mL, or about 9 mL, or about 10 mL, or about 11 mL, or about 12 mL, or about 13 mL, or about 14 mL, or about 15 mL, or about 16 mL, or about 17 mL, or about 18 mL, or about 19 mL, or about 20 mL, or about 21 mL, or about 22 mL, or about 23 mL, or about 24 mL, or about 25 mL, or about 26 mL, or about 27 mL, or about 28 mL, or about 30 mL, or about 35 mL, or about 36 mL, or about 37 mL, or about 38 mL, or about 39 mL, or about 40 mL, or about 41 mL, or about 42 mL, or about 43 mL, or about 44 mL, or about 45 mL, or about 46 mL, or about 47 mL, or about 48 mL, or about 49 mL, or about 50 mL, or about 51 mL, or about 52 mL, or about 53 mL, or about 54 mL, or about 55 mL, or about 56 mL, or about 57 mL, or about 58 mL, or about 59 mL, or about 60 mL, or about 61 mL, or about 62 mL, or about 63 mL, or about 64 mL, or about 65 mL, or about 66 mL, or about 67 mL, or about 68 mL, or about 69 mL, or about 70 mL, or about 71 mL, or about 72 mL, or about 73 mL, or about 74 mL, or about 75 mL, or about 76 mL, or about 77 mL, or about 78 mL, or about 79 mL, or about 80 mL, or about 81 mL, or about 82 mL, or about 83 mL, or about 84 mL, or about 85 mL, or about 86 mL, or about 87 mL, or about 88 mL, or about 89 mL, or about 90 mL, or about 91 mL, or about 92 mL, or about 93 mL, or about 94 mL, or about 95 mL, or about 96 mL, or about 97 mL, or about 98 mL, or about 99 mL, or about 100 mL, or about 110 mL, or about 120 mL, or about 130 mL, or about 140 mL, or about 150 mL, or about 160 mL, or about 170 mL, or about 180 mL, or about 190 mL, or about 200 mL, or about 210 mL, or about 220 mL, or about 230 mL, or about 240 mL, or about 250 mL, or about 260 mL, or about 270 mL, or about 280 mL, or about 290 mL, or about 300 mL, or about 310 mL, or about 320 mL, or about 330 mL, or about 340 mL, or about 350 mL, or about 360 mL, or about 370 mL, or about 380 mL, or about 390 mL, or about 400 mL, or about 410 mL, or about 420 mL, or about 430 mL, or about 440 mL, or about 450 mL, or about 460 mL, or about 470 mL, or about 480 mL, or about 490 mL, or about 500 mL, or about 510 mL, or about 520 mL, or about 530 mL, or about 540 mL, or about 550 mL, or about 560 mL, or about 570 mL, or about 580 mL, or about 590 mL, or about 600 mL, or about 610 mL, or about 620 mL, or about 630 mL, or about 640 mL, or about 650 mL, or about 660 mL, or about 670 mL, or about 680 mL, or about 690 mL, or about 700 mL, or about 710 mL, or about 720 mL, or about 730 mL, or about 740 mL, or about 750 mL, or about 760 mL, or about 770 mL, or about 780 mL, or about 790 mL, or about 800 mL, or about 810 mL, or about 820 mL, or about 830 mL, or about 840 mL, or about 850 mL, or about 860 mL, or about 870 mL, or about 880 mL, or about 890 mL, or about 900 mL, or about 910 mL, or about 920 mL, or about 930 mL, or about 940 mL, or about 950 mL, or about 960 mL, or about 970 mL, or about 980 mL, or about 990 mL, or about 1000 mL.

In some embodiments, the volume of a composition administered is, without limitation, 0.01 mL to 0.10 mL, or 0.10 mL to 1 mL, or 1 mL to 10 mL, or 10 mL to 100 mL, or 50 mL to 100 mL, or 100 mL to 150 mL, or 150 mL to 200 mL, or 200 mL to 250 mL, or 250 mL to 300 mL, or 300 mL to 350 mL, or 350 mL to 400 mL, or 400 mL to 450 mL, or 450 mL to 500 mL, or 500 mL to 550 mL, or 550 mL to 600 mL, or 600 mL to 650 mL, or 650 mL to 700 mL, or 700 mL to 750 mL, or 750 mL to 800 mL, or 800 mL to 850 mL, or 850 mL to 900 mL, or 900 mL to 950 mL, or 950 mL to 1000 mL, or 1 mL to 25 mL, or 1 mL to 50 mL, or 1 mL to 75 mL, or 1 mL to 100 mL, or 10 mL to 25 mL, or 10 mL 50 mL, or 10 mL to 75 mL, or 100 mL to 250 mL, or 100 mL to 500 mL, or 100 mL to 750 mL, or 100 mL to 1000 mL.

### Silk Fibroin Protein Fragments as Collagen Stimulating Compositions

Raw silk from silkworm Bombyx mori is composed of two primary proteins: silk fibroin (approximately 75%) and sericin (approximately 25%). Silk fibroin is a fibrous protein with a semi-crystalline structure that provides stiffness and strength. As used herein, the term “silk fibroin” means the fibers of the cocoon of Bombyx mori having a weight average molecular weight of about 370,000 Da. The crude silkworm fiber consists of a double thread of fibroin. The adhesive substance holding these double fibers together is sericin. The silk fibroin is composed of a heavy chain having a weight average molecular weight of about 350,000 Da (H chain), and a light chain having a weight average molecular weight about 25,000 Da (L chain).

Conversion of these fibrils silk fibroin into water-soluble silk fibroin protein fragments requires the addition of a concentrated heavy salt (e.g., 8-10 M lithium bromide), which interferes with inter- and intramolecular ionic and hydrogen bonding that would otherwise render the fibroin protein insoluble in water. Methods of making silk fibroin or silk fibroin fragments, and/or compositions thereof, are known and are described for example in U.S. Pat. Nos. 9,187,538, 9,511,012, 9,517,191, 9,522,107, 9,522,108, 9,545,369, and 10,166,177.

Provided herein are silk protein fragment (SPF) mixture solutions obtained by dissolving raw unscoured, partially scoured, or scoured silkworm fibers with a neutral lithium bromide salt. The raw silkworm fibers are processed under selected temperature and other conditions in order to remove any sericin and achieve the desired weight average molecular weight (Mw) and polydispersity (PD) of the fragment mixture. Select process parameters may be altered to achieve distinct final silk protein fragment characteristics depending upon the intended use. The resulting final fragment solution is silk fibroin protein fragments and water with PPM to non-detectable levels of process contaminants, levels acceptable in the pharmaceutical, medical and consumer cosmetic markets. The concentration, size and polydispersity of silk fibroin protein fragments in the solution may further be altered depending upon the desired use and performance requirements.

In an embodiment, silk protein fragment solutions useful for applications in collagen stimulating compositions and methods of making and using thereof are prepared according to the following steps: forming pieces of silk cocoons from the Bombyx mori silk worm; extracting the pieces at about 100° C. in a Na₂CO₃ water solution and for about 60 minutes, wherein a volume of the water equals about 0.4× raw silk weight and the amount of Na₂CO₃ is about 0.848× the weight of the pieces to form a silk fibroin extract; triple rinsing the silk fibroin extract at about 60° C. for about 20 minutes per rinse in a volume of rinse water, wherein the rinse water for each cycle equals about 0.2 L× the weight of the pieces; removing excess water from the silk fibroin extract; drying the silk fibroin extract; dissolving the dry silk fibroin extract in a LiBr solution, wherein the LiBr solution is first heated to about 100° C. to create a silk and LiBr solution and maintained; placing the silk and LiBr solution in a dry oven at about 100° C. for about 60 minutes to achieve complete dissolution and further fragmentation of the native silk protein structure into mixture with desired molecular weight and polydispersity; filtering the solution to remove any remaining debris from the silkworm; diluting the solution with water to result in a 1.0 wt. % silk solution; and removing solvent from the solution using Tangential Flow Filtration (TFF). In an embodiment, a 10 kDa membrane is utilized to purify the silk solution and create the final desired silk-to-water ratio. TFF can then be used to further concentrate the silk solution to a concentration of 2.0 wt. % silk in water.

Without wishing to be bound by any particular theory, varying extraction (i.e., time and temperature), LiBr (i.e., temperature of LiBr solution when added to silk fibroin extract or vice versa) and dissolution (i.e., time and temperature) parameters results in solvent and silk solutions with different viscosities, homogeneities, and colors. Also without wishing to be bound by any particular theory, increasing the temperature for extraction, lengthening the extraction time, using a higher temperature LiBr solution at emersion and over time when dissolving the silk and increasing the time at temperature (e.g., in an oven as shown here, or an alternative heat source) all resulted in less viscous and more homogeneous solvent and silk solutions.

In an embodiment, solutions of silk fibroin-based protein fragments having a weight average selected from between about 6 kDa to about 17 kDa are prepared according to following steps: degumming a silk source by adding the silk source to a boiling (100° C.) aqueous solution of sodium carbonate for a treatment time of between about 30 minutes to about 60 minutes; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 60° C. to about 140° C.; maintaining the solution of silk fibroin-lithium bromide in an oven having a temperature of about 140° C. for a period of at least 1 hour; removing the lithium bromide from the silk fibroin extract; and producing an aqueous solution of silk protein fragments, the aqueous solution comprising: fragments having a weight average molecular weight selected from between about 6 kDa to about 17 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay. The aqueous solution of silk fibroin-based protein fragments may be lyophilized.

In an embodiment, solutions of silk fibroin-based protein fragments having a weight average molecular weight selected from between about 17 kDa to about 39 kDa are prepared according to the following steps: adding a silk source to a boiling (100° C.) aqueous solution of sodium carbonate for a treatment time of between about 30 minutes to about 60 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of silk fibroin-based protein fragments, wherein the aqueous solution of silk fibroin-based protein fragments comprises lithium bromide residuals of between about 10 ppm and about 300 ppm, wherein the aqueous solution of silk protein fragments comprises sodium carbonate residuals of between about 10 ppm and about 100 ppm, wherein the aqueous solution of silk fibroin-based protein fragments comprises fragments having a weight average molecular weight selected from between about 17 kDa to about 39 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay.

In an embodiment, solutions of silk fibroin-based protein fragments having a weight average molecular weight selected from between about 39 kDa to about 80 kDa are prepared according to the following steps: adding a silk source to a boiling (100° C.) aqueous solution of sodium carbonate for a treatment time of about 30 minutes so as to result in degumming; removing sericin from the solution to produce a silk fibroin extract comprising non-detectable levels of sericin; draining the solution from the silk fibroin extract; dissolving the silk fibroin extract in a solution of lithium bromide having a starting temperature upon placement of the silk fibroin extract in the lithium bromide solution that ranges from about 80° C. to about 140° C.; maintaining the solution of silk fibroin-lithium bromide in a dry oven having a temperature in the range between about 60° C. to about 100° C. for a period of at least 1 hour; removing the lithium bromide from the silk fibroin extract; and producing an aqueous solution of silk fibroin-based protein fragments, wherein the aqueous solution of silk fibroin-based protein fragments comprises lithium bromide residuals of between about 10 ppm and about 300 ppm, sodium carbonate residuals of between about 10 ppm and about 100 ppm, fragments having a weight average molecular weight selected from between about 39 kDa to about 80 kDa, and wherein the aqueous solution of silk fibroin-based protein fragments comprises a polydispersity of between about 1.5 and about 3.0. The method may further comprise drying the silk fibroin extract prior to the dissolving step. The aqueous solution of silk fibroin-based protein fragments may comprise lithium bromide residuals of less than 300 ppm as measured using a high-performance liquid chromatography lithium bromide assay. The aqueous solution of silk fibroin-based protein fragments may comprise sodium carbonate residuals of less than 100 ppm as measured using a high-performance liquid chromatography sodium carbonate assay.

In an embodiment, the silk fibroin-based protein fragments in the solution are substantially devoid of sericin, have a weight average molecular weight selected from between about 6 kDa to about 17 kDa, and have a polydispersity selected from between about 1.5 and about 3.0. In an embodiment, the silk fibroin-based protein fragments in the solution are substantially devoid of sericin, have a weight average molecular weight selected from between about 17 kDa to about 39 kDa, and have a polydispersity selected from between about 1.5 and about 3.0. In an embodiment, the silk fibroin-based protein fragments in the solution are substantially devoid of sericin, have a weight average molecular weight selected from between about 39 kDa to about 80 kDa, and have a polydispersity selected from between about 1.5 and about 3.0.

As used herein, the terms “substantially sericin free” or “substantially devoid of sericin” refer to silk fibers in which a majority of the sericin protein has been removed. In an embodiment, silk fibroin that is substantially devoid of sericin refers to silk fibroin having from about 0.01 wt. % to about 10.0 wt. % sericin. In an embodiment, silk fibroin that is substantially devoid of sericin refers to silk fibroin having about 0.01 wt. % to about 9.0 wt. % sericin. In an embodiment, silk fibroin that is substantially devoid of sericin refers to silk fibroin having from about 0.01 wt. % to about 8.0 wt. % sericin. In an embodiment, silk fibroin that is substantially devoid of sericin refers to silk fibroin having from about 0.01 wt. % to about 7.0 wt. % sericin. In an embodiment, silk fibroin that is substantially devoid of sericin refers to silk fibroin having from about 0.01 wt. % to about 6.0 wt. % sericin. In an embodiment, silk fibroin that is substantially devoid of sericin refers to silk fibroin having from about 0.01 wt. % to about 5.0 wt. % sericin. In an embodiment, silk fibroin that is substantially devoid of sericin refers to silk fibroin having from about 0 wt. % to about 4.0 wt. % sericin. In an embodiment, silk fibroin that is substantially devoid of sericin refers to silk fibroin having from about 0.05 wt. % to about 4.0 wt. % sericin. In an embodiment, silk fibroin that is substantially devoid of sericin refers to silk fibroin having from about 0.1 wt. % to about 4.0 wt. % sericin. In an embodiment, silk fibroin that is substantially devoid of sericin refers to silk fibroin having from about 0.5 wt. % to about 4.0 wt. % sericin. In an embodiment, silk fibroin that is substantially devoid of sericin refers to silk fibroin having from about 1.0 wt. % to about 4.0 wt. % sericin. In an embodiment, silk fibroin that is substantially devoid of sericin refers to silk fibroin having from about 1.5 wt. % to about 4.0 wt. % sericin. In an embodiment, silk fibroin that is substantially devoid of sericin refers to silk fibroin having from about 2.0 wt. % to about 4.0 wt. % sericin. In an embodiment, silk fibroin that is substantially devoid of sericin refers to silk fibroin having from about 2.5 wt. % to about 4.0 wt. % sericin. In an embodiment, silk fibroin that is substantially devoid of sericin refers to silk fibroin having a sericin content from about 0.01 wt. % to about 0.1 wt. %. In an embodiment, silk fibroin that is substantially devoid of sericin refers to silk fibroin having a sericin content below about 0.1 wt. %. In an embodiment, silk fibroin that is substantially devoid of sericin refers to silk fibroin having a sericin content below about 0.05 wt. %. In an embodiment, when a silk source is added to a boiling (100° C.) aqueous solution of sodium carbonate for a treatment time of between about 30 minutes to about 60 minutes, a degumming loss of about 26.0 wt. % to about 31.0 wt. % is obtained.

Following are non-limiting examples of suitable ranges for various parameters in and for preparation of the silk solutions of the present disclosure. The silk solutions of the present disclosure may include one or more, but not necessarily all, of these parameters and may be prepared using various combinations of ranges of such parameters.

In an embodiment, the percent silk in the solution is, without limitation, less than 30 wt. %. In an embodiment, the percent silk in the solution is less than 25 wt. %. In an embodiment, the percent silk in the solution is less than 20 wt. %. In an embodiment, the percent silk in the solution is less than 19 wt. %. In an embodiment, the percent silk in the solution is less than 18 wt. %. In an embodiment, the percent silk in the solution is less than 17 wt. %. In an embodiment, the percent silk in the solution is less than 16 wt. %. In an embodiment, the percent silk in the solution is less than 15 wt. %. In an embodiment, the percent silk in the solution is less than 14 wt. %. In an embodiment, the percent silk in the solution is less than 13 wt. %. In an embodiment, the percent silk in the solution is less than 12 wt. %. In an embodiment, the percent silk in the solution is less than 11 wt. %. In an embodiment, the percent silk in the solution is less than 10 wt. %. In an embodiment, the percent silk in the solution is less than 9 wt. %. In an embodiment, the percent silk in the solution is less than 8 wt. %. In an embodiment, the percent silk in the solution is less than 7 wt. %. In an embodiment, the percent silk in the solution is less than 6 wt. %. In an embodiment, the percent silk in the solution is less than 5 wt. %. In an embodiment, the percent silk in the solution is less than 4 wt. %. In an embodiment, the percent silk in the solution is less than 3 wt. %. In an embodiment, the percent silk in the solution is less than 2 wt. %. In an embodiment, the percent silk in the solution is less than 1 wt. %. In an embodiment, the percent silk in the solution is less than 0.9 wt. %. In an embodiment, the percent silk in the solution is less than 0.8 wt. %. In an embodiment, the percent silk in the solution is less than 0.7 wt. %. In an embodiment, the percent silk in the solution is less than 0.6 wt. %. In an embodiment, the percent silk in the solution is less than 0.5 wt. %. In an embodiment, the percent silk in the solution is less than 0.4 wt. %. In an embodiment, the percent silk in the solution is less than 0.3 wt. %. In an embodiment, the percent silk in the solution is less than 0.2 wt. %. In an embodiment, the percent silk in the solution is less than 0.1 wt. %.

In an embodiment, the percent silk in the solution is, without limitation, greater than 0.1 wt. %. In an embodiment, the percent silk in the solution is greater than 0.2 wt. %. In an embodiment, the percent silk in the solution is greater than 0.3 wt. %. In an embodiment, the percent silk in the solution is greater than 0.4 wt. %. In an embodiment, the percent silk in the solution is greater than 0.5 wt. %. In an embodiment, the percent silk in the solution is greater than 0.6 wt. %. In an embodiment, the percent silk in the solution is greater than 0.7 wt. %. In an embodiment, the percent silk in the solution is greater than 0.8 wt. %. In an embodiment, the percent silk in the solution is greater than 0.9 wt. %. In an embodiment, the percent silk in the solution is greater than 1.0 wt. %. In an embodiment, the percent silk in the solution is greater than 2.0 wt. %. In an embodiment, the percent silk in the solution is greater than 3.0 wt. %. In an embodiment, the percent silk in the solution is greater than 4.0 wt. %. In an embodiment, the percent silk in the solution is greater than 5.0 wt. %. In an embodiment, the percent silk in the solution is greater than 6.0 wt. %. In an embodiment, the percent silk in the solution is greater than 7.0 wt. %. In an embodiment, the percent silk in the solution is greater than 8.0 wt. %. In an embodiment, the percent silk in the solution is greater than 9.0 wt. %. In an embodiment, the percent silk in the solution is greater than 10.0 wt. %. In an embodiment, the percent silk in the solution is greater than 11.0 wt. %. In an embodiment, the percent silk in the solution is greater than 12.0 wt. %. In an embodiment, the percent silk in the solution is greater than 13.0 wt. %. In an embodiment, the percent silk in the solution is greater than 14.0 wt. %. In an embodiment, the percent silk in the solution is greater than 15.0 wt. %. In an embodiment, the percent silk in the solution is greater than 16.0 wt. %. In an embodiment, the percent silk in the solution is greater than 17.0 wt. %. In an embodiment, the percent silk in the solution is greater than 18.0 wt. %. In an embodiment, the percent silk in the solution is greater than 19.0 wt. %. In an embodiment, the percent silk in the solution is greater than 20.0 wt. %. In an embodiment, the percent silk in the solution is greater than 25.0 wt. %.

In an embodiment, the percent silk in the solution ranges, without limitation, from about 0.1 wt. % to about 30.0 wt. %. In an embodiment, the percent silk in the solution ranges from about 0.1 wt. % to about 25.0 wt. %. In an embodiment, the percent silk in the solution ranges from about 0.1 wt. % to about 20.0 wt. %. In an embodiment, the percent silk in the solution ranges from about 0.1 wt. % to about 15.0 wt. %. In an embodiment, the percent silk in the solution ranges from about 0.1 wt. % to about 10.0 wt. %. In an embodiment, the percent silk in the solution ranges from about 0.1 wt. % to about 9.0 wt. %. In an embodiment, the percent silk in the solution ranges from about 0.1 wt. % to about 8.0 wt. %. In an embodiment, the percent silk in the solution ranges from about 0.1 wt. % to about 7.0 wt. %. In an embodiment, the percent silk in the solution ranges from about 0.1 wt. % to about 6.5 wt. %. In an embodiment, the percent silk in the solution ranges from about 0.1 wt. % to about 6.0 wt. %. In an embodiment, the percent silk in the solution ranges from about 0.1 wt. % to about 5.5 wt. %. In an embodiment, the percent silk in the solution ranges from about 0.1 wt. % to about 5.0 wt. %. In an embodiment, the percent silk in the solution ranges from about 0.1 wt. % to about 4.5 wt. %. In an embodiment, the percent silk in the solution ranges from about 0.1 wt. % to about 4.0 wt. %. In an embodiment, the percent silk in the solution ranges from about 0.1 wt. % to about 3.5 wt. %. In an embodiment, the percent silk in the solution ranges from about 0.1 wt. % to about 3.0 wt. %. In an embodiment, the percent silk in the solution ranges from about 0.1 wt. % to about 2.5 wt. %. In an embodiment, the percent silk in the solution ranges from about 0.1 wt. % to about 2.0 wt. %. In an embodiment, the percent silk in the solution ranges from about 0.1 wt. % to about 2.4 wt. %. In an embodiment, the percent silk in the solution ranges from about 0.5 wt. % to about 5.0 wt. %. In an embodiment, the percent silk in the solution ranges from about 0.5 wt. % to about 4.5 wt. %. In an embodiment, the percent silk in the solution ranges from about 0.5 wt. % to about 4.0 wt. %. In an embodiment, the percent silk in the solution ranges from about 0.5 wt. % to about 3.5 wt. %. In an embodiment, the percent silk in the solution ranges from about 0.5 wt. % to about 3.0 wt. %. In an embodiment, the percent silk in the solution ranges from about 0.5 wt. % to about 2.5 wt. %. In an embodiment, the percent silk in the solution ranges from about 1.0 wt. % to about 4.0 wt. %. In an embodiment, the percent silk in the solution ranges from about 1.0 wt. % to about 3.5 wt. %. In an embodiment, the percent silk in the solution ranges from about 1.0 wt. % to about 3.0 wt. %. In an embodiment, the percent silk in the solution ranges from about 1.0 wt. % to about 2.5 wt. %. In an embodiment, the percent silk in the solution ranges from about 1.0 wt. % to about 2.4 wt. %. In an embodiment, the percent silk in the solution ranges from about 1.0 wt. % to about 2 wt. %.

In an embodiment, the percent silk in the solution ranges from about 20.0 wt. % to about 30.0 wt. %. In an embodiment, the percent silk in the solution ranges from about 0.1 wt. % to about 10.0 wt. %. In an embodiment, the percent silk in the solution ranges from about 1.0 wt. % to about 10.0 wt. %. In an embodiment, the percent silk in the solution ranges from about 2 wt. % to about 10.0 wt. %. In an embodiment, the percent silk in the solution ranges from about 0.1 wt. % to about 6.0 wt. %. In an embodiment, the percent silk in the solution ranges from about 6.0 wt. % to about 10.0 wt. %. In an embodiment, the percent silk in the solution ranges from about 6.0 wt. % to about 8.0 wt. %. In an embodiment, the percent silk in the solution ranges from about 6.0 wt. % to about 9.0 wt. %. In an embodiment, the percent silk in the solution ranges from about 10.0 wt. % to about 20.0 wt. %. In an embodiment, the percent silk in the solution ranges from about 11.0 wt. % to about 19.0 wt. %. In an embodiment, the percent silk in the solution ranges from about 12.0 wt. % to about 18.0 wt. %. In an embodiment, the percent silk in the solution ranges from about 13.0 wt. % to about 17.0 wt. %. In an embodiment, the percent silk in the solution ranges from about 14.0 wt. % to about 16.0 wt. %. In an embodiment, the percent silk in the solution is about 1.0 wt. %. In an embodiment, the percent silk in the solution is about 1.5 wt. %. In an embodiment, the percent silk in the solution is about 2.0 wt. %. In an embodiment, the percent silk in the solution is about 2.4 wt. %. In an embodiment, the percent silk in the solution is 3.0 wt. %. In an embodiment, the percent silk in the solution is 3.5 wt. %. In an embodiment, the percent silk in the solution is about 4.0 wt. %. In an embodiment, the percent silk in the solution is about 4.5 wt. %. In an embodiment, the percent silk in the solution is about 5.0 wt. %. In an embodiment, the percent silk in the solution is about 5.5 wt. %. In an embodiment the percent silk in the solution is about 6.0 wt. %. In an embodiment, the percent silk in the solution is about 6.5 wt. %. In an embodiment, the percent silk in the solution is about 7.0 wt. %. In an embodiment, the percent silk in the solution is about 7.5 wt. %. In an embodiment, the percent silk in the solution is about 8.0 wt. %. In an embodiment, the percent silk in the solution is about 8.5 wt. %. In an embodiment, the percent silk in the solution is about 9.0 wt. %. In an embodiment, the percent silk in the solution is about 9.5 wt. %. In an embodiment, the percent silk in the solution is about 10.0 wt. %.

In an embodiment, the percent sericin in the solution is non-detectable to 30.0 wt. %. In an embodiment, the percent sericin in the solution is non-detectable to 5.0 wt. %. In an embodiment, the percent sericin in the solution is 1.0 wt. %. In an embodiment, the percent sericin in the solution is 2.0 wt. %. In an embodiment, the percent sericin in the solution is 3.0 wt. %. In an embodiment, the percent sericin in the solution is 4.0 wt. %. In an embodiment, the percent sericin in the solution is 5.0 wt. %. In an embodiment, the percent sericin in the solution is 10.0 wt. %. In an embodiment, the percent sericin in the solution is 30.0 wt. %.

In some embodiments, the silk fibroin protein based fragments of the present disclosure are shelf stable (they will not slowly or spontaneously gel when stored in an aqueous solution and there is no aggregation of fragments and therefore no increase in molecular weight over time), from 10 days to 3 years depending on storage conditions, percent silk, and number of shipments and shipment conditions. Additionally, pH may be altered to extend shelf-life and/or support shipping conditions by preventing premature folding and aggregation of the silk. In an embodiment, the stability of the LiBr-silk fragment solution is 0 to 1 year. In an embodiment, the stability of the LiBr-silk fragment solution is 0 to 2 years. In an embodiment, the stability of the LiBr-silk fragment solution is 0 to 3 years. In an embodiment, the stability of the LiBr-silk fragment solution is 0 to 4 years. In an embodiment, the stability of the LiBr-silk fragment solution is 0 to 5 years. In an embodiment, the stability of the LiBr-silk fragment solution is 1 to 2 years. In an embodiment, the stability of the LiBr-silk fragment solution is 1 to 3 years. In an embodiment, the stability of the LiBr-silk fragment solution is 1 to 4 years. In an embodiment, the stability of the LiBr-silk fragment solution is 1 to 5 years. In an embodiment, the stability of the LiBr-silk fragment solution is 2 to 3 years. In an embodiment, the stability of the LiBr-silk fragment solution is 2 to 4 years. In an embodiment, the stability of the LiBr-silk fragment solution is 2 to 5 years. In an embodiment, the stability of the LiBr-silk fragment solution is 3 to 4 years. In an embodiment, the stability of the LiBr-silk fragment solution is 3 to 5 years. In an embodiment, the stability of the LiBr-silk fragment solution is 4 to 5 years.

In an embodiment, the stability of a composition of the present disclosure is 10 days to 6 months. In an embodiment, the stability of a composition of the present disclosure is 6 months to 12 months. In an embodiment, the stability of a composition of the present disclosure is 12 months to 18 months. In an embodiment, the stability of a composition of the present disclosure is 18 months to 24 months. In an embodiment, the stability of a composition of the present disclosure is 24 months to 30 months. In an embodiment, the stability of a composition of the present disclosure is 30 months to 36 months. In an embodiment, the stability of a composition of the present disclosure is 36 months to 48 months. In an embodiment, the stability of a composition of the present disclosure is 48 months to 60 months.

In an embodiment, a composition of the present disclosure includes silk fibroin-based protein fragments having a weight average molecular weight selected from between 6 kDa to 17 kDa. In an embodiment, a composition of the present disclosure includes silk fibroin-based protein fragments having a weight average molecular weight selected from between 17 kDa to 39 kDa. In an embodiment, a composition of the present disclosure includes silk fibroin-based protein fragments having a weight average molecular weight selected from between 39 kDa to 80 kDa. In an embodiment, a composition of the present disclosure includes silk fibroin-based protein fragments having a weight average molecular weight selected from between 40 kDa to 65 kDa. In an embodiment, a composition of the present disclosure includes silk fibroin-based protein fragments having a weight average molecular weight selected from between 1 kDa to 5 kDa. In an embodiment, a composition of the present disclosure includes silk fibroin-based protein fragments having a weight average molecular weight selected from between 5 kDa to 10 kDa. In an embodiment, a composition of the present disclosure includes silk fibroin-based protein fragments having a weight average molecular weight selected from between 10 kDa to 15 kDa. In an embodiment, a composition of the present disclosure includes silk fibroin-based protein fragments having a weight average molecular weight selected from between 15 kDa to 20 kDa. In an embodiment, a composition of the present disclosure includes silk fibroin-based protein fragments having a weight average molecular weight selected from between 20 kDa to 25 kDa. In an embodiment, a composition of the present disclosure includes silk fibroin-based protein fragments having a weight average molecular weight selected from between 25 kDa to 30 kDa. In an embodiment, a composition of the present disclosure includes silk fibroin-based protein fragments having a weight average molecular weight selected from between 30 kDa to 35 kDa. In an embodiment, a composition of the present disclosure includes silk fibroin-based protein fragments having a weight average molecular weight selected from between 35 kDa to 40 kDa. In an embodiment, a composition of the present disclosure includes silk fibroin-based protein fragments having a weight average molecular weight selected from between 40 kDa to 45 kDa. In an embodiment, a composition of the present disclosure includes silk fibroin-based protein fragments having a weight average molecular weight selected from between 45 kDa to 50 kDa. In an embodiment, a composition of the present disclosure includes silk fibroin-based protein fragments having a weight average molecular weight selected from between 50 kDa to 55 kDa. In an embodiment, a composition of the present disclosure includes silk fibroin-based protein fragments having a weight average molecular weight selected from between 55 kDa to 60 kDa. In an embodiment, a composition of the present disclosure includes silk fibroin-based protein fragments having a weight average molecular weight selected from between 60 kDa to 65 kDa. In an embodiment, a composition of the present disclosure includes silk fibroin-based protein fragments having a weight average molecular weight selected from between 65 kDa to 70 kDa. In an embodiment, a composition of the present disclosure includes silk fibroin-based protein fragments having a weight average molecular weight selected from between 70 kDa to 75 kDa. In an embodiment, a composition of the present disclosure includes silk fibroin-based protein fragments having a weight average molecular weight selected from between 75 kDa to 80 kDa. In an embodiment, a composition of the present disclosure includes silk fibroin-based protein fragments having a weight average molecular weight selected from between 80 kDa to 85 kDa. In an embodiment, a composition of the present disclosure includes silk fibroin-based protein fragments having a weight average molecular weight selected from between 85 kDa to 90 kDa. In an embodiment, a composition of the present disclosure includes silk fibroin-based protein fragments having a weight average molecular weight selected from between 90 kDa to 95 kDa. In an embodiment, a composition of the present disclosure includes silk fibroin-based protein fragments having a weight average molecular weight selected from between 95 kDa to 100 kDa. In an embodiment, a composition of the present disclosure includes silk fibroin-based protein fragments having a weight average molecular weight selected from between 100 kDa to 105 kDa. In an embodiment, a composition of the present disclosure includes silk fibroin-based protein fragments having a weight average molecular weight selected from between 105 kDa to 110 kDa. In an embodiment, a composition of the present disclosure includes silk fibroin-based protein fragments having a weight average molecular weight selected from between 110 kDa to 115 kDa. In an embodiment, a composition of the present disclosure includes silk fibroin-based protein fragments having a weight average molecular weight selected from between 115 kDa to 120 kDa. In an embodiment, a composition of the present disclosure includes silk fibroin-based protein fragments having a weight average molecular weight selected from between 120 kDa to 125 kDa. In an embodiment, a composition of the present disclosure includes silk fibroin-based protein fragments having a weight average molecular weight selected from between 125 kDa to 130 kDa. In an embodiment, a composition of the present disclosure includes silk fibroin-based protein fragments having a weight average molecular weight selected from between 130 kDa to 135 kDa. In an embodiment, a composition of the present disclosure includes silk fibroin-based protein fragments having a weight average molecular weight selected from between 135 kDa to 140 kDa. In an embodiment, a composition of the present disclosure includes silk fibroin-based protein fragments having a weight average molecular weight selected from between 140 kDa to 145 kDa. In an embodiment, a composition of the present disclosure includes silk fibroin-based protein fragments having a weight average molecular weight selected from between 145 kDa to 150 kDa. In an embodiment, a composition of the present disclosure includes silk fibroin-based protein fragments having a weight average molecular weight selected from between 150 kDa to 155 kDa. In an embodiment, a composition of the present disclosure includes silk fibroin-based protein fragments having a weight average molecular weight selected from between 155 kDa to 160 kDa. In an embodiment, a composition of the present disclosure includes silk fibroin-based protein fragments having a weight average molecular weight selected from between 160 kDa to 165 kDa. In an embodiment, a composition of the present disclosure includes silk fibroin-based protein fragments having a weight average molecular weight selected from between 165 kDa to 170 kDa. In an embodiment, a composition of the present disclosure includes silk fibroin-based protein fragments having a weight average molecular weight selected from between 170 kDa to 175 kDa. In an embodiment, a composition of the present disclosure includes silk fibroin-based protein fragments having a weight average molecular weight selected from between 175 kDa to 180 kDa. In an embodiment, a composition of the present disclosure includes silk fibroin-based protein fragments having a weight average molecular weight selected from between 180 kDa to 185 kDa. In an embodiment, a composition of the present disclosure includes silk fibroin-based protein fragments having a weight average molecular weight selected from between 185 kDa to 190 kDa. In an embodiment, a composition of the present disclosure includes silk fibroin-based protein fragments having a weight average molecular weight selected from between 190 kDa to 195 kDa. In an embodiment, a composition of the present disclosure includes silk fibroin-based protein fragments having a weight average molecular weight selected from between 195 kDa to 200 kDa. In an embodiment, a composition of the present disclosure includes silk fibroin-based protein fragments having a weight average molecular weight selected from between 200 kDa to 205 kDa. In an embodiment, a composition of the present disclosure includes silk fibroin-based protein fragments having a weight average molecular weight selected from between 205 kDa to 210 kDa. In an embodiment, a composition of the present disclosure includes silk fibroin-based protein fragments having a weight average molecular weight selected from between 210 kDa to 215 kDa. In an embodiment, a composition of the present disclosure includes silk fibroin-based protein fragments having a weight average molecular weight selected from between 215 kDa to 220 kDa. In an embodiment, a composition of the present disclosure includes silk fibroin-based protein fragments having a weight average molecular weight selected from between 220 kDa to 225 kDa. In an embodiment, a composition of the present disclosure includes silk fibroin-based protein fragments having a weight average molecular weight selected from between 225 kDa to 230 kDa. In an embodiment, a composition of the present disclosure includes silk fibroin-based protein fragments having a weight average molecular weight selected from between 230 kDa to 235 kDa. In an embodiment, a composition of the present disclosure includes silk fibroin-based protein fragments having a weight average molecular weight selected from between 235 kDa to 240 kDa. In an embodiment, a composition of the present disclosure includes silk fibroin-based protein fragments having a weight average molecular weight selected from between 240 kDa to 245 kDa. In an embodiment, a composition of the present disclosure includes silk fibroin-based protein fragments having a weight average molecular weight selected from between 245 kDa to 250 kDa. In an embodiment, a composition of the present disclosure includes silk fibroin-based protein fragments having a weight average molecular weight selected from between 250 kDa to 255 kDa. In an embodiment, a composition of the present disclosure includes silk fibroin-based protein fragments having a weight average molecular weight selected from between 255 kDa to 260 kDa. In an embodiment, a composition of the present disclosure includes silk fibroin-based protein fragments having a weight average molecular weight selected from between 260 kDa to 265 kDa. In an embodiment, a composition of the present disclosure includes silk fibroin-based protein fragments having a weight average molecular weight selected from between 265 kDa to 270 kDa. In an embodiment, a composition of the present disclosure includes silk fibroin-based protein fragments having a weight average molecular weight selected from between 270 kDa to 275 kDa. In an embodiment, a composition of the present disclosure includes silk fibroin-based protein fragments having a weight average molecular weight selected from between 275 kDa to 280 kDa. In an embodiment, a composition of the present disclosure includes silk fibroin-based protein fragments having a weight average molecular weight selected from between 280 kDa to 285 kDa. In an embodiment, a composition of the present disclosure includes silk fibroin-based protein fragments having a weight average molecular weight selected from between 285 kDa to 290 kDa. In an embodiment, a composition of the present disclosure includes silk fibroin-based protein fragments having a weight average molecular weight selected from between 290 kDa to 295 kDa. In an embodiment, a composition of the present disclosure includes silk fibroin-based protein fragments having a weight average molecular weight selected from between 295 kDa to 300 kDa. In an embodiment, a composition of the present disclosure includes silk fibroin-based protein fragments having a weight average molecular weight selected from between 300 kDa to 305 kDa. In an embodiment, a composition of the present disclosure includes silk fibroin-based protein fragments having a weight average molecular weight selected from between 305 kDa to 310 kDa. In an embodiment, a composition of the present disclosure includes silk fibroin-based protein fragments having a weight average molecular weight selected from between 310 kDa to 315 kDa. In an embodiment, a composition of the present disclosure includes silk fibroin-based protein fragments having a weight average molecular weight selected from between 315 kDa to 320 kDa. In an embodiment, a composition of the present disclosure includes silk fibroin-based protein fragments having a weight average molecular weight selected from between 320 kDa to 325 kDa. In an embodiment, a composition of the present disclosure includes silk fibroin-based protein fragments having a weight average molecular weight selected from between 325 kDa to 330 kDa. In an embodiment, a composition of the present disclosure includes silk fibroin-based protein fragments having a weight average molecular weight selected from between 330 kDa to 335 kDa. In an embodiment, a composition of the present disclosure includes silk fibroin-based protein fragments having a weight average molecular weight selected from between 350 kDa to 340 kDa. In an embodiment, a composition of the present disclosure includes silk fibroin-based protein fragments having a weight average molecular weight selected from between kDa 340 to 345 kDa. In an embodiment, a composition of the present disclosure includes silk fibroin-based protein fragments having a weight average molecular weight selected from between kDa 345 to 350 kDa.

In an embodiment, a composition of the silk fibroin-based protein fragments in this disclosure has a polydispersity selected from between about 1 to about 5.0, In an embodiment, a composition of the silk fibroin-based protein fragments has a polydispersity selected from between about 1.5 to about 3.0. In an embodiment, a composition of the silk fibroin-based protein fragments has a polydispersity selected from between about 1 to about 1.5. In an embodiment, a composition of the silk fibroin-based protein fragments has a polydispersity selected from between about 1.5 to about 2.0. In an embodiment, a composition of the silk fibroin-based protein fragments has a polydispersity selected from between about 2.0 to about 2.5. In an embodiment, a composition of the silk fibroin-based protein fragments, has a polydispersity selected from between about is 2.0 to about 3.0. In an embodiment, a composition of the silk fibroin-based protein fragments has a polydispersity selected from between about is 2.5 to about 3.0.

In some embodiments, lyophilized silk powder can be resuspended in water, hexafluoroisopropanol (HFIP), or organic solution following storage to create silk solutions of varying concentrations, including higher concentration solutions than those produced initially. In another embodiment, the silk fibroin-based protein fragments are dried using a rototherm evaporator or other methods known in the art for creating a dry protein form containing less than 10% water by mass. In an embodiment, the solubility of silk fibroin-based protein fragments of the present disclosure in organic solutions ranges from about 50.0% to about 100%. In an embodiment, the solubility of silk fibroin-based protein fragments of the present disclosure in organic solutions ranges from about 60.0% to about 100%. In an embodiment, the solubility of silk fibroin-based protein fragments of the present disclosure in organic solutions ranges from about 70.0% to about 100%. In an embodiment, the solubility of silk fibroin-based protein fragments of the present disclosure in organic solutions ranges from about 80.0% to about 100%. In an embodiment, the solubility of silk fibroin-based protein fragments of the present disclosure in organic solutions ranges from about 90.0% to about 100%. In an embodiment, the silk fibroin-based fragments of the present disclosure are non-soluble in organic solutions.

In some embodiments, silk fibroin protein fragments useful for applications in collagen stimulating compositions and methods of making and using thereof also include an aqueous gel of the silk fibroin protein fragments. The gelation of silk fibroin protein fragment solutions may be induced by sonication, vortex, heating, solvent treatment (e.g. methanol, ethanol), electrogelation, ultrasonication, chemicals (e.g. vitamin C), or the like.

Silk peptide is an extract from natural silk fibroin hydrolysate. Silk peptide exhibits pearl luster and silky feel when incorporated into personal care products. The structure of silk peptide is similar to human hair and skin tissue. The silk peptides are serine rich polypeptides having 10 or more amino acid residues and weight average molecular weights as described herein. In some embodiments, the silk peptide extract can be easily absorbed by skin, for example human skin, provide nutrients for skin, and promote the metabolism of skin.

In some embodiments, silk fibroin protein fragment solutions useful for applications in collagen stimulating compositions and methods of making and using thereof also include low molecular weight silk fibroin peptides (weight average molecular weight of about 200 Da to 5 kDa). The low molecular weight silk fibroin peptides derived from silk fibroin protein hydrolysate can complement the natural moisturizing factors in the free amino acids to improve the hair scalp moisture content. In some embodiments, the low molecular weight silk fibroin peptides can penetrate deep into the hair follicle to repair, replenish water, nourish hair, improve the moisture balance, and prevent dandruff generation.

In some embodiments, silk fibroin protein fragment solutions useful for applications in collagen stimulating compositions and methods of making and using thereof also include silk fibroin protein amino acids derived from the hydrolyzed silk fibroin. In some embodiments, the silk fibroin amino acids are from commercially available hydrolyzed silk (CAS Number: 96690-41-4). The amino acid composition derived from the silk fibroin protein of Bombyx mori consists mainly of Gly (43%), Ala (30%), and Ser (12%).

In some embodiments, the collagen stimulating compositions and methods of making and using thereof optionally comprises plant extract that enhances the beneficial effects of silk fibroin protein fragments. In some embodiments, the plant extract is selected from the group consisting of extracts from rice, oat, almond, Camellia Sinensis (green tea) extract, Butyrospermum parkii (shea butter), coconut, papaya, mango, peach, lemon, wheat, rosemary, apricot, algae, grapefruit, sandalwood, lime, orange, Acacia concinna, Butea parviflora, Butea superb, Butea frondosa, Campanulata (fire tulip), Adansonia digitata (Baobab), Phoenix dactiyhfera (date), Hibiscus sabdariffa (hibiscus), Aframomum melegueta (African pepper), Khaya senegalensis (mahogany wood), Tamarindus indica (tamarind, or curcumin), Cyperus papyrus (papyrus), Ageratum spp., birch, burdock, horsetail, lavender, marjoram, nettle, tail cat, thyme, oak bark, echinacea, stinging nettle, witch hazel, hops, henna, chamomile, whitethorn, lime-tree blossom, almond, pine needles, horse chestnut, juniper, kiwi, melon, mallow, cuckoo flower, wild thyme, yarrow, melissa, rest harrow, coltsfoot, marshmallow, rice meristem, moringa, ginseng and ginger root, aloe vera, Aloe barbadensis leaf extract, Lavandula angustifolia (lavender) flower extract, Sambucus nigra (elderberry) fruit extract, Phoenix dactyhfera (date) seed extract, Avandula stoechas (spanish lavender) extract, Spiraea ulmaria (meadowsweet) leave extract, Chamomilla recutita (chamomile) leaf extract, and Symphytum officinale (comfrey) leaf extract and combination thereof. The extracts of these plants are obtained from seeds, roots, stem, leaves, flowers, bark, fruits, and/or whole plant.

In some embodiments, the plant extract is presented in the collagen stimulating compositions and methods of making and using thereof at a weight percent ranging from about 0.001 wt. % to about 10.0 wt. % by the total weight of the composition. In some embodiments, the plant extract is presented in the collagen stimulating compositions and methods of making and using thereof at a weight percent ranging from about 0.005 wt. % to about 5.0 wt. % by the total weight of the composition. In some embodiments, the plant extract is presented in the collagen stimulating compositions and methods of making and using thereof at a weight percent ranging from about 0.01 wt. % to about 2.0 wt. % by the total weight of the composition. In some embodiments, the plant extract is presented in the collagen stimulating compositions and methods of making and using thereof at a weight percent ranging from 0.0045 wt. % to 0.0055 wt. % by the total weight of the composition.

In some embodiments, the collagen stimulating compositions and methods of making and using thereof optionally comprises a UV filter that absorbs ultraviolet light of wavelengths between 290 to 329 nm. In some embodiments, the collagen stimulating compositions and methods of making and using thereof include an UV filter selected from the group consisting of para-aminobenzoic acid, ethyl para-aminobenzoate, amyl para-aminobenzoate, octyl para-aminobenzoate, ethylene glycol salicylate, phenyl salicylate, octyl salicylate, benzyl salicylate, butylphenyl salicylate, homomenthyl salicylate, benzyl cinnamate, 2-ethoxyethyl para-methoxycinnamate, octyl para-methoxycinnamate, glyceryl mono(2-ethylhexanoate) dipara-methoxycinnamate, isopropyl para-methoxycinnamate, diisopropyl-diisopropylcinnamic acid ester mixtures, urocanic acid, ethyl urocanate, hydroxymethoxybenzophenone, hydroxymethoxybenzophenonesulfonic acid and salts thereof, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl)benzotriazole. In some embodiments, the water soluble ultraviolet absorbent selected from the group consisting of 2-ethylhexyl-p-methoxycinnamate, 4-tert-butyl-4′-methoxydibenzoylmethane, octocrylene, 2,4-bis-[{4-(2-ethylhexyloxy)-2-hydroxy}-phenyl]-6-(4-methoxyphenyl)-1,3,5-triazine, methylene bis-benzotriazolyl tetramethylbutylphenol, 2,4,6-tris-[4-(2-ethylhexyloxycarbonyl)anilino]-1,3,5-triazine, diethylamino hydroxybenzoyl hexyl benzoate, oxybenzone, 2,2′-dihydroxy-4,4′-dimethoxy benzophenone, and combination thereof.

In some embodiments, the UV filter is selected from the group consisting of butyl methoxydibenzoylmethane, ethylhexyl methoxycinnamate, ethylhexyl salicylate, octocrylene, ethylhexyl methoxycinnamate, isoamyl-p-methoxycinnamate, ethylhexyltriazone, diethylhexyl butamido triazone, methylene bis-benzotriazolyl tetramethylbutylphenol, disodium phenyl dibenzimidazole tetrasulfonate, bis-ethylhexyloxyphenol methoxyphenyl triazine, benzophenone-3, and combination thereof.

In some embodiments, the collagen stimulating compositions and methods of making and using thereof comprises an inorganic pigment as UV filters selected from TiO₂, SiO₂, Fe₂O₃, ZrO₂, MnO, Al₂O₃, and combination thereof.

In some embodiments, the UV filter is presented in the composition at a weight percent ranging from about 0.001 wt. % to about 20.0 wt. % by the total weight of the collagen boosting composition. In some embodiments, the UV filter is presented in the composition at a weight percent ranging from about 0.01 wt. % to about 10.0 wt. % by the total weight of the composition. In some embodiments, the UV filter is presented in the composition at a weight percent ranging from about 0.05 wt. % to about 8.0 wt. % by the total weight of the composition.

In some embodiments, the collagen stimulating compositions and methods of making and using thereof optionally comprises an emollient selected from the group consisting of a hydrocarbon oil, a hydrocarbon wax, a silicone oil, an acetoglyceride ester, an ethoxylated glyceride, an alkyl ester of a fatty acid, an alkenyl ester of a fatty acid, a fatty acid, a fatty alcohol, a fatty alcohol ether, an ether-ester, lanolin, a lanolin derivative, a polyhydric alcohol, a polyether derivative, a polyhydric ester, a wax ester, a beeswax derivative, a vegetable wax, a natural or essential oil, a phospholipid, a sterol, an amide, and combination thereof.

In some embodiments, the emollients incorporated in the collagen stimulating compositions and methods of making and using thereof comprise ne or more of (1) hydrocarbon oils and waxes, e.g., mineral oil, petrolatum, paraffin, ozokerite, microcrystalline wax, polyethylene, squalene, and perhydrosqualene; (2) silicone oils, e.g., dimethyl polysiloxanes, methylphenyl polysiloxanes, water-soluble and alcohol-soluble silicone glycol copolymers; (3) acetoglyceride esters, e.g., acetylated monoglycerides; (4) ethoxylated glycerides, e.g., ethoxylated glyceryl monostearate; (5) alkyl esters of fatty acids having 10 to 20 carbon atoms, e.g., hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, methyl, isopropyl, butyl esters of fatty acids; (6) alkenyl esters of fatty acids having 10 to 20 carbon atoms, e.g., oleyl myristate, oleyl stearate, and oleyl oleate; (7) fatty acids having 10 to 20 carbon atoms, e.g., pelargonic, lauric, myristic, palmitic, stearic, isostearic, hydroxystearic, oleic, linoleic, ricinoleic, arachidic, behenic, and erucic acids; (8) fatty alcohols having 10 to 20 carbon atoms, e.g., lauryl, myristyl, cetyl, hexadecyl, stearyl, isostearyl, hydroxystearyl, oleyl, ricinoleyl, behenyl, erucyl alcohols, and 2-octyl dodecanol; (9) fatty alcohols ethers, e.g., ethoxylated fatty alcohols of 10 to 20 carbon atoms, lauryl, cetyl, stearyl, isostearyl, oleyl, and cholesterol alcohols having attached thereto from 1 to 50 ethylene oxide groups or 1 to 50 propylene oxide groups; (10) ether-esters, e.g. fatty acid esters of ethoxylated fatty alcohols; (11) lanolin and its derivatives, e.g., lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; (12) polyhydric alcohols and polyether derivatives, e.g., propylene glycol, dipropylene glycol, polypropylene glycols 2000 and 4000, polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide]homopolymers (weight average molecular weight of 100,000-5,000,000 Da), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C15-C18 vicinal glycol, and polyoxypropylene derivatives of trimethylolpropane; (13) polyhydric alcohol esters, e.g., ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters, sucrose cocoate, sucrose dilaurate, sucrose distearate, sucrose hexaerucate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tribehenate, sucrose tristearat; (14) wax esters, e.g., beeswax, spermaceti, myristyl myristate, and stearyl stearate; (15) beeswax derivatives, e.g., polyoxyethylene sorbitol beeswax which are reaction products of beeswax with ethoxylated sorbitol of varying ethylene oxide content; (16) vegetable waxes, e.g., carnauba and candelilla waxes; (17) natural or essential oils, e.g., citrus oil, non-citrus fruit oil, nut oils, oils having flavors, perfume or scents, canola oil, corn oil, neem oil, olive oil, cottonseed oil, coconut oil, fractionated coconut oil, palm oil, nut oils, safflower oil, sesame oil, soybean oil, peanut oil, almond oil, cashew oil, hazelnut oil, macadamia oil, pecan oil, pine nut oil, pistachio oil, walnut oil, grapefruit seed oil, lemon oil, orange oil, sweet orange oil, tangerine oil, lime oil, mandarin oil, omega 3 oil, flaxseed oil (linseed oil), apricot oil, avocado oil, carrot oil, cocoa butter oil, coconut oil, fractionated coconut oil, hemp oil, papaya seed oil, rice bran oil, shea butter oil, tea tree seed oil, and wheat germ oil, lavender oil, rosemary oil, tung oil, jojoba oil, poppy seed oil, shea butter, castor oil, mango oil, rose hip oil, tall oil chamomile oil, cinnamon oil, citronella oil, eucalyptus oil, fennel seed oil, jasmine oil, juniper berry oil, raspberry seed oil, lavender oil, primrose oil, lemon grass oil, nutmeg oil, patchouli oil, peppermint oil, pine oil, rose oil, rose hip oil, rosemary oil, eucalyptus oil, tea tree oil, rosewood oil, sandalwood oil, sassafras oil, spearmint oil, Ricinus communis (castor) seed oil, wintergreen oil; (18) phospholipids, e.g., lecithin and derivatives; (19) sterols, e.g., cholesterol and cholesterol fatty acid esters; and (20) fatty acid amides, ethoxylated fatty acid amides, and solid fatty acid alkanolamides, (21) lanolin, Therbroma cacao (cocoa) seed butter, petrolatum, Euphorbia cerifera (candelilla) wax, honey, geraniol, menthol, camphor, cetyl esters, mineral oil, salicylic acid, phenol, palmitoyl isoleucine,

In some embodiments, the collagen stimulating compositions and methods of making and using thereof optionally comprises a moisturizer selected from the group consisting of water-soluble, low molecular weight moisturizers, fat-soluble, low molecular weight moisturizers, water-soluble, high molecular weight moisturizers and fat-soluble, high molecular weight moisturizers, humectant, and combination thereof.

In some embodiments, the moisturizer comprises a humectant. As used herein, the term “humectant” refer to a hygroscopic substance used to keep things moist. A humectant attracts and retains the moisture in the air nearby via absorption, drawing the water vapor into or beneath the organism's or object's surface.

In some embodiments, the collagen stimulating compositions and methods of making and using thereof optionally comprises a water-soluble silk fibroin peptide as humectant. The amino peptides derived from the silk fibroin protein fragments can be easily absorbed by skin. In some embodiments, a water-soluble silk fibroin peptide may be added to the composition to give an enhanced after use feeling.

In some embodiments, amino acids derived from the silk fibroin protein fragments may be added to the collagen stimulating compositions and methods of making and using thereof as a conditioning agent (e.g. to exert excellent condition effects such as moist feel, softness, smoothness, gloss).

In some embodiments, the collagen stimulating compositions and methods of making and using thereof may comprise one or more additional humectant selected from the group consisting of honey, aloe vera, aloe vera leaf juice, aloe vera leaf extract, sorbitol, urea, lactic acid, sodium lactate, pyrrolidone carboxylic acid, trehalose, maltitol, alpha-hydroxy acids, sodium pyroglutamate, pyrolidonecarboxylate, N-acetyl-ethanolamine, sodium lactate, isopropanol, polyalkylene glycols (e.g., ethylene glycol, propylene glycol, hexylene glycol, 1,3-butylene glycol, dipropylene glycol, triethylene glycol), 1,3-propanediol, diethylene glycol monoethyl ether, glyceryl coconate, hydroxystearate, myristate, oleate, sodium hyaluronate, hyaluronic acid, chondroitin sulfuric acid, phospholipids, collagen, elastin, ceramides, lecithin sorbitol, PEG-4, and combination thereof.

In some embodiments, the collagen stimulating compositions and methods of making and using thereof optionally comprise polyhydric alcohols as moisturizer selected from the group consisting of ethylene glycol, propylene glycol, 1,3 butylene glycol, glycerin, sorbitol, polyethylene glycol, glutamine, mannitol, pyrrolidone-sodium carboxylate, (polymerization degree n=2 or more), polypropylene glycol (polymerization degree n=2 or more), polyglycerin (polymerization degree n=2 or more), lactic acid, lactate, and combination thereof.

In some embodiments, the collagen stimulating compositions and methods of making and using thereof optionally comprise fat-soluble, low molecular weight moisturizers selected from the group consisting of cholesterol and cholesterol ester. In some embodiments, the composition optionally comprises water-soluble, high molecular weight moisturizers selected from the group consisting of carboxyvinyl polymers, polyaspartate, tragacanth, xanthane gum, methyl cellulose, hydroxymethyl cellulose, hydroxyethyl cellulose, hydroxypropyl cellulose, carboxymethyl cellulose, water-soluble chitin, chitosan and dextrin. In some embodiments, the composition optionally comprises fat-soluble, high molecular weight moisturizers selected from the group consisting of polyvinylpyrrolidone-eicosene copolymers, polyvinylpyrrolidone-hexadecene copolymers, nitrocellulose, dextrin fatty acid ester and high molecular silicone.

Additional suitable moisturizers include polymeric moisturizers that are water soluble and/or water swellable in nature. In some embodiments, hyaluronic acid, or chitosan is combined with moisturizers to enhance their properties.

In some embodiments, the collagen stimulating compositions and methods of making and using thereof contains moisturizer at about 0.1 wt. % to about 30.0 wt. % by the total weight of the collagen boosting composition. In some embodiments, the composition contains moisturizer at about 0.5 wt. % to about 25.0 wt. % by the total weight of the collagen boosting composition. In some embodiments, the composition contains moisturizer at about 1.0 wt. % to about 20.0 wt. % by the total weight of the composition.

Compositions described herein may include an additional active agent, such as a drug. In some embodiments, the active agent can be one or more of enzyme inhibitors, anesthetic agents, medicinal neurotoxins, antioxidants, anti-infective agents, anti-inflammatory agents, vasodilators, ultraviolet (UV) light blocking agents, dyes (e.g., tattoo dye, ink or pigment), a reflective agent, hormones, immunosuppressants, and combinations thereof. The compositions described herein can include an active agent selected from the group consisting of enzyme inhibitors, anesthetic agents, medicinal neurotoxins (e.g., botulinum toxin and clostridium toxin), antioxidants, anti-infective agents (e.g., antibiotics), vasodilators, dyes (e.g., tattoo ink or pigment, reflective agents, anti-inflammatory agents, ultraviolet (UV) light blocking agents, dyes, hormones, immunosuppressants, and combinations thereof. In some embodiments, the immunosuppressant is rapamycin, or rapamycin-like compound. In some embodiments, the active agent may be an antibiotic selected from the group consisting of a penicillin (e.g., penicillin V, amoxicillin), an erythromycin (e.g., erythromycin stearate), a lincosamide (e.g., clindamycin), and a cephalosporin (e.g. cephalexin), and a combination thereof.

In some embodiments, the additional active agent may be a vasodilator selected from the group consisting of nitroglycerin, labetalol, thrazide, isosorbide dinitrate, pentaerythritol tetranitrate, digitalis, hydralazine, diazoxide, amrinone, L-arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di-isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside, and a combination thereof.

In some embodiments, the compositions described herein may include an additional active agent at a concentration, by weight, of about 0.01% to about 0.1%, or about 0.05% to about 0.15%, or about 0.1% to about 0.2%, or about 0.15% to about 0.25%, or about 0.2% to about 0.3%, or about 0.25% to about 0.35%, or about 0.3% to about 0.4%, or about 0.35% to about 0.45%, or about 0.4% to about 0.5%, or about 0.45% to about 0.55%, or about 0.5% to about 0.6%, or about 0.55% to about 0.65%, or about 0.6% to about 0.7%, or about 0.65% to about 0.75%, or about 0.7% to about 0.8%, or about 0.75% to about 0.85%, or about 0.8% to about 0.9%, or about 0.85% to about 0.95%, or about 1% to about 2%, or about 1.5% to about 2.5%, or about 2% to about 3%, or about 2.5% to about 3.5%, or about 3% to about 4%, or about 3.5% to about 4.5%, or about 4% to about 5%, or about 4.5% to about 5.5%, or about 5% to about 6%, or about 5.5% to about 6.5%, or about 6% to about 7%, or about 6.5% to about 7.5%, or about 7% to about 8%, or about 7.5% to about 8.5%, or about 8% to about 9%, or about 8.5% to about 9.5%, or about 9% to about 10%, or about 10% to about 15%, or about 15% to about 20%, or about 20% to about 25%, or about 25% to about 30%, or about 30% to about 35%, or about 35% to about 40%, or about 40% to about 45%, or about 45% to about 50%.

In some embodiments, the compositions described herein may include a fibrosis-inhibiting agent. In some embodiments, compositions described herein may further include a compound that acts to have an inhibitory effect on pathological processes in or around a treatment site. In certain aspects, the active agent may be selected from one of the following classes of compounds: anti-inflammatory agents (e.g., dexamethasone, cortisone, fludrocortisone, prednisone, prednisolone, 6α-methylprednisolone, triamcinolone, betamethasone, and aspirin).

In some embodiments, the collagen stimulating compositions and methods of making and using thereof optionally comprise a particle, wherein the particle may include polymeric particle, mica, silica, mud, and clay. The particles in the collagen stimulating compositions and methods of making and using thereof provide the benefits of smoothness, reduced friction, slippery feel whilst leaving the hair feeling clean, light and airy, and improved texture when spread on the hands and/or hair.

In some embodiments, the collagen stimulating compositions and methods of making and using thereof contains a polymeric particle formed of a polymer selected from the group consisting of an anionic and/or nonionic and/or zwitterionic polymer. In some embodiments, the composition contains a polymeric particle formed of a polymer selected from the group consisting of polystyrene, polyvinylacetate, polydivinylbenzene, polymethylmethacrylate, poly-n-butylacrylate, poly-n-butylmethacrylate, poly-2-ethylhexylmethyacrylate, 6,12-nylon, poyurethanes, epoxy resins, styrene/vinyl acetate copolymers, styrene/trimethylaminoethyl methacrylate chloride copolymers, and combinations thereof.

In some embodiments, the collagen stimulating compositions and methods of making and using thereof contains a cationically polymeric particle formed of a hydrophobic polymer selected from the group consisting of polyethylene homopolymers, ethylene-acrylic acid copolymer, polyamide polymer having a molecular weight in the range of from about 6,000 Da to about 12,000 Da, polyethylene-vinyl acetate copolymer, silicone-synthetic wax copolymer, silicone-natural wax copolymer, candelilla-silicone copolymer, ozokerite-silicone copolymer, synthetic paraffin wax-silicone copolymer, and combinations thereof.

In some embodiments, the collagen stimulating compositions and methods of making and using thereof contains swollen polymer particles for depositing discrete particles. In some embodiments, the swollen polymer particles are selected from the group consisting of particulate silicone polymers and surface-alkylated spherical silicon particles. In some embodiments, the silicone polymers forming the swollen polymer particles are selected from the group consisting of polydiorganosiloxanes, polymonoorganosiloxanes, and cross-linked polydimethyl siloxanes, crosslinked polymonomethyl siloxanes optionally having end groups including hydroxyl or methyl, and crosslinked polydimethyl siloxane (DC 2-9040 silicone fluid by Dow Corning). The polydisorganosiloxanes are preferably derived from suitable combinations of R₃SiO_{0.5} repeating units and R₂SiO repeating units. The polymonoorganosiloxanes are derived from R₁SiO_{1.5}. Each R independently represents an alkyl, alkenyl (e.g. vinyl), alkaryl, aralkyl, or aryl (e.g. phenyl) group. In some embodiments, R is a methyl group.

In some embodiments, the polymeric particles are nanoparticles having a median particle size of less than 1000 nm. In some embodiments, the polymeric particles have a median particle size of about 5 nm to about 600 nm. In some embodiments, the polymeric particles have a median particle size of about 10 nm to about 500 nm. In some embodiments, the polymeric particles have a median particle size of about 10 nm to about 400 nm. In some embodiments, the polymeric particles have a median particle size of about 20 nm to about 300 nm. In some embodiments, the polymeric particles have a median particle size of about 50 nm to about 600 nm.

In some embodiments, the collagen stimulating compositions and methods of making and using thereof contains clay particles forming a dispersion or a suspension in the dermatologically acceptable carrier as disclosed herein. Throughout this specification, the term “clay” is intended to mean fine-grained earthy materials that become plastic when mixed with water. The clay may be a natural, synthetic or chemically modified clay. Clays include hydrous aluminum silicates which contain impurities, e.g. potassium, sodium, magnesium, or iron in small amounts.

In one embodiment, the clay is a material containing from 38.8% to 98.2% of SiO₂ and from 0.3% to 38.0% of Al₂O₃, and further contains one or more of metal oxides selected from Fe₂O₃, CaO, MgO, TiO₂, ZrO₂, Na₂O and K₂O. In some embodiments, the clay has a layered structure comprising hydrous sheets of octahedrally coordinated aluminum, magnesium or iron, or of tetrahedrally coordinated silicon.

In one embodiment, the clay is selected from the group consisting of kaolin, talc, 2:1 phyllosilicates, 1:1 phyllosilicates, smectite, bentonite, montmorillonites (also known as bentonites), hectorites, volchonskoites, nontronites, saponites, beidelites, sauconites, and mixtures thereof. In one embodiment, the clay is kaolin or bentonite. In some embodiments, the clay is a synthetic hectorite. In another embodiment, the clay is a bentonite.

In some embodiments, the clays have a cation exchange capacity of from about 0.7 meq/100 g to about 150 meq/100 g. In some embodiments, the clays have a cation exchange capacity of from about 30 meq/100 g to about 100 meq/100 g.

In some embodiments, the collagen stimulating compositions and methods of making and using thereof optionally comprise a composite particle having an anionically charged clay electrostatically complexed with the cationically charged hair conditioning agents as disclosed herein.

Commercially available synthetic hectorites include those products sold under the trade names Laponite® RD, Laponite® RDS, Laponite® XLG, Laponite® XLS, Laponite® D, Laponite® DF, Laponite® DS, Laponite® S, and Laponite® JS (Southern Clay products, Texas, USA). Commercially available bentonites include those products sold under the trade names Gelwhite® GP, Gelwhite® H, Gelwhite® L, Mineral Colloid® BP, Mineral Colloid® MO, Gelwhite® MAS 100 (sc), Gelwhite® MAS 101, Gelwhite® MAS 102, Gelwhite® MAS 103, Bentolite® WH, Bentolite® L10, Bentolite® H, Bentolite® L, Permont® SX10A, Permont® SC20, and Permont® HN24 (Southern Clay Products, Texas, USA); Bentone® EW and Bentone® MA (Dow Corning); and Bentonite® USP BL 670 and Bentolite® H4430 (Whitaker, Clarke & Daniels). In some embodiments, the particles have a median particle size ranging from about 1 μm to about 100 μm. In some embodiments, the particles have a median particle size ranging from about 2 μm to about 50 μm. In some embodiments, the particles have a median particle size ranging from about 2 μm to about 20 μm. In some embodiments, the particles have a median particle size ranging from about 4 μm to about 10 μm. In some embodiments, the particles have a median particle size selected from: about 1 μm, about 1.1 μm, about 1.2 μm, about 1.3 μm, about 1.4 μm, about 1.5 μm, about 1.6 μm, about 1.7 μm, about 1.8 μm, about 1.9 m, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, about 3.0 μm, about 3.1 μm, about 3.2 m, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 m, about 4.6 μm, about 4.7 μm, about 4.8 μm, about 4.9 μm, about 5.0 μm, about 5.1 μm, about 5.2 μm, about 5.3 μm, about 5.4 μm, about 5.5 μm, about 5.6 μm, about 5.7 μm, about 5.8 m, about 5.9 μm, about 6.0 μm, about 6.1 μm, about 6.2 μm, about 6.3 μm, about 6.4 μm, about 6.5 μm, about 6.6 μm, about 6.7 μm, about 6.8 μm, about 6.9 μm, about 7.0 μm, about 7.1 m, about 7.2 μm, about 7.3 μm, about 7.4 μm, about 7.5 μm, about 7.6 μm, about 7.7 μm, about 7.8 μm, about 7.9 μm, about 8.0 μm, about 8.1 μm, about 8.2 μm, about 8.3 μm, about 8.4 m, about 8.5 μm, about 8.6 μm, about 8.7 μm, about 8.8 μm, about 8.9 μm, about 9.0 μm, about 9.1 μm, about 9.2 μm, about 9.3 μm, about 9.4 μm, about 9.5 μm, about 9.6 μm, about 9.7 m, about 9.8 μm, about 9.9 μm, and about 10.0 μm.

In some embodiments, the weight ratio of the cationically charged hair conditioning agent to the clay is from 0.05:1 to 20:1. In some embodiments, the weight ratio of the cationically charged hair conditioning agent to the clay is from 0.1:1 to 10:1. In some embodiments, the weight ratio of the cationically charged hair conditioning agent to the clay is from 0.2:1 to 5:1. In some embodiments, the weight ratio of the cationically charged hair conditioning agent to the clay is selected from 0.05:1, 0.1:1, 0.2:1, 0.5:1, 0.75:1, 1:1, 1.5:1, 2:1, 2.5:1, 3:1, 3.5:1, 4.0:1, 4.5:1, 5.0:1, 5.5:1, 6.0:1, 6.5:1, 7.0:1, 7.5:1, 8.0:1, 8.5:1, 9.0:1, 9.5:1, 10.0:1, 10.5:1, 11.0:1, 11.5:1, 12.0:1, 12.5:1, 13.0:1, 13.5:1, 14.0:1, 14.5:1, 15.0:1, 15.5:1, 16.0:1, 16.5:1, 17.0:1, 17.5:1, 18.0:1, 18.5:1, 19.0:1, 19.5:1, ND 20.0:1.

In some embodiments, the particle is present in the collagen stimulating compositions and methods of making and using thereof at a weight percent ranging from about 0.01 wt. % to about 10.0 wt. % by the total weight of the silk collagen boosting composition. In some embodiments, the particle is present in the collagen stimulating compositions and methods of making and using thereof at a weight percent ranging from about 0.1 wt. % to about 10.0 wt. % by the total weight of the silk collagen boosting composition. In some embodiments, the particle is present in the composition at a weight percent ranging from about 0.1 wt. % to about 2.0 wt. % by the total weight of the silk collagen boosting composition. In some embodiments, the particle is present in the composition at a weight percent ranging from about 1.0 wt. % to about 9.0 wt. % by the total weight of the silk collagen boosting composition. In some embodiments, the particle is present in the composition at a weight percent ranging from about 1.0 wt. % to about 5.0 wt. % by the total weight of the silk collagen boosting composition. In some embodiments, the particle is present in the composition at a weight percent selected from: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4 wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4 wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4 wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4 wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4 wt. %, about 9.5 wt. %, about 9.6 wt. %, about 9.7 wt. %, about 9.8 wt. %, about 9.9 wt. %, and about 10.0 wt. % by the total weight of the composition.

In some embodiments, the collagen stimulating compositions and methods of making and using thereof optionally comprise a colloidal stabilizer to maintain particle dispersive stability, particularly of larger sized particles. Suitable colloidal stabilizer is selected from the group consisting of propylene oxide-ethylene oxide copolymers or ethyleneoxide-propylenoxide graphted polyethylenimines, polyoxyethylene (20-80 units POE) isooctylphenyl ether, fatty alcohol ethoxylates, polyethoxylated polyterephthalate block co-polymers containing polyvinylpyrrolidone, copolymers containing vinylpyrolidone repeating units, and combinations thereof.

In some embodiments, collagen stimulating compositions and methods of making and using thereof comprises an emulsion as the dermatologically acceptable carrier. In some embodiments, the dermatologically acceptable carrier exists as a conventional emulsion. In some embodiments, the dermatologically acceptable carrier exits as a microemulsion. In some embodiments, the dermatologically acceptable carrier exits as a water-in-oil emulsion. In some embodiments, the dermatologically acceptable carrier exits as an oil-in-water emulsion. In some embodiments, the dermatologically acceptable carrier exits as a nano-emulsion. In some embodiments, the dermatologically acceptable carrier exits as a water-in-silicone oil emulsion. In some embodiments, the dermatologically acceptable carrier exits as a silicone oil-in-water emulsion.

As used herein, the conventional emulsions have one continuous phase and one disperse phase, which is present as very small spheres stabilized by coating with surfactants. Depending on the nature of the continuous phase, the emulsions are described as oil-in-water or water-in-oil. These emulsions are kinetically stable in the ideal case, i.e. they are retained even for a prolonged period, but not indefinitely. During temperature fluctuations in particular, they may have a tendency toward phase separation as a result of sedimentation, creaming, thickening or flocculation.

As used herein, the microemulsions are thermodynamically stable, isotropic, fluid, optically clear single liquid phase containing a ternary system having three ingredients of an oily component, an aqueous component and a surfactant. Microemulsions arise when a surfactant, or more frequently a mixture of a surfactant and a cosurfactant, reduces the oil/water interfacial tension to extremely low values, often in the range 10³ to 10⁹, preferably 10⁴ to 10⁶ N/m, such that the two insoluble phases remain dispersed by themselves in a homogeneous manner as a result of the thermal agitation. Microemulsions often have bicontinuous structures with equilibrium regions, so-called subphases in the order of magnitude from 100 to 1000 Angstroms. The microemulsion refers to either one state of an O/W (oil-in-water) type microemulsion in which oil is solubilized by micelles, or a bicontinuous microemulsion in which the number of associations of surfactant molecules are rendered infinite so that both the aqueous phase and oil phase have a continuous structure.

For properties, the microemulsion appears transparent or translucent and may exist as a solution in a monophasic state in which all the formulated ingredients and components are uniformly dissolved therein.

Regardless of manufacturing processes, microemulsions may take the same state if they have the same formulation components and prepared at the same temperature. Therefore, the above-described three ingredients (oil, water and surfactant) and the remaining ingredients may be added and mixed in any orders as appropriate and may be agitated using mechanical forces at any power to consequently yield a microemulsion having substantially the same state (in appearance, viscosity, feeling of use, etc.).

Bicontinuous microemulsions comprise two phases, a water phase and an oil phase, in the form of extended adjoining and intertwined domains at whose interface stabilizing interface-active surfactants are concentrated in a monomolecular layer. Bicontinuous micro emulsions form very readily, usually spontaneously due to the very low interfacial tension, when the individual components, water, oil and a suitable emulsifier system, are mixed. Since the domains have only very small extensions in the order of magnitude of nanometers in at least one dimension, the microemulsions appear visually transparent and are thermodynamically, i.e. indefinitely, stable in a certain temperature range depending on the emulsifier system used.

As used herein, the term nanoemulsions refer to emulsions presenting transparent or translucent appearances due to their nano particle sizes, e.g. less than 1000 nm.

Emulsifiers (e.g., surfactants) are substances which reduce the interfacial tension between liquid phases which are not miscible with one another, a polar phase, often water and a nonpolar, organic phase, and thus increase their mutual solubility. Surfactants have a characteristic structure feature of at least one hydrophilic and one hydrophobic structural unit. This structure feature is also referred to as amphiphilic.

Anionic, cationic, amphoteric and nonionic surfactants have conventionally been used as emulsifiers for production of emulsified cosmetic materials by emulsification of water and oily substances. However, since synthetic surfactants have been implicated in the destruction of skin surface tissue and constituting a cause of liver damage when entering the body, numerous naturally-derived protein-based emulsifiers including natural protein based emulsifiers have been employed because of their high safety.

Although emulsified cosmetic materials obtained using protein-based emulsifiers generally have a soft, moist feel during use, it is often the case finished products impart a crumbling feel and lack spreadability. The important factors for emulsifiers used in cosmetic products include not only safety and emulsifying power, but also feel during use. The disclosure provides the use of silk fibroin protein fragments as emulsifier (thereafter silk emulsifier) to stabilize the emulsion carrier for the collagen boosting composition disclosed herein.

In an embodiment, the collagen stimulating compositions and methods of making and using thereof comprises an emulsion as carrier having a silk emulsifier in the emulsifier system. Silk fibroin is an amphiphilic polymer with large hydrophobic domains occupying the major component of the polymer, which has a high molecular weight. The hydrophobic regions are interrupted by small hydrophilic spacers, and the N- and C-termini of the chains are also highly hydrophilic. The hydrophobic domains of the H-chain contain a repetitive hexapeptide sequence of Gly-Ala-Gly-Ala-Gly-Ser and repeats of Gly-Ala/Ser/Tyr dipeptides, which can form stable anti-parallel-sheet crystallites. The amino acid sequence of the L-chain is non-repetitive, so the L-chain is more hydrophilic and relatively elastic. The hydrophilic (Tyr, Ser) and hydrophobic (Gly, Ala) chain segments in silk fibroin molecules are arranged alternatively such that allows self-assembling of silk fibroin molecules.

In some embodiments, the emulsifier system comprises a silk emulsifier and a small molecule having high HLB value. The composition of hydrophobic repeating groups is one penta-peptide -Gly-Ala-Gly-Ala-Gly- for each hydrophilic -Ser-, the hydrophilic-hydrophobic balance (HLB) for the silk fibroin protein can be modified to a range from 7.95-16.74 in a hydrophilic environment created by the addition of a hydrophilic molecule having high HLB value (i.e. >10). This range of HLB value of the silk fibroin protein fragments allows the preparation of a wide range of emulsions from O/W type emulsions to W/O type emulsions. In some embodiments, the hydrophilic molecule having high HLB value is selected from the group consisting of glycerol HLB 11.28, butantetraol HLB 12.7, xylitol HLB 14.13, D-sorbitol HLB 15.55, inositol HLB 16.74, polysaccharide including hyaluronic acid, hyaluronate, carrageenan, pullulan, alginic acid, alginate, microbial exopolysaccharides, glucosamine, chondroitin sulfate, glycosaminoglycans, glucomannan, and combination thereof. In some embodiments, the emulsifier system comprises the silk emulsifier and glycerol.

In some embodiments, the silk emulsifier and hydrophilic molecule having high HLB value are incorporated in the emulsion carrier at a weight ratio of silk emulsifier to the hydrophilic molecule of 1:1 to 1:10. In some embodiments, the silk emulsifier and hydrophilic molecule having high HLB value are incorporated in the emulsion carrier at a weight ratio of silk emulsifier to the hydrophilic molecule selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:8, 1:9 and 1:10. In some embodiments, the silk emulsifier and hydrophilic molecule having high HLB value are incorporated in the emulsion carrier at a weight ratio of silk emulsifier to the hydrophilic molecule of 1:1. In some embodiments, the emulsifier system comprises the silk emulsifier and glycerol at a weight ratio of silk emulsifier to glycerol of 1:1 to 1:3. In some embodiments, the emulsifier system comprises the silk emulsifier and glycerol at a weight ratio of silk emulsifier to glycerol selected from: 1:1, 1:1. 1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3.0.

In an embodiment, this disclosure provides an aqueous solution of silk fibroin protein fragments or the aqueous gel of silk fibroin protein based fragments as described above as emulsifier (hereafter as silk emulsifier) for the emulsion carrier. The aqueous solution of silk fibroin protein fragments or the aqueous gel of silk fibroin protein fragments as described above may be admixed with an oily component to achieve uniform emulsification between the water in the aqueous solution or aqueous gel of the silk fibroin protein fragments and the oily component.

In some embodiments, the silk fibroin protein fragments used as emulsifier has a weight average molecular weight of greater than about 5 kDa. In some embodiments, the silk fibroin protein used as emulsifier has a weight average molecular weight selected from about 5 kDa to about 350 kDa. In some embodiments, the silk fibroin protein used as emulsifier has a weight average molecular weight selected from between about 20 kDa to about 80 kDa. In some embodiments, the silk fibroin protein used as emulsifier has a weight average molecular weight selected from between about 40 kDa to about 60 kDa. In other embodiments, any silk fibroin fragments described herein can be used as emulsifiers.

In some embodiments, the amount of the silk emulsifier presented in the emulsion carrier ranges from about 0.1 wt. % to about 15.0 wt. % by the total weight of the emulsion carrier. In some embodiments, the amount of the silk emulsifier presented in the emulsion carrier ranges from about 0.75 wt. % to about 10.0 wt. % by the total weight of the emulsion carrier. In some embodiments, the amount of the silk emulsifier presented in the emulsion carrier is selected from the group consisting of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.50 wt. %, about 12.75 wt. %, about 13.0 wt. %, about 13.25 wt. %, about 13.50 wt. %, about 13.75 wt. %, about 14.0 wt. %, about 14.25 wt. %, about 14.50 wt. %, about 14.75 wt. %, and about 15.0 wt. %.

Silk protein in the aqueous solution tends to fibrillate more readily by shear of vibration or stirring if it has a higher molecular weight. The fibrillated protein consists of water-insoluble masses causes reduction of pleasant feel during use of the cosmetic materials.

In some embodiments, the silk fibroin protein fragments are blended with hydrophilic substance with high HLB value to enhance the hydrophilic environment and such hydrophilic substance includes glycerol, butantetraol, xylitol, D-sorbitol, inositol polyethylene glycol, polyethylene oxide, polylactic acid, cellulose, chitin and polyvinyl alcohol to prevent silk fibroin solution from gelation. It is important to prevent fibroin transformation from random coils to β-sheet structure (fibrillate).

In some embodiments, a sucrose fatty ester based emulsifier having HLB value>10 is added to the silk fibroin protein as emulsion stabilizer to enhance silk fibroin protein emulsification efficiency.

In some embodiments, the emulsifying system for the collagen stimulating compositions and methods of making and using thereof may include a sucrose fatty ester based emulsifier and an aqueous solution of silk fibroin protein or the aqueous gel of silk fibroin protein.

In some embodiments, an aqueous solution or an aqueous gel containing silk fibroin protein fragments may be used as co-emulsifier for the collagen stimulating compositions, wherein the aqueous solution or gel of silk protein is obtained by dissolving unscoured, partially scoured or scoured spun silkworm fibers (cocoon filaments) with a neutral salt (e.g. lithium bromide). In some embodiments, the sucrose fatty ester is sucrose palmitate and sucrose laurate ester. In some embodiments, silk proteins may be employed as surfactants for the collagen stimulating compositions with enhanced emulsifying efficiency. In some embodiments, phospholipids (e.g. lecithin) may be used to complex with silk fibroin protein fragments derived co-emulsifiers to increase their emulsifying power (efficiency of surfactant).

In some embodiments, the collagen stimulating compositions containing microemulsion obtained using silk fibroin protein fragments-based emulsifier generally have good spreadability, a soft, and moist feel during use. In some embodiments, the emulsion carrier for the collagen stimulating compositions and methods of making and using thereof may further comprise one or more ionic surfactants as co-emulsifiers.

An ionic surfactant is a surfactant that is ionized to have an electric charge in an aqueous solution; depending on the type of the electric charge, it is classified into ampholytic surfactants, cationic surfactants, or anionic surfactants. When an anionic surfactant and an ampholytic surfactant, or an anionic surfactant and a cationic surfactant, are mixed in an aqueous solution, the interfacial tension against oil decreases.

An ampholytic surfactant has at least one cationic functional group and one anionic functional group, is cationic when the solution is acidic and anionic when the solution is alkaline, and assumes characteristics similar to a nonionic surfactant around the isoelectric point.

Ampholytic surfactants are classified, based on the type of the anionic group, into the carboxylic acid type, the sulfuric ester type, the sulfonic acid type, and the phosphoric ester type. For the present invention, the carboxylic acid type, the sulfuric ester type, and the sulfonic acid type are preferable. The carboxylic acid type is further classified into the amino acid type and the betaine type. Particularly preferable is the betaine type.

Specific examples include: imidazoline type ampholytic surfactants (for example, 2-undecyl-1-hydroxyethyl-1-carboxymethyl-4,5-dihydro-2-imidazolium sodium salt and 1-[2-(carboxymethoxy)ethyl]-1-(carboxymethyl)-4,5-dihydro-2-norcocoalkylimidazolium hydroxide disodium salt); and betaine type surfactants (for example, 2-heptadecyl-N-carboxymethyl-N-hydroxyethyl imidazolinium betaine, lauryldimethylarninoacetic acid betaine, alkyl betaine, amide betaine, and sulfobetaine).

Examples of the cationic surfactant include quaternary ammonium salts such as cetyltrimethylammonium chloride, stearyltrimethylammonium chloride, benenyltrimethylammonium chloride, behenyldimethylhydroxyethylammonium chloride, stearyldimethylbenzylammonium chloride, and cetyltrimethylammonium methylsulfate. Other examples include amide amine compounds such as stearic diethylaminoethylamide, stearic dimethylaminoethylamide, palmitic diethylaminoethylamide, palmitic dimethylaminoethylamide, myristic diethylaminoethylamide, myristic dimethylaminoethylamide, behenic diethylaminoethylamide, behenic dimethylaminoethylamide, stearic diethylaminopropylamide, stearic dimethylaminopropylamide, palmitic diethylaminopropylamide, palmitic dimethylaminopropylamide, myristic diethylaminopropylamide, myristic dimethylaminopropylamide, behenic diethylaminopropylamide, and behenic dimethylaminopropylamide.

In some embodiments, the emulsifier system for the collagen stimulating compositions and methods of making and using thereof may further comprise one or more anionic surfactants. Anionic surfactants are classified into the carboxylate type such as fatty acid soaps, N-acyl glutamates, and alkyl ether acetates, the sulfonic acid type such as α-olefin sulfonates, alkane sulfonates, and alkylbenzene sulfonates, the sulfuric ester type such as higher alcohol sulfuric ester salts, and phosphoric ester salts. Preferable are the carboxylate type, the sulfonic acid type, and the sulfuric ester salt type; particularly preferable is the sulfuric ester salt type.

In some embodiments, the anionic surfactant for the collagen stimulating compositions and methods of making and using thereof is selected from the group consisting of higher alkyl sulfuric acid ester salts (for example, sodium lauryl sulfate and potassium lauryl sulfate); alkyl ether sulfuric acid ester salts (e.g., POE-triethanolamine lauryl sulfate and sodium POE-lauryl sulfate); N-acyl sarcosinic acids (e.g., sodium lauroyl sarcosinate); higher fatty acid amide sulfonic acid salts (e.g., sodium N-myristoyl N-methyl taurate, Sodium N-cocoyl-N-methyl taurate, and Sodium jauroylmethyl taurate); phosphoric ester salts (e.g., sodium POE-oleyl ether phosphate and POE stearyl ether phosphoric acid); sulfosuccinates (e.g., sodium di-2-ethylhexylsulfosuccinate, sodium monolauroyl monoethanol amide polyoxyethylene sulfosuccinate, and sodium lauryl polypropylene glycol sulfosuccinate); alkyl benzene sulfonates (e.g., sodium linear dodecyl benzene sulfonate, triethanolamine linear dodecyl benzene sulfonate, and linear dodecyl benzene sulfonic acid); higher fatty acid ester sulfates (e.g., hydrogenated coconut oil aliphatic acid glyceryl sodium sulfate); N-acyl glutamates (e.g., mono sodium N-lauroylglutamate, disodium N-stearoylglutamate, and sodium N-myristoyl-L-glutamate); sulfated oils (e.g., turkey red oil); POE-alkyl ether carboxylic acid; POE-alkyl aryl ether carboxylate; α-olefin sulfonate; higher fatty acid ester sulfonates; sec-alcohol sulfates; higher fatty acid alkyl amide sulfates; sodium lauroyl monoethanolamine succinates; ditriethanolamine N-palmitoylaspartate; and sodium caseinate.

In some embodiments, the emulsifier system for the collagen stimulating compositions and methods of making and using thereof may further comprise one or more nonionic surfactants as co-emulsifiers. The nonionic surfactant preferably has an HLB value of 8.9-14. It is generally known that the solubility into water and the solubility into oil balance when the HLB is 7. That is, a surfactant preferable for the present invention would have medium solubility in oil/water.

The nonionic surfactants may include: (1) polyethylene oxide extended sorbitan monoalkylates (e.g., polysorbates); (2) polyalkoxylated alkanols; (3) polyalkoxylated alkylphenols include polyethoxylated octyl or nonyl phenols having HLB values of at least about 14, which are commercially available under the trade designations ICONOL® and TRITON®; (4) polaxamers. Surfactants based on block copolymers of ethylene oxide (EO) and propylene oxide (PO) may also be effective. Both EO-PO-EO blocks and PO-EO-PO blocks are expected to work well as long as the HLB is at least about 14, and preferably at least about 16. Such surfactants are commercially available under the trade designations PLURONIC® and TETRONIC® from BASF; (5) polyalkoxylated esters: polyalkoxylated glycols such as ethylene glycol, propylene glycol, glycerol, and the like may be partially or completely esterified, i.e. one or more alcohols may be esterified, with a (C8 to C22) alkyl carboxylic acid. Such polyethoxylated esters having an HLB of at least about 14, and preferably at least about 16, may be suitable for use in compositions of the present invention; (6) alkyl polyglucosides. This includes glucopon 425, which has a (C8 to C16) alkyl chain length; (7) sucrose fatty acid ester having high HLB value (8-18): sucrose cocoate, sucrose dilaurate, sucrose distearate, sucrose hexaerucate, sucrose hexaoleate/hexapalmitate/hexstearate, sucrose hexapalmitate, sucrose laurate, sucrose myristate, sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose trilaurate.

In some embodiments, the emulsifier system comprises a lipophilic nonionic surfactants selected from the group consisting of sorbitan fatty acid esters (e.g., sorbitan mono oleate monooleate, sorbitan mono isostearate monoisostearate, sorbitan mono laurate monolaurate, sorbitan mono palmitate monopalmitate, sorbitan mono stearate monostearate, sorbitan sesquioleate, sorbitan trioleate, diglyceryl sorbitan penta-2-ethylhexylate, diglyceryl sorbitan tetra-2-ethylhexylate); glyceryl and polyglyceryl aliphatic acids (e.g., mono cottonseed oil fatty acid glycerine, glyceryl monoerucate, glyceryl sesquioleate, glyceryl monostearate, α,α′-glyceryl oleate pyroglutamate, monostearate glyceryl malic acid); propylene glycol fatty acid esters (e.g., propylene glycol monostearate); hydrogenated castor oil derivatives; glyceryl alkylethers, and combination thereof.

In some embodiments, the emulsifier system comprises a hydrophilic nonionic surfactants selected from the group consisting of POE-sorbitan fatty acid esters (e.g., POE-sorbitan monooleate, POE-sorbitan monostearate, POE-sorbitan monooleate, and POE-sorbitan tetraoleate); POE sorbitol fatty acid esters (e.g., POE sorbitol monolaurate, POE-sorbitol monooleate, POE-sorbitolpentaoleate, and POE-sorbitol monostearate); POE-glyceryl fatty acid esters (e.g., POE-monooleates such as POE-glyceryl monostearate, POE-glyceryl monoisostearate, and POE glycerin glyceryl triisostearate); POE-fatty acid esters (e.g, POE-distearate, POE-monodioleate, and ethylene glycol distearate); POE-alkylethers (e.g., POE-lauryl ether, POE-oleyl ether, POE-stearyl ether, POE-behenyl ether, POE 2-octyl dodecyl ether, and POE-cholestanol ether); pluaronics (e.g., pluaronic); POE-POP-alkylethers (e.g, POE-POP-cetyl ether, POE-POP2-decyl tetradecyl ether, POE-POP-monobutyl ether, POE-POP-lanolin hydrate, and POE-POP glycerin glyceryl ether); tetra POE-tetra POP-ethylenediamino condensates (e.g., tetronic); POE-castor oil hydrogenated castor oil derivatives (e.g., POE-castor oil, POE-hydrogenated castor oil, POE-hydrogenated castor oil monoisostearate, POE-hydrogenated castor oil triisostearate, POE-hydrogenated castor oil monopyroglutamic monoisostearic diester, and POE-hydrogenated castor oil maleic acid); POE-beeswax-lanolin derivatives (e.g., POE-sorbitol beeswax); alkanol amides (e.g., palm oil fatty acid diethanol amide, laurate monoethanolamide, and fatty acid isopropanol amide); POE-propylene glycol fatty acid esters; POE-alkylamines; POE-fatty acid amides; sucrose fatty acid esters; alkyl ethoxydimethylamine oxides; and trioleyl phosphoric acid.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some embodiments, the emulsifier system comprises mono-glycerol derivatives and/or diglycerol derivatives. Specific examples include: monoglycerol derivatives such as monoglycerol monooctanoate, monooctyl monoglyceryl ether, monoglycerol monononanoate, monononyl monoglyceryl ether, monoglycerol monodecanoate, monodecyl monoglyceryl ether, monoglycerol monoundecylenate, monoundecylenyl glyceryl ether, monoglycerol monododecanoate, monododecyl monoglyceryl ether, monoglycerol monotetradecanoate, monoglycerol monohexadecanoate, monoglycerol monooleate, and monoglycerol monoisostearate, as well as diglycerol derivatives such as diglycerol monooctanoate, monooctyl diglyceryl ether, diglycerol monononanoate, monononyl diglyceryl ether, diglycerol monodecanoate, monodecyl diglyceryl ether, diglycerol monoundecylenate, monoundecylenyl glyceryl ether, diglycerol monododecanoate, monododecyl diglyceryl ether, diglycerol monotetradecanoate, diglycerol monohexadecanoate, diglycerol monooleate, and diglycerol monoisostearate.

In some embodiments, the emulsifier system comprises the silk emulsifier and one or more of sucrose laurate, and sucrose palmitate. In some embodiments, the emulsifier system comprises the silk emulsifier and sucrose laurate. In some embodiments, the emulsifier system comprises the silk emulsifier and sucrose palmitate. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate ranging from 1:1 to 1:3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate selected from: 1:1, 1:1.1, 1:1.2 and 1:1.3. In some embodiments, the emulsifier system comprises the silk emulsifier, sucrose laurate, and sucrose palmitate, wherein sucrose laurate, and sucrose palmitate in the emulsion carrier has a weight ratio of sucrose laurate to sucrose palmitate of 1:1.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0, wherein the silk emulsifier and the glycerol in the emulsion carrier has a weight ratio of silk emulsifier to glycerol selected from: 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, and 1:3.0.

In some embodiments, the emulsifier system comprises the silk emulsifier, glycerol, sucrose laurate, and sucrose palmitate, wherein sucrose laurate and sucrose palmitate in the emulsion carrier has a weight ratio of sucrose laurate to sucrose palmitate selected from: 1:1, 1:1.1, 1:1.2, and 1:1.3, wherein the silk emulsifier and the glycerol in the emulsion carrier has a weight ratio of silk emulsifier to glycerol selected from: 1:1, 1:2, and 1:3.0.

In some embodiments, the emulsifier system is incorporated in the emulsion carrier at a weight percent ranging from 0.1 wt. % to 5.0 wt. % by the total weight of the collagen boosting composition. In some embodiments, the emulsifier system is incorporated in the emulsion carrier at a weight percent ranging from 0.1 wt. % to 3.0 wt. % by the total weight of the collagen boosting composition. In some embodiments, the emulsifier system is incorporated in the emulsion carrier at a weight percent ranging from 0.1 wt. % to 2.0 wt. % by the total weight of the collagen boosting composition.

In some embodiments, the emulsion carrier comprises an oil phase emulsified with the emulsifier system containing the silk emulsifier as described above. The fatty materials may be useful for forming the oil phase. The fatty material is selected from the group consisting of hydrocarbon oils, silicon oil, higher fatty acids, higher alcohols, synthetic ester oils, silicone oils, liquid oils/fats, solid oils/fats, waxes, and combination thereof.

In an embodiment, the fatty material optionally comprises a wax. The wax is selected from the group consisting of polyethylene wax, polypropylene wax, beeswax, candelilla wax, paraffin wax, ozokerite, microcrystalline waxes, carnauba wax, cotton wax, esparto wax, carnauba wax, bayberry wax, tree wax, whale wax, montan wax, bran wax, lanolin, kapok wax, lanolin acetate, liquid lanolin, sugar cane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, lanolin fatty acid polyethylene glycol, POE hydrogenated lanolin alcohol ether, and combination thereof.

In an embodiment, the fatty material optionally comprises an ester oil. The ester oil is selected from the group consisting of cholesteryl isostearate, isopropyl palmitate, isopropyl myristate, neopentylglycol dicaprate, isopropyl isostearate, octadecyl myristate, cetyl 2-ethylhexanoate, cetearyl isononanoate, cetearyl octanoate, isononyl isononanoate, isotridecyl isononanoate, glyceryl tri-2-ethylhexanoate, glyceryl tri(caprylatelcaprate), diethylene glycol monoethyl ether oleate, dicaprylyl ether, caprylic acid/capric acid propylene glycol diester, and combination thereof.

In an embodiment, the fatty material optionally comprises a glyceride fatty ester. As used herein, the term “glyceride fatty esters” refers to the mono-, di-, and tri-esters formed between glycerol and long chain carboxylic acids such as C₆-C₃₀ carboxylic acids. The carboxylic acids may be saturated or unsaturated or contain hydrophilic groups such as hydroxyl. Preferred glyceride fatty esters are derived from carboxylic acids of carbon chain length ranging from C₁₀ to C₂₄, preferably C₁₀ to C₂₂ most preferably C₁₂ to C₂₀.

In an embodiment, the fatty material optionally comprises synthetic ester oils. In some embodiments, the synthetic ester oil is selected from the group consisting of isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glyceryl di-2-heptylundecanoate, trimethylolpropane tri-2-ethylhexylate, trimethylolpropane triisostearate, pentaneerythritol tetra-2-ethylhexylate, glyceryl tri-2-ethylhexylate, trimethylolpropane triisostearate, cetyl 2-ethylhexanoate, 2-ethylhexyl palmitate, glyceryl trimyristate, tri-2-heptylundecanoic glyceride, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl cebatate. 2-hexyldecyl myristate, 2-hexyldecyl palmitate, 2-hexyldecyl adipate, diisopropyl cebatate, 2-ethylhexyl succinate, ethyl acetate, butyl acetate, amyl acetate and triethyl|citrate, and combination thereof.

In an embodiment, the fatty material optionally comprises ether oil. In some embodiments, the ether oils are selected from the group consisting of alkyl-1,3-dimethylethyl ether, nonylphenyl ether, and combination thereof.

In an embodiment, the fatty material optionally comprises higher fatty acids. As used herein, the higher fatty acids have a carbon number ranging from 8 to 22. In some embodiments, the higher fatty acid is selected from the group consisting of lauric acid, myristic acid, palmitic acid, stearic acid, behenic acid, oleic acid, 12-hydroxystearic acid, undecylenic acid, tall oil, isostearic acid, linoleic acid, linolenic acid, eicosapentaenoic acid (EPA), docosahexaenoic acid (DHA), and combination thereof.

In an embodiment, the fatty material optionally comprises higher fatty alcohols. As used herein, the higher fatty alcohols have a carbon number ranging from 8 to 22. In some embodiments, the higher fatty acid is selected from the group consisting of straight chain alcohols (for example, lauryl alcohol, cetyl alcohol, stearyl alcohol, behenyl alcohol, myristyl alcohol, oleyl alcohol, and cetostearyl alcohol) and branched chain ethyl alcohols (for example, mono stearyl glyceryl ether (batyl alcohol), 2-decyltetradecynol, lanolin alcohol, cholesterol, phytosterol, hexyl dodecanol, isostearyl alcohol, and octyl dodecanol), and combination thereof.

In some embodiments, the fatty phase comprises liquid oils/fats. In some embodiments, the liquid oils/fats are selected from the group consisting of avocado oil, tsubaki oil, turtle oil, macademia nut oil, corn oil, mink oil, olive oil, rape seed oil, egg yolk oil, sesame seed oil, persic oil, wheat germ oil, sasanqua oil, castor oil, linseed oil, safflower oil, cotton seed oil, perilla oil, soybean oil, peanut oil, tea seed oil, kaya oil, rice bran oil, chinese wood oil, Japanese wood oil, jojoba oil, germ oil, triglycerol, glyceryl trioctanoate and glyceryl triisopalmitate, and combination thereof.

In some embodiments, the fatty phase comprises solid fats/oils. In some embodiments, the solid oils/fats are selected from the group consisting of cacao butter, coconut oil, horse tallow, hardened coconut oil, palm oil, beef tallow, sheep tallow, hardened beef tallow, palm kernel oil, pork tallow, beef bone tallow, Japanese core wax, hardened oil, neatsfoot tallow, Japanese wax and hydrogenated castor oil, and combination thereof.

In some embodiments, the fatty phase comprises vegetable oils. In some embodiments, the vegetable oils are selected from the group consisting of buriti oil, soybean oil, olive oil, tea tree oil, rosemary oil, jojoba oil, coconut oil, sesame seed oil, sesame oil, palm oil, avocado oil, babassu oil, rice oil, almond oil, argon oil, sunflower oil, and combination thereof. In some embodiments, the vegetable oil is selected from the group consisting of coconut oil, sunflower oil and sesame oil. In some embodiments, the oily component is selected from cocoa butter, palm stearin, sunflower oil, soybean oil and coconut oil.

In some embodiments, the oil phase for the collagen stimulating compositions and methods of making and using thereof comprises lipid material. In some embodiments, the lipid materials are selected from the group consisting of ceramides, phospholipids (e.g., soy lecithin, egg lecithin), glycolipids, and combination thereof.

In some embodiments, the oil phase for the collagen stimulating compositions and methods of making and using thereof comprises hydrocarbon oil. As used herein, the hydrocarbon oils have average carbon chain length less than 20 carbon atoms. Suitable hydrocarbon oils include cyclic hydrocarbons, straight chain aliphatic hydrocarbons (saturated or unsaturated), and branched chain aliphatic hydrocarbons (saturated or unsaturated). Straight chain hydrocarbon oils will typically contain from about 6 to about 16 carbon atoms, preferably from about 8 up to about 14 carbon atoms. Branched chain hydrocarbon oils can and typically may contain higher numbers of carbon atoms, e.g. from about 6 up to about 20 carbon atoms, preferably from about 8 up to about 18 carbon atoms. Suitable hydrocarbon oils of the invention will generally have a viscosity at ambient temperature (25 to 30° C.) of from 0.0001 to 0.5 Pa·s, preferably from 0.001 to 0.05 Pa·s, more preferably from 0.001 to 0.02 Pa·s.

In some embodiments, the hydrogen carbon oils are selected from the group consisting of liquid petrolatum, squalane, pristane, paraffin, isoparaffin, ceresin, squalene, mineral oil, light mineral oil, blend of light mineral oil and heavy mineral oil, polyisobutene, hydrogenated polyisobutene, terpene oil and combination thereof.

In some embodiments, the hydrogen carbon oils light mineral oil. As used herein, mineral oils are clear oily liquids obtained from petroleum oil, from which waxes have been removed, and the more volatile fractions removed by distillation. The fraction distilling between 250° C. to 300° C. is termed mineral oil, and it consists of a mixture of hydrocarbons, in which the number of carbon atoms per hydrocarbon molecule generally ranges from C10 to C40. Mineral oil may be characterized in terms of its viscosity, where light mineral oil is relatively less viscous than heavy mineral oil, and these terms are defined more specifically in the U.S. Pharmacopoeia, 22nd revision, p. 899 (1990). A commercially available example of a suitable light mineral oil for use in the invention is Sirius® M40 (carbon chain length C0-C28 mainly C12-C20, viscosity 4.3×10 Pa·s), available from Silkolene. Other hydrocarbon oils that may be used in the invention include relatively lower molecular weight hydrocarbons including linear saturated hydrocarbons such a tetradecane, hexadecane, and octadecane, cyclic hydrocarbons such as dioctylcyclohexane (e.g. CETIOL® S from Henkel), branched chain hydrocarbons (e.g. ISOPAR® and ISOPAR® V from Exxon Corp.).

In some embodiments, the fatty material for the oil phase is selected from the group consisting of neopentyl glycol diheptanoate, propylene glycol dicaprylate, dioctyl adipate, coco-caprylate/caprate, diethylhexyl adipate, diisopropyl dimer dilinoleate, diisostearyl dimer dilinoleate, Butyrospermum parkii (shea) butter, C12-C13 alkyl lactate, di-C12-C13 alkyl tartrate, tri-C12-C13 alkyl citrate, C12-C15 alkyl lactate, ppg dioctanoate, diethylene glycol dioctanoate, meadow foam oil, C12-15 alkyl oleate, tridecyl neopentanoate, cetearyl alcohol and polysorbate 60, C18-C26 triglycerides, cetearyl alcohol & cetearyl glucoside, acetylated lanolin, vp/eicosene copolymer, glyceryl hydroxystearate, C18-36 acid glycol ester, C18-36 triglycerides, glyceryl hydroxystearate and mixtures thereof, also suitable and preferred are cetyl alcohol & glyceryl stearate & PEG-75, stearate & ceteth-20 & steareth-20, lauryl glucoside & polyglyceryl-2 dipolyhydroxystearate, beheneth-25, polyamide-3 & pentaerythrityl tetra-di-t-butyl hydroxycinnamate, polyamide-4 and PEG-100 stearate, potassium cethylphosphate, stearic acid and hectorites.

In some embodiments, the fatty material for the oil phase is selected from the group consisting of liquid paraffin, liquid isoparaffin, neopentylglycol dicaprate, isopropyl isostearate, cetyl 2-ethylhesanoate, isononyl isononanoate, glyceryl tri(caprylatelcaprate), alky-1,3-dimethylbutyl ether, methyl polysiloxane having a molecular weight ranging from 100 to 500, decamethylcydopentasiloxane, octamethylcydotetrasiloxane, higher fatty acids having a carbon number ranging from 12 to 22, higher alcohols having a carbon number ranging from 12 to 22, ceramides, glycolipids, and terpene oil.

In some embodiments, the fatty material for the oil phase is selected from the group consisting of paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid, silicone oils and combination thereof.

In an embodiment, the fatty material for the oil phase is selected from the group consisting of vegetable oils including jojoba oil, olive oil, camella oil, avocado oil, cacao oil, sunflower oil, persic oil, palm oil, castor oil, buriti oil, medium chain triglycerides.

In an embodiment, the oily materials emulsifyable by the silk emulsifier is selected from the group consisting of a vegetable oil, isododecane, and isohexadecane, and one or more oily esters of fatty acids, wherein the vegetable oil is selected from jojoba oils and/or camellia oils, wherein said oily esters are selected from isononyl isononanoate and coco caprylate.

In some embodiments, the oil phase is present in the collagen stimulating compositions and methods of making and using thereof at a weight percent ranging from 1.0 wt. % to about 95 wt. % by the total weight of the collagen boosting composition. In some embodiments, the oil phase is present in the collagen boosting composition at a weight percent ranging from 45.0 wt. % to about 95 wt. % by the total weight of the collagen boosting composition. In some embodiments, the oil phase is present in the collagen boosting composition at a weight percent ranging from 45.0 wt. % to about 65.0 wt. % by the total weight of the collagen boosting composition. In some embodiments, the oil phase is present in the collagen boosting composition at a weight percent ranging from 5.0 wt. % to about 45 wt. % by the total weight of the collagen boosting composition. In some embodiments, the oil phase is present in the collagen boosting composition at a weight percent ranging from 5.0 wt. % to about 35 wt. % by the total weight of the collagen boosting composition. In some embodiments, the oil phase is present in the collagen boosting composition at a weight percent ranging from 10.0 wt. % to about 25 wt. % by the total weight of the collagen boosting composition.

In some embodiments, the oil phase is presented in the collagen stimulating compositions and methods of making and using thereof in a weight percent ranging from about 50.0 wt. % to 95.0 weight % by the total weight of the emulsion carrier. In some embodiments, the oil phase is presented in the collagen boosting composition in a weight percent ranging from about 5 wt. % to 45 weight % by the total weight of the emulsion carrier, because such a content allows the emulsion carrier to have a stability over a wider temperature range around the room temperatures and a good feeling.

In some embodiments, the aqueous phase for the emulsion carrier comprises water, an aqueous solution, a blend of alcohol and water, or a lyotropic liquid crystalline phase as aqueous carrier. Selection of the water contained in the collagen stimulating compositions and methods of making and using thereof of the present invention is not limited in particular; specific examples include purified water, ion-exchanged water, and tap water. In some embodiments, the aqueous further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous phase further comprise one or more low alcohol solvent including methanol, ethanol, and isopropanol.

The blend ratio of water and polyhydric alcohol is determined appropriately based on emulsion formulation types.

In some embodiments, the emulsion comprises from about 50 wt. % to about 98 wt. % of the aqueous phase by the total weight of the composition. In some embodiments, the emulsion comprises from about 60 wt. % to about 90 wt. % of the aqueous phase by the total weight of the composition. In some embodiments, the amount of the aqueous phase in the emulsion is selected from: about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. 00 about 97.0 wt. %, about 98.0 wt. %, by the total weight of the composition.

In some embodiments, the silk containing emulsifier system is present in the aqueous phase.

In some embodiments, the collagen stimulating compositions and methods of making and using thereof comprise viscosity modifiers and/or thickeners. In some embodiments, the thickener is selected from the group consisting of ethylene glycol monostearate, carbomer polymers, carboxyvinyl polymer, acrylic copolymers, methyl cellulose, copolymers of lactide and glycolide monomers, ethyl cellulose, hydroxyethyl cellulose, hydroxypropyl cellulose, carrageenan, hydrophobically modified hydroxy-ethyl-cellulose, laponite and water soluble salts of cellulose ethers such as sodium carboxymethylcellulose and sodium carboxymethyl hydroxyethyl cellulose, natural gums such as gum karaya, gum arabic, Guars, HP Guars, heteropolysaccharide gums (e.g., xanthan gum), and gum tragacanth.

In some embodiments, the thickener is selected from the group consisting of talc, fumed silica, polymeric polyether compound (e.g., polyethylene or polypropylene oxide (MW 300 to 1,000,000), capped with alkyl or acyl groups containing 1 to about 18 carbon atoms), ethylene glycol stearate, alkanolamides of fatty acids having from 16 to 22 carbon atoms, polyethylene glycol 3 distearate, polyacrylic acids (e.g., Carbopol® 420, Carbopol® 488 or Carbopol® 493), cross-linked polymers of acrylic acid, copolymers of acrylic acid with a hydrophobic monomer, copolymers of carboxylic acid-containing monomers and acrylic esters (e.g. Carbopol® 1342), cross-linked copolymers of acrylic acid and acrylate esters, polyacrylic acids cross-linked with polyfunctional agent (e.g., Carbopol® 910, Carbopol® 934, Carbopol® 940, Carbopol® 941 and Carbopol® 980, Ultrez® 10), and crystalline long chain acyl derivatives.

In some embodiments, the collagen stimulating compositions and methods of making and using thereof comprise from about 0.1 wt. % to about 15.0 wt. % of thickener/viscosity modifying agent by the total weight of the composition. In some embodiments, the collagen stimulating compositions and methods of making and using thereof comprise from about 0.1 wt. % to about 10.0 wt. % of thickener/viscosity modifying agent by the total weight of the composition. In some embodiments, the collagen stimulating compositions and methods of making and using thereof comprise from about 0.5 wt. % to about 6.0 wt. % of thickener/viscosity modifying agent by the total weight of the composition. In some embodiments, the collagen stimulating compositions and methods of making and using thereof comprise from about 0.9 wt. % to about 4.0 wt. % of thickener/viscosity modifying agent by the total weight of the composition. In some embodiments, the collagen stimulating compositions and methods of making and using thereof comprise about 2.0 wt. % of thickener/viscosity modifying agent by the total weight of the composition. In some embodiments, the amount of the thickener/viscosity modifying agent presented in the collagen stimulating compositions and methods of making and using thereof is selected from the group consisting of about 0.1 wt; about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.25 wt. %, about 1.50 wt. %, about 1.75 wt. %, about 2.0 wt. %, about 2.25 wt. %, about 2.5 wt. %, about 2.75 wt. %, about 3.0 wt. %, about 3.25 wt. %, about 3.5 wt. %, about 3.75 wt. %, about 4.0 wt. %, about 4.25 wt. %, about 4.5 wt. %, about 4.75 wt. %, about 5.0 wt. %, about 5.25 wt. %, about 5.5 wt. %, about 5.75 wt. %, about 6.0 wt. %, about 6.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.1 wt. %, about 10.2 wt. %, about 10.3 wt. %, about 10.4 wt. %, about 10.5 wt. %, about 10.6 wt. %, about 10.7 wt. %, about 10.8 wt. %, about 10.9 wt. %, about 11.0 wt. %, about 11.1 wt. %, about 11.2 wt. %, about 11.3 wt. %, about 11.4 wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4 wt. %, about 12.5 wt. %, about 12.6 wt. %, about 12.7 wt. %, about 12.8 wt. %, about 12.9 wt. %, about 13.0 wt. %, about 13.1 wt. %, about 13.2 wt. %, about 13.3 wt. %, about 13.4 wt. %, about 13.5 wt. %, about 13.6 wt. %, about 13.7 wt. %, about 13.8 wt. %, about 13.9 wt. %, about 14.0 wt. %, about 14.1 wt. %, about 14.2 wt. %, about 14.3 wt. %, about 14.4 wt. %, about 14.5 wt. %, about 14.6 wt. %, about 14.7 wt. %, about 14.8 wt. %, about 14.9 wt. %, about 15.0 wt. %, by the total weight of the composition.

In some embodiments, the collagen stimulating compositions and methods of making and using thereof comprise water, an aqueous solution, an alcohol, a blend of alcohol and water, or a lyotropic liquid crystalline phase as aqueous carrier. Selection of the water contained in the composition is not limited in particular; specific examples include purified water, ion-exchanged water, and tap water. In some embodiments, the collagen stimulating compositions and methods of making and using thereof further comprise one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the collagen stimulating compositions and methods of making and using thereof further comprise one or more low alcohol solvent including methanol, ethanol, and isopropanol.

In some embodiments, the collagen stimulating compositions and methods of making and using thereof comprise from about 50 wt. % to about 98 wt. % of the aqueous carrier by the total weight of the composition. In some embodiments, the collagen stimulating compositions and methods of making and using thereof comprise from about 60 wt. % to about 90 wt. % of the aqueous carrier by the total weight of the composition. In some embodiments, the amount of the aqueous carrier in the collagen stimulating compositions and methods of making and using thereof is selected from: about 50.0 wt. %, about 51.0 wt. %, about 52.0 wt. %, about 53.0 wt; about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the composition.

In some embodiments, the collagen stimulating compositions and methods of making and using thereof comprise a non-aqueous liquid carrier. Non-aqueous liquid carrier as used herein means that the liquid carrier is substantially free of water. In the present invention, “the liquid carrier being substantially free of water” means that: the liquid carrier is free of water; or, if the liquid carrier contains water, the level of water is very low. In the present invention, the level of water, if included, 1% or less, preferably 0.5% or less, more preferably 0.3% or less, still more preferably 0.1% or less, even more preferably 0% by weight of the composition.

In some embodiments, the non-aqueous liquid carrier comprises an oily material selected from the group consisting of mineral oil, hydrocarbon oils, hydrogenated polydecene, polyisobutene, isoparaffin, isododecane, isohexadecane, volatile silicone oil, non-volatile silicone oil, isohexadecane, squalene, squalene, ester oil and combination thereof. In some embodiments, the non-aqueous liquid carrier comprises an oily material selected from the group consisting of white mineral oils, squalane, hydrogenated polyisobutene, isohexadecane, and isododecane. In some embodiments, the non-aqueous liquid carrier comprises squalane and hydrogenated polyisobutene. In some embodiments, the non-aqueous liquid carrier comprises white mineral oils, isohexadecane, and isododecane. In some embodiments, the hydrocarbon oil is selected from the group consisting of liquid paraffin, liquid isoparaffin, squalene, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, permethyl-substituted isomers, e.g., the permethyl-substituted isomers of hexadecane and eicosane (e.g., 2,2,4,4,6,6,8,8-dimethyl-10-methylundecane and 2,2,4,4,6,6-dimethyl-8-methylnonane), copolymer of isobutylene and butane, poly-α-olefins (e.g., polymer of ethylene, propylene, 1-butene, 1-pentene, 1-hexene, 1-octene, 1-decene, 1-dodecene, 1-tetradecene), and combination thereof.

In some embodiments, the collagen stimulating compositions and methods of making and using thereof comprise an organic oil comprising a fatty ester oil selected from the group consisting of isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, oleyl adipate, isopropyl myristate, glycol stearate, and isopropyl laurate, isocetyl stearoyl stearate, diisopropyl adipate, tristearyl citrate, triolein, tristearin glyceryl dilaurate, C₈-C₁₀ triester of trimethylolpropane, tetraester of 3,3 diethanol-1,5 pentadiol, C₈-C₁₀ diester of adipic acid, ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, sorbitan fatty acid esters, and polyoxyethylene sorbitan fatty acid esters (e.g. polyoxyethylene (20) sorbitan monooleate, polysorbate 80, Tween 80@), and combination thereof.

In some embodiments, the non-aqueous liquid carrier comprises a volatile isoparaffin having from about 8 to about 20 carbon atoms. In some embodiments, the non-aqueous liquid carrier comprises a volatile isoparaffin having from about 8 to about 16 carbon atoms. In some embodiments, the non-aqueous liquid carrier comprises a volatile isoparaffin having from about 10 to about 16 carbon atoms. In some embodiments, the volatile isoparaffin is selected from the group consisting of trimer, tetramer, and pentamer of isobutene, and mixtures thereof. Commercially available isoparaffin hydrocarbons may have distributions of its polymerization degree, and may be mixtures of, for example, trimer, tetramer, and pentamer. What is meant by tetramer herein is that a commercially available isoparaffin hydrocarbons in which tetramer has the highest content, i.e., tetramer is included at a level of preferably 70% or more, more preferably 80% or more, still more preferably 85% or more.

In some embodiments, the volatile isoparaffin is a mixture of several grades of isoparaffins. In some embodiments, the volatile isoparaffin has a viscosity range selected from: about 0.5 mm²·s⁻¹ to about 50 mm²·s⁻¹, about 0.8 mm²·s⁻¹ to about 40 mm²·s⁻¹, about 1 mm²·s¹ to about 30 mm²·s⁻¹, about 1 mm²·s⁻¹ to about 20 mm²·s⁻¹, and about 1 mm²·s⁻¹ to about 10 mm² s⁻¹, at 37.8° C. When using two or more isoparaffin hydrocarbon solvents, it is preferred that the mixture of isoparaffin hydrocarbon solvents have the above viscosity.

In some embodiments, the non-aqueous liquid carrier comprises ester oil. In some embodiments, the ester oils have an HLB of 3 or less, and as liquid at room temperature. In some embodiments, the ester oil is selected from the group consisting of methyl palmitate, methyl stearate, methyl oleate, methyl linoleate, and methyl laurate. In an embodiment, the ester oil methyl stearate.

In some embodiments, the ester oil is included in the non-aqueous liquid carrier at a weight percent selected from: about 0.1 wt. % to about 25 wt. %, about 0.5 wt. % to about 15 wt. %, about 1.0 wt. % to about 10 wt. %, about 1.0 wt. % to about 5.0 wt. % by the total weight of the collagen boosting composition, in view of the balance between conditioned feel and product stability, and/or in view of prevent foaming.

In some embodiments, the non-aqueous liquid carrier comprises fatty esters selected from the group consisting of trimethyloylpropane tricaprylate/tricaprylate, C12-C15 alkyl benzoate, ethylhexyl stearate, ethylhexyl cocoate, decyl oleate, decyl cocoate, ethyl oleate, isopropyl myristate, ethylhexyl perlagonate, pentaerythrityl tetracaprylate/tetracaprate, PPG-3 benzyl ether myristate, propyiene glycol dicaprylate/dicaprate, ethylhexyl isostearate, ethylhexyl palmitate and natural oils such as Glycine soja, Helianthus annuus, Simmondsia chinensis, Carthamus tinctorius, Oenothera biennis and rapae oleum, and combination thereof.

In some embodiments, the non-aqueous liquid carrier comprises glyceride fatty ester. In some embodiments, the suitable glyceride fatty esters for use in hair oils of the invention have a viscosity at ambient temperature (25 to 30° C.) of from 0.01 to 0.8 Pa·s, preferably from 0.015 to 0.6 Pa·s, more preferably from 0.02 to 0.065 Pa·s.

In an embodiment, the fatty material comprises a glyceride fatty ester. As used herein, the term “glyceride fatty esters” refers to the mono-, di-, and tri-esters formed between glycerol and long chain carboxylic acids such as C6-C30 carboxylic acids. The carboxylic acids may be saturated or unsaturated or contain hydrophilic groups such as hydroxyl. Preferred glyceride fatty esters are derived from carboxylic acids of carbon chain length ranging from C10 to C24, preferably C10 to C22, most preferably C12 to C 20, most preferably C12 to C18. In some embodiments, glyceride fatty ester is a medium-chain triglyceride having C6-C12 fatty acid chain.

In some embodiments, glyceride fatty ester is sourced from varieties of vegetable and animal fats and oils, such as camellia oil, coconut oil, castor oil, safflower oil, sunflower oil, peanut oil, cottonseed oil, corn oil, olive oil, cod liver oil, almond oil, avocado oil, palm oil, sesame oil, lanolin and soybean oil. Synthetic oils include trimyristin, triolein and tristearin glyceryl dilaurate. Vegetable derived glyceride fatty esters include almond oil, castor oil, coconut oil, palm kernel oil, sesame oil, sunflower oil and soybean oil.

In some embodiments, the glyceride fatty ester is selected from coconut oil, sunflower oil, almond oil and mixtures thereof.

The non-aqueous liquid carrier is included at a level by weight of the collagen boosting composition of, from about 50% to about 99.9%, from about 60% to about 99.8%, more preferably from about 65% to about 98% by the total weight of the collagen boosting composition.

In some embodiments, the collagen stimulating compositions and methods of making and using thereof comprise an aqueous liquid carrier substantially free of non-silk surfactant. In some embodiments, the aqueous liquid carrier is selected from water, an aqueous solution, an alcohol, a blend of alcohol and water, or a lyotropic liquid crystalline phase. Selection of the water contained in the composition is not limited in particular; specific examples include purified water, ion-exchanged water, and tap water.

In some embodiments, the aqueous liquid carrier comprises one or more small molecule polyhydric alcohols selected from the group consisting of ethanediol, propanediol, glycerol, butanediol, butantetraol, xylitol, sorbitol, inositol, ethylene glycol, polyethylene glycol. In some embodiments, the aqueous liquid carrier comprises water and glycerol. In some embodiments, the aqueous liquid carrier comprises water and glycerol in a weight ratio of water to glycerol at 1:10. In some embodiments, the aqueous liquid carrier comprises water and glycerol in a weight ratio of water to glycerol selected from 1:10, 1:9, 1:8, 1:7, 1:6, 1:5, 1:4, 1:3, 1:2, and 1:1. In some embodiments, the aqueous liquid carrier comprises water and glycerol in a weight ratio of water to glycerol at 1:1. In some embodiments, the aqueous liquid carrier comprises water and glycerol in a weight ratio of water to glycerol at 1:10. In some embodiments, the aqueous liquid carrier comprises silk fibroin protein fragments and glycerol in a weight ratio of silk fibroin protein fragments to glycerol selected from 1:10, 1:9, 1:8, 1:7, 1:6, 1:5, 1:4, 1:3, 1:2, and 1:1. In some embodiments, the aqueous liquid carrier comprises silk fibroin protein fragments and glycerol in a weight ratio of silk fibroin protein fragments to glycerol at 1:1.

In some embodiments, the pH of the aqueous liquid phase is adjusted ranging from about 4.0 to about 9.0. In some embodiments, the pH of the aqueous liquid phase is adjusted ranging from about 4.5 to about 8.5. In some embodiments, the pH of the aqueous liquid phase is adjusted ranging from about 5.0 to about 7.0. The pH adjusting agent may include a buffer (e.g. PBS buffer), alkali metal salt, acid, citric acid, succinic acid, phosphoric acid, sodium hydroxide, ammonium hydroxide, ethanolamine, sodium carbonate, and combination thereof.

In some embodiments, the composition comprises from about 1.0 wt. % to about 99.0 wt. % of the aqueous liquid carrier by the total weight of the composition. In some embodiments, the composition comprises from about 5.0 wt. % to about 45.0 wt. % of the aqueous liquid carrier by the total weight of the composition. In some embodiments, the composition comprises from about 5.0 wt. % to about 35.0 wt. % of the aqueous liquid carrier by the total weight of the composition. In some embodiments, the composition comprises from about 10.0 wt. % to about 30.0 wt. % of the aqueous liquid carrier by the total weight of the composition. In some embodiments, the composition comprises from about 45.0 wt. % to about 95.0 wt. % of the aqueous liquid carrier by the total weight of the composition. In some embodiments, the composition comprises from about 60.0 wt. % to about 90.0 wt. % of the aqueous liquid carrier by the total weight of the composition. In some embodiments, the composition comprises from about 45.0 wt. % to about 75.0 wt. % of the aqueous liquid carrier by the total weight of the composition. In some embodiments, the composition comprises from about 60.0 wt. % to about 75.0 wt. % of the aqueous liquid carrier by the total weight of the composition. In some embodiments, the amount of the aqueous liquid carrier in the composition is selected from: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, about 33.0 wt. %, about 34.0 wt. %, about 35.0 wt. %, about 36.0 wt. %, about 37.0 wt. %, about 38.0 wt. %, about 39.0 wt. %, about 40.0 wt. %, about 41.0 wt. %, about 42.0 wt. %, about 43.0 wt. %, about 44.0 wt. %, about 45.0 wt. %, about 46.0 wt. %, about 47.0 wt. %, about 48.0 wt. %, about 49.0 wt. %, about 50.0 wt. %, about 51.0 wt. %, about 52.0 wt. %, about 53.0 wt. %, about 54.0 wt. %, about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, about 80.0 wt. %, about 81.0 wt. %, about 82.0 wt. %, about 83.0 wt. %, about 84.0 wt. %, about 85.0 wt. %, about 86.0 wt. %, about 87.0 wt. %, about 88.0 wt. %, about 89.0 wt. %, about 90.0 wt. %, about 91.0 wt. %, about 92.0 wt. %, about 93.0 wt. %, about 94.0 wt. %, about 95.0 wt. %, about 96.0 wt. %, about 97.0 wt. %, about 98.0 wt. %, by the total weight of the composition.

In some embodiments, the collagen stimulating compositions and methods of making and using thereof optionally comprise a natural or synthetic fragrant essential oil. In some embodiments, the fragrant essential oil is selected from the group consisting of eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil, almond oil, argan oil, avocado oil, cedar oil, wheat germ oil, bergamot oil, and combination thereof.

In some embodiments, the collagen stimulating compositions and methods of making and using thereof optionally comprise vitamins selected from the group selected from the group consisting of vitamin A, vitamin B, vitamin E, vitamin D, vitamin K, riboflavin, pyridoxin, coenzyme thiamine pyrophosphate, flavin adenine dinucleotide, folic acid, pyridoxal phosphate, tetradrofolic acid, and combination thereof.

In some embodiments, the collagen stimulating compositions and methods of making and using thereof contains vitamin and/or coenzymes at about 0.01 wt. % to about 8.0 wt. % by the total weight of the composition. In some embodiments, the composition contains vitamin and/or coenzymes at about 0.001 wt. % to about 10.0 wt. % by the total weight of the composition. In some embodiments, the composition contains vitamin and/or coenzymes at about 0.05 wt. % to about 5.0 wt. % by the total weight of the composition.

In some embodiments, the collagen stimulating compositions and methods of making and using thereof optionally comprise a preservative selected from the group consisting of triazoles, imidazoles, naphthalene derivatives, benzimidazoles, morphline derivatives, dithiocarbamates, benzisothiazoles, benzamides, boron compounds, formaldehyde donors, isothiazolones, thiocyanates, quaternary ammonium compounds, iodine derivates, phenol derivatives, micobicides, pyridines, dialkylthiocarbamates, nitriles, parabens, alkyl parabens, and salts thereof.

In some embodiments, the collagen stimulating compositions and methods of making and using thereof is formulated in a form selected from the group consisting of aqueous solution, ethanolic solution, oil, gel, emulsion, suspension, mousses, liquid crystal, solid, gels, lotions, creams, aerosol sprays, paste, foam and tonics. In some embodiments, the composition is in a form selected from the group consisting of a cream, spray, aerosol, mousse, or gel.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion compositions described herein, in controlled amounts, either with or without another active pharmaceutical ingredient. The construction and use of transdermal patches for the delivery of pharmaceutical agents is well known in the art. See, e.g., U.S. Pat. Nos. 5,023,252; 4,992,445 and 5,001,139, each of which is incorporated herein by reference in its entirety. Such patches may be constructed for continuous, pulsatile, or on demand delivery of pharmaceutical agents.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation, intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compositions disclosed herein may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the composition may be located in the body of the stent or graft, for example in microchannels or micropores. When implanted, the composition diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Composition disclosed herein may be administered intravascularly from a balloon used during angioplasty. Extravascular administration via the pericard or via advential application of compositions of the disclosure may also be performed to decrease restenosis.

Exemplary parenteral administration forms include solutions or suspensions in sterile aqueous solutions, for example, aqueous propylene glycol or dextrose solutions. Such dosage forms can be suitably buffered, if desired.

The disclosure also provides kits. The kits include a composition disclosed herein in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer. The kits are preferably for use in the treatment of the diseases and conditions described herein.

The following clauses describe certain embodiments.

Clause 1. A method of treatment or prevention of a disorder, disease, or condition alleviated by i) stimulating or modulating collagen expression in a subject in need thereof, and/or ii) stimulating or modulating claudin-1 expression in a subject in need thereof; and/or iii) stimulating or modulating one more anti-inflammatory genes in a subject in need thereof, the method comprising administering to the subject a composition comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 14 kDa and about 30 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5, wherein the concentration of silk fibroin fragments in the composition is from about 0.001% w/v to about 10% w/v.

Clause 2. The method of clause 1, wherein the composition further comprises 0 to 500 ppm lithium bromide.

Clause 3. The method of clause 1 or clause 2, wherein the composition further comprises 0 to 500 ppm sodium carbonate Clause 4. The method of any one of clauses 1 to 3, wherein the silk fibroin fragments have a polydispersity between 1 and about 1.5.

Clause 5. The method of any one of clauses 1 to 3, wherein the silk fibroin fragments have a polydispersity between about 1.5 and about 2.0.

Clause 6. The method of any one of clauses 1 to 3, wherein the silk fibroin fragments have a polydispersity between about 1.5 and about 3.0.

Clause 7. The method of any one of clauses 1 to 3, wherein the silk fibroin fragments have a polydispersity between about 2.0 and about 2.5.

Clause 8. The method of any one of clauses 1 to 3, wherein the silk fibroin fragments have a polydispersity between about 2.5 and about 3.0.

Clause 9. The method of any one of clauses 1 to 8, wherein the silk fibroin fragments do not spontaneously or gradually gelate and do not visibly change in color or turbidity when in an aqueous solution for at least 10 days prior to formulation into the composition.

Clause 10a. The method of any one of clauses 1 to 9, wherein the silk fibroin fragments are present in the composition at about 0.001% w/v to about 1% w/v. Clause 10b. The method of any one of clauses 1 to 9, wherein the silk fibroin fragments are present in the composition at about 0.001% w/v to about 2% w/v. Clause 10c. The method of any one of clauses 1 to 9, wherein the silk fibroin fragments are present in the composition at about 0.001% w/v to about 3% w/v. Clause 10d. The method of any one of clauses 1 to 9, wherein the silk fibroin fragments are present in the composition at about 0.001% w/v to about 4% w/v. Clause 10e. The method of any one of clauses 1 to 9, wherein the silk fibroin fragments are present in the composition at about 0.001% w/v to about 5% w/v. Clause 10f. The method of any one of clauses 1 to 9, wherein the silk fibroin fragments are present in the composition at about 0.001% w/v to about 6% w/v. Clause 10g. The method of any one of clauses 1 to 9, wherein the silk fibroin fragments are present in the composition at about 0.001% w/v to about 7% w/v. Clause 10 h. The method of any one of clauses 1 to 9, wherein the silk fibroin fragments are present in the composition at about 0.001% w/v to about 8% w/v. Clause 10i. The method of any one of clauses 1 to 9, wherein the silk fibroin fragments are present in the composition at about 0.001% w/v to about 9% w/v.

Clause 11a. The method of any one of clauses 1 to 9, wherein the silk fibroin fragments are present in the composition at about 0.01% w/v to about 1% w/v. Clause 11b. The method of any one of clauses 1 to 9, wherein the silk fibroin fragments are present in the composition at about 0.002% w/v to about 1% w/v. Clause 11c. The method of any one of clauses 1 to 9, wherein the silk fibroin fragments are present in the composition at about 0.003% w/v to about 1% w/v. Clause 11d. The method of any one of clauses 1 to 9, wherein the silk fibroin fragments are present in the composition at about 0.004% w/v to about 1% w/v. Clause 11e. The method of any one of clauses 1 to 9, wherein the silk fibroin fragments are present in the composition at about 0.005% w/v to about 1% w/v. Clause 11f. The method of any one of clauses 1 to 9, wherein the silk fibroin fragments are present in the composition at about 0.006% w/v to about 1% w/v. Clause 11g. The method of any one of clauses 1 to 9, wherein the silk fibroin fragments are present in the composition at about 0.007% w/v to about 1% w/v. Clause 11h. The method of any one of clauses 1 to 9, wherein the silk fibroin fragments are present in the composition at about 0.008% w/v to about 1% w/v. Clause 11i. The method of any one of clauses 1 to 9, wherein the silk fibroin fragments are present in the composition at about 0.009% w/v to about 1% w/v.

Clause 12a. The method of any one of clauses 1 to 9, wherein the silk fibroin fragments are present in the composition at about 0.025% w/v to about 1% w/v. Clause 12b. The method of any one of clauses 1 to 9, wherein the silk fibroin fragments are present in the composition at about 0.01% w/v to about 1% w/v. Clause 12c. The method of any one of clauses 1 to 9, wherein the silk fibroin fragments are present in the composition at about 0.02% w/v to about 1% w/v. Clause 12d. The method of any one of clauses 1 to 9, wherein the silk fibroin fragments are present in the composition at about 0.03% w/v to about 1% w/v. Clause 12e. The method of any one of clauses 1 to 9, wherein the silk fibroin fragments are present in the composition at about 0.04% w/v to about 1% w/v. Clause 12f. The method of any one of clauses 1 to 9, wherein the silk fibroin fragments are present in the composition at about 0.05% w/v to about 1% w/v. Clause 12g. The method of any one of clauses 1 to 9, wherein the silk fibroin fragments are present in the composition at about 0.06% w/v to about 1% w/v. Clause 12h. The method of any one of clauses 1 to 9, wherein the silk fibroin fragments are present in the composition at about 0.07% w/v to about 1% w/v. Clause 12i. The method of any one of clauses 1 to 9, wherein the silk fibroin fragments are present in the composition at about 0.08% w/v to about 1% w/v. Clause 12j. The method of any one of clauses 1 to 9, wherein the silk fibroin fragments are present in the composition at about 0.09% w/v to about 1% w/v.

Clause 13a. The method of any one of clauses 1 to 9, wherein the silk fibroin fragments are present in the composition at about 0.05% w/v to about 0.7% w/v. Clause 13b. The method of any one of clauses 1 to 9, wherein the silk fibroin fragments are present in the composition at about 0.01% w/v to about 0.7% w/v. Clause 13c. The method of any one of clauses 1 to 9, wherein the silk fibroin fragments are present in the composition at about 0.02% w/v to about 0.7% w/v. Clause 13d. The method of any one of clauses 1 to 9, wherein the silk fibroin fragments are present in the composition at about 0.03% w/v to about 0.7% w/v. Clause 13e. The method of any one of clauses 1 to 9, wherein the silk fibroin fragments are present in the composition at about 0.04% w/v to about 0.7% w/v. Clause 13f. The method of any one of clauses 1 to 9, wherein the silk fibroin fragments are present in the composition at about 0.06% w/v to about 0.7% w/v. Clause 13g. The method of any one of clauses 1 to 9, wherein the silk fibroin fragments are present in the composition at about 0.07% w/v to about 0.7% w/v. Clause 13h. The method of any one of clauses 1 to 9, wherein the silk fibroin fragments are present in the composition at about 0.08% w/v to about 0.7% w/v. Clause 13i. The method of any one of clauses 1 to 9, wherein the silk fibroin fragments are present in the composition at about 0.09% w/v to about 0.7% w/v. Clause 13j. The method of any one of clauses 1 to 9, wherein the silk fibroin fragments are present in the composition at about 0.1% w/v to about 0.7% w/v.

Clause 13k. The method of any one of clauses 1 to 9, wherein the silk fibroin fragments are present in the composition at about 0.1% w/v. Clause 13l. The method of any one of clauses 1 to 9, wherein the silk fibroin fragments are present in the composition at about 0.15% w/v. Clause 13m. The method of any one of clauses 1 to 9, wherein the silk fibroin fragments are present in the composition at about 0.2% w/v. Clause 13n. The method of any one of clauses 1 to 9, wherein the silk fibroin fragments are present in the composition at about 0.25% w/v. Clause 13o. The method of any one of clauses 1 to 9, wherein the silk fibroin fragments are present in the composition at about 0.3% w/v. Clause 13p. The method of any one of clauses 1 to 9, wherein the silk fibroin fragments are present in the composition at about 0.35% w/v. Clause 13q. The method of any one of clauses 1 to 9, wherein the silk fibroin fragments are present in the composition at about 0.4% w/v. Clause 13r. The method of any one of clauses 1 to 9, wherein the silk fibroin fragments are present in the composition at about 0.45% w/v. Clause 13s. The method of any one of clauses 1 to 9, wherein the silk fibroin fragments are present in the composition at about 0.5% w/v. Clause 13t. The method of any one of clauses 1 to 9, wherein the silk fibroin fragments are present in the composition at about 0.55% w/v. Clause 13u. The method of any one of clauses 1 to 9, wherein the silk fibroin fragments are present in the composition at about 0.6% w/v. Clause 13v. The method of any one of clauses 1 to 9, wherein the silk fibroin fragments are present in the composition at about 0.65% w/v. Clause 13x. The method of any one of clauses 1 to 9, wherein the silk fibroin fragments are present in the composition at about 0.7 or 0.75% w/v. Clause 13y. The method of any one of clauses 1 to 9, wherein the silk fibroin fragments are present in the composition at about 0.8 or 0.85% w/v. Clause 13z. The method of any one of clauses 1 to 9, wherein the silk fibroin fragments are present in the composition at about 0.9 or 0.95% w/v.

Clause 14a. The method of any one of clauses 1 to 13, wherein the composition is formulated as an injectable composition or as a topical composition. Clause 14b. The method of any one of clauses 1 to 13, wherein the composition is formulated as a gel, a jelly, a cream, a lotion, a foam, a slurry, an ointment, an oil, a paste, a suppository, a spray, a semisolid composition, a solid composition, a stick, or a mousse.

Clause 15. The method of any one of clauses 1 to 14, wherein the composition further comprises a pharmaceutically acceptable carrier.

Clause 16a. The method of clause 15, wherein the pharmaceutically acceptable carrier comprises an aqueous phase. Clause 16b. The method of clause 15, wherein the pharmaceutically acceptable carrier comprises one or more of a suspension, an emulsion, a powder, a solution, a dispersion, or an elixir.

Clause 17. The method of clause 15 or 16, wherein the pharmaceutically acceptable carrier comprises an oil-in-water emulsion or a water-in-oil emulsion.

Clause 18a. The method of any one of clauses 1 to 17, wherein the composition is formulated for administration to an epithelial surface. Clause 18b. The method of any one of clauses 1 to 17, wherein the composition is formulated for being administered by injection. Clause 18c. The method of any one of clauses 1 to 17, wherein the composition is formulated for being administered by subcutaneous injection, intradermal injection, transdermal injection, or subdermal injection. Clause 18d. The method of any one of clauses 1 to 17, wherein the composition is formulated for being administered by intramuscular injection, intravenous injection, intraperitoneal injection, intraosseous injection, intracardiac injection, intraarticular injection, or intracavernous injection. Clause 18e. The method of any one of clauses 1 to 17, wherein the composition is formulated for being administered by depot injection, by infiltration injection, by an indwelling catheter, or by microneedling. Clause 18f. The method of any one of clauses 1 to 17, wherein the composition is formulated for being administered transdermally.

Clause 19. The method of clause 18, wherein the epithelial surface is a superficial epidermal area, a stratum corneum, an eye surface, or an intestinal surface.

Clause 20. The method of any one of clauses 1 to 17, wherein the composition is formulated for reducing trans-epidermal water loss.

Clause 21. The method of any one of clauses 1 to 17, wherein the composition is formulated as a barrier formulation.

Clause 22. The method of any one of clauses 1 to 17, wherein the composition is formulated as a wound-closure formulation.

Clause 23. The method of any one of clauses 1 to 17, wherein the composition is formulated for preventing or reversing wrinkles in the subject, preventing or reversing age spots in the subject, preventing or reversing dry skin in the subject, or preventing or reversing uneven skin tone in the subject.

Clause 24. The method of any one of clauses 1 to 17, wherein the composition is formulated for preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject.

Clause 25a. The method of any one of clauses 1 to 17, wherein the disease, or condition comprises wrinkles, age spots, dry skin, uneven skin tone, skin sagging, skin aging, reduced skin tensile strength, photodamaged skin, or striae distensae (stretch marks). Clause 25b. The method of any one of clauses 1 to 17, wherein the disease, or condition comprises thyroid hormone-induced myocardial hypertrophy, or a tendon rupture, damage, or tear.

Clause 26. Use of a composition comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 14 kDa and about 30 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5, wherein the concentration of silk fibroin fragments in the composition is from about 0.001% w/v to about 10% w/v, in the manufacture of a medicament for the treatment or prevention of a disorder, disease, or condition alleviated by i) stimulating or modulating collagen expression in a subject in need thereof; and/or ii) stimulating or modulating claudin-1 expression in a subject in need thereof, and/or iii) stimulating or modulating one more anti-inflammatory genes in a subject in need thereof.

Clause 27. The use of clause 26, wherein the composition further comprises 0 to 500 ppm lithium bromide.

Clause 28. The use of clause 26 or clause 27, wherein the composition further comprises 0 to 500 ppm sodium carbonate Clause 29. The use of any one of clauses 26 to 28, wherein the silk fibroin fragments have a polydispersity between 1 and about 1.5.

Clause 30. The use of any one of clauses 26 to 28, wherein the silk fibroin fragments have a polydispersity between about 1.5 and about 2.0.

Clause 31. The use of any one of clauses 26 to 28, wherein the silk fibroin fragments have a polydispersity between about 1.5 and about 3.0.

Clause 32. The use of any one of clauses 26 to 28, wherein the silk fibroin fragments have a polydispersity between about 2.0 and about 2.5.

Clause 33. The use of any one of clauses 26 to 28, wherein the silk fibroin fragments have a polydispersity between about 2.5 and about 3.0.

Clause 34. The use of any one of clauses 26 to 33, wherein the silk fibroin fragments do not spontaneously or gradually gelate and do not visibly change in color or turbidity when in an aqueous solution for at least 10 days prior to formulation into the composition.

Clause 35. The use of any one of clauses 26 to 34, wherein the silk fibroin fragments are present in the composition at about 0.001% w/v to about 1% w/v.

Clause 36. The method of any one of clauses 26 to 34, wherein the silk fibroin fragments are present in the composition at about 0.01% w/v to about 1% w/v.

Clause 37. The use of any one of clauses 26 to 34, wherein the silk fibroin fragments are present in the composition at about 0.025% w/v to about 1% w/v.

Clause 38. The use of any one of clauses 26 to 34, wherein the silk fibroin fragments are present in the composition at about 0.05% w/v to about 0.7% w/v.

Clause 39. The use of any one of clauses 26 to 38, wherein the composition is formulated as an injectable composition or as a topical composition.

Clause 40. The use of any one of clauses 26 to 39, wherein the composition further comprises a pharmaceutically acceptable carrier.

Clause 41. The use of clause 40, wherein the pharmaceutically acceptable carrier comprises an aqueous phase.

Clause 42. The use of clause 40 or 41, wherein the pharmaceutically acceptable carrier comprises an oil-in-water emulsion or a water-in-oil emulsion.

Clause 43. The use of any one of clauses 26 to 42, wherein the composition is formulated for administration to an epithelial surface.

Clause 44. The use of clause 43, wherein the epithelial surface is a superficial epidermal area, a stratum corneum, an eye surface, or an intestinal surface.

Clause 45. The use of any one of clauses 26 to 42, wherein the composition is formulated for reducing trans-epidermal water loss.

Clause 46. The use of any one of clauses 26 to 42, wherein the composition is formulated as a barrier formulation.

Clause 47. The use of any one of clauses 26 to 42, wherein the composition is formulated as a wound-closure formulation.

Clause 48. The use of any one of clauses 26 to 42, wherein the composition is formulated for preventing or reversing wrinkles in the subject, preventing or reversing age spots in the subject, preventing or reversing dry skin in the subject, or preventing or reversing uneven skin tone in the subject.

Clause 49. The use of any one of clauses 26 to 42, wherein the composition is formulated for preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject.

Clause 50. The use of any one of clauses 26 to 42, wherein the disease, or condition comprises wrinkles, age spots, dry skin, uneven skin tone, skin sagging, skin aging, reduced skin tensile strength, photodamaged skin, or striae distensae (stretch marks).

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

General Procedures The compositions of this invention may be made by various methods known in the art.

Such methods include those of the following examples, as well as the methods specifically exemplified below.

### Example 1: Collagen Stimulation by Silk Fibroin

The Silk-Collagen Connection in Skin Health and Aging—Cosmeceuticals are on the rise. In response to rising demand for anti-aging skincare products and products suitable for use by consumers with sensitive skin types, the skincare industry has developed “cosmeceuticals.” These cosmetic products incorporate biologically active ingredients in an effort to enhance skin health as well as to beautify it; their purpose is to resolve the cause of skin imperfections rather than covering them up. The rising demand for cosmeceuticals is a result of the aging of the global population with a concomitant desire to retain youthful appearances; the past decade saw a rapid growth in population with a marked increase in the those aged 40 years and older, and the use of cosmetic products in these older age groups is also on the rise. As a result, demand for products that will prevent or reverse wrinkles, age spots, dry skin, and uneven skin tone has increased, spurring new formulation developments and industry growth. [Mordor Intelligence (2019). Cosmeceuticals Market—Segmented by Product Type (Skin Care, Hair Care, Injectable, Oral Care), Active Ingredients (Antioxidants, Botanicals, Exfoliants, Peptides, Retinoids), and Regions—Growth, Trends, and Forecast (2019-2024). Available at www.mordorintelligence.com/industry-reports/global-cosmeceuticals-market-industry. Accessed May 5, 2019. Archived at web.archive.org/web/20190506184300/https://www.mordorintelligence.com/industry-reports/global-cosmeceuticals-market-industry.]As they do not include drugs for the treatment of diseased skin conditions, these products are not regulated by agencies such as the US Food and Drug Administration (FDA), and do not require a doctor's prescription. [Martin K I, Glaser D A (2011) Cosmeceuticals: The new medicine of beauty. Missouri Medicine 108:1; Report Linker (2018) Global Cosmeceuticals Market Outlook 2022. Available at www.reportlinker.com/p01103487/Global-Cosmeceuticals-Market-Outlook.html. Accessed Mary 5, 2019. Archived at web.archive.org/web/20190506184758/https://www.reportlinker.com/p01103487/Global-Cosmeceuticals-Market-Outlook.html.] Due to their high popularity and accessibility, the global market for cosmeceuticals was USD $47B in 2017, and is expected to reach a value of $80B by 2023. [Mordor Intelligence (2019). Cosmeceuticals Market—Segmented by Product Type (Skin Care, Hair Care, Injectable, Oral Care), Active Ingredients (Antioxidants, Botanicals, Exfoliants, Peptides, Retinoids), and Regions—Growth, Trends, and Forecast (2019-2024). Available at www.mordorintelligence.com/industry-reports/global-cosmeceuticals-market-industry. Accessed May 5, 2019. Archived at web.archive.org/web/20190506184300/https://www.mordorintelligence.com/industry-reports/global-cosmeceuticals-market-industry.] In the US, the cosmeceutical market has had retail sales well in excess of $10B in recent years, and is continuing to grow. [Packaged Facts (2012) Cosmeceuticals in the US. 6^{th} ed. Available at www.packagedfacts.com/Cosmeceuticals-Edition-6281775/. Accessed May 5, 2019. Archived at https://web.archive.org/web/20190506183806/https://www.packagedfacts.com/Cosmeceuticals-Edition-6281775/.]

The role of collagen, fibroblasts, and the extracellular matrix in skin health and aging.

The dermis is the largest portion of the skin and is primarily composed of a dense, collagen-rich proteinaceous extracellular matrix (ECM) which is responsible for the strength, resiliency, and elasticity of the skin. [Rittié L, Fisher G J (2015) Natural and sun-induced aging of human skin. Cold Spring Harb Perspect Med 5: a015370; Quan T, Fisher G J (2015) Role of age-associated alterations of the dermal extracellular matrix microenvironment in human skin aging: A mini-review. Gerontology 61: 427-434.] For decades, scientists have known that the visible hallmarks of skin aging such as thinning, drying, and fine wrinkling, are reflective of increases in the degradation of skin collagen with age. [Smith J G, Davidson E A, Sams W M, Clark R D (1962) Alterations in human dermal connective tissue with age and chronic sun damage. J Invest Dermatol 39: 347-350; Lavker R M (1979) Structural alterations in exposed and unexposed aged skin. J Invest Dermatol 73: 59-66; Varani J et al (2000) Vitamin A antagonizes decreased cell growth and elevated collagen-degrading matrix metalloproteinases and stimulates collagen accumulation in naturally aged human skin. J Invest Dermatol 114: 480-486; Varani J et al (2000) Vitamin A antagonizes decreased cell growth and elevated collagen-degrading matrix metalloproteinases and stimulates collagen accumulation in naturally aged human skin. J Invest Dermatol 114: 480-486.] As the primary component of the skin's connective tissue, collagen plays a key role in maintaining skin strength and resiliency; its degeneration results in skin that is fragile, easily bruised, and has lost it general youthful appearance. [Ibid.] More specifically, effects of aging on the dermis involve deleterious alterations to the structure and organization of the collagen-based extracellular matrix. [RittiéL, Fisher G J (2015) Natural and sun-induced aging of human skin. Cold Spring Harb Perspect Med 5: a015370; Quan T, Fisher G J (2015) Role of age-associated alterations of the dermal extracellular matrix microenvironment in human skin aging: A mini-review. Gerontology 61: 427-434.]

This degeneration of collagen in skin occurs as a result of normal age-associated increases in the expression of collagen-degrading enzymes called matrix metalloproteinases (MMP) in conjunction with normal age-associated decreases in the expression of collagen itself. The increases in enzymatic MMP action lead to the accumulation of fragmented collagen fibrils in the dermal ECM over time. The loss of the structural integrity of the ECM that goes along with this collagen fragmentation is biologically translated to a loss of integrity of the shape of the dermal fibroblast cells that produce collagen via a phenomenon known as mechanotransduction. [RittiéL, Fisher G J (2015) Natural and sun-induced aging of human skin. Cold Spring Harb Perspect Med 5: a015370; Quan T, Fisher G J (2015) Role of age-associated alterations of the dermal extracellular matrix microenvironment in human skin aging: A mini-review. Gerontology 61: 427-434.] The interaction of dermal fibroblasts with their surrounding ECM occurs through transmembrane binding and signaling receptors known as integrins on the cell surface. In fibroblasts attached to a “stretched” collagen matrix experiencing appropriate mechanical stress such as the normal tissue tension seen in healthy, young skin, collagen production is high. However, collagen expression is suppressed in fibroblasts within more “relaxed” ECM environments such as is seen in the ECM of aged skin, with substantial accumulations of fragmented collagen. [Chiquet M (1999) Regulation of extracellular matrix gene expression by mechanical stress. Matrix Biol 18: 417-426.] Thus, the loss of proper fibroblast shape is linked to a loss in its cellular function, which leads to further reductions in collagen production and then increases in MMP expression. [RittiéL, Fisher G J (2015) Natural and sun-induced aging of human skin. Cold Spring Harb Perspect Med 5: a015370; Quan T, Fisher G J (2015) Role of age-associated alterations of the dermal extracellular matrix microenvironment in human skin aging: A mini-review. Gerontology 61: 427-434.] In addition to these collagen-based effects, the population (number) of dermal fibroblasts in skin is itself also reduced during aging. In young vs. old skin, the collagen content has been shown to be reduced by 68%, and the number of fibroblasts reduced by 35%. [Varani J et al (2000) Vitamin A antagonizes decreased cell growth and elevated collagen-degrading matrix metalloproteinases and stimulates collagen accumulation in naturally aged human skin. J Invest Dermatol 114: 480-486; Varani J et al (2006) Decreased collagen production in chronologically aged skin roles of age-dependent alteration in fibroblast function and defective mechanical stimulation. Am J Pathol 168: 1861-1868.] The ensuing feedback loop of changes in collagen and MMP expression and fibroblast cellular function fuels the changes in collagen homeostasis that lead to the visible hallmarks of aged skin as decreases in collagen matrix density perpetuate a downregulation cycle of ECM protein production. [RittiéL, Fisher G J (2015) Natural and sun-induced aging of human skin. Cold Spring Harb Perspect Med 5: a015370; Quan T, Fisher G J (2015) Role of age-associated alterations of the dermal extracellular matrix microenvironment in human skin aging: A mini-review. Gerontology 61: 427-434.]

Without wishing to be bound by any particular theory, it appears that it is the structural quality of the ECM rather than the age of dermal fibroblasts that is a key determinant of the appearance of skin aging. [Quan T et al (2013) Enhancing structural support of the dermal microenvironment activates fibroblasts, endothelial cells, and keratinocytes in aged human skin in vivo. J Invest Dermatol 133: 658-667.] Therefore, treatments of aged or damaged skin that promote ECM and fibroblast health may succeed in reversing age-dependent changes in skin appearance. In fact, studies have shown that the decreased collagen production observed in aged skin can be reversed somewhat by treatments that stimulate dermal fibroblasts. [Varani J et al (2000) Vitamin A antagonizes decreased cell growth and elevated collagen-degrading matrix metalloproteinases and stimulates collagen accumulation in naturally aged human skin. J Invest Dermatol 114: 480-486; Varani J et al (2006) Decreased collagen production in chronologically aged skin roles of age-dependent alteration in fibroblast function and defective mechanical stimulation. Am J Pathol 168: 1861-1868; Nusgens B V et al (2001) Topically applied vitamin C enhances the mRNA level of collagens I and III, their processing enzymes and tissue inhibitor of matrix metalloproteinase 1 in the human dermis. J Invest Dermatol 116: 853-859; Quan T et al (2013) Enhancing structural support of the dermal microenvironment activates fibroblasts, endothelial cells, and keratinocytes in aged human skin in vivo. J Invest Dermatol 133: 658-667; RittiéL, Fisher G J (2015) Natural and sun-induced aging of human skin. Cold Spring Harb Perspect Med 5: a015370.] Moreover, since the more pronounced changes in skin appearance that are observed in sun-damaged skin are also not the result of damage to the collagen-producing fibroblasts themselves, it is expected that reversals of this damage are also possible. [Smith J G, Davidson E A, Sams W M, Clark R D (1962) Alterations in human dermal connective tissue with age and chronic sun damage. J Invest Dermatol 39: 347-350; Lavker R M (1979) Structural alterations in exposed and unexposed aged skin. J Invest Dermatol 73: 59-66; Varani J et al (2000) Vitamin A antagonizes decreased cell growth and elevated collagen-degrading matrix metalloproteinases and stimulates collagen accumulation in naturally aged human skin. J Invest Dermatol 114: 480-486; Varani J (2001) Inhibition of Type I procollagen synthesis by damaged collagen in photoaged skin and by collagenase-degraded collagen in vitro. Am J Pathol 158: 931-942; Varani J et al (2006) Decreased collagen production in chronologically aged skin roles of age-dependent alteration in fibroblast function and defective mechanical stimulation. Am J Pathol 168: 1861-1868.]

Silk-based skincare promotes collagen expression, improving aging and damaged skin.

At its core, silk fiber is comprised of a natural protein known as fibroin. As the first implantable biomaterial utilized for skin ligation, silk fibroin boasts a well-established history of use and compatibility with human skin. In 2003, the authors reported on the ability of the silk fibroin protein to induce collagen production by fibroblasts; the culture of fibroblasts with a modified silk protein-based matrix promoted collagen expression as well as increased fibroblast cell density. [Chen J et al (2003) Human bone marrow stromal cell and ligament fibroblast responses on RGD-modified silk fibers. J Biomed Mater Res A 67: 559-570.] It is believed that a direct interaction between the silk fibroin and ECM-producing cells was responsible for these favorable outcomes.

As described herein, a liquid formulation of silk fibroin (ACTIVATED SILK™) has an effect on fibroblasts. That is, with the addition of silk fibroin, fibroblasts in culture were stimulated to produce over 20-30% more collagen than control fibroblasts (depending upon the concentration of added silk fibroin, see FIG. 2). Given the deleterious feedback loop described above, this demonstration represents an extremely promising discovery for the development of cosmeceutical treatments for aged and/or damaged skin.

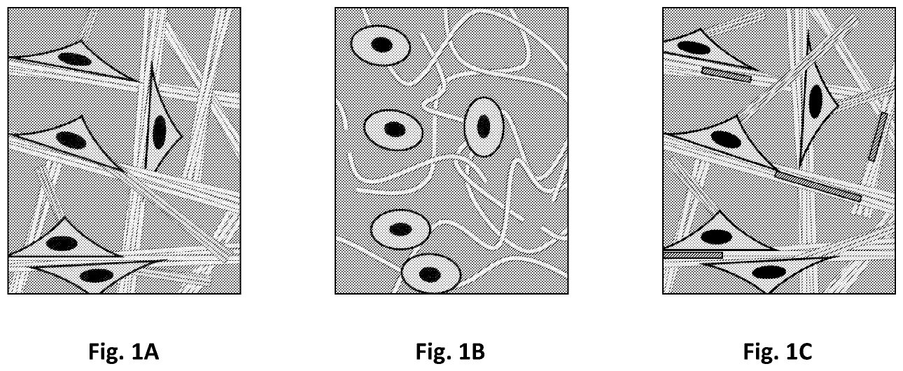

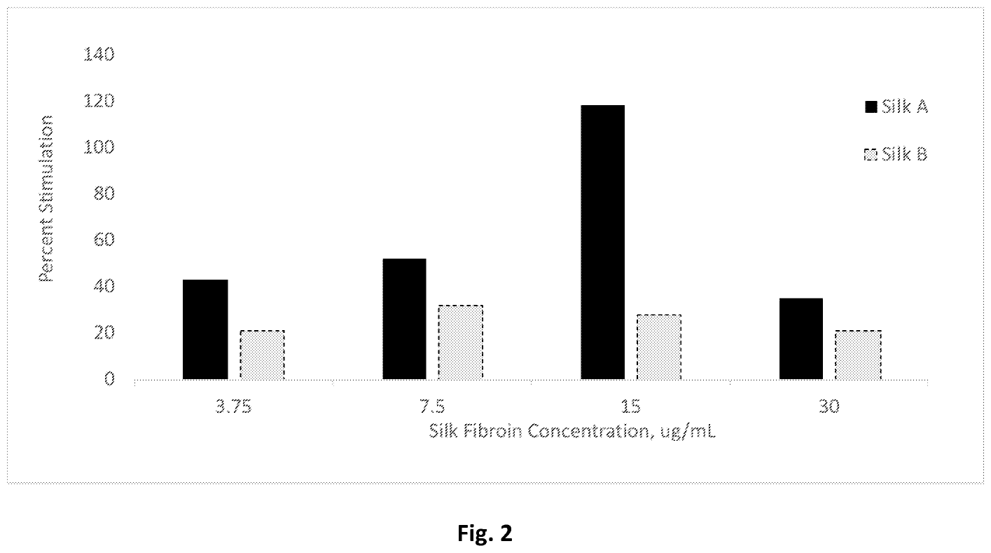

As a skincare ingredient, liquid silk fibroin is thought to temporarily elevate the skin's perceived concentration of ECM proteins. In addition to molecular signaling via interactions with integrins on fibroblasts, silk fibroin's protein sequence is dominated by hydrogen-rich amino acids that easily and rapidly bond with the amino acids present in collagen. Specifically, silk fibroin's β-sheet-rich structure is primarily comprised of reversible hydrogen bonds, and its protein sequence is governed by the non-polar amino acids glycine and alanine. [Marelli B et al (2012) Silk fibroin derived polypeptide-induced biomineralization of collagen. Biomaterials 33: 102-108; Schroeder W A et al (1955) The Amino Acid Composition of Bombyx mori Silk Fibroin and of Tussah Silk Fibroin. J Am Chem Soc 77: 3908-3913.] These hydrogen-rich amino acids easily and rapidly bond with the tightly packed polar and charged amino acids present in collagen that are responsible for the formation of healthy skin, muscle and bone. [Lodish H et al. (2000) Collagen: The Fibrous Proteins of the Matrix. Molecular Cell Biology. Macmillan Publishers, New York.] The bonding of collagen with silk fibroin is a naturally stable interaction that may further enhance the integrity and stability of the ECM. [Saxena T et al (2014) Chapter 3—Proteins and Poly(Amino Acids) A2—Kumbar, Sangamesh G. In Natural and Synthetic Biomedical Polymers, Laurencin C T, Deng M (eds), pp 43-65. Oxford: Elsevier.] An ensuing positive feedback biological loop stimulated by silk fibroin engagement of integrins and stabilization of ECM facilitates collagen production, leading to healthier, more youthful-looking skin (FIG. 1). ACTIVATED SILK™ fibroin is clinically proven to tighten and firm human skin.

ACTIVATED SILK™ is a liquid formulation of silk fibroin protein. The process for purifying and solubilizing silk fibroin protein is free from toxic chemicals, requiring only pure, silk cocoons, non-toxic salts, and water. This replaces harsher hydrolysis methods that are conventionally used for the preparation of silk with a green chemistry method that requires no wastewater management as both the salts used and the biodegradable ACTIVATED SILK™ are safe to enter waterways. This means that ACTIVATED SILK™ replaces synthetic and possibly hazardous ingredients that come into contact with human skin with one that is non-toxic, renewable, requires less energy to produce, and generates less waste. ACTIVATED SILK™ is also biocompatible, meaning that it is safe for contact with all skin types, even for those with highly-sensitive skin. In fact, silk in various forms has been used as wound dressings and graft scaffolds and has been found to improve wound healing. [Altman G H et al (2003) Silk-based biomaterials. Biomaterials 24:401-416. https://www.ncbi.nlm.nih.gov/pmc/articles/PMC4573254/; Thurber A E, Omenetto F G, Kaplan D L (2015) In vivo bioresponses to silk proteins. Biomaterials 71:145-157. www.ncbi.nlm.nih.gov/pmc/articles/PMC4573254/pdf/nihms717107.pdf.]

According to the US Environmental Protection Agency (EPA), green chemistry is the use of chemistry for source reduction—that is, reducing pollution at its source by minimizing or eliminating the hazards of chemical reagents, solvents, and products. This is achieved by the design of chemical products and processes that reduce or eliminate the use or generation of such hazardous substances. Green chemistry principles apply throughout the lifecycle of a chemical product, including its manufacture, use, and disposal.

The fibroin units of the liquid silk have the ability to self-assemble into robust biomaterials with a variety of secondary structures, meaning that silk can polymerize into higher ordered polymers without the need for solvents, plasticizers, or catalysts that typically have deleterious effects on living biology and the environment. Furthermore, the protein's hydrophobic nature and tendency to crystallize lend it resiliency to changes in temperature and moisture and provides the opportunity to promote the formation of structures such as gels and films. [Li A B et al (2015) Silk-based stabilization of biomacromolecules. J Control Release 219: 416-430. https://www.ncbi.nlm.nih.gov/pmc/articles/PMC4656123/.] Unlike drugs and biological molecules, where pH fluctuations can drastically inhibit efficacy, silk fibroin's hydrogen-rich amino acid structure is not negatively affected by pH changes. [Schroeder W A et al (1955) The Amino Acid Composition of Bombyx mori Silk Fibroin and of Tussah Silk Fibroin. J Am Chem Soc 77: 3908-3913.] It is hypothesized that low pH is capable of “untwisting” collagen's mechanical structure [Coffey J W et al (1976) Digestion of native collagen, denatured collagen, and collagen fragments by extracts of rat liver lysosomes. J Biol Chem 251: 5280-5282; Fine N A et al (2015) SERI surgical scaffold, prospective clinical trial of a silk-derived biological scaffold in two-stage breast reconstruction: 1-year data. Plastic Reconst Surg 135: 339-351], such as that located in the dense stratum corneum layer of the skin. As a result, protonation strategies for enhanced transport into the epidermis and intra-dermis should not impede ACTIVATED SILK™'s functionality. Clinical skincare trials support this hypothesis, with decreased appearance of fine lines and wrinkles observed as early as seven days following application of low pH cosmeceutical serums and eye treatments made with ACTIVATED SILK™.

ACTIVATED SILK™ can fulfill roles as a hydrant, emulsifier, exfoliant, cleanser, gel/filler, carrier for bioactives such as vitamin C or for (phthalate-free) fragrances, and even a bacteriostatic agent. Thus, ACTIVATED SILK™ represents a highly effective active ingredient in skincare.

### Example 2: Effect of Activated Silk on Collagen Production: Study Description

Study aim: assess the effect of the test item (ACTIVATED SILK™) on the collagen concentration of a fibroblast culture, 24 hours after treatment. Primary human fibroblast cells (passage 5) were seeded 50000 cells/cm² in 24-well culture plates and incubated overnight (37° C., 5% CO₂). The medium was discarded and replaced by 500 μL of the various concentrations of the test article or reference items. Plates were incubated for 24 hours (37° C., 5% CO₂). The culture medium was removed, cells were rinsed and recovered. Intracellular and extracellular collagen were quantified with the Sirius red dye (exhibits a specific affinity for the triple helical (Gly-X-Y)n structure of native collagen). The absorbance of the dye-collagen complex was measured spectrophotometrically at 540 nm. The total protein was assessed, after sonication, using the Bradford method.

Test system: cells—primary human fibroblasts prepared according to the current working instruction; before the study, the cells are cultivated in medium DMDM 4.5 g/l glucose, 2 mM L-glutamine or stabilized glutamine, 10% heat inactivated foetal calf serum (FCS), penicillin 50 UI/ml, 50 μg/ml streptomycin. During the study, FCS is reduced to 1% for both the reference item and the test item dilution. Cells are exempt of mycoplasma. Assessment of mycoplasma was performed according to the current working instruction.

Reference items: negative control: 1% heat inactivated FCS culture medium; positive control: transforming growth factor β1 (TGFβ) 20 ng/ml in 1% FCS culture medium.

### Material and Reagents

Materials: 24 wells plates for cell culture; 96 wells plate for absorbance reading; cells scrapper; ultrasonic probe; MULTISKAN EX plate reader (Thermo life sciences)—reading range 0-3.5 units of Absorbance—linearity range 0-2.000 units of Absorbance; conventional material used in cell culture laboratory.

Reagents: culture medium: DMEM 4.5 g/l glucose, 2 mM L glutamine or stabilized glutamine, 10% heat inactivated FCS, 50 IU/ml penicillin, 50 μg/ml streptomycin)—stored at 5° C.±3° C.; Dulbecco's PBS Ca²⁺ and Mg²⁺ free—stored at room temperature 20° C.±5° C.; Direct Red 80 CAS 2610-10-8—stored at room temperature 20° C.±5° C.; Protease inhibitor cocktail—stored at 5° C.±3° C.; Bradford reagent—stored at 5° C.±3° C.; BSA solution (bovine albumin serum)—stored at 5° C.±3° C.; HCl—stored at room temperature 20° C.±5° C.; NaOH—Stored at room temperature 20° C.±5° C.; TGF R31—stored at −20° C.±5° C.; 3 mg/ml collagen solution—stored at 5° C.±3° C.

Series definition: 8 concentrations of the test item were tested. The collagen assessment was performed on the 4 highest concentrations non cytotoxic. Each test item or reference item condition is tested on at least three culture wells.

### Test Protocol

Cells seeding: cells were seeded at 50000 cells/cm² in 24 wells culture plates then were incubated overnight (37° C., 5% CO₂).

Contact between cells and test item: test item and reference items dilutions were performed in 1% FCS culture medium. The medium was discarded and replaced by 500 μl of the various concentrations of the test item or reference items. Wells for the negative control were filled with 1% FCS culture medium. The plates were incubated for 48 hours±1 hour (37° C., 5% CO₂).

Assessment of the collagen synthesis and the cell density: the culture medium from each well was removed and the cell layer was rinsed with 500 μL of 2× concentrated protease inhibitor cocktail. All, medium+inhibitor, was pooled in the same tube.

The cell layer was recovered by scraping in 500 μL of 1× concentrated protease inhibitor cocktail and the well was rinsed again with 500 μL of 1× cocktail. The two volumes, which constitute the extracellular matrix, were pooled in the same tube and treated by ultrasonic probe for 40 seconds.

Intracellular and extracellular collagen were quantified with the Sirius red dye (Direct Red 80) which exhibits a specific affinity for the triple helical (Gly-X-Y)n structure of native collagen. The absorbance of the complex dye-collagen is measured with a spectrophotometer at 540 nm. A calibration range is established between 0 and 10 μg of collagen.

The total protein quantity was assessed using the Bradford method (Bradford et al Anal Biochem 1976; 72:248-54) with a calibration range established from 0 to 400 μg/ml BSA solution in PBS. 30 μl of each sample (dilution of the test item, reference items, standard) were mixed with 280 μl of Bradford reagent in a 96-wells plate. The plate is incubated about 15 minutes at room temperature away from light. The absorbances were measured at 620 nm against Bradford reagent as blank.

With the addition of silk fibroin, fibroblasts in a culture were stimulated to produce over 20-30% more collagen than control fibroblasts depending upon the concentration of added silk fibroin; also collagen production is dependent on the silk composition (FIG. 2). Intracellular collagen production at various silk concentrations is shown as a function of silk type. Percent stimulation is the increase in collagen formation compared to the negative control. Silk average MW compositions: silk A=low MW (average weight average molecular weight selected from between about 14 kDa and about 30 kDa); silk B=mid MW (average weight average molecular weight selected from between about 39 kDa and about 54 kDa).

### Results Calculation and Interpretation

Cell density: protein concentration is calculated according to the established calibration curve, Absorbance=f (protein amount in μg). It is expressed in μg of protein per well.

Determination of collagen: the amount of collagen by wells are determined according to the established calibration curve (Absorbance=f (collagen amount in μg). It is expressed in μg of collagen per well. The results are expressed by a ratio between the amount of collagen and the amount of protein in the well.

### Example 3: The Effect of Mid and Low Molecular Weight (MW) Silks on Collagen Synthesis in Human Dermal Fibroblasts

### I. Study Objective

To determine the effect of silk in the production of collagen from human dermal fibroblasts

### II. Test Item and Concentration Calculation

Formulation: Liquid; Storage conditions: Fridge (4° C.); Test item nature: Cosmetic ingredient. The concentration of Test items is shown below (Table 3-1). Information linked to the identification, purity, and stability of the test item is under the manufacturing department's responsibility.

TABLE 3-1
Concentration of Test Items

| | 1^{st} lot | | 2^{nd} lot | |
|---|---|---|---|---|
| Stock Size | Mid | Low | Mid | Low |
| Lot number | Lot 21041 | Lot 21034 | Lot 21158 | Lot 21188 |
| MW | 50179 | 27439 | 51261 | 29162 |
| % W/V | 6.16% | 6.05% | 6.2% | 5.85% |
| μM | 1227.61 | 2204.89 | 1209.5 | 2006.04 |
| Final conc in μM | 120 | 240 | 120 | 240 |
| Final conc in | 0.6 | 0.7 | 0.6 | 0.7 |
| % W/V (g/100 ml) | | | | |

### III. Study Principle

Fibroblasts are the main cells in the dermal compartment of the skin. They are specialized in collagen synthesis. There are many different types of collagens: only a few of them play a role in the skin health. Collagens 1 and 3 are crucial for skin wellness and beauty. In term of abundance, collagen 1 constitutes 70% of the dermal extracellular matrix and confers resistance to tension and traction. (Hui Hui Wong et al. Sci Rep. 2020; 10: 19723.) The reduction of collagen 1 gene expression in skin fibroblasts limits collagen 1 level in the dermis, which causes wrinkle formation (Hui Hui Wong et al. Sci Rep. 2020; 10: 19723.). Therefore, promoting collagen 1 synthesis is key to aesthetics and anti-aging treatments (Hui Hui Wong et al. Sci Rep. 2020; 10: 19723; DM Reilly et al. Plast. Aesthet. Res. 2021; 8:2). On the other hand, collagen 4, especially COL4A1 subunit, plays a prominent role in diseases (Ana Maria Abreu-Velez et al. N Am J Med Sci. 2012 January; 4(1): 1-8).

Retinol, commonly used in cosmeceutical treatment, does not exert a significant biological effect on the skin. Therefore, to have crucial roles in skin health, retinol is required to transform into retinoic acid (20 times more potent than retinol) (Malwina Zasada M. et al. Dermatol Alergol. 2019 August; 36(4): 392-397). Different concentrations of retinoic acid have been shown to stimulate or inhibit collagen 1 production by the dermal fibroblasts (Varani J. et al. J Invest Dermatol. 1990 May; 94(5):717-23). Moreover, retinol and retinoic acid are restricted by their instability (light and air-sensitive) and toxicity. These limitations complicate the formulating, storage, and application of a product containing retinol and retinoic acid. Therefore, there is an unmet need for new cosmetic active ingredients that boost collagen, stabilize formulation, and are safe and easy to use.

The study aims to assess the efficacy of the test items on collagen production using normal human dermal fibroblast. The total (intracellular and extracellular) collagen was quantified with the Sirius red: absorbance of the complex dye-collagen was determined in spectrophotometer of 540 nm wavelength. Quantitative collagen 1 genes expression analysis was performed using the real-time PCR standard method. Flow cytometry was used to detect/confirm collagen protein isoforms following in vitro human dermal fibroblast stimulation with test items.

### IV. Study Course and Methods

Study duration: Mar. 30, 2021 to Aug. 30, 2021.

For experiments, primary cultures of human dermal fibroblasts were used when they reached >70% confluence.

### In Vitro Model: Extracellular Matrix Generation

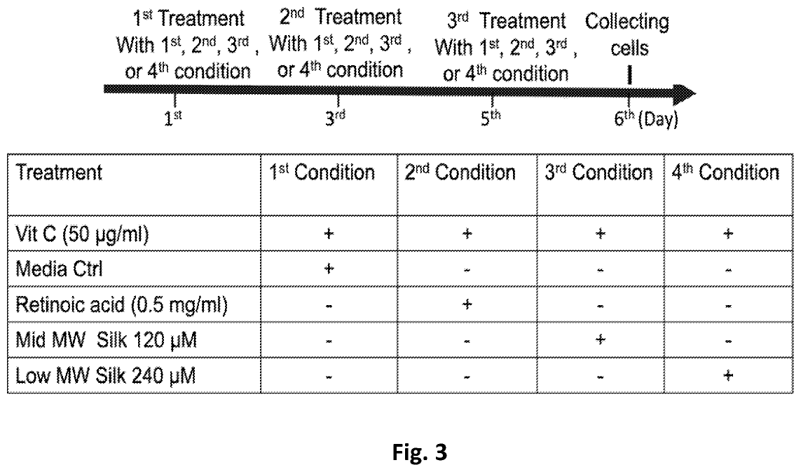

Primary human dermal fibroblasts were seeded into each well in DMEM 1 g/L glucose media (Genesee Scientific), containing 1% FBS (Genesee Scientific), 2 mM glutamine, 100 U/ml penicillin, and 100 μg/ml streptomycin (Cytiva), and cultured overnight at 37° C., 5% CO₂. The next day, medium supplemented with 50 μg/ml ascorbic acid (also known as Vit C, Sigma Aldrich) together with each treatment condition were added to each culture to promote the self-assembly of extracellular matrix: Medium was replaced with fresh medium every 2 days for a total of 5 days. The timeline indicates experiment chronology and treatment conditions are listed in FIG. 3: The positive control treatment was TGF-β (10 ng/ml, Tonbo Biosciences)+Vit C (50 μg/ml, Sigma Aldrich) Ghetti, M. et al. Br J Dermatol. 2018 August; 179(2): 381-393.

### In Vitro Model: Collagen Production

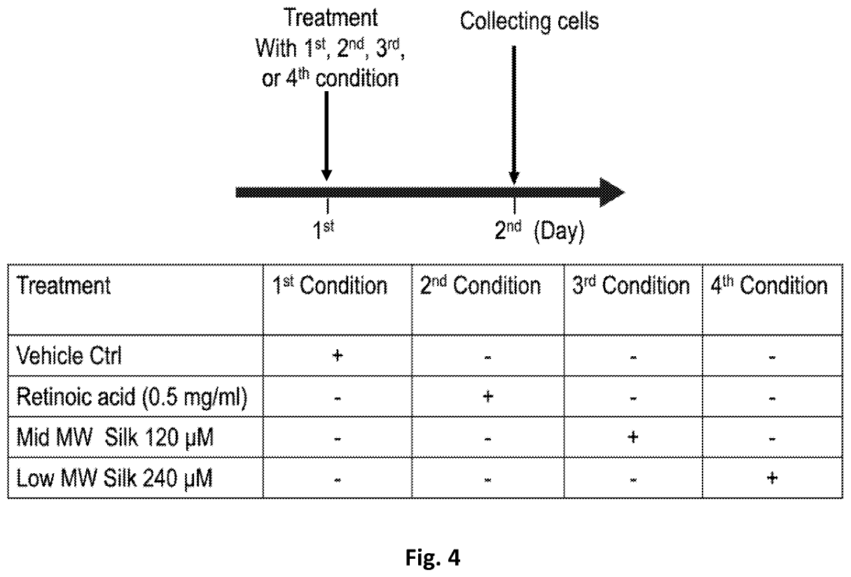

Primary human dermal fibroblasts were seeded into each well in DMEM 1 g/L glucose media (Genesee Scientific), containing 1% FBS (Genesee Scientific), 2 mM glutamine, 100 U/ml penicillin, and 100 μg/ml streptomycin (Cytiva), and cultured overnight at 37° C./5% CO₂. The next day, medium supplemented the treatment condition as shown in FIG. 4. The positive control treatment was TGF-β (10 ng/ml)+Vit C (20 μg/ml).

### Flow Cytometry

The following antibodies were used for flow cytometry staining: anti-collagen 1 (Abcam), anti-COL4A1 (Santa Cruz Biotechnology) followed by reaction with secondary antibodies (Thermo Fisher Scientific): Alexa Fluor™ 647-conjugated secondary, Alexa Fluor™—594 conjugated secondary, and Alexa Fluor™ 488-conjugated secondary.

### Quantitative RT-PCR

Total RNA was isolated from cells using Quick-RNA MicroPrep Kit (Genesee Scientific). RNA was reverse transcribed using the qPCRBIO cDNA Synthesis Kit (Genesee Scientific), and the resulting cDNA was amplified using qPCRBIO SyGreen Blue Mix Lo-ROX (Genesee Scientific). PCR was performed with primers as shown in Table 3-2

TABLE 3-2
Quantitative RT-PCR Primers

| | |
|---|---|
| GAPDH fwd⁷ | CAAGAGCACAAGAGGAAGAGAG |
| GAPDH rev⁷ | CTACATGGCAACTGTGAGGAG |
| COL1A1 fwd⁸ | AGGGCCAAGACGAAGACATC |
| COL1A1 rev⁸ | AGATCACGTCATCGCACAACA |

⁷Clément Guillot, C. et al. BMC Cancer. 2014; 14: 603.
⁸Kim, Cho-Rong et al. Int J Mol Med. 2017 January; 39(1): 31-38.

### Immunofluorescence

Cells were incubated overnight at 4° C. with anti-collagen 1 (Novus Biologicals) followed by reaction with Alexa Fluor™ 488-conjugated secondary Ab (Thermo Fisher Scientific). Nuclei were counterstained with Hoechst 33342, washed in PBS, and mounted with Antifade Mounting Media (Thermo Fisher Scientific).

### Sirius Red Dye Staining and Spectrophotometric Analysis

In brief, cells were fixed in 400 paraformaldehyde, carefully washed with tap water, and incubated in the Sirius red (0.1%, Electron Microscopy Sciences) at room temperature for 1 hr. The staining solution was removed, and the cells were washed two times with 0.50 v/v acetic acid. For spectrophotometric analysis, Sirius red was eluted in 0.1 N sodium hydroxide, and the optical density at 540 nm was determined using a Varioskan Lux Spectrophotometer (Thermo Fisher Scientific). (Xu Q et al. Am J Physiol Renal Physiol. 2007 August; 293(2):F631-40.

### Statistical Analysis

For comparisons between multiple groups, the overall differences were analyzed by ANOVA with Bonferroni multiple comparison.

### Materials

| Item | Vendor | Catalog number |
|---|---|---|
| Sirius Red | Electron Microscopy Sciences | 26357-02 |
| ProLong ™ Gold Antifade Mountant | Thermo Fisher Scientific | P36934 |
| L-Ascorbic acid | Sigma Aldrich | A4403-100MG |
| Collagen I Antibody | Novus Biologicals | NB600-408−0.1mg |
| Retinoic acid | Sigma Aldrich | R2625-100MG |
| Dulbecco's Modified Eagle's Medium | Genesee Scientific | 25-500L |
| Recombinant Human TGF-β1 | Tonbo Biosciences | 21-8369-u010 |
| GenClone ™ Fetal Bovine Serum | Genesee Scientific | 25-550 |
| qPCRBIO cDNA Synthesis Kit | Genesee Scientific | 17-700B |
| qPCRBIO SyGreen Blue Mix Lo-ROX | Genesee Scientific | 17-505B |
| Penicillin/Streptomycin/Glutamine solution, HyClone ™ | Cytiva | 16777-169 |
| Collagen I Antibody | Abcam | ab260043 |
| Alexa Fluor ™ 647 Goat Anti-Rabbit SFX Kit | Thermo Fisher Scientific | A31634 |
| Goat anti-Rabbit IgG (H + L) Cross-Adsorbed | Thermo Fisher Scientific | A27034 |
| Secondary Antibody, Alexa Fluor 488 | | |
| Goat anti-Mouse IgG (H + L) Highly Cross-Adsorbed | Thermo Fisher Scientific | A-11032 |
| Secondary Antibody, Alexa Fluor 594 | | |
| Anti-COL4A1 Antibody | Santacruz | sc-517572 |
| Quick-RNA MiniPrep Kit | Genesee Scientific | 11-327M |
| Hoechst 33342 | Thermo Fisher Scientific | H3570 |
| 16% Paraformaldehyde | Electron Microscopy Sciences | 15710 |

### V. Results

In this study, it was hypothesized that silk upregulated collagen production in human dermal fibroblasts. To test this hypothesis, an in vitro model was used to generate an extracellular matrix by co-treating the human dermal fibroblasts with silk and Vit C, a booster of collagen synthesis and extracellular matrix formation (DePhillipo, N N et al. Orthop J Sports Med. 2018 October; 6(10); Pullar J M et al. Nutrients. 2017 August; 9(8): 866) (FIG. 3). Preliminary data showed that Mid MW silk with Vit C and Low MW silk with Vit C increased total collagen levels in normal human dermal fibroblasts (FIGS. 5A-5B).

To examine the functional role of silk as a collagen activator, Vit C was excluded from the experimental model as shown in FIG. 4. The data showed that Mid and Low MW silk treatment enhanced the total collagen production and suggested Mid and Low MW silks' potential role as a single ingredient for collagen-boosting products (FIG. 6).

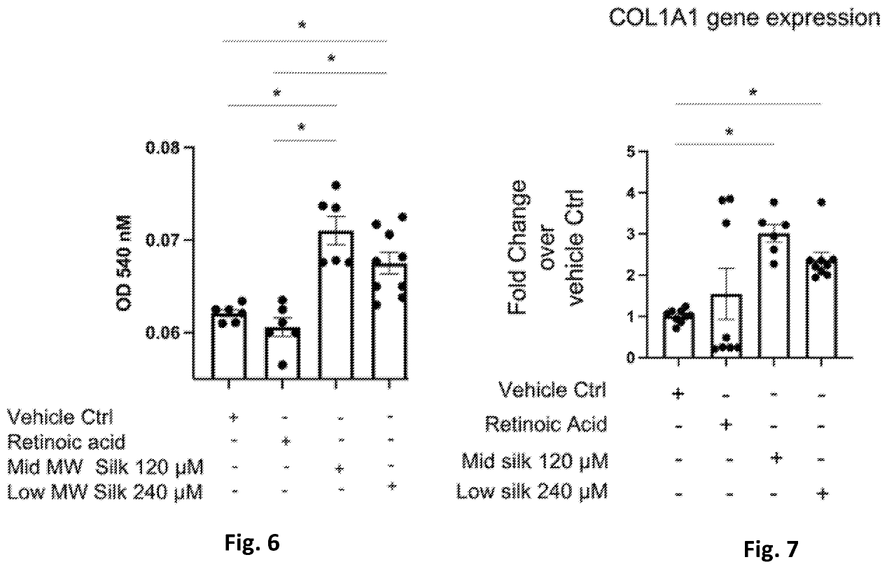

The collagen-boosting properties were then investigated in detail. It was found that the human dermal fibroblasts treated with Mid and Low MW silks upregulated COL1A1 gene expression, a gene encodes the pro-al chains of type I collagen (FIG. 7) compared to vehicle control. This data indicated that the silk peptides controlled intracellular COL1A1 gene expression.

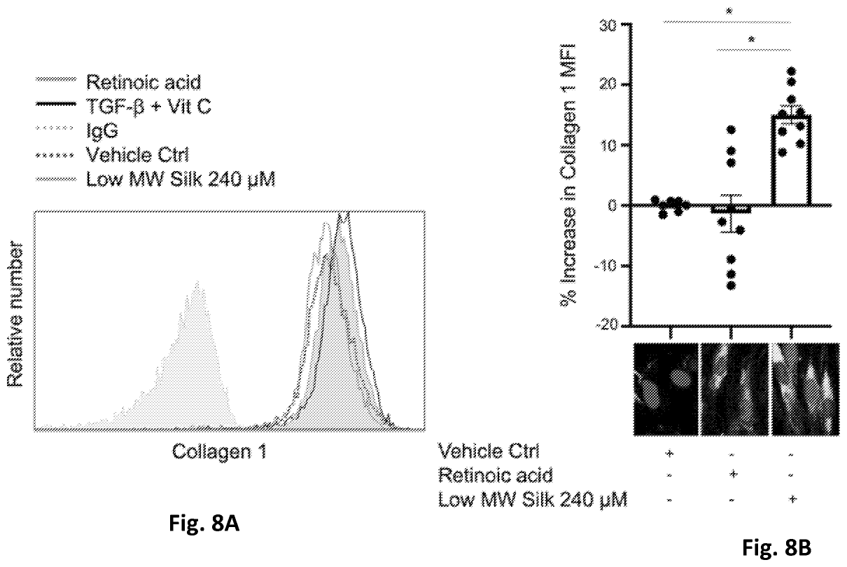

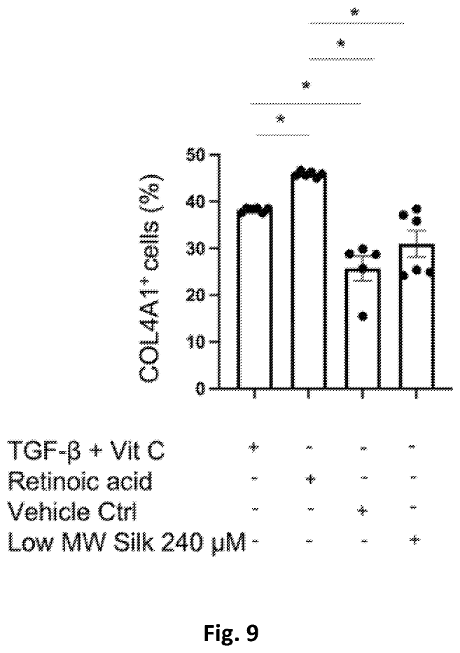

Flow cytometry analysis further confirmed that Low MW silk significantly upregulated collagen 1 protein expression while not modulating COL4A1, a subunit of collagen 4, protein expression (FIGS. 8A-8B). On the other hand, Mid MW silk (120 μM) did not increase collagen 1 and collagen 4 protein expression (data not shown). Interestingly, retinoic acid, a collagen enhancer molecule, robustly increased COL4A1 positive cells and inconsistently increased/decreased collagen 1 protein expression in normal human dermal fibroblast (FIGS. 8A-8B and FIG. 9). In addition, normal human fibroblasts co-treated with TGF-β and Vit C (a positive Ctrl) increased COL4A1 positive cells (to a lesser extent than retinoic acid) and collagen 1 protein expression (FIG. 8A and FIG. 9). It was concluded that Low MW silk selectively induced intracellular collagen 1 synthesis in human dermal fibroblasts.

### VI. Conclusion

Under the retained experimental conditions, Low MW silk shows a positive effect on collagen synthesis.

All patents, patent applications, and published references cited herein are hereby incorporated by reference in their entirety. While the methods of the present disclosure have been described in connection with the specific embodiments thereof, it will be understood that it is capable of further modification. Further, this application is intended to cover any variations, uses, or adaptations of the methods of the present disclosure, including such departures from the present disclosure as come within known or customary practice in the art to which the methods of the present disclosure pertain.

### Example 4: Permeation Analysis Using Tissue Cross Sections

After incubating the collected tissues for 18-24 hours in 10% formalin, the formalin was replaced with DPBS. Tissues were dehydrated in a series of graded ethanol (70-95%), dehydrated in xylene and embedded in paraffin. Slides containing cross sections were prepared per standard procedures. Three sections from each tissue were prepared on each slide. One unstained, deparafinized slide per tissue was prepared for fluorescent permeation analysis. Slides were rehydrated in dH2O for 5-10 minutes. DAPI stock solution was diluted 1:47,000 in dH2O and slides were incubated in diluted solution for 10 minutes. Slides were rinsed 3× in dH2O. Tissue sections were covered with Immuno-mount mounting solution (Thermo cat #9990402) and coverslips were applied. Slides were imaged on the Olympus VS100 slide scanner using a 10× objective to visualize fluorescent signal.

### Permeation Analysis Using Tissue Cross Sections

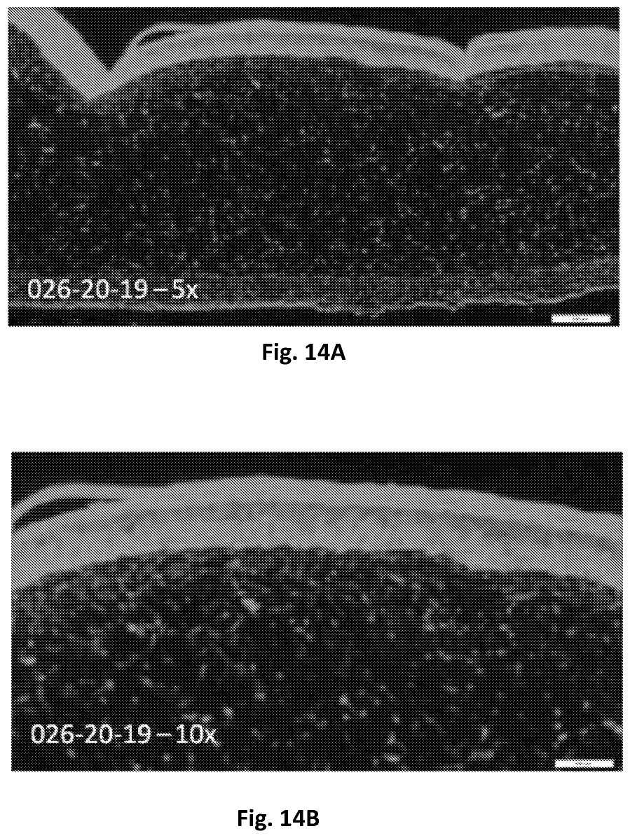

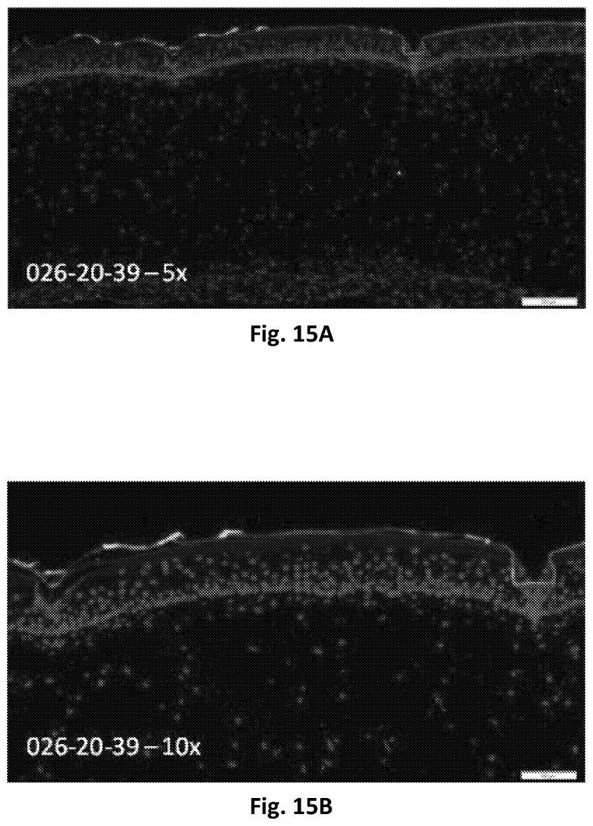

At each timepoint, EFT-400 tissues treated with fluorescently labeled test materials were fixed in neutral buffered formalin and slides containing cross sections were prepared using standard histological methods. Unstained, deparaffinized slides were counterstained with DAPI (to visualize nuclei) and imaged using an Olympus VS-120 automated slide scanner system with an XM10 fluorescent camera. All sections were imaged using DAPI (455 nm), FITC (518 nm), and TRITC (615 nm) filters. The dH2O control tissues, which contain no fluorescently labeled material, were used to establish a scaling threshold by which to evaluate the fluorescent signal in the treated tissues. FIGS. 14A and 14B illustrate the cross sections of EFT-400 tissues exposed to low MW Silk (RITC labeled) for 2×5 hrs counterstained with DAPI. 5× magnification image (FIG. 14A) shows full tissue thickness and 10× magnification image (FIG. 14B) focuses on epidermis. FIGS. 15A and 15B illustrate the cross sections of EFT-400 tissues exposed to mid MW Silk (FITC labeled) for 2×5 hrs counterstained with DAPI. 5× magnification image (FIG. 15A) shows full tissue thickness and 10× magnification image (FIG. 15B) focuses on epidermis.

The results of this study show evidence for time-dependent permeation of the RITC-labeled low MW silk test material in EFT-400 tissues. By contrast, almost no permeation was observed in EFT-400 tissues treated with FITC labeled mid MW silk. There is very good correlation in this study between the observations made in the images of tissue cross sections and the quantification of the fluorescent signal measured in the culture media collected from EFT-400 tissues at each time point.

### Example 5: Petrolatum Replacement

Petrolatum is FDA-approved for OTC use as a skin-protectant. Its occlusive nature creates a physical barrier that prevents moisture loss from the skin, and may sooth cuts and abrasions, treat rashes and eczema, etc. However, there exist concerns with petrolatum, such as that it is made from crude oil, it may be toxic to many forms of life, its extraction may fuel climate change, it does not hydrate the skin, it can be greasy and heavy, and its manufacturing process includes polycyclic aromatic hydrocarbons (PAHs) that have potential links to breast cancer.

Silk fibroin compositions described herein can act as a skin barrier, and/or be formulated as a skin barrier formulation. Without wishing to be bound by any particular theory, it is believed that Claudin-1 reduction disrupts tight junction function leading to epidermal barrier defects, for example in atopic dermatitis. Benedetto et al, JACI, 2010, Bergmann et al, Scientific Reports, 2020. Also without wishing to be bound by any particular theory, it is believe that Claudin-1 deficient mice have severe dehydration, wrinkled skin, and increased epidermal permeability. Furuse et al, JCB, 2002.

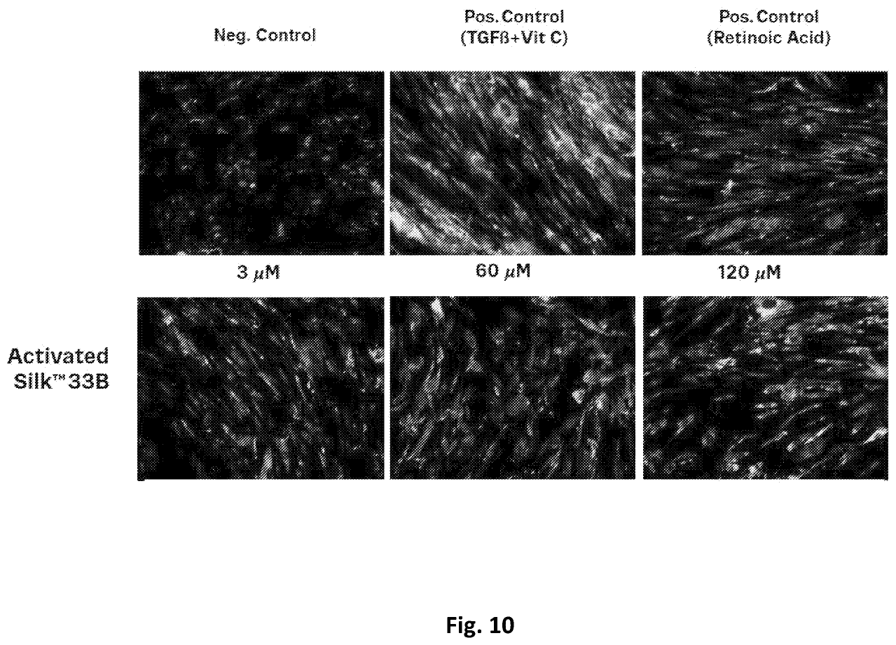

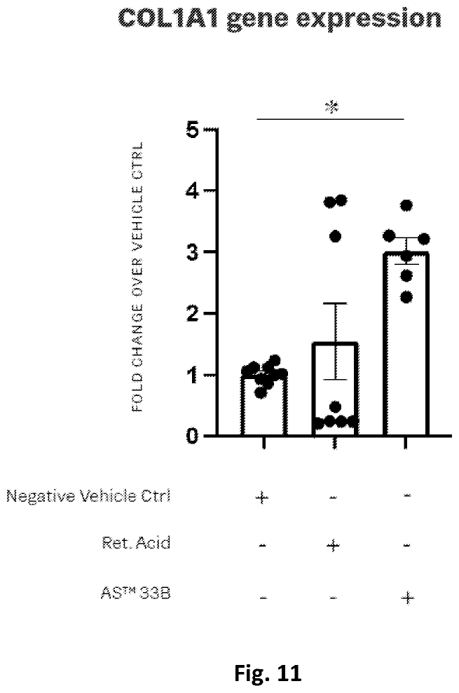

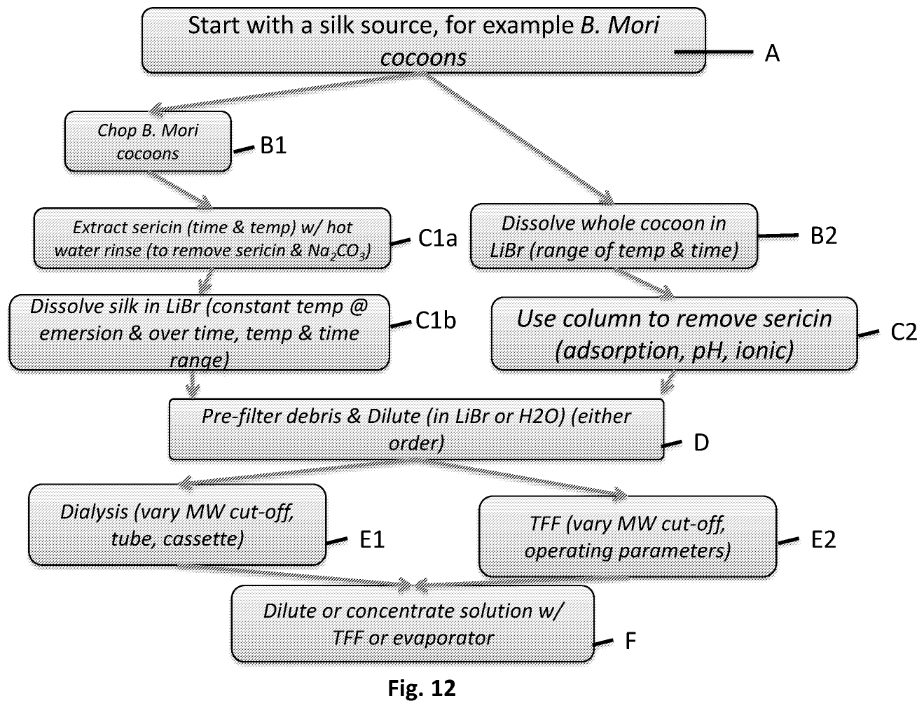

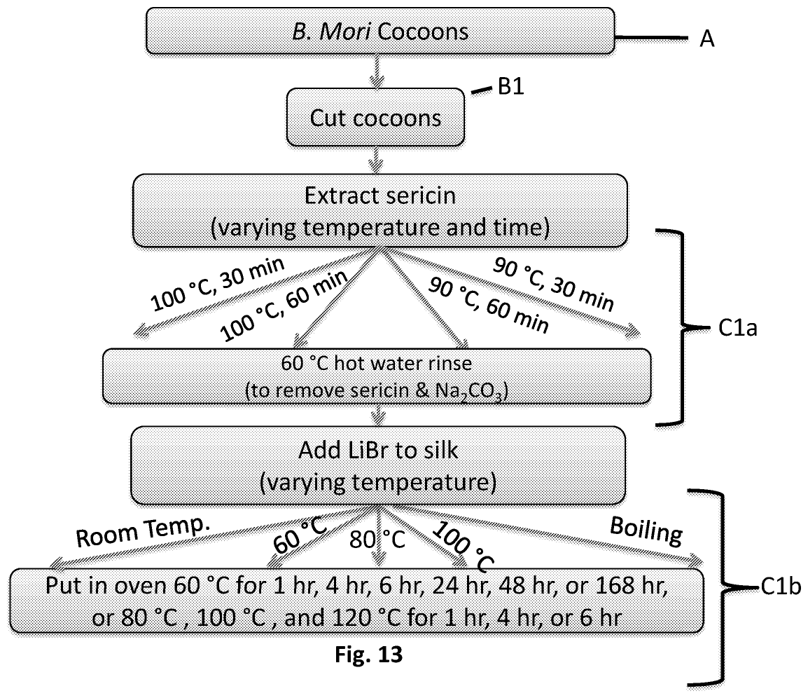

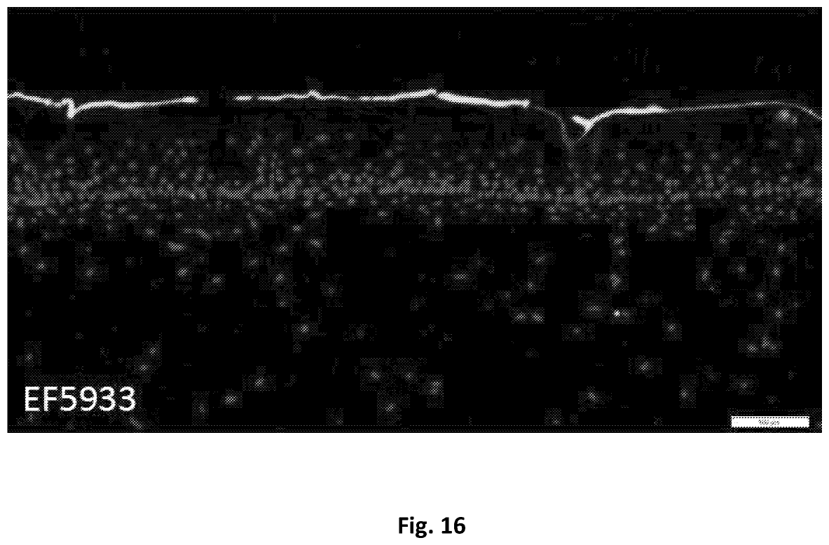

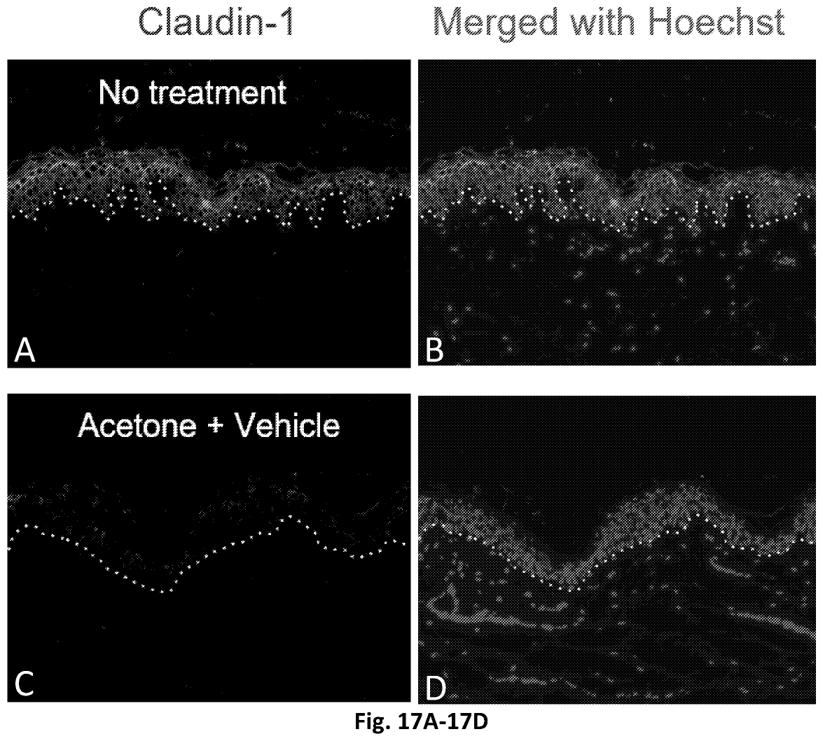

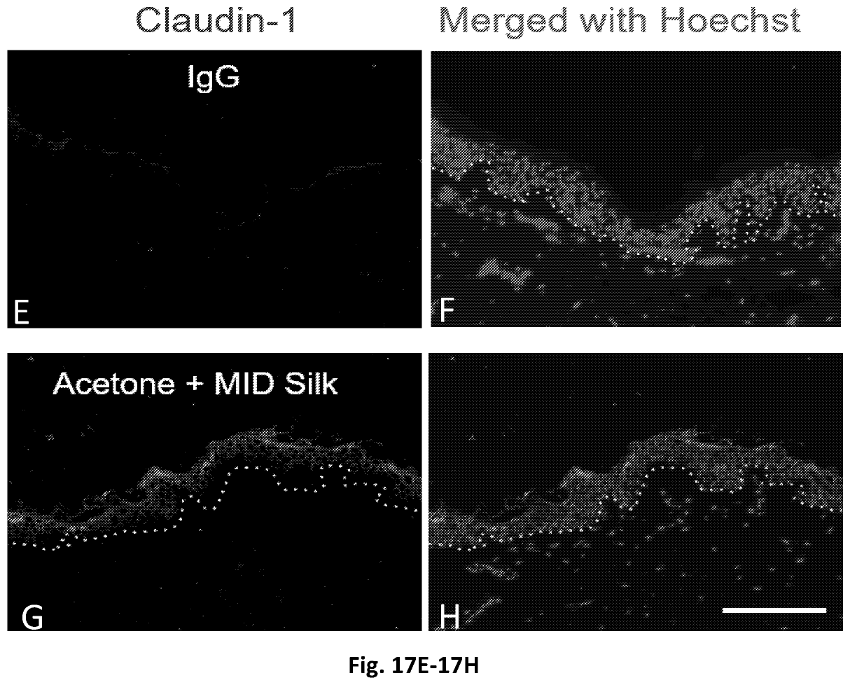

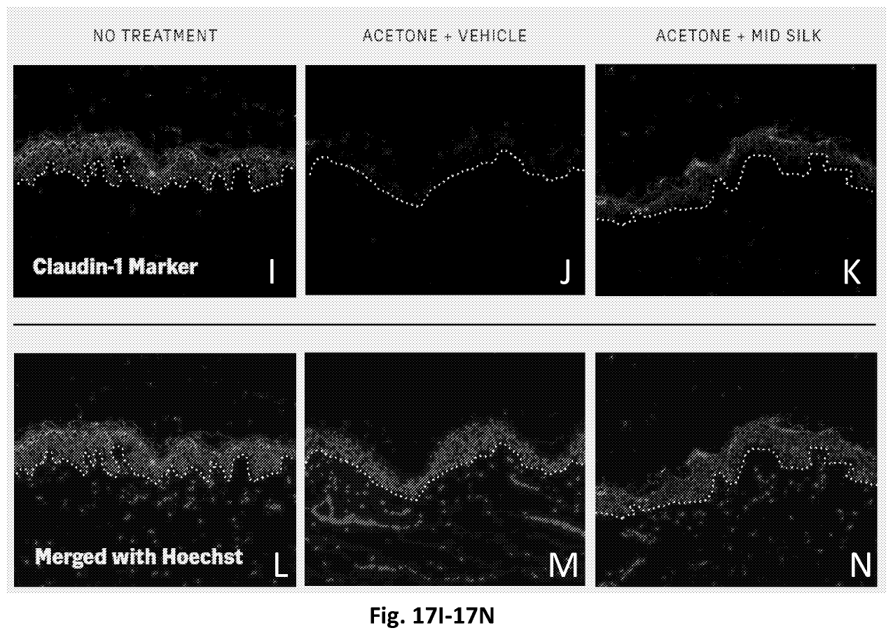

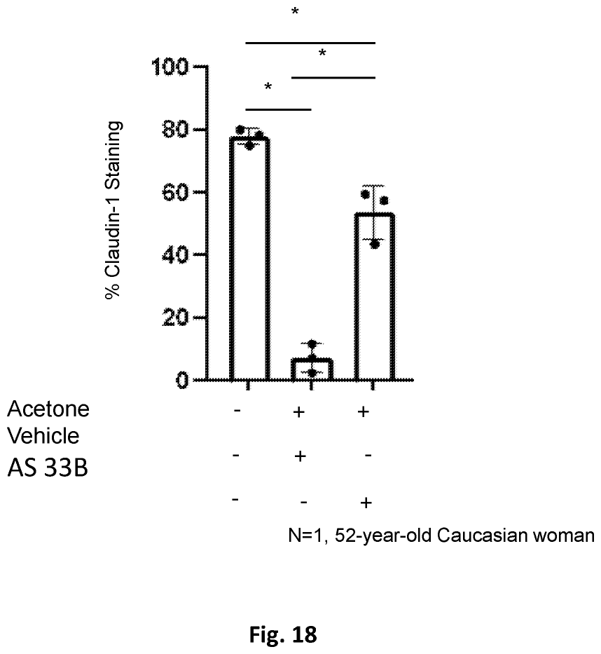

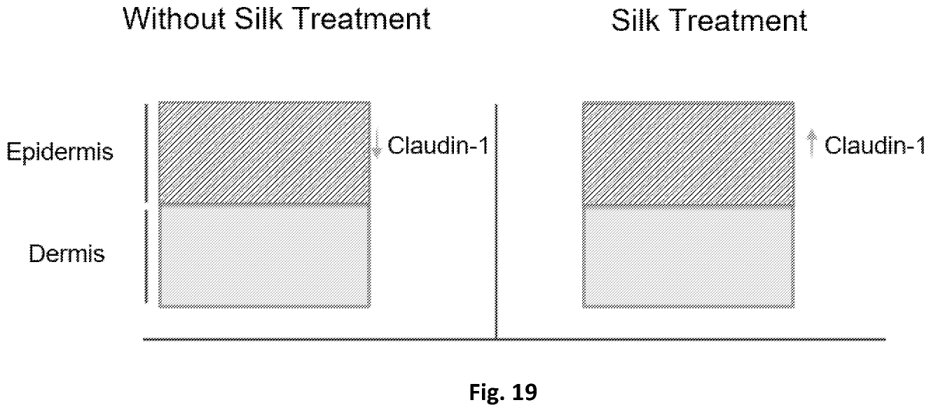

FIG. 16 is a microscopic cross-section of silk fibroin described herein (Activated Silk™)-treated EpiDermFT tissue; fluorescence imaging of fluorescently tagged silk fibroin. FIGS. 17A-17N illustrates that silk fibroin described herein restores claudin-1 expression in damaged human skin (N=1, 52-year-old Caucasian woman). FIG. 18 illustrates that silk fibroin described herein restores claudin-1 expression in damaged human skin. FIG. 19 illustrates how silk fibroin described herein restores claudin-1 expression to improve skin barrier. FIG. 10 illustrates how silk fibroin described herein stimulates collagen production in human dermal fibroblasts; silk fibroin described herein exhibit similar stimulation of Collagen 1 in human dermal fibroblasts as retinoic acid. FIG. 11 illustrates how silk fibroin described herein upregulates COL1A1 gene expression in human dermal fibroblasts; silk fibroin described herein upregulates COL1A1 gene expression in human dermal fibroblasts; Quantitative PCR on COL1A1 in silk- and retinoic acid-treated human dermal fibroblasts at 8 h after treatment; n=2 per group; >8-fold increase in TGF-β+Vit C treated human dermal fibroblasts (served as a positive Ctrl) ASTS.

Without wishing to be bound by any particular theory, it is believed that silk fibroin described herein, for example, and without limitation, Activated Silk™ 33B, is retained on the surface of the stratum corneum. Silk fibroin described herein can be used for skin barrier applications. In some embodiments, silk fibroin described herein is retained in the stratum corneum layer after 5× rinses with H2O

FIGS. 17A-17N illustrate that silk fibroin restores claudin-1 expression in damaged human skin.

All patents, patent applications, and published references cited herein are hereby incorporated by reference in their entirety. While the methods of the present disclosure have been described in connection with the specific embodiments thereof, it will be understood that it is capable of further modification. Further, this application is intended to cover any variations, uses, or adaptations of the methods of the present disclosure, including such departures from the present disclosure as come within known or customary practice in the art to which the methods of the present disclosure pertain.

### Example 6: Dosage of Silk Polypeptide Compositions to Treat Skin Conditions

### Introduction

Skin provides the physical barrier that regulates loss of fluid and electrolytes by maintaining a highly stratified structure. This barrier in the epidermis forms the outermost layer of the stratified epithelium and is predominantly composed of keratinocytes. Keratinocyte cells undergo different stages of differentiation in response to calcium, and as a result, move from a basal layer to the top-most layer of the epithelium. Part of the skin barrier process includes formation of skin tight junctions between the keratinocytes, which in turn maintains skin integrity. Members of the claudin protein family, specifically Claudin-1, are well known to play a crucial role in this tight junction formation and maintaining cell-cell adhesion and integrity.

Fibroblast are found in the dermal compartment of skin and are responsible for collagen production. Reduction of collagen 1 in the skin causes wrinkles and deterioration of skin health. Boosting of collagen 1 will restore skin health and lead to the disappearance of wrinkles. Retinol, which is used as a cosmeceutical to address collagen 1 production in the skin, must be converted to retinoic acid to be effective while it is unstable and has increased toxicity. Here the use and dosage of silk formulations that promote the upregulation of claudin-1 is described, collagen, keratinocyte cell migration to promote wound healing and provide some details about the potential mechanism of action. Silk was found to promote skin barrier function and downregulated genes that promote inflammation. Hence, the described silk polypeptide compositions could be used to treat a wide range of skin conditions.

### Results and Discussion

### Silk Upregulates Expression of Claudin-1 in Keratinocyte Cultures.

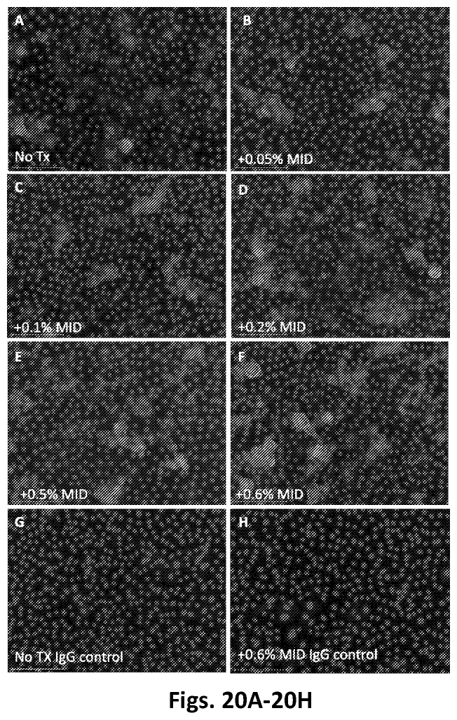

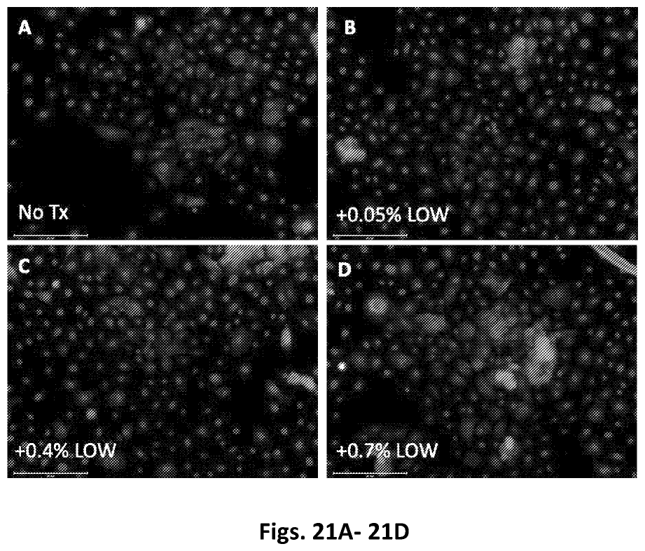

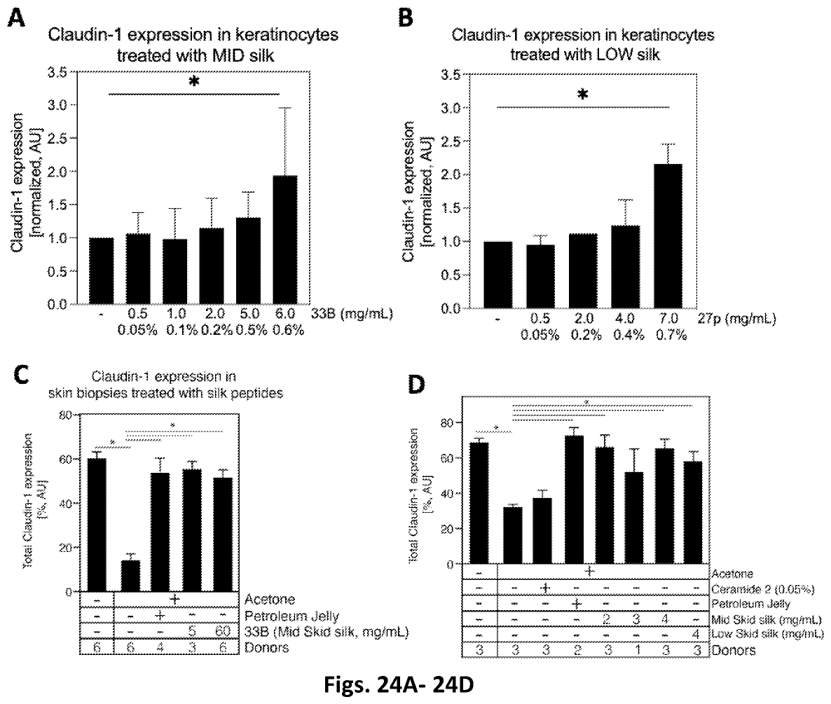

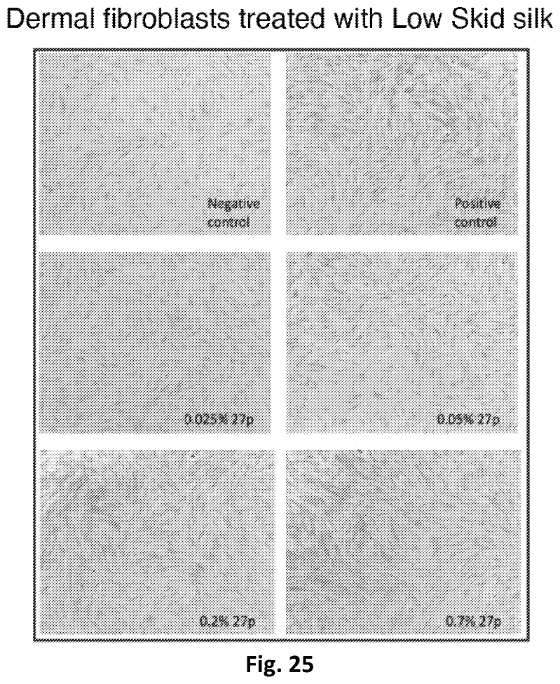

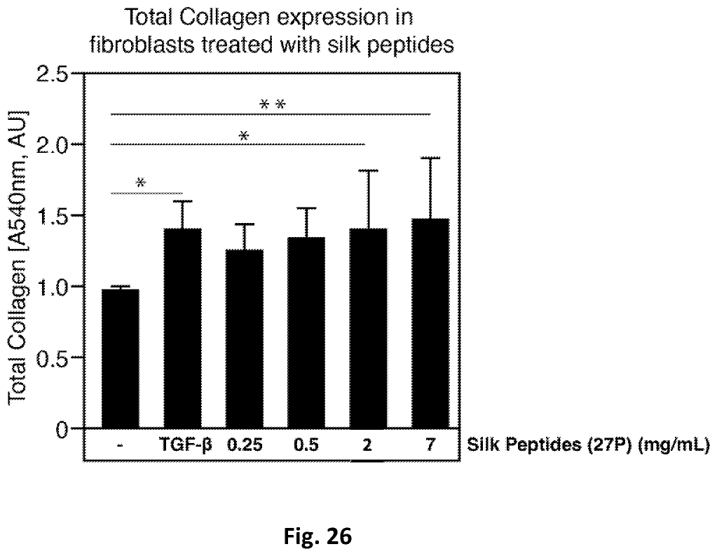

To explore the effect of silk treatment on skin barrier function, the focus was on on the tight junction protein, claudin-1 since it is crucial for proper skin barrier function. To test the effect of soluble silk polypeptides on claudin-1 expression, a dose response assessment of MID SKID and LOW SKID silk was performed with a primary keratinocyte cell system. Keratinocytes are an essential population of cells that are found in the skin and are responsible for the formation of the epidermis. Malfunction of keratinocytes leads to various skin diseases such as psoriasis and atopic dermatitis. Primary keratinocytes were treated with a concentration range of MID SKID silk (0.05%-0.6% w/v) and LOW SKID silk (0.05%-0.7% w/v) and stained with human anti-mouse monoclonal claudin-1 primary antibody (sc-81796, Santa Cruz) for claudin-1 expression (FIGS. 20A-20H, 24A). When the 33B silk polypeptide concentration reached 6 mg/mL (0.6% w/v) claudin-1 expression was upregulated (FIGS. 20, 24A). Preliminary results showed that addition of 7 mg/mL (0.7% w/v) of 27p silk polypeptide resulted in increased claudin-1 expression (FIGS. 21, 24B). It was concluded that mid skid silk at a concentration of 6 mg/mL upregulates claudin-1 expression in primary keratinocyte cell cultures and has potential to promote skin barrier function.

### Silk Upregulates Expression of Claudin-1 in Skin Biopsies.

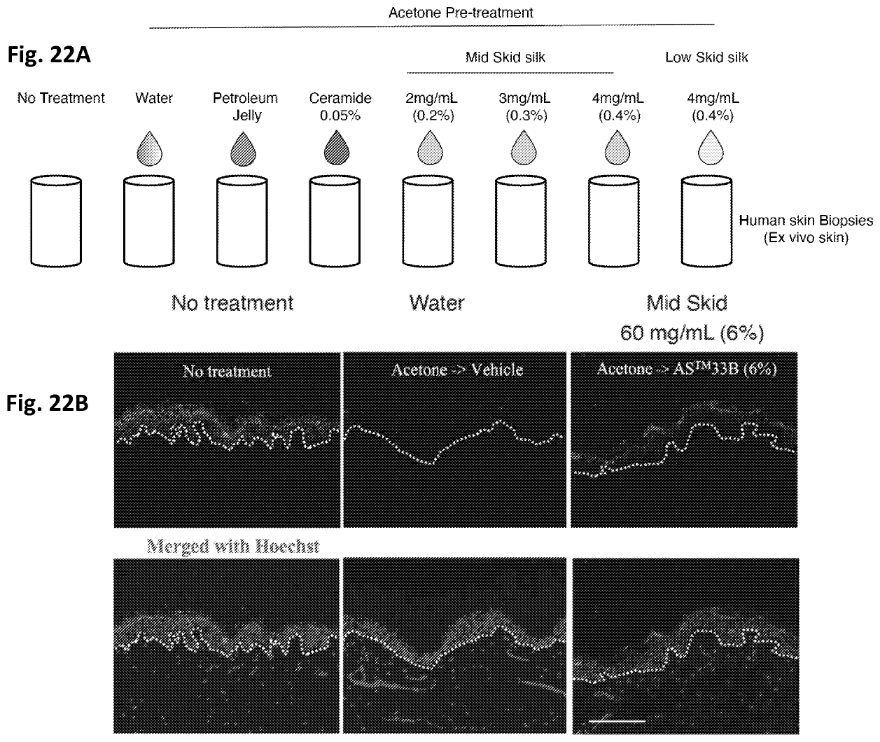

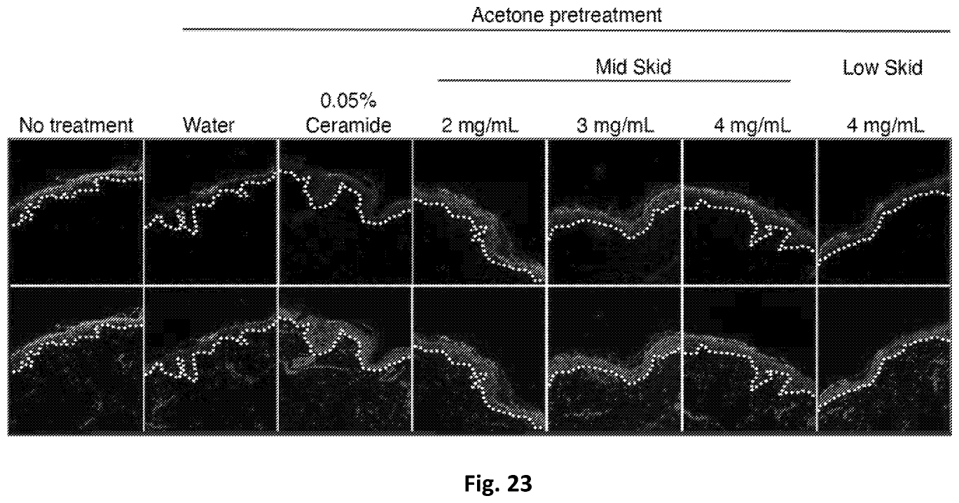

To investigate whether the effect of mid skid (33B) silk polypeptides in primary keratinocyte cultures impacts claudin-1 in the human skin, skin biopsies were used. The biopsies were taken from female skin donors with an age range of 30-60 years. Skin biopsies were treated with ≥99.5% % acetone to deplete the claudin-1 (FIGS. 22A-22B). Reduced claudin-1 immunoreactivity was noted in acetone-treated human skin compared to untreated biopsies, demonstrating the success of the model establishment. After the acetone treatment, skin biopsies were treated with water, petroleum jelly, ceramide and two concentrations of mid skid silk and one concentration of low skid silk. Data from 30- to 60-year-old female human skin donors show that 33B silk polypeptide composition at a minimal concentration of 2 mg/mL restored claudin-1 expression in acetone-pretreated skin at levels comparable to petroleum jelly (FIGS. 23, 24C, 24D) Low skid (27P) silk at 4 mg/mL also restored claudin-1 levels (FIGS. 23, 24C, 24D). Ceramide didn't have any noticeable effect on claudin-1 in the system (FIGS. 23, 24D).

### Silk Upregulates Expression of Collagen in Dermal Fibroblast Cultures.

Collagen is known to play a crucial role in skin health, with collagen-1 constituting ˜70% of the dermal extracellular matrix. The effect of Low Skid (27P) silk polypeptides on collagen production in human dermal fibroblasts was therefore tested as a dose-response study. Primary dermal fibroblasts were treated with a concentration range of Low Skid (27P) silk polypeptides (0.025%-0.7% w/v) and assessed for total collagen using sirius red spectrophotometric analysis. Since TGF-β is a well-known promoter of collagen production, TGF-β treated fibroblast cells were used as a positive control. Sirius red dye stained cells were visualized using bright-field microscopy and additionally quantified by spectrometric analysis at OD 540. Results show significantly increased collagen production in human fibroblast cell cultures treated with 0.2% and 0.7% Low Skid (27P) silk polypeptides (FIGS. 25 & 26) in the absence of vitamin C. However, no significant differences in collagen production were observed with 27p treated fibroblast cells in the presence of 20 μL/mL vitamin C (data not shown).

### Example 7: Low Skid (27P) Silk Polypeptides Accelerate Cell Migration in Wound Closure In Vitro Models

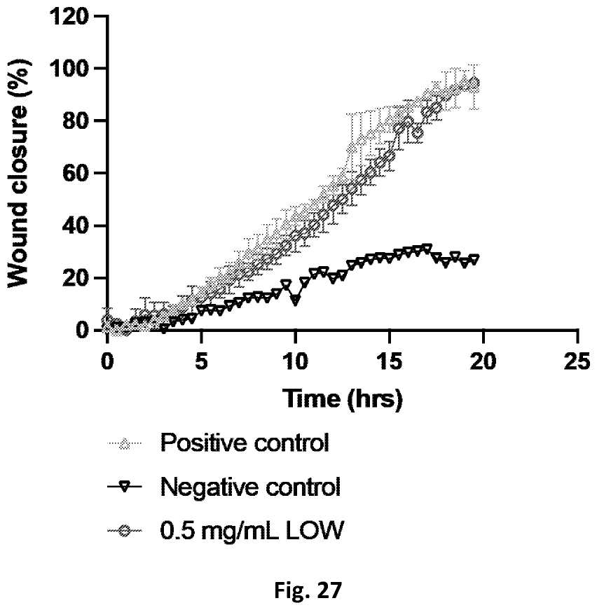

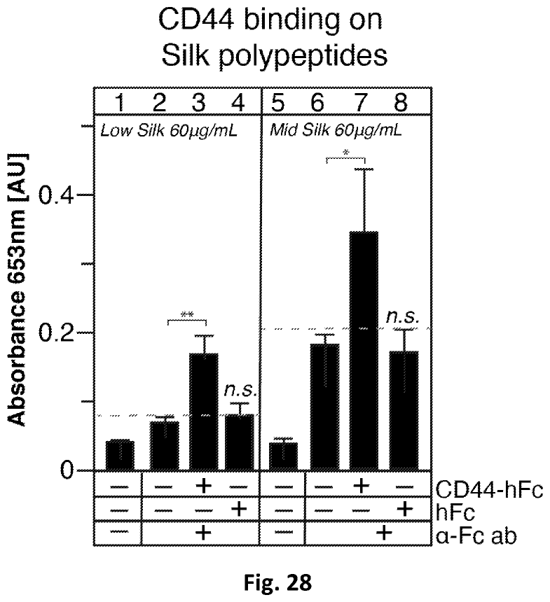

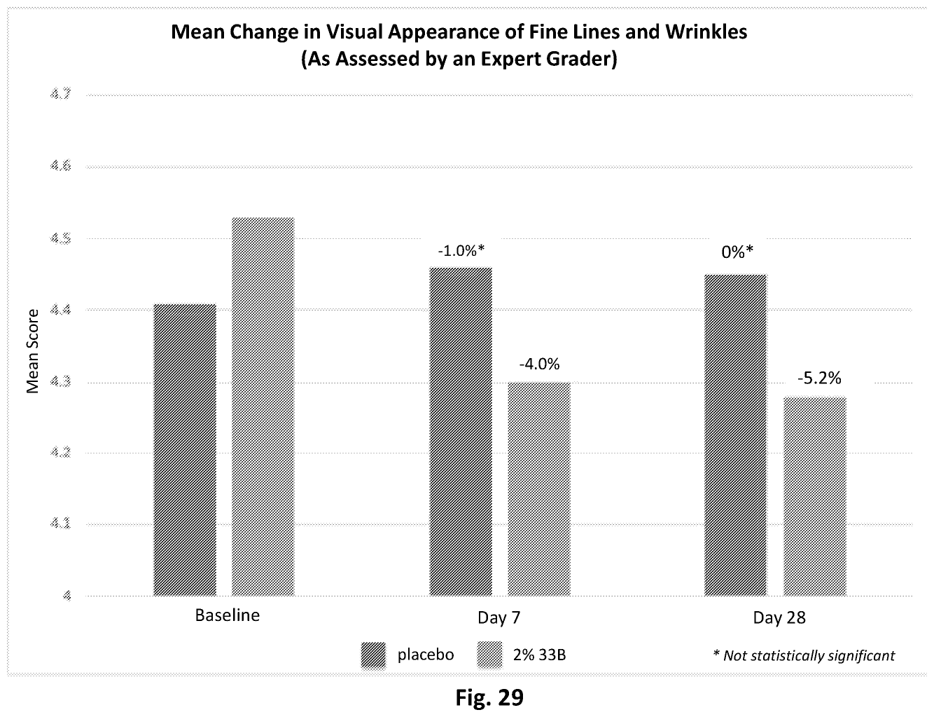

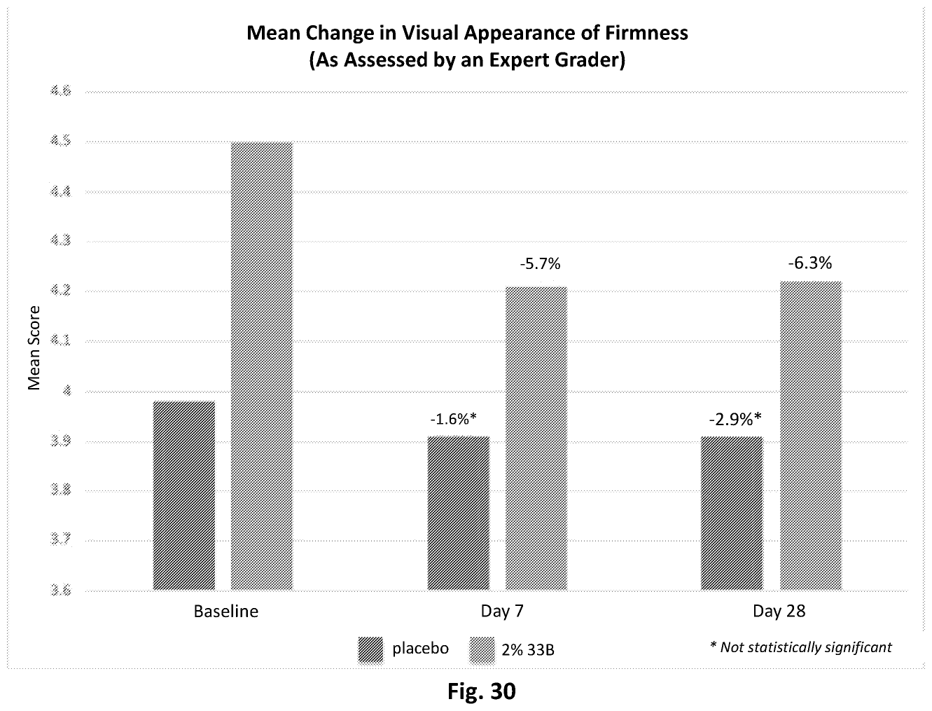

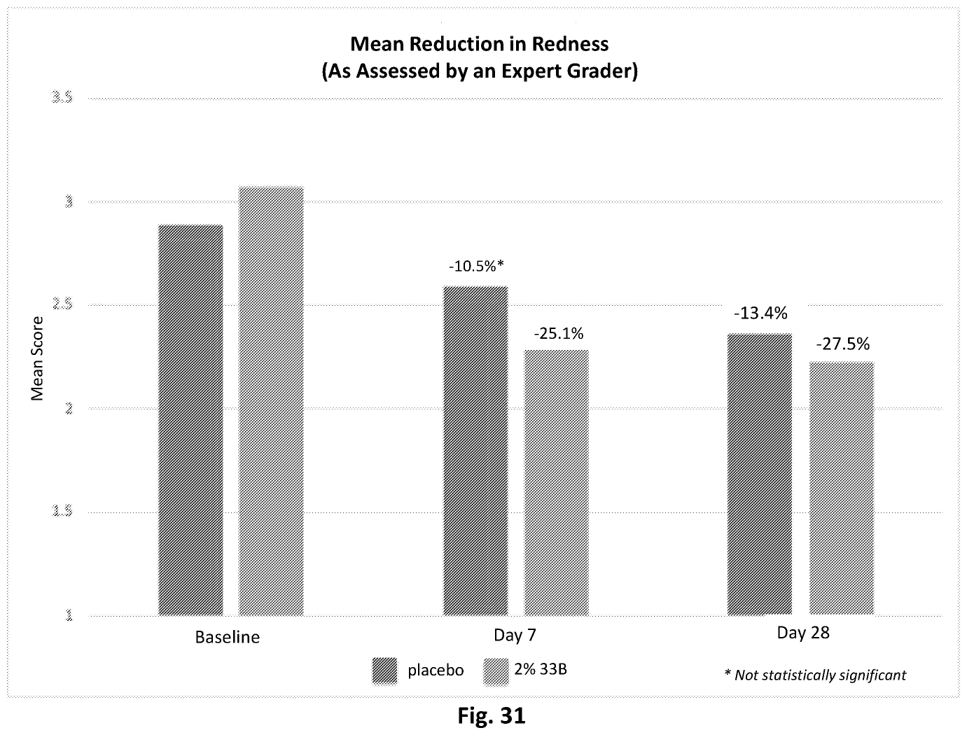

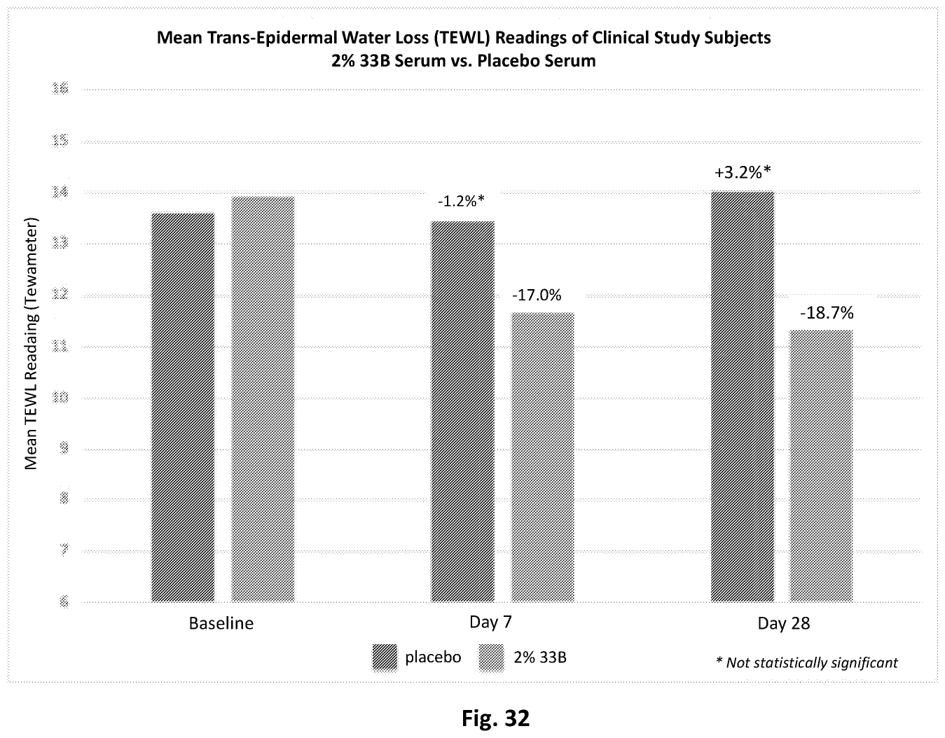

One crucial function of skin epithelia is wound healing. Wound healing requires cell division and migration to fill in the gaps that are created with the breakage of the skin. To test whether silk compositions affect any aspect of wound healing wound closure in vitro assays were used. First, a layer of human primary keratinocytes was created. A scratch was generated that disrupted the continuum of the keratinocyte layer and cells were allowed to occupy the free space. When keratinocytes were treated with medium that contained low skid (27P) silk polypeptides they migrated and filled in the available space of the scratch as fast as the keratinocytes treated with media that contained serum and growth factors (FIG. 27). Hence, it was concluded that low skid (27P) silk polypeptides activate signaling pathways in epithelial cells that accelerate wound healing.

### Example 8: CD44 Receptor Interacts with Low (27P) and Mid (33B) Silk In Vitro

The CD44 receptor has been implicated in wound healing and collagen regulation. To test whether silk polypeptides can interact with human CD44 receptor a solid phase protein-protein interaction assay was developed that is based on the principles of Enzyme-Linked Immunosorbent Assay (ELISA). Briefly, silk polypeptides were immobilized on the surface of the 96-well plate and interaction with a human CD44-hFc construct was measured. The Fc moiety was used to detect bound CD44 on silk with a secondary anti-human Fc IgG. When silk was treated with an isolated human Fc fragment binding was non-significant (FIG. 28). Contrary to this, CD44-hFc displayed significantly higher binding on both immobilized low skid (27P) and mid skid (33B) silk. This result demonstrates that low (27P) and mid (33B) skid silk polypeptides can interact with CD44 in vitro.

### Example 9: Silk Downregulates Expression of Genes Involved in Inflammatory Response in Skin

Another mechanism that regulates skin and epithelial homeostasis is inflammation. To investigate whether silk formulations affect inflammatory pathways skin biopsies were treated with mid skid (33B) silk polypeptides and looked for differential expression of genes that regulate the mechanisms of inflammation. Skin biopsies were treated with 99.5% acetone and then 5 mg/mL and 60 mg/mL mid skid (33B) silk polypeptides were applied. After treatment RNA was extracted and analyzed for gene expression. Several genes involved in inflammation were downregulated in the skin biopsies treated with 5 mg/mL and 60 mg/mL mid skid (33B) silk polypeptides in relation to skin biopsies treated with vehicle (water) only (Table 2). These results show that mid skid (33B) silk polypeptides can down-regulate inflammation and promote skin health.

TABLE 2
Transcriptomic analysis of skin biopsies treated with mid skin (33B) silk. The
genes on this table are involved in inflammatory pathways and were downregulated in skin
biopsies after treatment with 99.5% acetone and a subsequent treatment with mid skid (33B) silk
at 5 mg/mL (0.5%) (TrtA) and 60 mg/mL (6%) (TrtB).

| | | TrtA | TrtA | TrtB | TrtB |
|---|---|---|---|---|---|
| Gene | Description | log2fold | p value | Log2fold | P value |
| ADA2 | Studies suggest that it acts as a growth factor, which means | −1.17 | 0.0124 | −1.51 | 0.004 |
| | that it stimulates cell growth and division. In particular, the | | | | |
| | enzyme appears to be involved in the growth and | | | | |
| | development of certain immune system cells, including | | | | |
| | macrophages, which are a type of white blood cell that plays | | | | |
| | a critical role in inflammation. Inflammation is a normal | | | | |
| | immune system response to injury and foreign invaders (such | | | | |
| | as bacteria). Some macrophages are pro-inflammatory, | | | | |
| | meaning they promote inflammation, while others are anti- | | | | |
| | inflammatory, meaning they reduce inflammation. | | | | |
| CD209 | This gene encodes a C-type lectin that functions in cell | −1.54 | 0.0059 | −1.38 | 0.026 |
| | adhesion and pathogen recognition. This receptor recognizes | | | | |
| | a wide range of evolutionarily divergent pathogens with a | | | | |
| | large impact on public health, including leprosy and | | | | |
| | tuberculosis mycobacteria, the Ebola, hepatitis C, HIV-1 and | | | | |
| | Dengue viruses, and the SARS-COV acute respiratory | | | | |
| | syndrome coronavirus. The protein is organized into four | | | | |
| | distinct domains: a C-terminal carbohydrate recognition | | | | |
| | domain, a flexible tandem-repeat neck domain, a | | | | |
| | transmembrane region and an N-terminal cytoplasmic domain | | | | |
| | involved in internalization. This gene is closely related in | | | | |
| | terms of both sequence and function to a neighboring gene, | | | | |
| | CLEC4M (Gene ID: 10332), also known as L-SIGN. The two | | | | |
| | genes differ in viral recognition and expression patterns, with | | | | |
| | this gene showing high expression on the surface of dendritic | | | | |
| | cells. Polymorphisms in the neck region are associated with | | | | |
| | protection from HIV-1 infection, while single nucleotide | | | | |
| | polymorphisms in the promoter of this gene are associated | | | | |
| | with differing resistance and susceptibility to and severity of | | | | |
| | infectious disease, including rs4804803, which is associated | | | | |
| | with SARS severity. [provided by RefSeq, May 2020] | | | | |
| CD300E | This gene encodes a member of the CD300 glycoprotein | −0.99 | 0.0141 | −1.15 | 0.019 |
| | family of cell surface proteins expressed on myeloid cells. | | | | |
| | The protein interacts with the TYRO protein tyrosine kinase- | | | | |
| | binding protein and is thought to act as an activating receptor. | | | | |
| | [provided by RefSeq, November 2012] | | | | |
| CR1 | Membrane immune adherence receptor that plays a critical | −2.97 | 0.0188 | −2.68 | 0.021 |
| | role in the capture and clearance of complement-opsonized | | | | |
| | pathogens by erythrocytes and monocytes/macrophages | | | | |
| | (PubMed: 2963069). Mediates the binding by these cells of | | | | |
| | particles and immune complexes that have activated | | | | |
| | complement to eliminate them from the circulation | | | | |
| | (PubMed: 2963069). Acts also in the inhibition of | | | | |
| | spontaneous complement activation by impairing the | | | | |
| | formation and function of the alternative and classical | | | | |
| | pathway C3/C5 convertases, and by serving as a cofactor for | | | | |
| | the cleavage by factor I of C3b to iC3b, C3c and C3d, g, and | | | | |
| | of C4b to C4c and C4d (PubMed: 2972794, 8175757). Also | | | | |
| | plays a role in immune regulation by contributing, upon | | | | |
| | ligand binding, to the generation of regulatory T cells from | | | | |
| | activated helper T cells (PubMed: 25742728). | | | | |
| | ( CR1_HUMAN, P17927) | −1.03 | 0.0274 | −1.28 | 0.005 |
| F13A1 | This gene encodes the coagulation factor XIII A subunit. | | | | |
| | Coagulation factor XIII is the last zymogen to become | | | | |
| | activated in the blood coagulation cascade. Plasma factor XIII | | | | |
| | is a heterotetramer composed of 2 A subunits and 2 B | | | | |
| | subunits. The A subunits have catalytic function, and the B | | | | |
| | subunits do not have enzymatic activity and may serve as | | | | |
| | plasma carrier molecules. Platelet factor XIII is comprised | | | | |
| | only of 2 A subunits, which are identical to those of plasma | | | | |
| | origin. Upon cleavage of the activation peptide by thrombin | | | | |
| | and in the presence of calcium ion, the plasma factor XIII | | | | |
| | dissociates its B subunits and yields the same active enzyme, | | | | |
| | factor XIIIa, as platelet factor XIII. This enzyme acts as a | | | | |
| | transglutaminase to catalyze the formation of gamma- | | | | |
| | glutamyl-epsilon-lysine crosslinking between fibrin | | | | |
| | molecules, thus stabilizing the fibrin clot. It also crosslinks | | | | |
| | alpha-2-plasmin inhibitor, or fibronectin, to the alpha chains | | | | |
| | of fibrin. Factor XIII deficiency is classified into two | | | | |
| | categories: type I deficiency, characterized by the lack of both | | | | |
| | the A and B subunits; and type II deficiency, characterized by | | | | |
| | the lack of the A subunit alone. These defects can result in a | | | | |
| | lifelong bleeding tendency, defective wound healing, and | | | | |
| | habitual abortion. [provided by RefSeq, July 2008] | | | | |
| KL | This gene encodes a type-I membrane protein that is related | −1.99 | 0.0082 | −2.04 | 0.003 |
| | to beta-glucosidases. Reduced production of this protein has | | | | |
| | been observed in patients with chronic renal failure (CRF), | | | | |
| | and this may be one of the factors underlying the degenerative | | | | |
| | processes (e.g., arteriosclerosis, osteoporosis, and skin | | | | |
| | atrophy) seen in CRF. Also, mutations within this protein | | | | |
| | have been associated with ageing and bone loss. [provided by | | | | |
| | RefSeq, July 2008] | | | | |
| PDGFRA | Tyrosine-protein kinase that acts as a cell-surface receptor for | −0.88 | 0.0374 | −0.78 | 0.034 |
| | PDGFA, PDGFB and PDGFC and plays an essential role in | | | | |
| | the regulation of embryonic development, cell proliferation, | | | | |
| | survival and chemotaxis. Depending on the context, promotes | | | | |
| | or inhibits cell proliferation and cell migration. Plays an | | | | |
| | important role in the differentiation of bone marrow-derived | | | | |
| | mesenchymal stem cells. Required for normal skeleton | | | | |
| | development and cephalic closure during embryonic | | | | |
| | development. Required for normal development of the | | | | |
| | mucosa lining the gastrointestinal tract, and for recruitment of | | | | |
| | mesenchymal cells and normal development of intestinal villi. | | | | |
| | Plays a role in cell migration and chemotaxis in wound | | | | |
| | healing. Plays a role in platelet activation, secretion of | | | | |
| | agonists from platelet granules, and in thrombin-induced | | | | |
| | platelet aggregation. Binding of its cognate ligands— | | | | |
| | homodimeric PDGFA, homodimeric PDGFB, heterodimers | | | | |
| | formed by PDGFA and PDGFB or homodimeric PDGFC— | | | | |
| | leads to the activation of several signaling cascades; the | | | | |
| | response depends on the nature of the bound ligand and is | | | | |
| | modulated by the formation of heterodimers between | | | | |
| | PDGFRA and PDGFRB. Phosphorylates PIK3R1, PLCG1, | | | | |
| | and PTPN11. Activation of PLCG1 leads to the production of | | | | |
| | the cellular signaling molecules diacylglycerol and inositol | | | | |
| | 1,4,5-trisphosphate, mobilization of cytosolic Ca(2+) and the | | | | |
| | activation of protein kinase C. Phosphorylates PIK3R1, the | | | | |
| | regulatory subunit of phosphatidylinositol 3-kinase, and | | | | |
| | thereby mediates activation of the AKTI signaling pathway. | | | | |
| | Mediates activation of HRAS and of the MAP kinases | | | | |
| | MAPK1/ERK2 and/or MAPK3/ERK1. Promotes activation | | | | |
| | of STAT family members STATI, STAT3 and STAT5A | | | | |
| | and/or STAT5B. Receptor signaling is down-regulated by | | | | |
| | protein phosphatases that dephosphorylate the receptor and its | | | | |
| | down-stream effectors, and by rapid internalization of the | | | | |
| | activated receptor | −1.34 | 0.0015 | −1.26 | 0.002 |
| TLR4 | Cooperates with LY96 and CD14 to mediate the innate | | | | |
| | immune response to bacterial lipopolysaccharide (LPS) | | | | |
| | (PubMed: 27022195). Acts via MYD88, TIRAP and TRAF6, | | | | |
| | leading to NF-kappa-B activation, cytokine secretion and the | | | | |
| | inflammatory response (PubMed: 9237759, | | | | |
| | PubMed: 10835634, PubMed: 27022195, PubMed: 21393102). | | | | |
| | Also involved in LPS-independent inflammatory responses | | | | |
| | triggered by free fatty acids, such as palmitate, and Ni(2+). | | | | |
| | Responses triggered by Ni(2+) require non-conserved | | | | |
| | histidines and are, therefore, species-specific | | | | |
| | (PubMed: 20711192). Both M. tuberculosis HSP70 (dnaK) | | | | |
| | and HSP65 (groEL-2) act via this protein to stimulate NF- | | | | |
| | kappa-B expression (PubMed: 15809303). In complex with | | | | |
| | TLR6, promotes sterile inflammation in | | | | |
| | monocytes/macrophages in response to oxidized low-density | | | | |
| | lipoprotein (oxLDL) or amyloid-beta 42. In this context, the | | | | |
| | initial signal is provided by oxLDL- or amyloid-beta 42- | | | | |
| | binding to CD36. This event induces the formation of a | | | | |
| | heterodimer of TLR4 and TLR6, which is rapidly internalized | | | | |
| | and triggers inflammatory response, leading to the NF-kappa- | | | | |
| | B-dependent production of CXCLI, CXCL2 and CCL9 | | | | |
| | cytokines, via MYD88 signaling pathway, and CCL5 | | | | |
| | cytokine, via TICAMI signaling pathway, as well as IL1B | | | | |
| | secretion. Binds electronegative LDL (LDL(−)) and mediates | | | | |
| | the cytokine release induced by LDL(−) (PubMed: 23880187). | | | | |
| | Stimulation of monocytes in vitro with M. tuberculosis PstS1 | | | | |
| | induces p38 MAPK and ERK1/2 activation primarily via | | | | |
| | TLR2, but also partially via this receptor | | | | |
| | (PubMed: 16622205, 10835634, 15809303, 17478729, 2003 | | | | |
| | 7584, 20711192, 23880187, 27022195, 9237759). Activated | | | | |
| | by the signaling pathway regulator NMI which acts as | | | | |
| | damage-associated molecular patterns (DAMPs) in response | | | | |
| | to cell injury or pathogen invasion, therefore promoting | | | | |
| | nuclear factor NF-kappa-B activation (PubMed: 29038465). | | | | |
| | (TLR4_HUMAN, 000206) | | | | |

### Example 10: Topically-Applied Silk Results in Reduction of Trans-Epidermal Water Loss (TEWL)

At the concentration evaluated in the study (20%), Activated Silk 33B significantly reduced trans-epidermal water loss, validating that the benefits observed at the cellular and ex-vivo skin levels translate to measurable skin barrier benefits when topically applied. (Study participants using the placebo serum did not experience any reduction in TEWL).

Study participants using 33B also experienced significant improvements in skin texture and firmness, as well as reduction in redness, fine lines and wrinkles, as assessed by an expert grader. Self-perception feedback pertaining to the ingredient performance in a serum format was also promising (Table 1).

TABLE 1
Self-Perception Questionnaire responses after application of S33B-18.

| | Initial | 7 | 28 | |
|---|---|---|---|---|
| Question | (1x) | days | days | [symbol] |
| Q1. Skin appears replenished, healthier and younger looking after using the product. | 55.88 | 84.38 | 93.75 | [symbol] |
| Q2. Skin looks and feels significantly more hydrated after using the product. | 73.53 | 78.13 | 84.38 | [symbol] |
| Q3. Skin looks and feels significantly smoother after using the product. | 58.82 | 84.38 | 90.63 | [symbol] |
| Q4. Skin's roughness and dryness are reduced after using the product | 61.76 | 81.25 | 93.75 | [symbol] |
| Q5. Skin radiance is significantly improved after using the product. | 58.82 | 62.5 | 84.38 | [symbol] |
| Q6. Test product reduces skin redness and has a soothing and calming effect on the redness of my skin. | 55.88 | 67.74 | 78.13 | [symbol] |
| Q7. Test product significantly improved skin's overall appearance after using the product. | 52.94 | 78.13 | 93.75 | [symbol] |
| Q8. Skin overall health is significantly improved after using the test product. | 55.88 | 67.74 | 90.63 | [symbol] |
| Q9. Skin brightness is significantly improved after using the product. | 67.65 | 71.88 | 84.38 | [symbol] |
| Q10. Skin clarity is significantly improved after using the product, leaving a healthy and even tone. | 58.82 | 84.38 | 78.13 | [symbol] |
| Q11. Skin texture is significantly improved after using the product. | 55.88 | 70.97 | 93.75 | [symbol] |
| Q12. The appearance of fine lines and wrinkles is significantly reduced after using the product. | 44.12 | 68.75 | 81.25 | [symbol] |
| Q13. Dark spots/areas of excess pigmentation appear dramatically reduced after using the product. | 32.35 | 53.13 | 75 [symbol] | [symbol] |
| Q14. Skin looks and feels significantly firmer after using the product. | 52.94 | 84.38 | 87.5 [symbol] | [symbol] |
| Q15. Skin looks and feels significantly tighter after using the product. | 61.76 | 78.13 | 87.5 [symbol] | [symbol] |
| Q16. Skin looks and feels significantly more plump after using the product. | 58.82 | 71.88 | 87.5 [symbol] | [symbol] |
| Q17. Skin discolorations are significantly reduced after using the product. | 41.18 | 50 | 68.75 | [symbol] |
| Q18. Test product is gentle enough for everyday use. | 91.18 | 96.88 | 100 | [symbol] |
| Q19. Test product absorbs easily into skin. | 97.06 | 100 | 93.75 | [symbol] |
| Q20. I would recommend this product to a friend. | 79.41 | 87.1 | 90.63 | [symbol] |

[symbol]

indicates data missing or illegible when filed

Activated Silk 33B can be formulated into a stable, aqueous-based formulation at a level proven to improve skin barrier function (2%).

### Test Materials:

Serum, S33B-18 (containing 2% Activated Silk 33B)
Serum, NSS-144-75 (placebo serum lacking silk)

### Study Protocol

The objective of this study was to evaluate the efficacy of a serum containing 33B on fine lines/wrinkles, texture, redness, hyperpigmentation, firmness, hydration, elasticity, and barrier (trans epidermal water loss or TEWL) over a 28-day use period.

The same parameters were evaluated in a second study using a placebo control, to ensure beneficial effects were not due to other ingredients in the formulation apart from 33B. Consumer perception information was also collected in the study evaluating 33B. Subjects were instructed to use the respective test articles twice a day (morning and evening), on a freshly cleansed face. Study assessments consisting of the following were done at Baseline, Day 7 and Day 28:
Fine lines/wrinkles, hyperpigmentation, firmness and redness (visually assessed by an expert clinical grader)
Tactile texture/smoothness (assessed by an expert clinical grader)
Skin barrier via trans epidermal water loss (TEWL) (measured via Tewameter® TM 300 (Courage & Khazaka; Cologne, Germany)).
Skin surface hydration (measured via Corneometer® CM 825 (Courage & Khazaka; Cologne, Germany)).
Elasticity and firmness (measured via Cutometer® MPA 580 (Courage & Khazaka; Cologne, Germany)).
Skin surface lines and wrinkles were collected via silicone replicas and assessed via profilometry.
Self-perception questionnaire (33B serum study only).

### Results

Improvements in fine lines/wrinkles, firmness and redness (visually assessed by an expert clinical grader) are summarized below (improvement of hyperpigmentation was not statistically significant)

### Fine Lines and Wrinkles

2% 33B significantly reduced the appearance of fine lines and wrinkles as compared to the placebo control (assessed by an expert grader).

TABLE 3
Scoring scale utilized by expert grader to evaluate fine lines and wrinkles.
SCORING SCALE

| | TYPE OF | NONE | (1-3) | | | (4-6) | | | (7-9) | | |
|---|---|---|---|---|---|---|---|---|---|---|---|
| DESCRIPTORS | GRADING | 0 | 1 | 2 | 3 | 4 | 5 | 6 | 7 | 8 | 9 |

| | | | | | |
|---|---|---|---|---|---|
| Global Fine | Visual | No fine | Mild fine | Moderate fine | Severe fine |
| lines/Wrinkles | | lines/wrinkles | lines/wrinkles | lines/wrinkles | lines/wrinkles |

### Firmness

2% 33B significantly improved the appearance of skin firmness as compared to the placebo control (assessed by an expert grader).

TABLE 4
Scoring scale utilized by expert grader to evaluate skin firmness.
SCORING SCALE

| | TYPE OF | NONE | (1-3) | | | (4-6) | | | (7-9) | | |
|---|---|---|---|---|---|---|---|---|---|---|---|
| DESCRIPTORS | GRADING | 0 | 1 | 2 | 3 | 4 | 5 | 6 | 7 | 8 | 9 |

| | | | | | |
|---|---|---|---|---|---|
| Firmness | Visual | Firm taut | Moderately | Mildly firm | Loose, lax |
| | | appearance | firm | | |

### Redness

2% 33B significantly reduced the appearance of redness as compared to the placebo control (assessed by an expert grader).

TABLE 5
Scoring scale utilized by expert grader to evaluate redness.
SCORING SCALE

| | TYPE OF | NONE | (1-3) | | | (4-6) | | | (7-9) | | |
|---|---|---|---|---|---|---|---|---|---|---|---|
| DESCRIPTORS | GRADING | 0 | 1 | 2 | 3 | 4 | 5 | 6 | 7 | 8 | 9 |

| | | | | | |
|---|---|---|---|---|---|
| Redness | Visual | No redness | Slight redness | Moderate | Extensive |
| | | | | redness | redness |

Statistically significant improvement was seen for tactile evaluation of texture (smoothness) on Day 28 for both the 33B and placebo studies.

Statistically significant reduction in trans-epidermal water loss (TEWL) was observed at both the 7- and 28-day data points for the study group using the 33B serum. No improvement was seen in the placebo group.

Compared to baseline, skin surface hydration (measured via Corneometer®) was improved to a statistically significant extent after 7 and 28 days among subjects using the serum containing 33B. Significant improvement in the placebo group was observed at the 28-day data point only.

Elasticity and firmness (measured by Cutometer®) was not improved in either group. Two of eight parameters evaluating skin surface lines and wrinkles (measured by profilometry of silicone molds) showed statistically significant improvement after application of the serum containing 2% 33B.

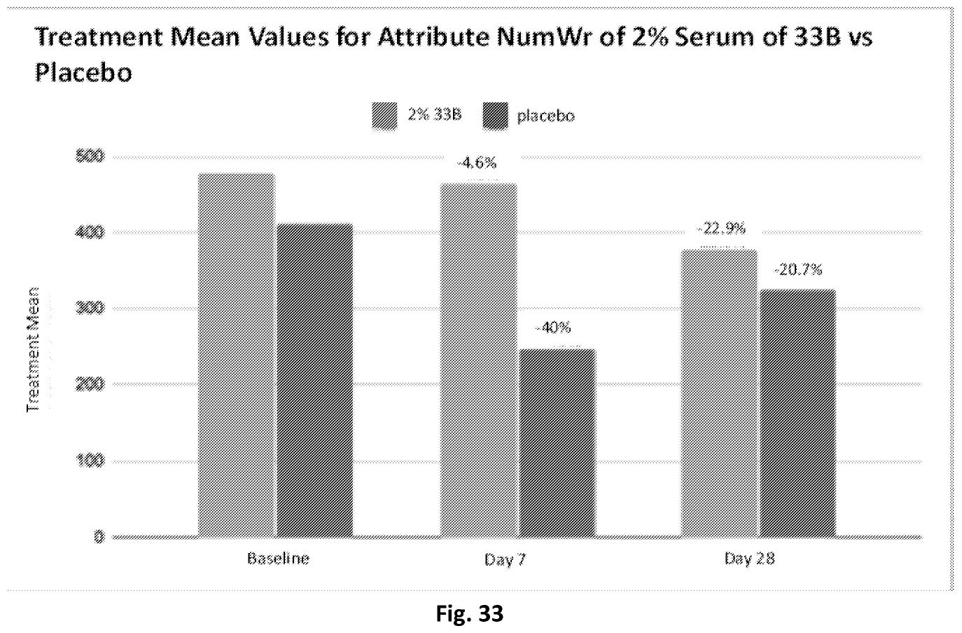

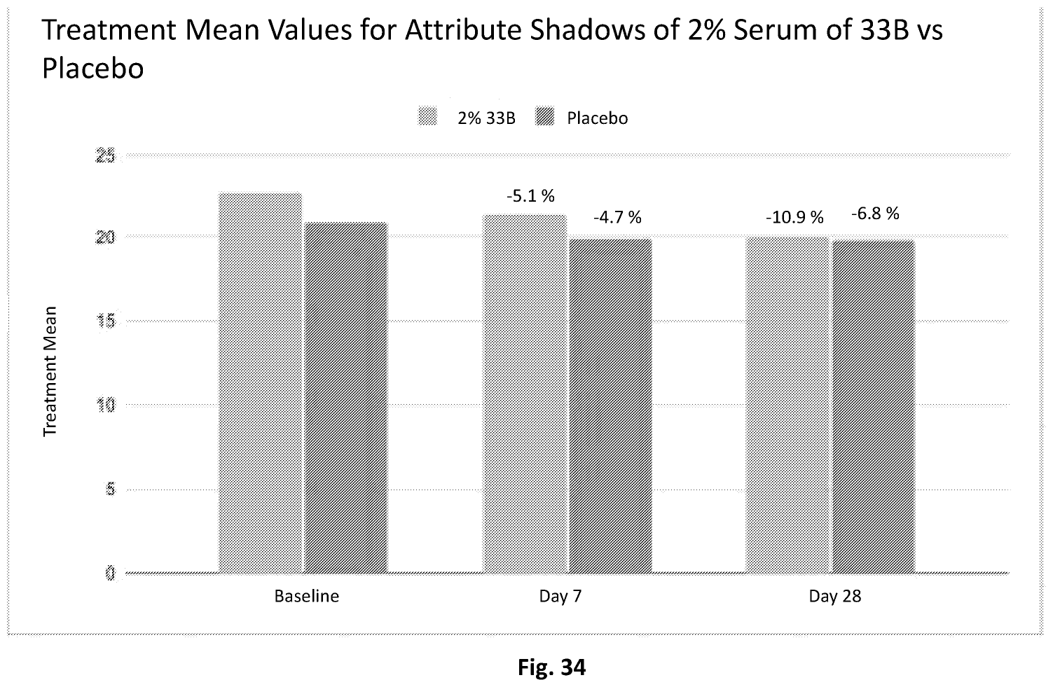

Number of wrinkles (NumWr) was significantly reduced for both the test serum and the placebo (data plotted in FIG. 33)

Shadows were significantly reduced for the test group using 33B; reduction in shadows for the placebo group was not statistically significant (data plotted in FIG. 34).

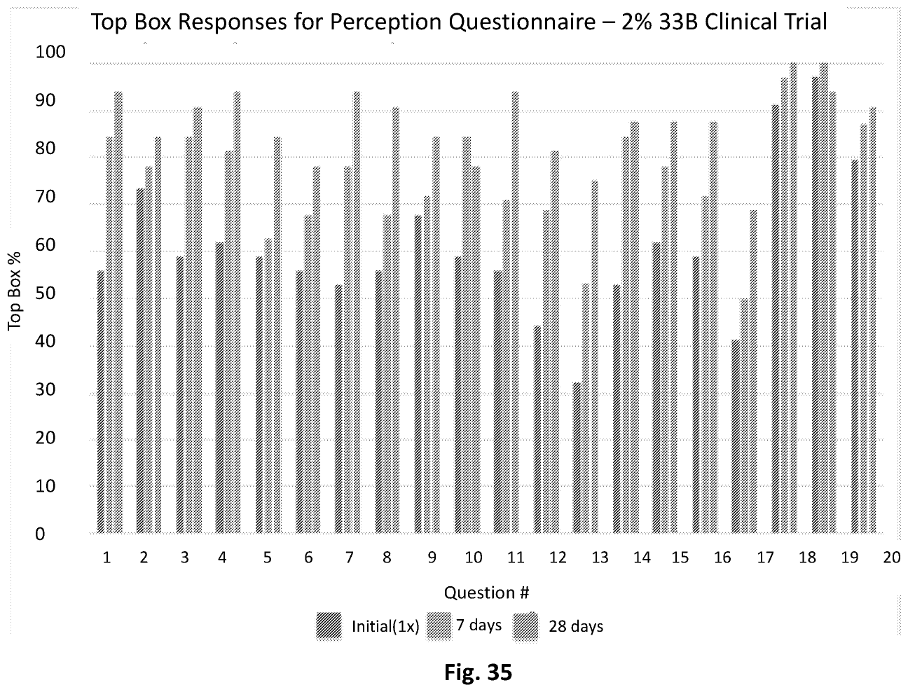

The Self-perception Questionnaires in the 33B serum study showed favorable results. Table 1 and FIG. 35 show which statements were statistically significant based on the percent of subjects choosing top box responses (Strongly Agree and Agree).

### Conclusions

At the concentration evaluated in this study (2%), Activated Silk 33B significantly reduces trans-epidermal water loss, validating the claim that it provides a benefit to the skin barrier. Additionally, claims regarding hydration, reduction in redness, reduction in appearance of fine lines/wrinkles, and improvement in appearance of skin texture are substantiated. Self-perception feedback pertaining to the ingredient performance in a serum format is also promising.

### Example 11: Primary Keratinocyte Claudin-1 Expression

Primary human neonatal epidermal keratinocytes were cultured in wells of 48-well tissue-culture treated plates with Epilife medium (Gibco) containing 1×EDGS (Epilife Defined Growth Supplement, Gibco), 100 U/mL penicillin (Genesee Scientific), 100 μg/mL streptomycin (Genesee Scientific) and 60 μM CaCl₂). Cells between passage 2-5 were used for experiments and incubated at 37° C. with 5% CO₂ till 80-90% confluence was reached. Cells were then gently washed 2 times with 1× D-PBS and then treated with serum-free Epilife media (only 100 U/mL penicillin, 100 g/mL streptomycin, 60 μM CaCl₂)) for 30 mins at 37° C., 5% CO₂. Serum-free Epilife medium containing the respective concentrations of MID SKID silk (0.05, 0.1, 0.2, 0.5, 0.6% w/v) or LOW SKID silk (0.05, 0.2, 0.4, 0.7% w/v) were added after 2× washes with 1× D-PBS and the cells were incubated for 24 hrs at 37° C., 5% CO₂. No treatment cells were treated with serum-free Epilife medium containing water (vehicle control).

For immunofluorescence staining, after the 24 hrs treatment, cells were washed 3× with 1× D-PBS and fixed with 4% paraformaldehyde (diluted in 1× D-PBS) for 10 mins at room temperature (RT) in the dark. Cells were then washed 2× with 1× D-PBS, permeabilized with 0.03% Triton-X (diluted in 1× D-PBS) for 10 mins at RT, and then blocked with super blocking solution (normal goat serum, normal donkey serum, 10% BSA, fish skin gelatin, 100% Triton-X in 1× D-PBS) for 30 mins at RT. Human anti-mouse monoclonal claudin-1 primary antibody (1:300; sc-81796, Santa Cruz) diluted in 50% blocking solution in 1× D-PBS was added to fixed cells and incubated for 1 hr at RT or overnight at 4° C. IgG control cells were treated with normal mouse IgG₂ₐ antibody (Santa Cruz; sc-3878) as negative control for primary antibody binding. Following 2 washes with 1× D-PBS, Alexa-Fluorm 594-conjugated goat anti-mouse IgG secondary Ab (1:400; Thermo Fisher Scientific) was added to cells and incubated in the dark for 1 hr at RT. After a 1× D-PBS wash, nuclei were counterstained with Hoechst 33342, washed again with 1× D-PBS and mounted with SlowFadem Diamond Antifade Mountant with DAPI (Thermo Fisher Scientific) for DAPI and TX-Red fluorescence imaging. Experiments for mid silk (33B) were performed with at least 3 biological repeats and cells were imaged at 20× magnification to visualize DAPI-stained nuclei and TX-Red stained Claudin-1. Preliminary experiments for low silk (27p) were performed with one biological repeat, 2 technical repeats and images were processed similar to 33B experiments. Statistical analysis was performed using an ordinary one-way ANOVA with multiple comparisons between MID SKID silk treated samples and untreated samples and considered significant if p<0.05.

### Example 12: Skin Biopsy Claudin-1 Expression

Briefly, healthy skin was pretreated with acetone for 5 minutes to create the initial insult. Petroleum jelly, 20 μl of Mid Skid silk (AS™ 33B) at 2, 3, 4, 5, and 60 mg/ml or vehicle control were applied on skin biopsies for 24 h. Afterward, the skin sample were washed with Dulbecco's phosphate buffered saline (DPBS) solution, fixed with 4% paraformaldehyde, and processed for cryostat sectioning by iHisto INC. Sections were incubated overnight at 4° C. with anti-claudin-1 antibody (Thermo Fisher Scientific), followed by reaction with AF-647-conjugated secondary antibody (Thermo Fisher Scientific). Nuclei were counterstained with Hoechst 33342, washed in PBS, and mounted with Antifade Mounting Media (Thermo Fisher Scientific). The claudin-1 positive area was measured using ImageJ software. Data are expressed as percentage±SEM from at least two separate microscopic fields, 200×, from 1 to 6 donors per condition. To calculate the normalized expression of claudin-1 the following formula was used:

% claudin-1 stain=total area of claudin1 staining total epidermis area×100

For comparisons between multiple groups, the overall differences were analyzed by ANOVA with Bonferroni multiple comparison tests.

### Example 13: Collagen Expression in Dermal Fibroblasts

Primary human dermal fibroblasts were propagated at 37° C. with 5% CO₂ to 65-70% confluence in fibroblast expansion medium (FEM, Gibco) containing 1×LSGS (Low serum growth supplement, Gibco) and 100 U/mL penicillin (Genesee Scientific), 100 μg/mL streptomycin (Genesee Scientific), 292 μg/mL L-glutamine (in 100 μM citrate buffer). For experiments, fibroblast cells between passage 2-6 were seeded into wells of 48-well tissue culture treated plates to grow overnight at 37° C., 5% CO₂ in DMEM 1 g/L glucose medium (Genesee Scientific) containing 1% FBS (Genesee Scientific), 2 mM glutamine, 100 U/mL penicillin, 100 μg/mL streptomycin (Cytvia) to reach 80-90% confluence. Cells were then gently washed 2 times with 1× D-PBS and then treated with serum-free DMEM media (only 2 mM glutamine, 100 U/mL penicillin, 100 μg/mL streptomycin) for 30 mins at 37° C., 5% CO₂. Serum-free DMEM medium containing the respective concentrations of LOW SKID silk (0.25, 0.5, 2.0, 7.0 mg/mL) were added after 2× washes with 1× D-PBS and the cells were incubated for 24 hrs at 37° C., 5% CO₂. Negative control cells were treated with serum-free DMEM medium containing water (vehicle control), while positive control cells were treated with serum-free DMEM medium containing 10 ng/mL TGF-β1. All treatment conditions were additionally tested in the absence and presence of 20 μg/mL vitamin C (L-ascorbic acid).

To measure total collagen production using sirius red spectrophotometric analysis, fibroblast cells (after 24 hrs treatment) were washed 2× with 1× D-PBS and fixed in 4% paraformaldehyde (diluted in 1× D-PBS) for 10 mins at room temperature (RT) in the dark. Cells were then washed 2× with tap water and stained with Sirius red dye (0.1%) for 1 hr at RT. Cells were then quickly washed 2× with 0.5% v/v acetic acid (acidified water). For spectrophotometric analysis, the sirius red dye was eluted in 0.1 N NaOH (less than 1 minute) and optical density at 540 nm was measured using Varioskan Lux Spectrophotometer (Thermo Fisher Scientific). Mildly stained fixed fibroblast cells were imaged at 4× magnification using a bright-field microscope (EVOS M7000, Thermo Fisher Scientific). Data from 4 biological repeats and 2 technical repeats were averaged. Test for statistical significance was performed using 2-way ANOVA (Dunnetts' multiple comparisons test) between treated and negative control samples and was considered significant if p<0.05.

### Example 14: Low Skid Silk Accelerates Keratinocyte Migration in a Wound Closure Assay

Primary human neonatal epidermal keratinocytes were cultured between passage 2-4 in 24-well tissue-culture treated plates with M-154 medium (M154500, Gibco) containing 1× HKGS (Human Keratinocyte Growth Supplement, S001K), 0.25 μg/mL Gentamycin, 10 μg/mL Amphotericin, 70 μM Ca2+. Cell cultures were incubated in 37° C., 5% CO₂ till 90-95% confluence was reached. Cells were then washed gently with 1× D-PBS and scratched uniformly with 20 μL pipette tip to create a linear wound in the wells. Cells were gently washed with 1× D-PBS. Treatment conditions were as follows: negative control contained serum-free M-154 media (only gentamycin, amphotericin, water, 70 μM Ca2+); positive control contained complete M-154 media with 1×HKGS, antibiotics and 70 μM Ca2+; test sample contained 0.5 mg/mL 27p diluted in serum-free M-154 media with antibiotics and 70 μM Ca2+. Respective treatment conditions were added to cells following scratch and 1× D-PBS wash and allowed to incubate at 37° C., 5% CO2 for 20 hrs. Cells were imaged at 30 min intervals for 20 hrs with a live imaging bright-field microscope (Cytation 1, Agilent BioTek). Image stacks of cells over 20 hrs were analyzed using ImageJ (Wound Healing size tool plugin) to measure the wound area in each frame. Data was plotted as wound closure (%) (per equation below) over time (hrs); error bars represent standard deviation in wound closure (%) measured in at least 2 wells of cells treated with each condition.

$$Wound closure ( \% ) = \frac{Wound area ( time 0 hr ) - Wound area ( time t hr )}{Wound area ( time 0 hr )}$$

### Example 15: CD44 Interaction with Silk Polypeptides

For the solid phase protein-protein interaction assays Multiwell Immune Plate were used, Naxisorp 96 well (Sigma-Aldrich, M9410-1CS, Lot 20182). The wells were coated with 200 μL of silk solution that has been centrifuged at max speed at 20,000× g. Plates with silk were incubated overnight at 4° C. Wells were washed with 200 μL TBS 1× three times. 200 μL of blocking solution (BLOTTO in TBS, Thermo Scientific REF 37530 LOTXB344941—Diluted 1/20 in 1×TBS) were added in each well and incubated at 37° C. for 1 h. Wells were washed with 200 μL TBS 1× three times. 100 μL of a 200 nM solution of CD44-hFc in 1×TBS were added in each well and the plate was incubated at 37° C. for 1 h. Wells were washed with 200 μL TBS 1× three times. Anti-human IgG (Fc-specific) Peroxidase antibody was diluted 1:50,000 in 1×TBS. 100 μL of 1:50,000 Anti-human IgG (Fc-specific) Peroxidase antibody dilution was added in each well and the plate was incubated at 37° C. for 1 h. Wells were washed with 200 μL TBS 1× three times. 100 μL of Ultrasensitive TMB solution was added in each well and the absorbance at 653 nm was measured every 1 min for 60 min.

### Materials Used:

Blocking solution: Blocker BLOTTO in TBS, Thermo Scientific REF 37530 LOTXB344941—Dilute 1/20 in 1×TBS.
Recombinant Human CD44 Protein (Fc-Tag), Sino Biological, 12211-H02H, LC13AP3001 50 μg.
Anti-human IgG (Fc-specific) Peroxidase antibody produced in goat, affinity isolated antibody, Sigma-Aldrich A0170-1 mL, Lot 0000154644, Source 0000141524
Human IgG, Fc Fragment Purified Protein, Millipore, Catalogue number AG714, Lot Number 3853811.
TBS Tablets, EMD Millipore, 524750-1EA, Lot: 3780735
Ultrasensitive TMB solution (Millipore, ES022-500 mL, Lot Number 3739113)

### Example 16: Preparation of 33B Serum for Clinical Study

In a 1000-mL beaker, RO/DI water (566.94 g, 94.49%) was stirred with an overhead stirrer equipped with a 4-blade pitched impeller stir shaft. The water was simultaneously heated to 60-65° C. on a hot plate. Once the desired temperature was reached, hydroxyethyl cellulose (5.28 g, 0.88%) was sifted into the stirring water. The heating apparatus was turned off, allowing the mixture to slightly cool while stirring over the subsequent >30 minutes. Once the hydroxyethyl cellulose had dissolved, Geogard Ultra (a blend comprised of gluconolactone and sodium benzoate, ulprospector.com) (5.40 g, 0.90%) was added. The solution was stirred until homogenous. Stir speed was reduced to 200 rpm. Silk solution, containing 6% solids (19.98 g, 3.33%, equating to 20.00% 33B+1.33% water) was added. The mixture was stirred for about 5 minutes, intermittently hand stirring with a spatula to aid mixing. The mixture was then adjusted to pH 5.0 using a 25% aqueous solution of sodium hydroxide (2.40 g, 0.40%. Once homogenous, stirring was stopped and the product was packaged into frosted glass dropper bottles.

The above procedure was duplicated to produce a placebo serum, omitting the addition of silk solution (an equivalent volume of water was added in lieu of silk).

### Clinical Study

A single-blind, home use study was conducted by Princeton Consumer Research over a 28-day period. The study was entitled “A Clinical Study to Determine the Efficacy of a Test Article to Improve the Signs of Aging over a 28-Day Use Period.” A total of 34 female participants were enrolled in the test arm of the study (with 32 completing the study), while 23 female participants were enrolled in the placebo arm of the study (with 22 completing the study). Subjects were issued either the 33B serum or placebo serum with the instruction to apply twice a day (morning and evening) on a freshly cleansed face. Study assessments were completed at baseline, day 7 and day 28 and included the following:
Fine lines/wrinkles, hyperpigmentation, firmness and redness were visually assessed by a clinical grader
Tactile texture/smoothness were assessed by the clinical grader.
Skin barrier via trans epidermal water loss (TEWL) were measured with Tewameter® TM 300 (Courage & Khazaka).
Skin surface hydration was measured with Corneometer® CM 825 (Courage & Khazaka).
Subjects completed a Self-Perception Questionnaire (SPQ) assessing skin attributes, product efficacy and perceived improvement.

### Example 17: Anti-Inflammatory Data

TABLE 1
Analysis Result for Treatment A

| | | log2Fold | |
|---|---|---|---|
| Ensembl ID | #N/A | Change | P value |
| ENSG00000263503 | MAPK8IP1P2 | −22.0508 | 5.67E−07 |
| ENSG00000285668 | AC126544.2 | −21.4155 | 1.19E−06 |
| ENSG00000263586 | HID1-AS1 | −5.75416 | 0.006534 |
| ENSG00000135744 | AGT | −5.75233 | 0.003856 |
| ENSG00000259675 | AC018618.1 | −5.70572 | 0.01021 |
| ENSG00000260088 | AL445483.1 | −5.70565 | 0.005133 |
| ENSG00000249894 | AC024581.1 | −5.47337 | 0.016908 |
| ENSG00000276514 | RF02271 | −5.36936 | 0.010726 |
| ENSG00000281852 | LINC00891 | −5.29772 | 0.014305 |
| ENSG00000242715 | CCDC169 | −5.28585 | 0.015504 |
| ENSG00000228363 | AC015971.1 | −5.21423 | 0.039025 |
| ENSG00000124253 | PCK1 | −5.20202 | 0.015684 |
| ENSG00000270716 | BNIP3P15 | −5.1948 | 0.015431 |
| ENSG00000235641 | LINC00484 | −5.13769 | 0.033092 |
| ENSG00000275022 | MIR6753 | −5.12748 | 0.021439 |
| ENSG00000089558 | KCNH4 | −5.11548 | 0.022135 |
| ENSG00000213763 | ACTBP2 | −5.11339 | 0.019051 |
| ENSG00000239572 | AC108749.1 | −5.0631 | 0.035554 |
| ENSG00000130829 | DUSP9 | −5.02344 | 0.034017 |
| ENSG00000207975 | MIR181B1 | −4.92277 | 0.041403 |
| ENSG00000167414 | GNG8 | −4.88913 | 0.030102 |
| ENSG00000279858 | AC068880.4 | −4.88235 | 0.031889 |
| ENSG00000272783 | AC147067.2 | −4.87412 | 0.046626 |
| ENSG00000223838 | AC007091.1 | −4.86659 | 0.032527 |
| ENSG00000188693 | CYP51A1-AS1 | −4.84343 | 0.037259 |
| ENSG00000273100 | AL596442.2 | −4.84085 | 0.042755 |
| ENSG00000216480 | AL078604.1 | −4.77077 | 0.02109 |
| ENSG00000276656 | MIR6083 | −4.76869 | 0.043063 |
| ENSG00000224153 | LINC02054 | −4.70999 | 0.047734 |
| ENSG00000256069 | A2MP1 | −4.70457 | 0.035602 |
| ENSG00000239739 | AC026316.2 | −4.67638 | 0.046725 |
| ENSG00000180015 | AC093909.1 | −4.3346 | 0.032857 |
| ENSG00000248424 | OR51K1P | −4.25586 | 0.028804 |
| ENSG00000225569 | CCT4P2 | −4.2312 | 0.025686 |
| ENSG00000272386 | AC015802.5 | −4.20046 | 0.02649 |
| ENSG00000262003 | AC087392.1 | −4.17712 | 0.049171 |
| ENSG00000177350 | RPL13AP3 | −4.12633 | 0.029032 |
| ENSG00000236536 | AC003986.3 | −4.10561 | 0.033853 |
| ENSG00000283913 | AL512662.2 | −4.01732 | 0.012396 |
| ENSG00000218027 | AL512329.1 | −4.00809 | 0.039604 |
| ENSG00000266507 | MIR4479 | −3.94959 | 0.04613 |
| ENSG00000207944 | MIR574 | −3.93001 | 0.044879 |
| ENSG00000244411 | KRTAP5-7 | −3.88995 | 0.013853 |
| ENSG00000251108 | YBX1P5 | −3.82395 | 0.014155 |
| ENSG00000259275 | AC087477.2 | −3.80145 | 0.01878 |
| ENSG00000173809 | TDRD12 | −3.78415 | 0.001317 |
| ENSG00000163661 | PTX3 | −3.77413 | 0.021133 |
| ENSG00000206028 | Z99774.1 | −3.7623 | 0.013871 |
| ENSG00000162739 | SLAMF6 | −3.65943 | 0.024173 |
| ENSG00000229871 | RPSAP20 | −3.55188 | 0.027538 |
| ENSG00000224810 | AL355482.1 | −3.55015 | 0.000281 |
| ENSG00000283283 | AC013268.4 | −3.47723 | 0.011034 |
| ENSG00000275322 | AC103746.1 | −3.41997 | 0.024386 |
| ENSG00000255836 | AC131206.1 | −3.35874 | 0.028989 |
| ENSG00000280054 | AC004241.5 | −3.29555 | 0.008832 |
| ENSG00000229991 | AKR1B1P1 | −3.23034 | 0.047824 |
| ENSG00000101307 | SIRPB1 | −3.18207 | 0.039981 |
| ENSG00000279205 | AC092162.3 | −3.12453 | 0.025956 |
| ENSG00000179899 | PHC1P1 | −3.04682 | 0.032553 |
| ENSG00000235997 | LINC01936 | −3.00996 | 0.024622 |
| ENSG00000225265 | TAF1A-AS1 | −2.9982 | 0.037395 |
| ENSG00000139549 | DHH | −2.99721 | 0.012052 |
| ENSG00000203710 | CR1 | −2.97221 | 0.018846 |
| ENSG00000250796 | AC112484.2 | −2.94046 | 0.020183 |
| ENSG00000231440 | AL358176.4 | −2.88925 | 0.013681 |
| ENSG00000173597 | SULT1B1 | −2.85822 | 0.026893 |
| ENSG00000203635 | AC144450.1 | −2.78 | 0.033041 |
| ENSG00000224769 | MUC20P1 | −2.75446 | 0.003822 |
| ENSG00000128645 | HOXD1 | −2.74369 | 0.004276 |
| ENSG00000178115 | GOLGA8Q | −2.70808 | 0.03141 |
| ENSG00000273489 | AC008264.2 | −2.65879 | 0.008641 |
| ENSG00000258611 | AC087641.1 | −2.59551 | 0.025183 |
| ENSG00000119535 | CSF3R | −2.58782 | 0.01326 |
| ENSG00000239823 | RF00019 | −2.54203 | 0.014128 |
| ENSG00000111729 | CLEC4A | −2.46318 | 0.034774 |
| ENSG00000180644 | PRF1 | −2.44965 | 0.045031 |
| ENSG00000243806 | RPL7P18 | −2.4431 | 0.014341 |
| ENSG00000129596 | CDO1 | −2.39214 | 0.000353 |
| ENSG00000200105 | RNU6-251P | −2.36789 | 0.044095 |
| ENSG00000249840 | GAPDHP76 | −2.33777 | 0.031992 |
| ENSG00000269895 | AP000654.1 | −2.31157 | 0.03861 |
| ENSG00000179930 | ZNF648 | −2.27911 | 0.017483 |
| ENSG00000232309 | AL390856.1 | −2.27516 | 0.047183 |
| ENSG00000158445 | KCNB1 | −2.26053 | 0.010028 |
| ENSG00000254088 | SLC2A3P4 | −2.24539 | 0.047248 |
| ENSG00000267474 | AC008569.2 | −2.23929 | 0.049999 |
| ENSG00000205309 | NT5M | −2.19805 | 0.023033 |
| ENSG00000183773 | AIFM3 | −2.19145 | 0.042436 |
| ENSG00000121410 | A1BG | −2.17699 | 0.03513 |
| ENSG00000115705 | TPO | −2.15636 | 0.006343 |
| ENSG00000213279 | Z97192.2 | −2.15328 | 0.034362 |
| ENSG00000110077 | MS4A6A | −2.09217 | 0.00283 |
| ENSG00000197406 | DIO3 | −2.05434 | 0.000432 |
| ENSG00000110079 | MS4A4A | −2.03935 | 0.037766 |
| ENSG00000228918 | LINC01344 | −2.01612 | 0.01738 |
| ENSG00000272576 | AC027271.1 | −1.99879 | 0.041263 |
| ENSG00000184113 | CLDN5 | −1.99844 | 0.001982 |
| ENSG00000133116 | KL | −1.99188 | 0.008241 |
| ENSG00000235920 | AC073109.1 | −1.97495 | 0.038267 |
| ENSG00000235111 | Z97192.3 | −1.96743 | 0.013427 |
| ENSG00000172322 | CLEC12A | −1.94986 | 0.02961 |
| ENSG00000107984 | DKK1 | −1.94714 | 0.016723 |
| ENSG00000233251 | AC007743.1 | −1.9443 | 0.023635 |
| ENSG00000162174 | ASRGL1 | −1.93739 | 0.010271 |
| ENSG00000167588 | GPD1 | −1.93149 | 0.045901 |
| ENSG00000155816 | FMN2 | −1.92513 | 0.027285 |
| ENSG00000159387 | IRX6 | −1.91128 | 0.002151 |
| ENSG00000005108 | THSD7A | −1.90591 | 0.006217 |
| ENSG00000213203 | GIMAP1 | −1.89601 | 0.000449 |
| ENSG00000258498 | DIO3OS | −1.88086 | 0.000368 |
| ENSG00000186205 | 1-Mar | −1.86335 | 0.032953 |
| ENSG00000154016 | GRAP | −1.83693 | 0.002363 |
| ENSG00000186466 | AQP7P1 | −1.8056 | 0.039959 |
| ENSG00000276409 | CCL14 | −1.79384 | 0.036836 |
| ENSG00000133800 | LYVE1 | −1.78602 | 7.57E-05 |
| ENSG00000134668 | SPOCD1 | −1.77151 | 0.017644 |
| ENSG00000118432 | CNR1 | −1.76952 | 0.031878 |
| ENSG00000235795 | AC093157.2 | −1.76892 | 0.046717 |
| ENSG00000188511 | C22orf34 | −1.75867 | 0.000991 |
| ENSG00000122679 | RAMP3 | −1.75854 | 0.036568 |
| ENSG00000007038 | PRSS21 | −1.74426 | 0.044318 |
| ENSG00000211445 | GPX3 | −1.72246 | 0.002658 |
| ENSG00000128709 | HOXD9 | −1.68039 | 0.006699 |
| ENSG00000261786 | AC006058.1 | −1.67366 | 0.044202 |
| ENSG00000259660 | DNM1P47 | −1.66986 | 0.019854 |
| ENSG00000149948 | HMGA2 | −1.66248 | 0.007287 |
| ENSG00000108018 | SORCS1 | −1.66177 | 0.019979 |
| ENSG00000279354 | AC090373.1 | −1.64535 | 0.034124 |
| ENSG00000224349 | AL365226.1 | −1.62291 | 0.049286 |
| ENSG00000010327 | STAB1 | −1.61379 | 0.00136 |
| ENSG00000235033 | AL590999.1 | −1.61057 | 0.040417 |
| ENSG00000182308 | DCAF4L1 | −1.60882 | 0.03325 |
| ENSG00000163364 | LINC01116 | −1.59888 | 0.015789 |
| ENSG00000279742 | AP000974.1 | −1.58849 | 0.019985 |
| ENSG00000230185 | C9orf147 | −1.58678 | 0.035531 |
| ENSG00000019169 | MARCO | −1.55654 | 0.049665 |
| ENSG00000224397 | SMIM25 | −1.55332 | 0.02134 |
| ENSG00000090659 | CD209 | −1.54892 | 0.005956 |
| ENSG00000213088 | ACKR1 | −1.54792 | 0.016766 |
| ENSG00000278769 | AC090510.3 | −1.53751 | 0.023132 |
| ENSG00000170989 | S1PR1 | −1.52944 | 0.000498 |
| ENSG00000198844 | ARHGEF15 | −1.50977 | 0.000738 |
| ENSG00000250510 | GPR162 | −1.50127 | 0.01597 |
| ENSG00000100302 | RASD2 | −1.49681 | 0.011439 |
| ENSG00000138028 | CGREF1 | −1.49071 | 0.012187 |
| ENSG00000223756 | TSSC2 | −1.48724 | 0.002294 |
| ENSG00000255282 | WTAPP1 | −1.48261 | 0.040384 |
| ENSG00000168993 | CPLX1 | −1.47928 | 0.001635 |
| ENSG00000152475 | ZNF837 | −1.47521 | 0.032358 |
| ENSG00000261468 | AC096921.2 | −1.47172 | 0.003533 |
| ENSG00000157782 | CABP1 | −1.46657 | 0.009963 |
| ENSG00000260105 | AOC4P | −1.45878 | 0.033075 |
| ENSG00000042062 | RIPOR3 | −1.44543 | 0.012126 |
| ENSG00000265750 | AC090772.3 | −1.42666 | 0.021028 |
| ENSG00000230910 | AL391807.1 | −1.42522 | 0.030579 |
| ENSG00000130300 | PLVAP | −1.42216 | 0.002484 |
| ENSG00000245213 | AC105285.1 | −1.41323 | 0.04917 |
| ENSG00000152760 | TCTEX1D1 | −1.39577 | 0.008088 |
| ENSG00000196329 | GIMAP5 | −1.37696 | 0.021661 |
| ENSG00000100060 | MFNG | −1.36632 | 0.019354 |
| ENSG00000184497 | TMEM255B | −1.35546 | 1.37E-05 |
| ENSG00000019102 | VSIG2 | −1.35544 | 0.020233 |
| ENSG00000196569 | LAMA2 | −1.35503 | 0.025727 |
| ENSG00000064205 | WISP2 | −1.35289 | 0.00254 |
| ENSG00000085276 | MECOM | −1.35046 | 0.00469 |
| ENSG00000261371 | PECAM1 | −1.34525 | 0.00205 |
| ENSG00000136869 | TLR4 | −1.34467 | 0.001594 |
| ENSG00000101331 | CCM2L | −1.34127 | 0.039983 |
| ENSG00000205038 | PKHD1L1 | −1.33005 | 0.007945 |
| ENSG00000018280 | SLC11A1 | −1.32281 | 0.025192 |
| ENSG00000104967 | NOVA2 | −1.31708 | 0.000858 |
| ENSG00000166148 | AVPR1A | −1.30899 | 0.039646 |
| ENSG00000104903 | LYL1 | −1.30178 | 0.026112 |
| ENSG00000157152 | SYN2 | −1.30038 | 0.032122 |
| ENSG00000189056 | RELN | −1.29275 | 0.026715 |
| ENSG00000260314 | MRC1 | −1.27853 | 0.034041 |
| ENSG00000106538 | RARRES2 | −1.27601 | 0.007023 |
| ENSG00000151948 | GLT1D1 | −1.27348 | 0.039314 |
| ENSG00000101230 | ISM1 | −1.26283 | 0.019504 |
| ENSG00000160801 | PTH1R | −1.24884 | 0.002045 |
| ENSG00000103710 | RASL12 | −1.23842 | 0.005814 |
| ENSG00000066056 | TIE1 | −1.23528 | 0.001939 |
| ENSG00000139910 | NOVA1 | −1.2264 | 0.021432 |
| ENSG00000189058 | APOD | −1.22552 | 0.014508 |
| ENSG00000234171 | RNASEH1-AS1 | −1.22458 | 0.019043 |
| ENSG00000160999 | SH2B2 | −1.22181 | 0.039472 |
| ENSG00000037280 | FLT4 | −1.21268 | 8.48E-05 |
| ENSG00000137491 | SLCO2B1 | −1.21247 | 0.021217 |
| ENSG00000145014 | TMEM44 | −1.212 | 0.00976 |
| ENSG00000118407 | FILIP1 | −1.20856 | 0.004372 |
| ENSG00000148541 | FAM13C | −1.20694 | 0.006625 |
| ENSG00000169291 | SHE | −1.20259 | 0.001949 |
| ENSG00000154065 | ANKRD29 | −1.20216 | 0.034075 |
| ENSG00000135835 | KIAA1614 | −1.19937 | 0.006096 |
| ENSG00000132514 | CLEC10A | −1.18198 | 0.037844 |
| ENSG00000108001 | EBF3 | −1.18122 | 0.005688 |
| ENSG00000179314 | WSCD1 | −1.17937 | 0.049373 |
| ENSG00000133574 | GIMAP4 | −1.17859 | 0.032366 |
| ENSG00000115590 | IL1R2 | −1.17671 | 0.03615 |
| ENSG00000093072 | ADA2 | −1.17615 | 0.012459 |
| ENSG00000146374 | RSPO3 | −1.17489 | 0.046924 |
| ENSG00000099998 | GGT5 | −1.17141 | 0.034719 |
| ENSG00000133687 | TMTC1 | −1.16394 | 0.000183 |
| ENSG00000079337 | RAPGEF3 | −1.16236 | 0.004515 |
| ENSG00000186994 | KANK3 | −1.16228 | 0.008757 |
| ENSG00000143127 | ITGA10 | −1.15302 | 0.024342 |
| ENSG00000128596 | CCDC136 | −1.14507 | 0.037324 |
| ENSG00000165507 | DEPP1 | −1.14494 | 0.00079 |
| ENSG00000124615 | MOCS1 | −1.13672 | 0.019922 |
| ENSG00000105499 | PLA2G4C | −1.1357 | 0.01593 |
| ENSG00000203883 | SOX18 | −1.1203 | 0.008396 |
| ENSG00000172889 | EGFL7 | −1.10246 | 0.000901 |
| ENSG00000126106 | TMEM53 | −1.10212 | 0.039924 |
| ENSG00000163072 | NOSTRIN | −1.09744 | 0.035064 |
| ENSG00000154654 | NCAM2 | −1.09459 | 0.015895 |
| ENSG00000163083 | INHBB | −1.09417 | 0.021285 |
| ENSG00000129538 | RNASE1 | −1.08777 | 0.049114 |
| ENSG00000105538 | RASIP1 | −1.08117 | 0.01155 |
| ENSG00000160191 | PDE9A | −1.08115 | 0.043778 |
| ENSG00000184254 | ALDH1A3 | −1.07868 | 0.001492 |
| ENSG00000133561 | GIMAP6 | −1.0717 | 0.005997 |
| ENSG00000128052 | KDR | −1.04673 | 0.005896 |
| ENSG00000246982 | Z84485.1 | −1.04167 | 0.006985 |
| ENSG00000174059 | CD34 | −1.03601 | 0.003197 |
| ENSG00000240583 | AQP1 | −1.03385 | 0.040376 |
| ENSG00000124491 | F13A1 | −1.0304 | 0.027424 |
| ENSG00000235272 | RAMACL | −1.02949 | 0.013489 |
| ENSG00000128567 | PODXL | −1.02869 | 0.000961 |
| ENSG00000071282 | LMCD1 | −1.02515 | 0.015751 |
| ENSG00000185551 | NR2F2 | −1.01683 | 0.006217 |
| ENSG00000162804 | SNED1 | −1.00627 | 0.007915 |
| ENSG00000220785 | MTMR9LP | −1.00563 | 0.019619 |
| ENSG00000271811 | Z97200.1 | −1.00533 | 0.008469 |
| ENSG00000102445 | RUBCNL | −1.00446 | 0.007129 |
| ENSG00000128656 | CHN1 | −1.0036 | 0.025595 |
| ENSG00000230630 | DNM3OS | −1.00297 | 0.022166 |
| ENSG00000162367 | TAL1 | −1.00127 | 0.030133 |
| ENSG00000124440 | HIF3A | −0.99951 | 0.018956 |
| ENSG00000186407 | CD300E | −0.99566 | 0.014179 |
| ENSG00000107281 | NPDC1 | −0.99312 | 0.011051 |
| ENSG00000105639 | JAK3 | −0.97183 | 0.003845 |
| ENSG00000159212 | CLIC6 | −0.9675 | 0.034295 |
| ENSG00000047648 | ARHGAP6 | −0.9663 | 0.022995 |
| ENSG00000140961 | OSGIN1 | −0.96599 | 0.03994 |
| ENSG00000241399 | CD302 | −0.965 | 0.014939 |
| ENSG00000106991 | ENG | −0.96389 | 5.88E-05 |
| ENSG00000064692 | SNCAIP | −0.96179 | 0.015148 |
| ENSG00000101445 | PPP1R16B | −0.95799 | 0.005851 |
| ENSG00000144668 | ITGA9 | −0.95346 | 0.010955 |
| ENSG00000198873 | GRK5 | −0.95071 | 0.004402 |
| ENSG00000152953 | STK32B | −0.9484 | 0.011846 |
| ENSG00000125089 | SH3TC1 | −0.94522 | 0.010345 |
| ENSG00000198890 | PRMT6 | −0.94372 | 0.037577 |
| ENSG00000090376 | IRAK3 | −0.93992 | 0.025741 |
| ENSG00000167680 | SEMA6B | −0.93399 | 0.001772 |
| ENSG00000125810 | CD93 | −0.9339 | 0.00247 |
| ENSG00000120156 | TEK | −0.9309 | 0.040504 |
| ENSG00000165757 | JCAD | −0.92804 | 0.010901 |
| ENSG00000267787 | AC027097.2 | −0.92565 | 0.015109 |
| ENSG00000166923 | GREM1 | −0.9152 | 0.004004 |
| ENSG00000151067 | CACNA1C | −0.9141 | 0.042233 |
| ENSG00000003436 | TFPI | −0.90451 | 0.003795 |
| ENSG00000158715 | SLC45A3 | −0.90148 | 0.031608 |
| ENSG00000280604 | AJ239328.1 | −0.89647 | 0.035313 |
| ENSG00000053918 | KCNQ1 | −0.88856 | 0.036068 |
| ENSG00000147113 | DIPK2B | −0.88397 | 0.01396 |
| ENSG00000134853 | PDGFRA | −0.88332 | 0.037488 |
| ENSG00000251322 | SHANK3 | −0.88067 | 0.005547 |
| ENSG00000164867 | NOS3 | −0.87695 | 0.029842 |
| ENSG00000163521 | GLB1L | −0.86916 | 0.0433 |
| ENSG00000081479 | LRP2 | −0.86847 | 0.042102 |
| ENSG00000089327 | FXYD5 | −0.86651 | 0.031693 |
| ENSG00000046889 | PREX2 | −0.86569 | 0.009785 |
| ENSG00000128928 | IVD | −0.85669 | 0.007346 |
| ENSG00000130508 | PXDN | −0.85482 | 0.004033 |
| ENSG00000116962 | NID1 | −0.85339 | 0.003378 |
| ENSG00000069122 | ADGRF5 | −0.85224 | 0.006718 |
| ENSG00000139567 | ACVRL1 | −0.85099 | 0.002515 |
| ENSG00000154736 | ADAMTS5 | −0.84807 | 0.039819 |
| ENSG00000128917 | DLL4 | −0.84356 | 0.009519 |
| ENSG00000135636 | DYSF | −0.8435 | 0.000198 |
| ENSG00000166341 | DCHS1 | −0.84196 | 0.020595 |
| ENSG00000111058 | ACSS3 | −0.83704 | 0.044792 |
| ENSG00000073849 | ST6GAL1 | −0.8313 | 0.020596 |
| ENSG00000184489 | PTP4A3 | −0.83041 | 0.005984 |
| ENSG00000107551 | RASSF4 | −0.82529 | 0.007881 |
| ENSG00000113555 | PCDH12 | −0.82453 | 0.006154 |
| ENSG00000198624 | CCDC69 | −0.81442 | 0.019647 |
| ENSG00000106511 | MEOX2 | −0.81297 | 0.046135 |
| ENSG00000050555 | LAMC3 | −0.80629 | 0.008085 |
| ENSG00000151702 | FLI1 | −0.80616 | 0.039495 |
| ENSG00000170464 | DNAJC18 | −0.79506 | 0.023526 |
| ENSG00000153071 | DAB2 | −0.7857 | 0.023363 |
| ENSG00000095370 | SH2D3C | −0.78371 | 0.029986 |
| ENSG00000133121 | STARD13 | −0.77676 | 0.017838 |
| ENSG00000179776 | CDH5 | −0.77616 | 0.030118 |
| ENSG00000144152 | FBLN7 | −0.77387 | 0.01076 |
| ENSG00000163513 | TGFBR2 | −0.76574 | 0.014892 |
| ENSG00000079102 | RUNX1T1 | −0.75958 | 0.032009 |
| ENSG00000147408 | CSGALNACT1 | −0.75935 | 0.015245 |
| ENSG00000141337 | ARSG | −0.75914 | 0.002806 |
| ENSG00000143842 | SOX13 | −0.75884 | 0.0417 |
| ENSG00000124212 | PTGIS | −0.75266 | 0.022486 |
| ENSG00000162817 | C1orf115 | −0.75254 | 0.022225 |
| ENSG00000072163 | LIMS2 | −0.74233 | 0.021527 |
| ENSG00000105419 | MEIS3 | −0.74047 | 0.045384 |
| ENSG00000160993 | ALKBH4 | −0.73848 | 0.034887 |
| ENSG00000221968 | FADS3 | −0.73366 | 0.041994 |
| ENSG00000249669 | CARMN | −0.72979 | 0.011157 |
| ENSG00000151892 | GFRA1 | −0.72349 | 0.037259 |
| ENSG00000085662 | AKR1B1 | −0.71726 | 0.043757 |
| ENSG00000214357 | NEURL1B | −0.7157 | 0.016707 |
| ENSG00000138759 | FRAS1 | −0.7148 | 0.00622 |
| ENSG00000149564 | ESAM | −0.71337 | 0.030743 |
| ENSG00000131634 | TMEM204 | −0.71302 | 0.012634 |
| ENSG00000178175 | ZNF366 | −0.70367 | 0.039246 |
| ENSG00000184584 | TMEM173 | −0.70241 | 0.047849 |
| ENSG00000129925 | TMEM8A | −0.70199 | 0.033016 |
| ENSG00000142303 | ADAMTS10 | −0.70134 | 0.033523 |
| ENSG00000159433 | STARD9 | −0.69593 | 0.00585 |
| ENSG00000111752 | PHC1 | −0.69337 | 0.023906 |
| ENSG00000146021 | KLHL3 | −0.68396 | 0.020243 |
| ENSG00000100968 | NFATC4 | −0.68391 | 0.047028 |
| ENSG00000162733 | DDR2 | −0.68251 | 0.027196 |
| ENSG00000241684 | ADAMTS9-AS2 | −0.6804 | 0.009106 |
| ENSG00000177464 | GPR4 | −0.68002 | 0.046528 |
| ENSG00000081189 | MEF2C | −0.67877 | 0.014591 |
| ENSG00000108950 | FAM20A | −0.67214 | 0.029761 |
| ENSG00000161940 | BCL6B | −0.66966 | 0.046746 |
| ENSG00000108784 | NAGLU | −0.65944 | 0.048947 |
| ENSG00000063176 | SPHK2 | −0.65555 | 0.037991 |
| ENSG00000204131 | NHSL2 | −0.65411 | 0.015462 |
| ENSG00000071242 | RPS6KA2 | −0.64066 | 0.025866 |
| ENSG00000178878 | APOLD1 | −0.63901 | 0.026477 |
| ENSG00000152583 | SPARCL1 | −0.63645 | 0.045308 |
| ENSG00000176058 | TPRN | −0.63372 | 0.016588 |
| ENSG00000127920 | GNG11 | −0.61684 | 0.033095 |
| ENSG00000146122 | DAAM2 | −0.61432 | 0.020331 |
| ENSG00000157214 | STEAP2 | −0.61047 | 0.030626 |
| ENSG00000161791 | FMNL3 | −0.60887 | 0.01292 |
| ENSG00000063127 | SLC6A16 | −0.60038 | 0.044271 |
| ENSG00000177374 | HIC1 | −0.59836 | 0.030923 |
| ENSG00000116691 | MIIP | −0.59797 | 0.043463 |
| ENSG00000182240 | BACE2 | −0.59032 | 0.023137 |
| ENSG00000169733 | RFNG | −0.58893 | 0.045546 |
| ENSG00000167191 | GPRC5B | −0.58201 | 0.041185 |
| ENSG00000182809 | CRIP2 | −0.57954 | 0.045032 |
| ENSG00000176438 | SYNE3 | −0.57534 | 0.013956 |
| ENSG00000076706 | MCAM | −0.57357 | 0.040837 |
| ENSG00000197256 | KANK2 | −0.57107 | 0.024757 |
| ENSG00000117298 | ECE1 | −0.55844 | 0.022998 |
| ENSG00000106397 | PLOD3 | −0.52931 | 0.04839 |
| ENSG00000134686 | PHC2 | −0.5261 | 0.025195 |
| ENSG00000106624 | AEBP1 | −0.522 | 0.044444 |
| ENSG00000168918 | INPP5D | −0.4921 | 0.040113 |
| ENSG00000142798 | HSPG2 | −0.47913 | 0.016321 |
| ENSG00000135862 | LAMC1 | −0.47335 | 0.038534 |
| ENSG00000069431 | ABCC9 | −0.4604 | 0.042558 |
| ENSG00000166401 | SERPINB8 | 0.425156 | 0.040477 |
| ENSG00000121552 | CSTA | 0.544333 | 0.045353 |
| ENSG00000183023 | SLC8A1 | 0.564958 | 0.033455 |
| ENSG00000164687 | FABP5 | 0.617093 | 0.005704 |
| ENSG00000172575 | RASGRP1 | 0.644104 | 0.022965 |
| ENSG00000147592 | LACTB2 | 0.698567 | 0.049764 |
| ENSG00000147400 | CETN2 | 0.706373 | 0.028006 |
| ENSG00000272398 | CD24 | 0.830809 | 0.035717 |
| ENSG00000226383 | LINC01876 | 0.86393 | 0.015709 |
| ENSG00000152503 | TRIM36 | 0.945406 | 0.047174 |
| ENSG00000270277 | AC009948.2 | 0.950338 | 0.006447 |
| ENSG00000164128 | NPY1R | 1.039285 | 0.037191 |
| ENSG00000187775 | DNAH17 | 1.041152 | 0.0261 |
| ENSG00000185479 | KRT6B | 1.091759 | 0.049866 |
| ENSG00000170465 | KRT6C | 1.15352 | 0.003421 |
| ENSG00000188761 | BCL2L15 | 1.229252 | 0.042521 |
| ENSG00000254983 | AC025300.1 | 1.232753 | 0.02884 |
| ENSG00000168703 | WFDC12 | 1.274246 | 0.016966 |
| ENSG00000266237 | AC121320.1 | 1.366068 | 0.041328 |
| ENSG00000185130 | HIST1H2BL | 1.403462 | 0.047129 |
| ENSG00000207175 | RNU1-67P | 1.410814 | 0.024918 |
| ENSG00000235183 | SRP14P3 | 1.454147 | 0.015554 |
| ENSG00000174599 | TRAMIL1 | 1.495897 | 0.025472 |
| ENSG00000147488 | ST18 | 1.503935 | 0.026464 |
| ENSG00000189057 | FAM111B | 1.510514 | 0.047422 |
| ENSG00000145103 | ILDR1 | 1.51206 | 0.019211 |
| ENSG00000180332 | KCTD4 | 1.547112 | 0.017305 |
| ENSG00000237631 | AL161454.1 | 1.57058 | 0.023815 |
| ENSG00000154162 | CDH12 | 1.572793 | 0.030092 |
| ENSG00000241794 | SPRR2A | 1.576552 | 0.046433 |
| ENSG00000238719 | RNU7-96P | 1.58342 | 0.04983 |
| ENSG00000232886 | AF212831.1 | 1.629205 | 0.046803 |
| ENSG00000171345 | KRT19 | 1.683192 | 0.024358 |
| ENSG00000265413 | AP001094.2 | 1.691578 | 0.016471 |
| ENSG00000110848 | CD69 | 1.723593 | 0.020457 |
| ENSG00000263823 | AC009831.1 | 1.728217 | 0.021206 |
| ENSG00000196091 | MYBPC1 | 1.77376 | 0.043146 |
| ENSG00000175084 | DES | 1.789694 | 0.049342 |
| ENSG00000163017 | ACTG2 | 1.93823 | 0.015385 |
| ENSG00000262714 | AC007342.5 | 2.0075 | 0.030785 |
| ENSG00000225329 | LHFPL3-AS2 | 2.266581 | 0.040219 |
| ENSG00000258850 | AL450442.1 | 2.417 | 0.042732 |
| ENSG00000252769 | RNU6-943P | 2.497797 | 0.019334 |
| ENSG00000187105 | HEATR4 | 2.532414 | 0.019343 |
| ENSG00000260549 | MT1L | 2.729623 | 0.006871 |
| ENSG00000279853 | AC004453.2 | 2.732032 | 0.017626 |
| ENSG00000205784 | ARRDC5 | 2.779949 | 0.048281 |
| ENSG00000205791 | LOH12CR2 | 2.797572 | 0.021944 |
| ENSG00000229586 | TNPO1P3 | 2.830789 | 0.006513 |
| ENSG00000239941 | AC108718.1 | 2.889199 | 0.043783 |
| ENSG00000205847 | OR7E91P | 2.999665 | 0.048525 |
| ENSG00000275185 | AC130324.3 | 3.052598 | 0.049335 |
| ENSG00000231665 | OGFOD1P1 | 3.071211 | 0.027051 |
| ENSG00000199963 | RNU6-605P | 3.822897 | 0.026833 |
| ENSG00000272912 | AL356608.1 | 3.862344 | 0.028469 |
| ENSG00000257900 | AL162632.1 | 4.257849 | 0.044317 |
| ENSG00000234647 | AL606970.3 | 4.427793 | 0.030897 |
| ENSG00000165186 | PTCHD 1 | 4.453748 | 0.031022 |
| ENSG00000170231 | FABP6 | 4.492289 | 0.034401 |

TABLE 2
Analysis Results for Treatment B

| Ensembl ID | #N/A | log2Fold Change | P value |
|---|---|---|---|
| ENSG00000151892 | GFRA1 | −1.32221 | 3.88E−05 |
| ENSG00000165474 | GJB2 | 1.998723 | 3.24E−05 |
| ENSG00000196616 | ADH1B | −2.34169 | 3.35E−05 |
| ENSG00000230630 | DNM3OS | −1.57004 | 1.67E−05 |
| ENSG00000169432 | SCN9A | −1.63498 | 1.00E−04 |
| ENSG00000079102 | RUNX1T1 | −1.31846 | 0.000145 |
| ENSG00000140519 | RHCG | 2.657206 | 0.000143 |
| ENSG00000007908 | SELE | 2.526293 | 0.000352 |
| ENSG00000103888 | CEMIP | −1.10131 | 0.000333 |
| ENSG00000138356 | AOX1 | −1.26699 | 0.000378 |
| ENSG00000171659 | GPR34 | −6.17357 | 0.000344 |
| ENSG00000179144 | GIMAP7 | −1.69088 | 0.000377 |
| ENSG00000179639 | FCER1A | −2.1795 | 0.000281 |
| ENSG00000181634 | TNFSF15 | 2.418527 | 0.000349 |
| ENSG00000002587 | HS3ST1 | 1.525045 | 0.009108 |
| ENSG00000006042 | TMEM98 | −0.76049 | 0.018427 |
| ENSG00000006118 | TMEM132A | 1.291593 | 0.014434 |
| ENSG00000006210 | CX3CL1 | 1.278715 | 0.009312 |
| ENSG00000006327 | TNFRSF12A | 1.258901 | 0.001108 |
| ENSG00000006534 | ALDH3B1 | −0.94469 | 0.045664 |
| ENSG00000010610 | CD4 | −0.95687 | 0.036771 |
| ENSG00000010818 | HIVEP2 | 0.614205 | 0.042518 |
| ENSG00000011347 | SYT7 | 0.873369 | 0.027179 |
| ENSG00000023445 | BIRC3 | 1.077344 | 0.022292 |
| ENSG00000025708 | TYMP | 0.966378 | 0.014275 |
| ENSG00000033867 | SLC4A7 | 0.782185 | 0.01909 |
| ENSG00000035664 | DAPK2 | 0.680838 | 0.010423 |
| ENSG00000050730 | TNIP3 | 2.416764 | 0.043461 |
| ENSG00000052344 | PRSS8 | 0.587169 | 0.048358 |
| ENSG00000057149 | SERPINB3 | 2.22796 | 0.037447 |
| ENSG00000058804 | NDC1 | 0.546639 | 0.033555 |
| ENSG00000062038 | CDH3 | 0.620979 | 0.034534 |
| ENSG00000064270 | ATP2C2 | 0.565592 | 0.039556 |
| ENSG00000064692 | SNCAIP | 0.979883 | 0.04439 |
| ENSG00000065413 | ANKRD44 | −0.5516 | 0.035352 |
| ENSG00000067057 | PFKP | 0.725304 | 0.035775 |
| ENSG00000069431 | ABCC9 | −0.60597 | 0.008963 |
| ENSG00000071246 | VASH1 | −1.08894 | 0.015429 |
| ENSG00000072195 | SPEG | 0.788761 | 0.043493 |
| ENSG00000075618 | FSCN1 | 0.702523 | 0.023189 |
| ENSG00000076662 | ICAM3 | −1.52712 | 0.024599 |
| ENSG00000077420 | APBB1IP | −1.29515 | 0.002342 |
| ENSG00000078269 | SYNJ2 | 0.632911 | 0.047794 |
| ENSG00000079841 | RIMS1 | 4.182089 | 0.00324 |
| ENSG00000081479 | LRP2 | −0.99904 | 0.025572 |
| ENSG00000089472 | HEPH | −1.33761 | 0.011905 |
| ENSG00000090020 | SLC9A1 | 0.80385 | 0.01075 |
| ENSG00000090659 | CD209 | −1.38714 | 0.026772 |
| ENSG00000091073 | DTX2 | 0.48646 | 0.039965 |
| ENSG00000091106 | NLRC4 | −2.87202 | 0.015085 |
| ENSG00000091656 | ZFHX4 | −0.85725 | 0.007634 |
| ENSG00000092421 | SEMA6A | 0.733199 | 0.011208 |
| ENSG00000093072 | ADA2 | −1.51831 | 0.004379 |
| ENSG00000093134 | VNN3 | 2.624008 | 0.025062 |
| ENSG00000095637 | SORBS1 | −0.59844 | 0.033373 |
| ENSG00000099953 | MMP11 | −2.05831 | 0.020214 |
| ENSG00000100055 | CYTH4 | −0.92225 | 0.015989 |
| ENSG00000100473 | COCH | −1.80908 | 0.037108 |
| ENSG00000100906 | NFKBIA | 0.898054 | 0.010834 |
| ENSG00000100949 | RABGGTA | 0.71891 | 0.043735 |
| ENSG00000101198 | NKAIN4 | 4.429388 | 0.017603 |
| ENSG00000101230 | ISM1 | −1.64043 | 0.001588 |
| ENSG00000101336 | HCK | −0.94469 | 0.012772 |
| ENSG00000102879 | CORO1A | −1.20645 | 0.019151 |
| ENSG00000103044 | HAS3 | 1.256155 | 0.002567 |
| ENSG00000104043 | ATP8B4 | −1.23721 | 0.002577 |
| ENSG00000104059 | FAM189A1 | 2.30923 | 0.014665 |
| ENSG00000104856 | RELB | 0.56671 | 0.038481 |
| ENSG00000104894 | CD37 | −1.23199 | 0.026094 |
| ENSG00000104998 | IL27RA | 0.780757 | 0.044846 |
| ENSG00000105383 | CD33 | −1.50378 | 0.033147 |
| ENSG00000105472 | CLEC11A | −0.85223 | 0.017967 |
| ENSG00000106113 | CRHR2 | −3.1186 | 0.007054 |
| ENSG00000106123 | EPHB6 | −0.72146 | 0.049111 |
| ENSG00000106366 | SERPINE1 | 1.653201 | 0.000663 |
| ENSG00000106772 | PRUNE2 | −0.70799 | 0.022949 |
| ENSG00000106952 | TNFSF8 | −2.46752 | 0.005619 |
| ENSG00000107611 | CUBN | −0.76066 | 0.019665 |
| ENSG00000108342 | CSF3 | 2.993755 | 0.019962 |
| ENSG00000109193 | SULT1E1 | −2.66331 | 0.000867 |
| ENSG00000109684 | CLNK | −1.82058 | 0.007501 |
| ENSG00000110077 | MS4A6A | −2.11348 | 0.001631 |
| ENSG00000110318 | CEP126 | −0.72738 | 0.027331 |
| ENSG00000110484 | SCGB2A2 | 3.604507 | 0.00201 |
| ENSG00000111012 | CYP27B1 | 1.386392 | 0.012117 |
| ENSG00000112299 | VNN1 | 2.514857 | 0.006809 |
| ENSG00000112964 | GHR | −0.63163 | 0.026862 |
| ENSG00000115008 | IL1A | 1.666825 | 0.042285 |
| ENSG00000115221 | ITGB6 | 1.090528 | 0.023013 |
| ENSG00000116285 | ERRFI1 | 0.793579 | 0.041379 |
| ENSG00000116514 | RNF19B | 0.622973 | 0.041995 |
| ENSG00000116678 | LEPR | −0.89384 | 0.013895 |
| ENSG00000116996 | ZP4 | 5.41556 | 0.036558 |
| ENSG00000117245 | KIF17 | 1.4139 | 0.003754 |
| ENSG00000117266 | CDK18 | 1.282353 | 0.009008 |
| ENSG00000117394 | SLC2A1 | 1.133847 | 0.00494 |
| ENSG00000117707 | PROX1 | −0.82749 | 0.025569 |
| ENSG00000118492 | ADGB | −4.84588 | 0.02764 |
| ENSG00000118503 | TNFAIP3 | 1.128561 | 0.020563 |
| ENSG00000119121 | TRPM6 | 0.807392 | 0.029124 |
| ENSG00000119630 | PGF | 0.947312 | 0.009108 |
| ENSG00000119714 | GPR68 | 0.734348 | 0.045941 |
| ENSG00000120093 | HOXB3 | −0.88695 | 0.009484 |
| ENSG00000120280 | CXorf21 | −1.75574 | 0.037339 |
| ENSG00000120332 | TNN | −3.10685 | 0.022637 |
| ENSG00000120337 | TNFSF18 | 2.513386 | 0.047854 |
| ENSG00000120549 | KIAA1217 | 0.509776 | 0.047799 |
| ENSG00000121594 | CD80 | 1.60636 | 0.037252 |
| ENSG00000121742 | GJB6 | 0.601349 | 0.022805 |
| ENSG00000122420 | PTGFR | −1.0241 | 0.035899 |
| ENSG00000123892 | RAB38 | 0.531157 | 0.033301 |
| ENSG00000123977 | DAW1 | 4.068912 | 0.027416 |
| ENSG00000124102 | PI3 | 3.885679 | 0.026816 |
| ENSG00000124116 | WFDC3 | 1.173262 | 0.030659 |
| ENSG00000124205 | EDN3 | −5.35062 | 0.00535 |
| ENSG00000124253 | PCK1 | −4.13208 | 0.026692 |
| ENSG00000124491 | F13A1 | −1.28627 | 0.005974 |
| ENSG00000124935 | SCGB1D2 | 5.336927 | 0.000801 |
| ENSG00000125144 | MT1G | 1.276601 | 0.041612 |
| ENSG00000125355 | TMEM255A | −1.01987 | 0.043226 |
| ENSG00000125510 | OPRL1 | −2.83216 | 0.006388 |
| ENSG00000126860 | EVI2A | −1.47836 | 0.026935 |
| ENSG00000128342 | LIF | 1.09545 | 0.034413 |
| ENSG00000128408 | RIBC2 | −1.86534 | 0.04493 |
| ENSG00000128578 | STRIP2 | 0.892106 | 0.04061 |
| ENSG00000128596 | CCDC136 | −1.29884 | 0.02299 |
| ENSG00000128815 | WDFY4 | −0.99764 | 0.037415 |
| ENSG00000129521 | EGLN3 | 0.817881 | 0.031911 |
| ENSG00000129538 | RNASE1 | −1.15889 | 0.023141 |
| ENSG00000129596 | CDO1 | −1.24114 | 0.032321 |
| ENSG00000129667 | RHBDF2 | 0.581517 | 0.047031 |
| ENSG00000130066 | SAT1 | 1.047721 | 0.017339 |
| ENSG00000130513 | GDF15 | 2.116514 | 0.020483 |
| ENSG00000130821 | SLC6A8 | 0.84266 | 0.008037 |
| ENSG00000130822 | PNCK | 1.164101 | 0.001886 |
| ENSG00000131941 | RHPN2 | 1.17192 | 0.040975 |
| ENSG00000133019 | CHRM3 | 1.737227 | 0.038156 |
| ENSG00000133048 | CHI3L1 | 0.900197 | 0.00885 |
| ENSG00000133083 | DCLK1 | −1.1124 | 0.018682 |
| ENSG00000133110 | POSTN | −1.13464 | 0.025534 |
| ENSG00000133116 | KL | −2.04286 | 0.003049 |
| ENSG00000133142 | TCEAL4 | −0.84579 | 0.000897 |
| ENSG00000133657 | ATP13A3 | 0.688031 | 0.039217 |
| ENSG00000134070 | IRAK2 | 0.9283 | 0.034916 |
| ENSG00000134107 | BHLHE40 | 0.650695 | 0.046059 |
| ENSG00000134201 | GSTM5 | −1.43483 | 0.032872 |
| ENSG00000134222 | PSRC1 | 1.220644 | 0.029476 |
| ENSG00000134343 | ANO3 | 3.560206 | 0.000764 |
| ENSG00000134532 | SOX5 | −0.80356 | 0.01503 |
| ENSG00000134853 | PDGFRA | −0.78472 | 0.034063 |
| ENSG00000135077 | HAVCR2 | −1.0104 | 0.007644 |
| ENSG00000135114 | OASL | −2.35659 | 0.008454 |
| ENSG00000135480 | KRT7 | 2.248511 | 0.011165 |
| ENSG00000135744 | AGT | −4.68136 | 0.006514 |
| ENSG00000136869 | TLR4 | −1.26519 | 0.002682 |
| ENSG00000136960 | ENPP2 | −0.69348 | 0.042416 |
| ENSG00000137077 | CCL21 | −1.23759 | 0.041645 |
| ENSG00000137331 | IER3 | 1.210882 | 0.006661 |
| ENSG00000137965 | IFI44 | −0.88097 | 0.022657 |
| ENSG00000138074 | SLC5A6 | 0.830577 | 0.046314 |
| ENSG00000138172 | CALHM2 | −0.90259 | 0.017585 |
| ENSG00000138185 | ENTPD1 | −0.72346 | 0.044386 |
| ENSG00000138772 | ANXA3 | 0.937087 | 0.038208 |
| ENSG00000138829 | FBN2 | 2.053294 | 0.006206 |
| ENSG00000139549 | DHH | −3.52427 | 0.002281 |
| ENSG00000139910 | NOVA1 | −1.34799 | 0.004682 |
| ENSG00000139970 | RTN1 | −1.20148 | 0.022401 |
| ENSG00000141338 | ABCA8 | −0.99723 | 0.018259 |
| ENSG00000141837 | CACNA1A | 2.130846 | 0.038401 |
| ENSG00000142512 | SIGLEC10 | −1.68994 | 0.007619 |
| ENSG00000143320 | CRABP2 | 0.742995 | 0.043736 |
| ENSG00000143341 | HMCN1 | −0.69607 | 0.041157 |
| ENSG00000143546 | S100A8 | 1.936459 | 0.038223 |
| ENSG00000143867 | OSR1 | −1.10693 | 0.032889 |
| ENSG00000143891 | GALM | −1.21573 | 0.021309 |
| ENSG00000144891 | AGTR1 | −1.02364 | 0.019455 |
| ENSG00000145428 | RNF175 | −2.05119 | 0.028706 |
| ENSG00000145569 | OTULINL | −0.97241 | 0.018869 |
| ENSG00000145934 | TENM2 | 0.560885 | 0.043357 |
| ENSG00000146232 | NFKBIE | 0.835246 | 0.012019 |
| ENSG00000147443 | DOK2 | −1.41434 | 0.016684 |
| ENSG00000147588 | PMP2 | −3.41117 | 0.001044 |
| ENSG00000148541 | FAM13C | −1.14472 | 0.022865 |
| ENSG00000148737 | TCF7L2 | −0.47378 | 0.039594 |
| ENSG00000148948 | LRRC4C | −5.76674 | 0.000949 |
| ENSG00000149534 | MS4A2 | −1.94466 | 0.001805 |
| ENSG00000149596 | JPH2 | 1.268681 | 0.000844 |
| ENSG00000150471 | ADGRL3 | −1.19562 | 0.005957 |
| ENSG00000150551 | LYPD1 | 3.683677 | 0.009646 |
| ENSG00000150594 | ADRA2A | −0.98525 | 0.007975 |
| ENSG00000150681 | RGS18 | −3.58323 | 0.049172 |
| ENSG00000151651 | ADAM8 | 1.306417 | 0.000878 |
| ENSG00000153012 | LGI2 | −3.37886 | 0.000996 |
| ENSG00000154258 | ABCA9 | −1.38898 | 0.007958 |
| ENSG00000154262 | ABCA6 | −1.26147 | 0.004414 |
| ENSG00000154654 | NCAM2 | −0.95426 | 0.047176 |
| ENSG00000155380 | SLC16A1 | 0.843222 | 0.030287 |
| ENSG00000155659 | VSIG4 | −2.68234 | 0.004079 |
| ENSG00000155846 | PPARGC1B | 0.802633 | 0.019311 |
| ENSG00000155897 | ADCY8 | 5.46317 | 0.018764 |
| ENSG00000155926 | SLA | −1.27487 | 0.001536 |
| ENSG00000156206 | CFAP161 | 1.371251 | 0.038852 |
| ENSG00000156968 | MPV17L | 1.144928 | 0.030553 |
| ENSG00000157214 | STEAP2 | −0.76009 | 0.004214 |
| ENSG00000158125 | XDH | 0.86154 | 0.041556 |
| ENSG00000158477 | CD1A | −1.48446 | 0.010577 |
| ENSG00000158714 | SLAMF8 | −1.26379 | 0.014846 |
| ENSG00000159399 | HK2 | 0.874653 | 0.047559 |
| ENSG00000160161 | CILP2 | 2.080393 | 0.040843 |
| ENSG00000160255 | ITGB2 | −0.87891 | 0.037086 |
| ENSG00000160801 | PTH1R | −1.085 | 0.012452 |
| ENSG00000162367 | TAL1 | −0.83524 | 0.030348 |
| ENSG00000162458 | FBLIM1 | 0.900245 | 0.006128 |
| ENSG00000162511 | LAPTM5 | −0.72874 | 0.027665 |
| ENSG00000162783 | IER5 | 0.52941 | 0.040976 |
| ENSG00000162891 | IL20 | 1.563929 | 0.032905 |
| ENSG00000162999 | DUSP19 | −1.00631 | 0.04579 |
| ENSG00000163106 | HPGDS | −1.78635 | 0.002598 |
| ENSG00000163202 | LCE3D | 1.229664 | 0.007918 |
| ENSG00000163364 | LINC01116 | −2.01462 | 0.001159 |
| ENSG00000163435 | ELF3 | 1.084921 | 0.041451 |
| ENSG00000163600 | ICOS | −4.26397 | 0.04897 |
| ENSG00000163817 | SLC6A20 | 1.13516 | 0.04121 |
| ENSG00000163874 | ZC3H12A | 0.970914 | 0.008588 |
| ENSG00000163993 | S100P | 1.078644 | 0.015178 |
| ENSG00000164086 | DUSP7 | 0.594341 | 0.030262 |
| ENSG00000164093 | PITX2 | 3.128949 | 0.002055 |
| ENSG00000164125 | FAM198B | −0.77034 | 0.027308 |
| ENSG00000164465 | DCBLD1 | 0.693181 | 0.021494 |
| ENSG00000164532 | TBX20 | 4.476152 | 0.046393 |
| ENSG00000164626 | KCNK5 | 0.751519 | 0.008243 |
| ENSG00000164647 | STEAP1 | −0.88539 | 0.018555 |
| ENSG00000165124 | SVEP1 | −1.18067 | 0.012634 |
| ENSG00000165168 | CYBB | −1.57528 | 0.002202 |
| ENSG00000165186 | PTCHD1 | 5.188262 | 0.004175 |
| ENSG00000165424 | ZCCHC24 | −0.57526 | 0.040242 |
| ENSG00000165646 | SLC18A2 | −1.39222 | 0.010601 |
| ENSG00000166016 | ABTB2 | 0.929961 | 0.0317 |
| ENSG00000166148 | AVPR1A | −1.0199 | 0.049461 |
| ENSG00000166444 | ST5 | 0.692779 | 0.047178 |
| ENSG00000166501 | PRKCB | −1.19027 | 0.004266 |
| ENSG00000166869 | CHP2 | −1.06893 | 0.025068 |
| ENSG00000166923 | GREM1 | −0.97734 | 0.021582 |
| ENSG00000167046 | AL357033.1 | −1.39789 | 0.040742 |
| ENSG00000167208 | SNX20 | −1.27325 | 0.012173 |
| ENSG00000167600 | CYP2S1 | 1.122143 | 0.045619 |
| ENSG00000167772 | ANGPTL4 | 1.089528 | 0.005596 |
| ENSG00000167850 | CD300C | −2.53135 | 0.010959 |
| ENSG00000168405 | CMAHP | −0.93167 | 0.001794 |
| ENSG00000168484 | SFTPC | 3.21244 | 0.027007 |
| ENSG00000168539 | CHRM1 | −1.61421 | 0.04102 |
| ENSG00000168658 | VWA3B | 5.039705 | 0.033509 |
| ENSG00000169031 | COL4A3 | 1.818289 | 0.02676 |
| ENSG00000169258 | GPRIN1 | 0.965488 | 0.041968 |
| ENSG00000169402 | RSPH10B2 | −2.70638 | 0.048716 |
| ENSG00000169403 | PTAFR | 0.79061 | 0.019202 |
| ENSG00000169435 | RASSF6 | −0.82012 | 0.026676 |
| ENSG00000169554 | ZEB2 | −0.53565 | 0.024672 |
| ENSG00000169862 | CTNND2 | −2.44175 | 0.042656 |
| ENSG00000170231 | FABP6 | 5.051062 | 0.003684 |
| ENSG00000170412 | GPRC5C | 0.727253 | 0.020582 |
| ENSG00000170465 | KRT6C | 1.064631 | 0.008998 |
| ENSG00000170961 | HAS2 | −1.0146 | 0.049566 |
| ENSG00000171345 | KRT19 | 2.732616 | 0.00047 |
| ENSG00000171517 | LPAR3 | 0.669821 | 0.014472 |
| ENSG00000171772 | SYCE1 | −5.78577 | 0.019889 |
| ENSG00000171777 | RASGRP4 | −1.45742 | 0.036383 |
| ENSG00000171819 | ANGPTL7 | −4.83588 | 0.023268 |
| ENSG00000172216 | CEBPB | 0.566303 | 0.044831 |
| ENSG00000172367 | PDZD3 | 1.229735 | 0.040873 |
| ENSG00000172476 | RAB40A | −1.57229 | 0.010013 |
| ENSG00000172752 | COL6A5 | −1.2029 | 0.047348 |
| ENSG00000172901 | LVRN | −1.08334 | 0.020994 |
| ENSG00000172987 | HPSE2 | −1.26563 | 0.020139 |
| ENSG00000173597 | SULT1B1 | −2.155 | 0.049151 |
| ENSG00000174276 | ZNHIT2 | 1.054499 | 0.044075 |
| ENSG00000174482 | LINGO2 | 3.262592 | 0.048625 |
| ENSG00000175567 | UCP2 | −1.08769 | 0.043546 |
| ENSG00000175643 | RMI2 | −1.34791 | 0.035325 |
| ENSG00000175899 | A2M | −0.83363 | 0.034642 |
| ENSG00000176399 | DMRTA1 | 2.220599 | 0.01829 |
| ENSG00000177359 | AC024940.1 | 2.00458 | 0.001395 |
| ENSG00000177606 | JUN | 0.766636 | 0.018491 |
| ENSG00000177640 | CASC2 | −1.86958 | 0.003927 |
| ENSG00000178662 | CSRNP3 | −0.89081 | 0.032182 |
| ENSG00000179431 | FJX1 | 1.272649 | 0.010967 |
| ENSG00000179580 | RNF151 | 2.337534 | 0.041208 |
| ENSG00000179593 | ALOX15B | 0.956841 | 0.007661 |
| ENSG00000180549 | FUT7 | −3.61988 | 0.038086 |
| ENSG00000181036 | FCRL6 | 4.225177 | 0.026776 |
| ENSG00000181322 | NME9 | −4.50617 | 0.01033 |
| ENSG00000181649 | PHLDA2 | 1.070873 | 0.043291 |
| ENSG00000182197 | EXT1 | 0.555698 | 0.02407 |
| ENSG00000182578 | CSF1R | −1.32431 | 0.004623 |
| ENSG00000182636 | NDN | −0.9871 | 0.043312 |
| ENSG00000183625 | CCR3 | −2.02192 | 0.023512 |
| ENSG00000183691 | NOG | 1.207521 | 0.043148 |
| ENSG00000184148 | SPRR4 | −3.57852 | 0.016804 |
| ENSG00000184785 | SMIM10 | −1.47347 | 0.043705 |
| ENSG00000184949 | FAM227A | −0.98083 | 0.0151 |
| ENSG00000185022 | MAFF | 0.72208 | 0.043387 |
| ENSG00000185043 | CIB1 | 0.77811 | 0.014062 |
| ENSG00000185215 | TNFAIP2 | 1.230296 | 0.024957 |
| ENSG00000185477 | GPRIN3 | −1.01412 | 0.008311 |
| ENSG00000185499 | MUC1 | 0.928034 | 0.010308 |
| ENSG00000185610 | DBX2 | −4.32694 | 0.019551 |
| ENSG00000185745 | IFIT1 | −1.53898 | 0.010774 |
| ENSG00000186188 | FFAR4 | −2.51302 | 0.024415 |
| ENSG00000186407 | CD300E | −1.15194 | 0.019641 |
| ENSG00000186417 | GLDN | −1.12334 | 0.002728 |
| ENSG00000187242 | KRT12 | −3.9608 | 0.041535 |
| ENSG00000187479 | C11orf96 | 0.781848 | 0.04517 |
| ENSG00000187510 | PLEKHG7 | 2.402051 | 0.010702 |
| ENSG00000187775 | DNAH17 | 1.557119 | 0.000704 |
| ENSG00000187950 | OVCH1 | −2.75139 | 0.017068 |
| ENSG00000187957 | DNER | 2.48235 | 0.048883 |
| ENSG00000188921 | HACD4 | −0.79432 | 0.034766 |
| ENSG00000189221 | MAOA | −0.65218 | 0.047316 |
| ENSG00000189410 | SH2D5 | 1.474806 | 0.007918 |
| ENSG00000189423 | USP32P3 | 1.401408 | 0.037907 |
| ENSG00000196091 | MYBPC1 | 1.862106 | 0.039549 |
| ENSG00000196159 | FAT4 | −0.77194 | 0.007454 |
| ENSG00000197406 | DIO3 | −1.56382 | 0.013093 |
| ENSG00000197471 | SPN | −1.41704 | 0.009554 |
| ENSG00000197496 | SLC2A10 | −0.87122 | 0.031024 |
| ENSG00000197599 | CCDC154 | 1.192769 | 0.026495 |
| ENSG00000197696 | NMB | 0.992214 | 0.035393 |
| ENSG00000198019 | FCGR1B | −5.13921 | 0.028286 |
| ENSG00000198113 | TOR4A | 0.738898 | 0.020026 |
| ENSG00000198133 | TMEM229B | −1.74205 | 0.049357 |
| ENSG00000198719 | DLL1 | 0.983359 | 0.015225 |
| ENSG00000198743 | SLC5A3 | 0.78782 | 0.048257 |
| ENSG00000198984 | MIR345 | −3.05678 | 0.031076 |
| ENSG00000199867 | RF00019 | 3.969279 | 0.044762 |
| ENSG00000200648 | RNU6-226P | 3.924728 | 0.046277 |
| ENSG00000203685 | STUM | −1.01085 | 0.007433 |
| ENSG00000203710 | CR1 | −2.68407 | 0.021416 |
| ENSG00000203724 | C1orf53 | −4.98962 | 0.034453 |
| ENSG00000204021 | LIPK | −0.71852 | 0.0077 |
| ENSG00000204131 | NHSL2 | −0.77068 | 0.031825 |
| ENSG00000204385 | SLC44A4 | 1.79047 | 0.015432 |
| ENSG00000204472 | AIF1 | −2.17051 | 0.00303 |
| ENSG00000204936 | CD177 | 2.660617 | 0.000524 |
| ENSG00000205221 | VIT | −1.22314 | 0.02709 |
| ENSG00000205420 | KRT6A | 0.67047 | 0.041662 |
| ENSG00000206073 | SERPINB4 | 1.905943 | 0.042618 |
| ENSG00000206538 | VGLL3 | −0.93621 | 0.004365 |
| ENSG00000206870 | RNU6-398P | −4.69792 | 0.032615 |
| ENSG00000207175 | RNU1-67P | 1.140537 | 0.038104 |
| ENSG00000207646 | MIR655 | −4.13076 | 0.039316 |
| ENSG00000207924 | MIR196A2 | −2.75244 | 0.042008 |
| ENSG00000211448 | DIO2 | −0.9421 | 0.035835 |
| ENSG00000211514 | MIR454 | −3.57525 | 0.034002 |
| ENSG00000212576 | RNA5SP467 | −2.70773 | 0.036865 |
| ENSG00000213366 | GSTM2 | −0.97056 | 0.034789 |
| ENSG00000213763 | ACTBP2 | −4.04329 | 0.032941 |
| ENSG00000214856 | KRT16P1 | 2.751225 | 0.030663 |
| ENSG00000215068 | AC025171.2 | −1.38453 | 0.017257 |
| ENSG00000215853 | RPTN | −1.16983 | 0.029925 |
| ENSG00000223086 | RNA5SP155 | 1.393708 | 0.035892 |
| ENSG00000223949 | ROR1-AS1 | 3.617439 | 0.049764 |
| ENSG00000223991 | AC104809.1 | −5.29754 | 0.020528 |
| ENSG00000224014 | AL390728.3 | 2.635868 | 0.029055 |
| ENSG00000224043 | CCNT2-AS1 | −1.66882 | 0.024906 |
| ENSG00000224631 | RPS27AP16 | 1.174144 | 0.041663 |
| ENSG00000224794 | AL022326.1 | −4.92944 | 0.019394 |
| ENSG00000225568 | AC093155.1 | 1.356816 | 0.027717 |
| ENSG00000225670 | CADM3-AS1 | −1.26552 | 0.021554 |
| ENSG00000225857 | AL162431.1 | 4.145279 | 0.029835 |
| ENSG00000226977 | HMGN1P24 | 2.334911 | 0.025554 |
| ENSG00000227165 | WDR11-AS1 | 2.735438 | 0.031354 |
| ENSG00000227218 | AL157935.1 | 4.38376 | 0.012234 |
| ENSG00000227456 | LINC00310 | 1.090748 | 0.001404 |
| ENSG00000227755 | AP000344.1 | −3.01801 | 0.04054 |
| ENSG00000227908 | FLJ31104 | 1.729262 | 0.049251 |
| ENSG00000228403 | AC035139.1 | −5.1104 | 0.013189 |
| ENSG00000229586 | TNPO1P3 | 1.969826 | 0.025438 |
| ENSG00000229989 | MIR181A1HG | −1.19091 | 0.00756 |
| ENSG00000230581 | ACTG1P14 | 0.927086 | 0.041583 |
| ENSG00000230638 | AL445933.1 | −5.03144 | 0.013676 |
| ENSG00000231440 | AL358176.4 | −2.62875 | 0.020687 |
| ENSG00000231530 | AL157932.1 | −4.08256 | 0.034522 |
| ENSG00000231971 | AL078590.2 | 2.102171 | 0.019766 |
| ENSG00000232202 | AC098824.1 | −4.73267 | 0.036094 |
| ENSG00000232388 | SMIM26 | 0.68801 | 0.043088 |
| ENSG00000233101 | HOXB-AS3 | −2.23498 | 0.034054 |
| ENSG00000233421 | LINC01783 | 2.95317 | 0.029141 |
| ENSG00000233435 | AGGF1P2 | 1.779254 | 0.045084 |
| ENSG00000233487 | RPSAP69 | −2.20333 | 0.043987 |
| ENSG00000233621 | LINC01137 | 0.708855 | 0.037161 |
| ENSG00000233716 | AC074367.1 | −1.2453 | 0.037839 |
| ENSG00000233806 | LINC01237 | −0.69182 | 0.032398 |
| ENSG00000233896 | PDYN-AS1 | 5.087673 | 0.026049 |
| ENSG00000233942 | AC004012.1 | −4.625 | 0.012003 |
| ENSG00000234409 | CCDC188 | −3.5305 | 0.013156 |
| ENSG00000234502 | FYTTD1P1 | 4.803838 | 0.025342 |
| ENSG00000235335 | AC016723.1 | 4.878611 | 0.044438 |
| ENSG00000235568 | NFAM1 | −1.5152 | 0.005055 |
| ENSG00000235641 | LINC00484 | −5.03187 | 0.019796 |
| ENSG00000235961 | PNMA6A | −4.82821 | 0.008845 |
| ENSG00000236780 | LINC01829 | 1.790914 | 0.023459 |
| ENSG00000236806 | RPL7AP15 | −2.88958 | 0.003902 |
| ENSG00000237036 | ZEB1-AS1 | −0.82909 | 0.028945 |
| ENSG00000237476 | LINC01637 | 4.407879 | 0.048278 |
| ENSG00000237515 | SHISA9 | 4.304333 | 0.021746 |
| ENSG00000237927 | AL078604.2 | 1.197206 | 0.048734 |
| ENSG00000239791 | AC002310.2 | 2.731137 | 0.040482 |
| ENSG00000239930 | AP001625.3 | 4.61281 | 0.033237 |
| ENSG00000240950 | AC021074.1 | −2.34646 | 0.046496 |
| ENSG00000240972 | MIF | 0.60293 | 0.041358 |
| ENSG00000241399 | CD302 | −0.77945 | 0.040563 |
| ENSG00000241641 | RPS23P6 | −1.51583 | 0.011548 |
| ENSG00000242986 | RPL21P99 | −2.55448 | 0.01924 |
| ENSG00000243244 | STON1 | −0.74999 | 0.027029 |
| ENSG00000243927 | MRPS6 | 0.674442 | 0.042996 |
| ENSG00000244378 | RPS2P45 | 1.144366 | 0.042653 |
| ENSG00000244513 | AC109587.1 | −2.07744 | 0.017517 |
| ENSG00000245213 | AC105285.1 | −1.45609 | 0.020498 |
| ENSG00000246982 | Z84485.1 | −0.83928 | 0.046183 |
| ENSG00000247095 | MIR210HG | 0.764819 | 0.035783 |
| ENSG00000247699 | AC008609.1 | 5.382604 | 0.014764 |
| ENSG00000248583 | AC119751.3 | −5.10367 | 0.038507 |
| ENSG00000248642 | OR10J2P | −5.4446 | 0.014333 |
| ENSG00000249790 | AC092490.1 | 3.626207 | 0.001142 |
| ENSG00000250346 | EEF1GP2 | −4.45313 | 0.029017 |
| ENSG00000250508 | AP000808.1 | −4.71553 | 0.030079 |
| ENSG00000250971 | AC108474.1 | 4.198206 | 0.02993 |
| ENSG00000251095 | AC097478.1 | −0.86983 | 0.011886 |
| ENSG00000251108 | YBX1P5 | −2.95235 | 0.017887 |
| ENSG00000251143 | AP002490.1 | 0.981476 | 0.04065 |
| ENSG00000251363 | LINC02315 | 2.251264 | 0.043457 |
| ENSG00000251400 | ALDH7A1P1 | −2.7412 | 0.025698 |
| ENSG00000251417 | AC145285.2 | −4.48565 | 0.01121 |
| ENSG00000252212 | RNU2-58P | −2.62154 | 0.013747 |
| ENSG00000253468 | AP003355.1 | 2.424096 | 0.027111 |
| ENSG00000253632 | AC084026.2 | 5.281195 | 0.012042 |
| ENSG00000254245 | PCDHGA3 | −0.92054 | 0.04222 |
| ENSG00000254423 | AC087203.1 | −4.86137 | 0.049384 |
| ENSG00000254602 | AP000662.1 | −1.00133 | 0.031361 |
| ENSG00000255417 | MTCO2P15 | 4.895983 | 0.022358 |
| ENSG00000255836 | AC131206.1 | −2.79331 | 0.043596 |
| ENSG00000256646 | AC010132.3 | −4.74653 | 0.03306 |
| ENSG00000257178 | AC103702.1 | −1.60556 | 0.036244 |
| ENSG00000257743 | MGAM2 | 5.01328 | 0.016592 |
| ENSG00000258302 | AC025034.1 | 2.390493 | 0.038052 |
| ENSG00000259134 | LINC00924 | −4.16536 | 0.024316 |
| ENSG00000259194 | AC020891.1 | −3.75635 | 0.026847 |
| ENSG00000259390 | AC022196.1 | −3.97116 | 0.04704 |
| ENSG00000259660 | DNM1P47 | −2.00289 | 0.008841 |
| ENSG00000259746 | HSPE1P3 | −1.32527 | 0.024099 |
| ENSG00000260220 | CCDC187 | −1.93768 | 0.0452 |
| ENSG00000260549 | MT1L | 2.915732 | 0.001764 |
| ENSG00000260578 | AC110597.1 | −2.61116 | 0.034146 |
| ENSG00000260586 | AC064799.2 | 0.900611 | 0.037322 |
| ENSG00000260599 | AC011467.1 | −2.50319 | 0.046118 |
| ENSG00000260763 | AC106799.3 | −4.88351 | 0.017759 |
| ENSG00000260846 | FRG2HP | −1.21239 | 0.048054 |
| ENSG00000260871 | AC093510.2 | 3.862024 | 0.042048 |
| ENSG00000260919 | AC100835.1 | 1.97055 | 0.038489 |
| ENSG00000261039 | LINC02544 | 4.476152 | 0.046393 |
| ENSG00000261618 | LINC02605 | 0.901403 | 0.047047 |
| ENSG00000261775 | AC012435.2 | −1.18098 | 0.037224 |
| ENSG00000265531 | FCGR1CP | −4.96538 | 0.014079 |
| ENSG00000265750 | AC090772.3 | −1.92615 | 0.00413 |
| ENSG00000265972 | TXNIP | −1.20786 | 0.000745 |
| ENSG00000266803 | AC127540.1 | −1.83865 | 0.045041 |
| ENSG00000267287 | AC068473.3 | −1.71738 | 0.042088 |
| ENSG00000267288 | AC138150.2 | 1.245129 | 0.045497 |
| ENSG00000267361 | SEC24AP1 | −1.2189 | 0.040094 |
| ENSG00000267702 | AP005131.6 | −1.09784 | 0.009712 |
| ENSG00000268906 | AC011473.3 | −1.00378 | 0.048921 |
| ENSG00000269397 | AC011503.2 | −1.57644 | 0.019406 |
| ENSG00000270077 | AP003117.1 | −1.23992 | 0.039366 |
| ENSG00000270716 | BNIP3P15 | −5.08661 | 0.010068 |
| ENSG00000270948 | MTDHP1 | −3.33315 | 0.016368 |
| ENSG00000271141 | AC010680.4 | −0.86066 | 0.045593 |
| ENSG00000271811 | Z97200.1 | −1.1396 | 0.001626 |
| ENSG00000271856 | LINC01215 | 4.23757 | 0.007378 |
| ENSG00000272636 | DOC2B | 0.718935 | 0.041045 |
| ENSG00000272717 | AC112236.2 | 3.049693 | 0.031769 |
| ENSG00000273291 | AC092042.3 | −5.03727 | 0.013607 |
| ENSG00000273554 | AC136616.1 | 4.5383 | 0.039998 |
| ENSG00000273604 | EPOP | 1.077336 | 0.009869 |
| ENSG00000273628 | AL354798.1 | −4.13742 | 0.026874 |
| ENSG00000274215 | AC106028.4 | 5.157934 | 0.006648 |
| ENSG00000274964 | AC026356.1 | −2.94555 | 0.006213 |
| ENSG00000275183 | LENG9 | 0.830744 | 0.04776 |
| ENSG00000275897 | AC021491.4 | −2.06423 | 0.016856 |
| ENSG00000275993 | SIK1B | 0.842547 | 0.016424 |
| ENSG00000276107 | AC037198.1 | −1.06776 | 0.009263 |
| ENSG00000276317 | AL357033.3 | −2.24551 | 0.007249 |
| ENSG00000276409 | CCL14 | −2.18517 | 0.008489 |
| ENSG00000276696 | RF00019 | −4.7248 | 0.005051 |
| ENSG00000277443 | MARCKS | 0.835941 | 0.033194 |
| ENSG00000278642 | AC015813.4 | −4.8736 | 0.046208 |
| ENSG00000278769 | AC090510.3 | −1.32179 | 0.028459 |
| ENSG00000278876 | AC145207.9 | 0.837003 | 0.02638 |
| ENSG00000279125 | AC091953.3 | −4.76742 | 0.032557 |
| ENSG00000279174 | AC104581.3 | 1.747844 | 0.045459 |
| ENSG00000279289 | AL136164.3 | 0.883653 | 0.03773 |
| ENSG00000279725 | AL391005.1 | 1.235254 | 0.04738 |
| ENSG00000279757 | AC132068.1 | −1.33079 | 0.04499 |
| ENSG00000279853 | AC004453.2 | 2.410185 | 0.020357 |
| ENSG00000279903 | AP006248.3 | 3.045908 | 0.00642 |
| ENSG00000280032 | AP002800.1 | 0.895734 | 0.029341 |
| ENSG00000280106 | AC008555.8 | −1.16538 | 0.022208 |
| ENSG00000280222 | AL365209.1 | −3.34092 | 0.001528 |
| ENSG00000281769 | LINC01230 | −4.87002 | 0.045991 |
| ENSG00000281852 | LINC00891 | −5.19097 | 0.008401 |
| ENSG00000282943 | AC004784.1 | 1.463839 | 0.00795 |
| ENSG00000283259 | AC242022.1 | 4.106126 | 0.021417 |
| ENSG00000283283 | AC013268.4 | −2.95729 | 0.010107 |
| ENSG00000283913 | AL512662.2 | −3.38767 | 0.009404 |
| ENSG00000283973 | AC099795.1 | 1.609431 | 0.041626 |
| ENSG00000284138 | ATP6V0CP4 | 1.076565 | 0.009723 |
| ENSG00000284748 | AL513220.1 | 4.622587 | 0.029931 |
| ENSG00000285567 | AC074051.5 | −4.60987 | 0.048159 |
| ENSG00000285719 | AL356275.2 | −4.27803 | 0.043619 |
| ENSG00000285825 | AP003501.3 | 3.245747 | 0.020509 |
| ENSG00000285878 | AP002961.1 | 4.774234 | 0.047986 |
| ENSG00000286048 | #N/A | 5.225498 | 0.019455 |

TABLE 3
Treatment A and Treatment B Results

| | | | log2Fold | | log2Fold | |
|---|---|---|---|---|---|---|
| | | | Change | p value | Change | p value |
| Combined | trtA | trtB | (trtA) | (trtA) | (trt B) | (trtB) |
| A1BG | A1BG | | −2.176992834 | 0.035129908 | | |
| ADA2 | ADA2 | ADA2 | −1.176153431 | 0.012458846 | −1.518305647 | 0.004379081 |
| ADAM8 | | ADAM8 | | | 1.306416922 | 0.000877789 |
| ALDH3B1 | | ALDH3B1 | | | −0.944687186 | 0.045663532 |
| APBB1IP | | APBB1IP | | | −1.295147506 | 0.002341974 |
| ATP8B4 | | ATP8B4 | | | −1.237211351 | 0.002577479 |
| BIRC3 | | BIRC3 | | | 1.077343836 | 0.022291624 |
| CD177 | | CD177 | | | 2.660616828 | 0.000524175 |
| CD1A | | CD1A | | | −1.484455099 | 0.010576659 |
| CD209 | CD209 | CD209 | −1.548920818 | 0.005956087 | −1.387135065 | 0.026771659 |
| CD300C | | CD300C | | | −2.531351521 | 0.010959152 |
| CD300E | CD300E | CD300E | −0.995659398 | 0.014179478 | −1.151941403 | 0.019641037 |
| CD33 | | CD33 | | | −1.503779794 | 0.033147314 |
| CD34 | CD34 | | −1.03600684 | 0.003196933 | | |
| CD4 | | CD4 | | | −0.956869167 | 0.036771421 |
| CD93 | CD93 | | −0.93389813 | 0.002470079 | | |
| CD80 | | CD80 | | | 1.606360495 | 0.037251921 |
| CHI3L1 | | CHI3L1 | | | 0.900196693 | 0.008850429 |
| CLEC10A | CLEC10A | | −1.181978344 | 0.037844257 | | |
| CLEC12A | CLEC12A | | −1.949860983 | 0.029609802 | | |
| CLEC4A | CLEC4A | | −2.463182774 | 0.034773764 | | |
| CR1 | CR1 | CR1 | −2.972211715 | 0.018846039 | −2.684068687 | 0.021416215 |
| CSF1R | | CSF1R | | | −1.324312414 | 0.004623112 |
| CSF3 | | CSF3 | | | 2.99375543 | 0.019962023 |
| CSF3R | CSF3R | | −2.587819636 | 0.013259602 | | |
| CYBB | | CYBB | | | −1.575281165 | 0.002201764 |
| DUSP7 | | DUSP7 | | | 0.594340854 | 0.030261604 |
| DUSP9 | DUSP9 | | −5.023435764 | 0.03401719 | | |
| F13A1 | F13A1 | F13A1 | −1.030395734 | 0.027423728 | −1.286271066 | 0.005973549 |
| FABP5 | FABP5 | | 0.617092568 | 0.005703928 | | |
| FCER1A | | FCER1A | | | −2.179504136 | 0.000281011 |
| FCGR1B | | FCGR1B | | | −5.139209781 | 0.028286452 |
| FSCN1 | | FSCN1 | | | 0.702522699 | 0.023189337 |
| GFRA1 | GFRA1 | GFRA1 | −0.723492066 | 0.037259099 | −1.322206845 | 0.0000388 |
| GHR | | GHR | | | −0.63162604 | 0.02686156 |
| HAVCR2 | | HAVCR2 | | | −1.010396837 | 0.007644379 |
| HCK | | HCK | | | −0.944693685 | 0.01277202 |
| ICAM3 | | ICAM3 | | | −1.527119956 | 0.024598515 |
| ICOS | | ICOS | | | −4.263973212 | 0.048970235 |
| IFIT1 | | IFIT1 | | | −1.538980174 | 0.01077363 |
| IL1A | | IL1A | | | 1.666824887 | 0.042284893 |
| IL1R2 | IL1R2 | | −1.176708886 | 0.036150426 | | |
| IL20 | | IL20 | | | 1.563928574 | 0.032904891 |
| IL27RA | | IL27RA | | | 0.780757109 | 0.044845594 |
| INPP5D | INPP5D | | −0.49209701 | 0.040113013 | | |
| IRAK2 | | IRAK2 | | | 0.928300488 | 0.034916227 |
| IRAK3 | IRAK3 | | −0.939924843 | 0.025740822 | | |
| ITGB2 | | ITGB2 | | | −0.878908416 | 0.03708595 |
| JAK3 | JAK3 | | −0.971828065 | 0.003845435 | | |
| JUN | | JUN | | | 0.766635764 | 0.018491381 |
| KL | KL | KL | −1.991876757 | 0.008240776 | −2.042862851 | 0.003049351 |
| KLHL3 | KLHL3 | | −0.683963477 | 0.020243319 | | |
| LIF | | LIF | | | 1.095449833 | 0.034412923 |
| MAOA | | MAOA | | | −0.65217635 | 0.047315854 |
| MEF2C | MEF2C | | −0.678770928 | 0.014590763 | | |
| MIF | | MIF | | | 0.602929675 | 0.041357906 |
| MRC1 | MRC1 | | −1.278529814 | 0.034040819 | | |
| MS4A2 | | MS4A2 | | | −1.94466299 | 0.001805287 |
| MUC1 | | MUC1 | | | 0.92803421 | 0.010307665 |
| NDC1 | | NDC1 | | | 0.546638514 | 0.033555191 |
| NDN | | NDN | | | −0.987095908 | 0.043311595 |
| NFAM1 | | NFAM1 | | | −1.515198722 | 0.005055059 |
| NFKBIA | | NFKBIA | | | 0.898054214 | 0.010834279 |
| NFKBIE | | NFKBIE | | | 0.835245897 | 0.012019494 |
| NLRC4 | | NLRC4 | | | −2.87201538 | 0.015085426 |
| NOS3 | NOS3 | | −0.876950928 | 0.029841648 | | |
| NPDC1 | NPDC1 | | −0.993124992 | 0.011050918 | | |
| OASL | | OASL | | | −2.356590252 | 0.00845375 |
| PDGFRA | PDGFRA | PDGFRA | −0.883315726 | 0.037488469 | −0.78472058 | 0.034062646 |
| PECAM1 | PECAM1 | | −1.345251452 | 0.00204955 | | |
| PI3 | | PI3 | | | 3.885679494 | 0.026815936 |
| PRKCB | | PRKCB | | | −1.190274126 | 0.004266041 |
| PTAFR | | PTAFR | | | 0.790609858 | 0.019202319 |
| PTX3 | PTX3 | | −3.774134918 | 0.021132793 | | |
| RAPGEF3 | RAPGEF3 | | −1.162363208 | 0.004515442 | | |
| RASGRP1 | RASGRP1 | | 0.644104497 | 0.022965359 | | |
| RASGRP4 | | RASGRP4 | | | −1.457415259 | 0.036382584 |
| RELB | | RELB | | | 0.566710253 | 0.038480664 |
| RPS6KA2 | RPS6KA2 | | −0.64065565 | 0.02586569 | | |
| RNF19B | | RNF19B | | | 0.62297291 | 0.0419949 |
| S100A8 | | S100A8 | | | 1.936458801 | 0.03822337 |
| S100P | | S100P | | | 1.078643774 | 0.015177744 |
| S1PR1 | S1PR1 | | −1.529436529 | 0.000497651 | | |
| SERPINB3 | | SERPINB3 | | | 2.227959787 | 0.037446847 |
| SIGLEC10 | | SIGLEC10 | | | −1.689936092 | 0.007619078 |
| SIRPB1 | SIRPB1 | | −3.182074693 | 0.039981357 | | |
| SLAMF6 | SLAMF6 | | −3.659431109 | 0.024172901 | | |
| SLC11A1 | SLC11A1 | | −1.322813212 | 0.025192294 | | |
| STING1 | STING1 | | | | | |
| TEK | TEK | | −0.930895145 | 0.040503873 | | |
| TLR4 | TLR4 | TLR4 | −1.344666266 | 0.00159401 | −1.265189491 | 0.002682013 |
| TRIM36 | TRIM36 | | 0.945405621 | 0.047173659 | | |
| TNFAIP3 | | TNFAIP3 | | | 1.128560803 | 0.020562736 |
| TNFRSF12A | | TNFRSF12A | | | 1.258901074 | 0.001107975 |
| TNFSF15 | | TNFSF15 | | | 2.418527187 | 0.000349074 |
| TNFSF18 | | TNFSF18 | | | 2.513386432 | 0.047853557 |
| TNFSF8 | | TNFSF8 | | | −2.467523467 | 0.005619429 |
| TXNIP | | TXNIP | | | −1.207855518 | 0.000744715 |
| VNN1 | | VNN1 | | | 2.514856691 | 0.006808972 |
| XDH | | XDH | | | 0.861539959 | 0.0415556 |

TABLE 4
Anti-Inflammation Gene Summaries

| | | TrtA | TrtA | TrtB | TrtB |
|---|---|---|---|---|---|
| Gene | Description | log2fold | p value | Log2fold | P value |
| ADA2 | Studies suggest that it acts as a growth factor, which | −1.17615 | 0.012459 | −1.51831 | 0.004379 |
| | means that it stimulates cell growth and division. In | | | | |
| | particular, the enzyme appears to be involved in the | | | | |
| | growth and development of certain immune system cells, | | | | |
| | including macrophages, which are a type of white blood | | | | |
| | cell that plays a critical role in inflammation. | | | | |
| | Inflammation is a normal immune system response to | | | | |
| | injury and foreign invaders (such as bacteria). Some | | | | |
| | macrophages are pro-inflammatory, meaning they | | | | |
| | promote inflammation, while others are anti- | | | | |
| | inflammatory, meaning they reduce inflammation. | | | | |
| CD209 | This gene encodes a C-type lectin that functions in cell | −1.54892 | 0.005956 | −1.38714 | 0.026772 |
| | adhesion and pathogen recognition. This receptor | | | | |
| | recognizes a wide range of evolutionarily divergent | | | | |
| | pathogens with a large impact on public health, including | | | | |
| | leprosy and tuberculosis mycobacteria, the Ebola, | | | | |
| | hepatitis C, HIV-1 and Dengue viruses, and the SARS- | | | | |
| | CoV acute respiratory syndrome coronavirus. The | | | | |
| | protein is organized into four distinct domains: a C- | | | | |
| | terminal carbohydrate recognition domain, a flexible | | | | |
| | tandem-repeat neck domain, a transmembrane region and | | | | |
| | an N-terminal cytoplasmic domain involved in | | | | |
| | internalization. This gene is closely related in terms of | | | | |
| | both sequence and function to a neighboring gene, | | | | |
| | CLEC4M (Gene ID: 10332), also known as L-SIGN. | | | | |
| | The two genes differ in viral recognition and expression | | | | |
| | patterns, with this gene showing high expression on the | | | | |
| | surface of dendritic cells. Polymorphisms in the neck | | | | |
| | region are associated with protection from HIV-1 | | | | |
| | infection, while single nucleotide polymorphisms in the | | | | |
| | promoter of this gene are associated with differing | | | | |
| | resistance and susceptibility to and severity of infectious | | | | |
| | disease, including rs4804803, which is associated with | | | | |
| | SARS severity. [provided by RefSeq, May 2020] | | | | |
| CD300E | This gene encodes a member of the CD300 glycoprotein | −0.99566 | 0.014179 | −1.15194 | 0.019641 |
| | family of cell surface proteins expressed on myeloid | | | | |
| | cells. The protein interacts with the TYRO protein | | | | |
| | tyrosine kinase-binding protein and is thought to act as | | | | |
| | an activating receptor. [provided by RefSeq, November | | | | |
| | 2012] | | | | |
| CR1 | Membrane immune adherence receptor that plays a | −2.97221 | 0.018846 | −2.68407 | 0.021416 |
| | critical role in the capture and clearance of complement- | | | | |
| | opsonized pathogens by erythrocytes and | | | | |
| | monocytes/macrophages (PubMed: 2963069). Mediates | | | | |
| | the binding by these cells of particles and immune | | | | |
| | complexes that have activated complement to eliminate | | | | |
| | them from the circulation (PubMed: 2963069). Acts also | | | | |
| | in the inhibition of spontaneous complement activation | | | | |
| | by impairing the formation and function of the | | | | |
| | alternative and classical pathway C3/C5 convertases, and | | | | |
| | by serving as a cofactor for the cleavage by factor I of | | | | |
| | C3b to iC3b, C3c and C3d, g, and of C4b to C4c and C4d | | | | |
| | (PubMed: 2972794, 8175757). Also plays a role in | | | | |
| | immune regulation by contributing, upon ligand binding, | | | | |
| | to the generation of regulatory T cells from activated | | | | |
| | helper T cells (PubMed: 25742728). | | | | |
| | (CR1_HUMAN, P17927) | | | | |
| F13A1 | This gene encodes the coagulation factor XIII A subunit. | −1.0304 | 0.027424 | −1.28627 | 0.005974 |
| | Coagulation factor XIII is the last zymogen to become | | | | |
| | activated in the blood coagulation cascade. Plasma factor | | | | |
| | XIII is a heterotetramer composed of 2 A subunits and 2 | | | | |
| | B subunits. The A subunits have catalytic function, and | | | | |
| | the B subunits do not have enzymatic activity and may | | | | |
| | serve as plasma carrier molecules. Platelet factor XIII is | | | | |
| | comprised only of 2 A subunits, which are identical to | | | | |
| | those of plasma origin. Upon cleavage of the activation | | | | |
| | peptide by thrombin and in the presence of calcium ion, | | | | |
| | the plasma factor XIII dissociates its B subunits and | | | | |
| | yields the same active enzyme, factor XIIIa, as platelet | | | | |
| | factor XIII. This enzyme acts as a transglutaminase to | | | | |
| | catalyze the formation of gamma-glutamyl-epsilon- | | | | |
| | lysine crosslinking between fibrin molecules, thus | | | | |
| | stabilizing the fibrin clot. It also crosslinks alpha-2- | | | | |
| | plasmin inhibitor, or fibronectin, to the alpha chains of | | | | |
| | fibrin. Factor XIII deficiency is classified into two | | | | |
| | categories: type I deficiency, characterized by the lack of | | | | |
| | both the A and B subunits; and type II deficiency, | | | | |
| | characterized by the lack of the A subunit alone. These | | | | |
| | defects can result in a lifelong bleeding tendency, | | | | |
| | defective wound healing, and habitual abortion. | | | | |
| | [provided by RefSeq, July 2008] | | | | |
| KL | This gene encodes a type-I membrane protein that is | −1.99188 | 0.008241 | −2.04286 | 0.003049 |
| | related to beta-glucosidases. Reduced production of this | | | | |
| | protein has been observed in patients with chronic renal | | | | |
| | failure (CRF), and this may be one of the factors | | | | |
| | underlying the degenerative processes (e.g., | | | | |
| | arteriosclerosis, osteoporosis, and skin atrophy) seen in | | | | |
| | CRF. Also, mutations within this protein have been | | | | |
| | associated with ageing and bone loss. [provided by | | | | |
| | RefSeq, July 2008] | | | | |
| PDGFRA | Tyrosine-protein kinase that acts as a cell-surface | −0.88332 | 0.037488 | −0.78472 | 0.034063 |
| | receptor for PDGFA, PDGFB and PDGFC and plays an | | | | |
| | essential role in the regulation of embryonic | | | | |
| | development, cell proliferation, survival and chemotaxis. | | | | |
| | Depending on the context, promotes or inhibits cell | | | | |
| | proliferation and cell migration. Plays an important role | | | | |
| | in the differentiation of bone marrow-derived | | | | |
| | mesenchymal stem cells. Required for normal skeleton | | | | |
| | development and cephalic closure during embryonic | | | | |
| | development. Required for normal development of the | | | | |
| | mucosa lining the gastrointestinal tract, and for | | | | |
| | recruitment of mesenchymal cells and normal | | | | |
| | development of intestinal villi. Plays a role in cell | | | | |
| | migration and chemotaxis in wound healing. Plays a role | | | | |
| | in platelet activation, secretion of agonists from platelet | | | | |
| | granules, and in thrombin-induced platelet aggregation. | | | | |
| | Binding of its cognate ligands - homodimeric PDGFA, | | | | |
| | homodimeric PDGFB, heterodimers formed by PDGFA | | | | |
| | and PDGFB or homodimeric PDGFC -leads to the | | | | |
| | activation of several signaling cascades; the response | | | | |
| | depends on the nature of the bound ligand and is | | | | |
| | modulated by the formation of heterodimers between | | | | |
| | PDGFRA and PDGFRB. Phosphorylates PIK3R1, | | | | |
| | PLCG1, and PTPN11. Activation of PLCG1 leads to the | | | | |
| | production of the cellular signaling molecules | | | | |
| | diacylglycerol and inositol 1,4,5-trisphosphate, | | | | |
| | mobilization of cytosolic Ca(2+) and the activation of | | | | |
| | protein kinase C. Phosphorylates PIK3R1, the regulatory | | | | |
| | subunit of phosphatidylinositol 3-kinase, and thereby | | | | |
| | mediates activation of the AKT1 signaling pathway. | | | | |
| | Mediates activation of HRAS and of the MAP kinases | | | | |
| | MAPK1/ERK2 and/or MAPK3/ERK1. Promotes | | | | |
| | activation of STAT family members STAT1, STAT3 | | | | |
| | and STAT5A and/or STAT5B. Receptor signaling is | | | | |
| | down-regulated by protein phosphatases that | | | | |
| | dephosphorylate the receptor and its down-stream | | | | |
| | effectors, and by rapid internalization of the activated | | | | |
| | receptor | | | | |
| TLR4 | Cooperates with LY96 and CD14 to mediate the innate | −1.34467 | 0.001594 | −1.26519 | 0.002682 |
| | immune response to bacterial lipopolysaccharide (LPS) | | | | |
| | (PubMed: 27022195). Acts via MYD88, TIRAP and | | | | |
| | TRAF6, leading to NF-kappa-B activation, cytokine | | | | |
| | secretion and the inflammatory response | | | | |
| | (PubMed: 9237759, PubMed: 10835634, | | | | |
| | PubMed: 27022195, PubMed: 21393102). Also involved | | | | |
| | in LPS-independent inflammatory responses triggered by | | | | |
| | free fatty acids, such as palmitate, and Ni(2+). Responses | | | | |
| | triggered by Ni(2+) require non-conserved histidines and | | | | |
| | are, therefore, species-specific (PubMed: 20711192). | | | | |
| | Both M. tuberculosis HSP70 (dnaK) and HSP65 (groEL- | | | | |
| | 2) act via this protein to stimulate NF-kappa-B | | | | |
| | expression (PubMed: 15809303). In complex with TLR6, | | | | |
| | promotes sterile inflammation in | | | | |
| | monocytes/macrophages in response to oxidized low- | | | | |
| | density lipoprotein (oxLDL) or amyloid-beta 42. In this | | | | |
| | context, the initial signal is provided by oxLDL- or | | | | |
| | amyloid-beta 42-binding to CD36. This event induces | | | | |
| | the formation of a heterodimer of TLR4 and TLR6, | | | | |
| | which is rapidly internalized and triggers inflammatory | | | | |
| | response, leading to the NF-kappa-B-dependent | | | | |
| | production of CXCL1, CXCL2 and CCL9 cytokines, via | | | | |
| | MYD88 signaling pathway, and CCL5 cytokine, via | | | | |
| | TICAM1 signaling pathway, as well as IL1B secretion. | | | | |
| | Binds electronegative LDL (LDL(−)) and mediates the | | | | |
| | cytokine release induced by LDL(−) | | | | |
| | (PubMed: 23880187). Stimulation of monocytes in vitro | | | | |
| | with M.tuberculosis PstS1 induces p38 MAPK and | | | | |
| | ERK1/2 activation primarily via TLR2, but also partially | | | | |
| | via this receptor | | | | |
| | (PubMed: 16622205, 10835634, 15809303, 17478729, | | | | |
| | 20037584, 20711192, 23880187, 27022195, 9237759). | | | | |
| | Activated by the signaling pathway regulator NMI which | | | | |
| | acts as damage-associated molecular patterns (DAMPs) | | | | |
| | in response to cell injury or pathogen invasion, therefore | | | | |
| | promoting nuclear factor NF-kappa-B activation | | | | |
| | (PubMed: 29038465). (TLR4_HUMAN, O00206) | | | | |

1. A method of treatment or prevention of a disorder, disease, or condition alleviated by
i) stimulating or modulating collagen expression in a subject in need thereof, and/or
ii) stimulating or modulating claudin-1 expression in a subject in need thereof; and/or
iii) stimulating or modulating one more anti-inflammatory genes in a subject in need thereof,
the method comprising administering to the subject a composition comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 14 kDa and about 30 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5,
wherein the concentration of silk fibroin fragments in the composition is from about 0.001% w/v to about 10% w/v.

2. The method of claim 1, wherein the composition further comprises 0 to 500 ppm lithium bromide.

3. The method of claim 1 or claim 2, wherein the composition further comprises 0 to 500 ppm sodium carbonate

4. The method of any one of claims 1 to 3, wherein the silk fibroin fragments have a polydispersity between 1 and about 1.5.

5. The method of any one of claims 1 to 3, wherein the silk fibroin fragments have a polydispersity between about 1.5 and about 2.0.

6. The method of any one of claims 1 to 3, wherein the silk fibroin fragments have a polydispersity between about 1.5 and about 3.0.

7. The method of any one of claims 1 to 3, wherein the silk fibroin fragments have a polydispersity between about 2.0 and about 2.5.

8. The method of any one of claims 1 to 3, wherein the silk fibroin fragments have a polydispersity between about 2.5 and about 3.0.

9. The method of any one of claims 1 to 8, wherein the silk fibroin fragments do not spontaneously or gradually gelate and do not visibly change in color or turbidity when in an aqueous solution for at least 10 days prior to formulation into the composition.

10. The method of any one of claims 1 to 9, wherein the silk fibroin fragments are present in the composition at about 0.001% w/v to about 1% w/v.

11. The method of any one of claims 1 to 9, wherein the silk fibroin fragments are present in the composition at about 0.01% w/v to about 1% w/v.

12. The method of any one of claims 1 to 9, wherein the silk fibroin fragments are present in the composition at about 0.025% w/v to about 1% w/v.

13. The method of any one of claims 1 to 9, wherein the silk fibroin fragments are present in the composition at about 0.05% w/v to about 0.7% w/v.

14. The method of any one of claims 1 to 13, wherein the composition is formulated as an injectable composition or as a topical composition.

15. The method of any one of claims 1 to 14, wherein the composition further comprises a pharmaceutically acceptable carrier.

16. The method of claim 15, wherein the pharmaceutically acceptable carrier comprises an aqueous phase.

17. The method of claim 15 or 16, wherein the pharmaceutically acceptable carrier comprises an oil-in-water emulsion or a water-in-oil emulsion.

18. The method of any one of claims 1 to 17, wherein the composition is formulated for administration to an epithelial surface.

19. The method of claim 18, wherein the epithelial surface is a superficial epidermal area, a stratum corneum, an eye surface, or an intestinal surface.

20. The method of any one of claims 1 to 17, wherein the composition is formulated for reducing trans-epidermal water loss.

21. The method of any one of claims 1 to 17, wherein the composition is formulated as a barrier formulation.

22. The method of any one of claims 1 to 17, wherein the composition is formulated as a wound-closure formulation.

23. The method of any one of claims 1 to 17, wherein the composition is formulated for preventing or reversing wrinkles in the subject, preventing or reversing age spots in the subject, preventing or reversing dry skin in the subject, or preventing or reversing uneven skin tone in the subject.

24. The method of any one of claims 1 to 17, wherein the composition is formulated for preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject.

25. The method of any one of claims 1 to 17, wherein the disease, or condition comprises wrinkles, age spots, dry skin, uneven skin tone, skin sagging, skin aging, reduced skin tensile strength, photodamaged skin, or striae distensae (stretch marks).

26. Use of a composition comprising silk fibroin fragments having an average weight average molecular weight selected from between about 1 kDa and about 5 kDa, between about 5 kDa and about 10 kDa, between about 6 kDa and about 17 kDa, between about 10 kDa and about 15 kDa, between about 15 kDa and about 20 kDa, between about 17 kDa and about 39 kDa, between about 14 kDa and about 30 kDa, between about 20 kDa and about 25 kDa, between about 25 kDa and about 30 kDa, between about 30 kDa and about 35 kDa, between about 35 kDa and about 40 kDa, between about 39 kDa and about 54 kDa, between about 39 kDa and about 80 kDa, between about 40 kDa and about 45 kDa, between about 45 kDa and about 50 kDa, between about 60 kDa and about 100 kDa, and between about 80 kDa and about 144 kDa, and a polydispersity between 1 and about 5, wherein the concentration of silk fibroin fragments in the composition is from about 0.001% w/v to about 10% w/v, in the manufacture of a medicament for the treatment or prevention of a disorder, disease, or condition alleviated by
i) stimulating or modulating collagen expression in a subject in need thereof, and/or
ii) stimulating or modulating claudin-1 expression in a subject in need thereof; and/or
iii) stimulating or modulating one more anti-inflammatory genes in a subject in need thereof.

27. The use of claim 26, wherein the composition further comprises 0 to 500 ppm lithium bromide.

28. The use of claim 26 or claim 27, wherein the composition further comprises 0 to 500 ppm sodium carbonate

29. The use of any one of claims 26 to 28, wherein the silk fibroin fragments have a polydispersity between 1 and about 1.5.

30. The use of any one of claims 26 to 28, wherein the silk fibroin fragments have a polydispersity between about 1.5 and about 2.0.

31. The use of any one of claims 26 to 28, wherein the silk fibroin fragments have a polydispersity between about 1.5 and about 3.0.

32. The use of any one of claims 26 to 28, wherein the silk fibroin fragments have a polydispersity between about 2.0 and about 2.5.

33. The use of any one of claims 26 to 28, wherein the silk fibroin fragments have a polydispersity between about 2.5 and about 3.0.

34. The use of any one of claims 26 to 33, wherein the silk fibroin fragments do not spontaneously or gradually gelate and do not visibly change in color or turbidity when in an aqueous solution for at least 10 days prior to formulation into the composition.

35. The use of any one of claims 26 to 34, wherein the silk fibroin fragments are present in the composition at about 0.001% w/v to about 1% w/v.

36. The method of any one of claims 26 to 34, wherein the silk fibroin fragments are present in the composition at about 0.01% w/v to about 1% w/v.

37. The use of any one of claims 26 to 34, wherein the silk fibroin fragments are present in the composition at about 0.025% w/v to about 1% w/v.

38. The use of any one of claims 26 to 34, wherein the silk fibroin fragments are present in the composition at about 0.05% w/v to about 0.7% w/v.

39. The use of any one of claims 26 to 38, wherein the composition is formulated as an injectable composition or as a topical composition.

40. The use of any one of claims 26 to 39, wherein the composition further comprises a pharmaceutically acceptable carrier.

41. The use of claim 40, wherein the pharmaceutically acceptable carrier comprises an aqueous phase.

42. The use of claim 40 or 41, wherein the pharmaceutically acceptable carrier comprises an oil-in-water emulsion or a water-in-oil emulsion.

43. The use of any one of claims 26 to 42, wherein the composition is formulated for administration to an epithelial surface.

44. The use of claim 43, wherein the epithelial surface is a superficial epidermal area, a stratum corneum, an eye surface, or an intestinal surface.

45. The use of any one of claims 26 to 42, wherein the composition is formulated for reducing trans-epidermal water loss.

46. The use of any one of claims 26 to 42, wherein the composition is formulated as a barrier formulation.

47. The use of any one of claims 26 to 42, wherein the composition is formulated as a wound-closure formulation.

48. The use of any one of claims 26 to 42, wherein the composition is formulated for preventing or reversing wrinkles in the subject, preventing or reversing age spots in the subject, preventing or reversing dry skin in the subject, or preventing or reversing uneven skin tone in the subject.

49. The use of any one of claims 26 to 42, wherein the composition is formulated for preventing or reversing skin sagging in the subject, preventing or reversing skin aging in the subject, preventing or reversing reduced skin tensile strength in the subject, preventing or reversing photodamaged skin in the subject, or preventing or reversing striae distensae (stretch marks) in the subject.

50. The use of any one of claims 26 to 42, wherein the disease, or condition comprises wrinkles, age spots, dry skin, uneven skin tone, skin sagging, skin aging, reduced skin tensile strength, photodamaged skin, or striae distensae (stretch marks).
